(19) United States
(12) Patent Application Publication
(10) Pub. No.: US 2025/0066377 A1
(43) Pub. Date: Feb. 27, 2025

(54) AMINO CHROMEN-2-ONE MODULATORS OF POLRMT

(71) Applicant: Pretzel Therapeutics, Inc., Waltham, MA (US)

(72) Inventors: Andrew Griffin, L'Île Bizard, Quebec (CA); Paul S. Charifson, Carrabassett Valley, ME (US); Jeremy Green, Waltham, MA (US); Gabriel Martinez Botella, Wayland, MA (US)

(73) Assignee: Pretzel Therapeutics, Inc., Waltham, MA (US)

(21) Appl. No.: 18/720,692

(22) PCT Filed: Dec. 19, 2022
(86) PCT No.: PCT/US2022/053360

§ 371 Date: Jun. 17, 2024

Related U.S. Application Data
(60) Provisional application No. 63/290,756, filed on Dec. 17, 2021.
(60) Provisional application No. 63/326,565, filed on Apr. 1, 2022.
(60) Provisional application No. 63/358,671, filed on Jul. 6, 2022.

(51) Int. Cl. C07D 491/052; A61K 31/436; A61K 31/4545; A61K 31/496; A61K 31/5377; C07D 519/00

(52) U.S. Cl. CPC C07D 491/052; A61K 31/436; A61K 31/4545; A61K 31/496; A61K 31/5377; C07D 519/00

(57) ABSTRACT

The present invention provides novel amino chromen-2-one compounds that are inhibitors of mitochondrial RNA polymerase for treating various diseases such as cancer and others associated with metabolic disorders and mitochondrial dysfunction.

### FIELD OF THE INVENTION

The present invention relates to novel POLRMT modulators, their prodrugs, their pharmaceutically acceptable salts, and pharmaceutical compositions thereof. The present invention also relates to methods of using such compounds and compositions, including to inhibit or promote POLRMT, and to treat various neurodegenerative and metabolic disorders, cancer, and also disorders related to aging and mitochondrial diseases.

### BACKGROUND OF THE INVENTION

Human mitochondrial RNA polymerase, POLRMT (also referred to as h-mtRNAP), is a nuclear-encoded single-subunit DNA-dependent RNA polymerase. POLRMT is 1230 amino acids in length and consists of three distinct regions: (1) a C-terminal polymerase domain (CTD) (residues 648-1230); (2) an N-terminal domain (NTD) (residues 369-647); and (3) an N-terminal extension (NTE) (residues 1-368). See, e.g., Arnold, J. J., et al., “Human mitochondrial RNA polymerase: Structure-function, mechanism and inhibition,” Biochim. Biophys. Acta, 1819, 948-960 (2012). It is structurally related to the single-subunit RNA polymerase encoded by bacteriophage T7. The CTD is also known as the catalytic domain due to its function of catalyzing nucleotide incorporation into a growing RNA molecule during transcription. This domain is highly conserved across species, whereas by contrast the NTE demonstrates significant sequence variability, suggesting organism-specific roles for this domain of POLRMT. Regarding the POLRMT NTD, structurally it resembles the N-terminal domain (also called the promoter-binding domain) of T7 RNA polymerase. However, for promoter-specific transcription initiation POLRMT requires assistance from additional transcription factors, whereas T7 RNA polymerase does not.

A primary biological role of POLRMT is to transcribe the mitochondrial genome to produce the RNAs needed for expression of mitochondrial DNA (mtDNA). Initiation, elongation, and termination are the three steps of mitochondrial transcription. Each of a light-strand promoter (LSP) and two heavy-strand promoters (HSP-1 and HSP-2) on the mtDNA contains a transcription initiation site. See, e.g., Basu, U., et al., “Structure, mechanism, and regulation of mitochondrial DNA transcription initiation,” J Biol. Chem., 295(52), 18406-425 (2020). For promoter-specific transcription initiation, POLRMT requires two transcription factors, TFAM (transcription factor A mitochondrial) and TFB2M (transcription factor B mitochondrial). See id. Various models suggest different mechanisms by which the initiation complex structure with POLRMT, TFAM, and TFB2M comes together to cover the promoter DNA for initiation of transcription. In one current model TFAM recruits POLRMT to the promoter site to form a protein-protein pre-initiation complex, to which TFB2M binds to form the initiation complex, which covers the promoter DNA. See id. During initiation, the RNA is elongated to about 8-10 nucleotides in length. Conformational changes occur at that point, including promoter release and displacement of the initiation factors, converting the initiation complex into an elongation complex at which time transcription occurs. See id.

The mitochondrial genome encodes the various subunits of the electron transport chain. See, e.g., Shokolenko, I. N., et al., “Maintenance and expression of mammalian mitochondrial DNA,” Annu. Rev. Biochem., 85, 133-160 (2016). Specifically, transcription of the mitochondrial genome is necessary for the expression of 13 subunits of the oxidative phosphorylation (OXPHOS) system, as well as two rRNAs and 22 tRNAs. See, e.g., Shokolenko, I. N., et al., “Mitochondrial transcription in mammalian cells,” Frontiers in Bioscience, Landmark, 22, 835-853 (2017). Thus, POLRMT is essential for biogenesis of the OXPHOS system, resulting in ATP production. This, in turn, is vital for energy homeostasis in the cell.

Dysregulation of POLRMT and the OXPHOS system have been implicated in various disease states, in particular cancer. Cancer is now the second leading cause of death in the United States, with projections indicating that almost two million new cases will be diagnosed in 2022 and over 600,000 deaths will be the result of cancer. See Siegel, R. L. et al., “Cancer statistics 2022.” CA Cancer J. Clin. (72) 7-33 (2022). High rates of OXPHOS have been shown to support growth in cancer cell lines, including in a subset of diffuse large B cell lymphoma cells. See, e.g., DeBeradinis, R. J., “A mitochondrial power play in lymphoma,” Cancer Cell, 22, 423-24 (2012). Noteworthy is the observation that metabolic heterogeneity exists not only between different types of cancer, but also among tumors of the same type. Similarly, in a study using melanoma cell lines representative of various stages of tumor progression and that collectively mimic the mixture of cells found in a tumor, it was found that metastatic cells demonstrated a high OXPHOS capacity. Rodrigues, M. F., et al., “Enhanced OXPHOS, glutaminolysis and β-oxidation constitute the metastatic phenotype of melanoma cells,” Biochem. J. 473: 703-715 (2016). These data suggest mitochondria play a role as cells progress toward metastasis, possibly to provide the energy needed for tumor cell migration and invasion.

Relatedly, overexpression of POLRMT has been linked to multiple types of cancers, suggesting that it plays a role in tumor growth. Supporting this hypothesis is, for example, a study involving acute myeloid leukemia (AML) cells, which are known to have high oxidative phosphorylation and mitochondrial mass, as well as low respiratory chain spare reserve capacity. POLRMT knockdown AML cells demonstrated a reduction in POLRMT levels, decreased oxidative phosphorylation, and increased cell death as compared to control AML cells. See Bralha, F. N., et al., “Targeting mitochondrial RNA polymerase in acute myeloid leukemia,” Oncotarget, 6(35), 37216-228 (2015). In other work, injection into nude mice of a human breast cancer cell line that overexpresses POLRMT resulted in increased tumor growth, independent of tumor angiogenesis, suggesting that POLRMT should be considered a tumor promoter or metabolic oncogene. Salem, A. F., et al. “Mitochondrial biogenesis in epithelial cancer cells promotes breast cancer tumor growth and confers autophagy resistance,” Cell Cycle, 11(22), 4174-80 (2012). Recently, the expression of POLRMT in non-small cell lung cancer (NSCLC) has been examined. See Zhou, T. et al., “The requirement of mitochondrial RNA polymerase for non-small cell lung cancer cell growth,” Cell Death and Disease, 12, 751 (2021). While POLRMT mRNA and protein were detected in normal human lung tissue, their levels were significantly higher in cancer tissue. Similar results were obtained when comparing primary lung epithelial cells to NSCLC cells. Using short hairpin RNA (shRNA) to silence POLRMT mRNA and downregulate POLRMT protein resulted in inhibition of NSCLC cell viability, proliferation, migration, and invasion. Moreover, silencing of POLRMT significantly induced apoptosis activation in both primary and established NSCLC cells. Injection of POLRMT shRNA in an adeno-associated virus construct into tumors effectively inhibited NSCLC xenograft growth in mice. Taken together, these data suggest that POLRMT could be an oncogenic gene for NSCLC.

The development of multidrug resistance (MDR) to numerous cancers is associated with poor prognosis and presents significant challenges in the treatment of this disease. Because such resistance encompasses drugs having different structures and mechanisms of action, identifying and targeting a single biochemical pathway that could re-sensitize MDR cancer cells to established chemotherapy would provide a promising treatment strategy. See Yu, H.-J., “Targeting mitochondrial metabolism and RNA polymerase POLRMT to overcome multidrug resistance in cancer,” Front. Chem., 9:775226 (2021). A main reason for the development of MDR is enhanced drug efflux from and decreased drug accumulation in MDR cells due to ATP-dependent protein transporters that pump drugs out of cells. Inhibiting POLRMT and consequently the production of the proteins essential for the OXPHOS system could compromise ATP production and, in turn, the ATP-dependent efflux of chemotherapeutic agents from cancer cells.

Consistent with the findings that the OXPHOS system and POLRMT may be involved in the etiology of and in some cases overexpressed in some cancers, small-molecule inhibitors of POLRMT have been developed. See, e.g., EP 3 598 972 A1; WO 2019/057821 A1; and WO 2020/188049 A1. Some of these inhibitors have been shown to be useful in inhibiting cancer cell proliferation without affecting control cells. See Bonekamp, N. A., et al., “Small-molecule inhibitors of human mitochondrial DNA transcription,” Nature, 588, 712-716 (2020). The cancer cell toxicity was correlated to a considerable increase in the levels of mono- and diphosphate nucleotides with a concomitant decrease in nucleotide triphosphate levels, all the result of a debilitated OXPHOS system. Similarly, treatment with POLRMT inhibitors caused a decrease in citric-acid cycle intermediates and ultimately cellular amino acid levels, the result of which is a state of severe energy and nutrient depletion. See id. Such inhibitors also produced a decrease in tumor volume in mice with no significant toxicity in control animals. Specifically, mtDNA transcript levels in tumor cells were decreased as compared to transcript levels in differentiated tissue. These data highlight the importance of mtDNA expression in rapidly dividing cells as opposed to post-mitotic tissue, a distinction that may be capitalized on using POLRMT inhibitors that are capable of modulating mtDNA transcription and ultimately the OXPHOS system.

While mitochondria are an emerging target for cancer treatment, the resistance mechanisms induced by chronic inhibition of mitochondrial function are poorly understood. In view of the challenges presented by drug resistance in cancer chemotherapy, the development of such resistance to small molecule inhibitors of POLRMT has been investigated. See Mennuni, M. et al., “Metabolic resistance to the inhibition of mitochondrial transcription revealed by CRISPER-Cas9 screen,” EMBO reports, 23: e53054 1-18 (2022). Using a CRISPR-Cas9 whole-genome screen, loss of genes belonging to von Hippel-Lindau (VHL) and mammalian target of rapamycin complex 1 (mTORC1) were the pathways that caused resistance to acute treatment with a POLRMT inhibitor. See id. at pp. 1-2. Moreover, dose-escalated chronic treatment of cells with this molecule resulted in drug-resistant cells that had increased levels of mtDNA, thereby giving rise to increased levels of mitochondrial transcripts and proteins. See id. at p. 5. The drug-resistant cells maintained higher levels of nucleotide levels, tricarboxylic acid cycle intermediates, and amino acids. See id. at p. 7. Notably, the drug-resistant cells did not have mutations in POLRMT that compromise inhibitor binding to the polymerase. See id. The development of resistance to POLRMT inhibitors underscores the importance and need for the development of other POLRMT inhibitors to understand and treat cancers of varying types.

Alterations in the OXPHOS system also have been implicated in the development of insulin resistance and ultimately Type-2 diabetes. In studies involving apoptosis inducing factor (AIF) knockout mice, a primary OXPHOS defect that produced OXPHOS deficiency revealed an increase in insulin sensitivity and resistance to diabetes and obesity. See Pospisilik, J. A., et al., “Targeted deletion of AIF decreases mitochondrial oxidative phosphorylation and protects from obesity and diabetes,” Cell, 131, 476-91 (2007). Correlated with these phenotypic changes were the metabolic alterations of increased glucose uptake and enhanced fuel utilization. Manipulation of the OXPHOS system with POLRMT modulators affords the potential for further understanding the physiological mechanisms involved in diseases such as diabetes and for the development of novel treatments for intervention of such metabolic disorders.

In addition to its critical role in transcription, POLRMT acts as the primase for mtDNA replication, thus playing a part in the regulation of mtDNA levels. Human mtDNA is a circular double-stranded DNA that is packaged in DNA-protein structures called mitochondrial nucleoids, for which TFAM is the most abundant structural component. See, e.g., Filograna, R., et al., “Mitochondrial DNA copy number in human disease: the more the better?” FEBS Letters, 595, 976-1002 (2021). TFAM facilitates mtDNA compaction, which results in regulating the accessibility of the DNA to cellular replication and transcription components. With respect to mtDNA replication, POLRMT is part of the mtDNA replisome along with the hexameric helicase TWINKLE, the heterotrimeric DNA polymerase gamma (POLγ) and the tetrameric mitochondrial single-stranded DNA-binding protein (mtSSB). See id. Its function in this replisome is to synthesize the RNA primers required for the initiation of the synthesis of both strands of mtDNA. While there may be many mechanisms by which mtDNA levels may be regulated, including modulation of POLRMT, what is known to date is that mtDNA copy number can be manipulated through modulation of TFAM expression.

While the correlation is not completely straightforward, changed levels of mtDNA have been implicated in neurogenerative disorders, cancer, and aging. See e.g., Filograna, R., et al., “Mitochondrial DNA copy number in human disease: the more the better?” FEBS Letters, 595, 976-1002 (2021). Particularly challenging is the attempt to understand the relationship between mtDNA copy number and cancer. It appears that such copy number can correlate with both increased and decreased disease burden. As such, tumor type and stage of disease may be important factors in determining the role of mtDNA copy number in the diagnosis and/or prognosis of cancer. With respect to aging, most data show a reduction in mtDNA levels in the older population. That being said, other study data are inconsistent as to the relationship between mtDNA copy number and longevity. By contrast, there appears to be a clearer correlation between neurodegeneration in Alzheimer's disease and reduction in mtDNA levels. Complicating the understanding of the relationship between mtDNA levels and disease is the role that mtDNA mutations have on various disorders. While accumulation of mtDNA mutations appears to occur in almost all types of cancer, it is unclear whether such mutations are causative of the cancer or merely a by-product of rapid replication in fast-dividing cells. Nonetheless, since POLRMT plays a key role in mtDNA replication, POLMRT modulation may provide an effective mechanism by which to understand various disease states and how to slow or alter the progression of disease.

Mutations affecting POLRMT may also cause human disease. See Olihovi, M., et al., “POLRMT mutations impair mitochondrial transcription causing neurological disease.” Nat. Commun., 12, 1135 (2021). POLRMT variants have been identified in a number of unrelated families. Patients present with multiple phenotypes, including global developmental delay, hypotonia, short stature, and speech/intellectual disability in childhood. POLRMT modulation may provide a mechanism to slow or alter the progression of disease.

POLRMT is of fundamental importance for both expression and replication of the human mitochondrial genome. While aspects of POLRMT biochemistry are known, its full physiological role in mitochondrial gene expression and homeostasis, as well as its underlying impact in the etiology of various disease states, remains unclear. Its dysfunction and/or deregulation impacts mitochondrial metabolism, sometimes through the OXPHOS system, which ultimately contributes to many metabolic, degenerative and age-related diseases such as cancer, diabetes, obesity, and Alzheimer's disease. Pharmacological inhibition of POLRMT is one means by which to gain a further understanding of the role of this polymerase in cell physiology and the development of disease. Regulation of metabolic mechanisms, including oxidative phosphorylation, with POLRMT modulators affords an opportunity for intervention in complex disorders. In view of the numerous and varied roles of POLRMT, the need exists for potent and specific modulators of POLRMT.

### SUMMARY OF THE INVENTION

Provided are compounds, pharmaceutically acceptable salts of the compounds, and prodrugs of the compounds; pharmaceutical compositions comprising the compounds or their salts or prodrugs; and methods of using the compounds, salts of the compounds, prodrugs of the compounds, or pharmaceutical compositions of the compounds, their salts, or their prodrugs to treat various neurodegenerative and metabolic disorders, cancer, and also disorders related to aging and mitochondrial diseases. The compounds and their pharmaceutically acceptable salts are particularly useful as modulators of POLRMT.

In one embodiment, the present invention is directed to a compound, a prodrug thereof, or a pharmaceutically acceptable salt thereof, represented by formula (1):

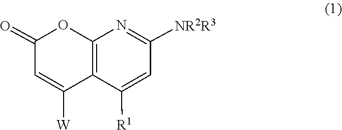

wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² and R³ are independently hydrogen, C₁-C₄ alkoxy, C₃-C₆ cycloalkyl, or C₁-C₄ alkyl optionally substituted with one or more substituents selected from the group consisting of deuterium, fluoro, C₁-C₄ alkoxy, cyano, C(O)OR⁴, and C(O)NR⁴R⁵;
or R² and R³ together with their connecting nitrogen form a 4- to 7-membered heterocyclic ring, a 7- to 12-membered spiro ring, or a 6- to 12-membered fused heterocyclic ring, wherein each ring optionally contains another heteroatom that is N, O, or S, and each ring is optionally substituted with one to four groups each independently selected from the group consisting of fluoro, chloro, C₁-C₄ alkoxy, C₁-C₄ alkyl-C₁-C₄ alkoxy, hydroxy, cyano, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁴ is hydrogen or C₁-C₃ alkyl; and
R⁵ is R⁴ or C₁-C₃ alkyl optionally substituted with C(O)OR⁴, C(O)NR⁴R⁴, or OR⁴.

A further embodiment of the present invention are compounds of the invention (that is, compounds of formula (1), (2), and (3)), prodrugs of the compounds, or their pharmaceutically acceptable salts wherein one or more hydrogen is substituted with a deuterium atom.

Additional embodiments of the invention are pharmaceutical compositions comprising a compound of the invention or a pharmaceutically acceptable salt thereof, or a prodrug thereof and one or more pharmaceutically acceptable excipient.

Further embodiments of the invention are methods of treating a disease, such methods comprising administering to a subject in need thereof a therapeutically effective amount of a compound of the invention, a prodrug thereof, or a pharmaceutically acceptable salt thereof. In some embodiments, the disease is selected from the group consisting of adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, NASH, and related metabolic syndromes such as NAFLD. In some embodiments, the disease is related to aging or a mitochondrial disorder.

Additional embodiments of the invention are methods of treating neurodegenerative disorders and metabolic disorders, such as those identified in Bonekamp, N. A. et al. “Small-molecule inhibitors of human mitochondrial DNA transcription” Nature, 588, 712-716 (2020), Filograna, R. et al, “Mitochondrial DNA copy number in human disease: the more the better?” FEBS Lett., 595, 976-1002 (2021), Wrendenber, A. et al. “Respiratory chain dysfunction in skeletal muscle does not cause insulin resistance” Biochem. Biophys. Res. Comm., 350, 202-207 (2006), Pospililik, J. A. et al. “Targeted deletion of AIF decreases mitochondrial oxidative phosphorylation and protects from obesity and diabetes” Cell, 131, 476-491 (2007), PCT Published Application WO2019/057821A1 and references therein.

Further embodiments of the invention are methods of treating disease of aging.

### DETAILED DESCRIPTION OF THE INVENTION

Modulators of POLRMT are useful in compositions and methods suitable for treating many disorders, such as cancer, neurodegenerative disorders, metabolic disorders, as well as diseases related to aging and mitochondrial diseases. Provided herein are compounds of formula (1), (2), and (3), pharmaceutically acceptable salts thereof, prodrugs thereof, and pharmaceutical compositions comprising such compounds, their salts, or their prodrugs that are useful in treating a condition or disease, such as cancer, neurodegenerative disorders, and metabolic disorders.

### Definitions

The term “alkyl” as used herein refers to both branched- and straight-chain saturated aliphatic hydrocarbon groups having the specified number of carbon atoms in a specified range. For example the term “C₁-C₆ alkyl” means linear or branched chain alkyl groups, including all possible isomers, having 1, 2, 3, 4, 5, or 6 carbon atoms.

The term “alkoxy” or “alkoxyl” as used herein refers to an —O-alkyl group. For example, the term “C₁-C₄ alkoxyl” mean —O—C₁-C₄ alkyl. Examples of alkoxyl include methoxyl, ethoxyl, propoxyl (e.g., n-propoxyl and isopropoxyl), and the like.

The term “C₁-C₄ alkyl-C₁-C₄ alkoxy” as used herein refers to alkoxy groups that are attached to alkyl groups. For example, a methoxy-methyl group refers to —CH₂—OCH₃.

The term “halogen” as used herein refers to fluorine (“fluoro”), chlorine (“chloro”), bromine (“bromo”), and iodine (“iodo”).

The term “haloalkoxy” or “haloalkoxyl” as used herein refers to an —O-alkyl group in which at least one of the hydrogen atoms of alkyl group is replaced with a halogen atom. Examples of haloalkoxyl include trifluoromethoxyl, 2,2,2-trifluoroethoxyl, and the like.

The term “acyl” or “alkanoyl” as used herein refers to an —C(O)-alkyl group. For example, the term “C₁-C₆ alkanoyl” means —C(O)—C₁-C₆ alkyl. Examples of alkanoyl include acetyl, propionyl, butyryl, and the like.

The term “keto” as used herein refers to a —C(O)— group. The nomenclature for a keto group may include “oxo” or “one”.

The term “carboxyl” or “carboxy,” as used herein, refers to —C(O)OH or the corresponding “carboxylate” anion, such as a carboxylic acid salt.

The term “alkylcarboxylate,” as used herein, refers to —C(O)O-alkyl.

The term “oxo” as used herein refers to a group which consists of oxygen which is double bonded to carbon or any other element.

The term “substituted” means that any one or more hydrogen atoms on the designated atom or group is replaced with one or more substituents other than hydrogen, provided that the designated atom's normal valence is not exceeded.

The term “optionally substituted” or “optional substituents” as used herein means that the groups are either unsubstituted or substituted with one or more of the substituents specified. When the groups are substituted with more than one substituent, the substituents may be the same or different. Furthermore, when using the terms “independently,” “independently are,” and “independently selected from” means that the groups may be the same or different.

The term “optionally substituted amino” means an amino optionally substituted by 1 or 2 substituents selected from C₁-C₆ alkyl, C₃-C₇ cycloalkyl, C₂-C₆ alkenyl, C₂-C₆ alkynyl, aryl, aryl C₁-C₆ alkyl, optionally substituted C₁-C₆ alkanoyl, heterocyclyl, an optionally substituted acyl, optionally substituted alkylcarboxylate, optionally substituted carbamoyl, or optionally substituted sulfonyl. Examples of the “optionally substituted amino” include amino, methylamino, ethylamino, propylamino, isopropylamino, butylamino, isobutylamino, sec-butylamino, tert-butylamino, cyclopropylamino, cyclobutylamino, 1-propenylamino (allylamino), phenylamino, naphthylamino (e.g., 1-naphthylamino, 2-naphthylamino), benzylamino, naphthylmethylamino, 2-phenylethylamino, pyridylamino, dimethylamino, diethylamino, N-methyl-N-ethylamino, N-methyl-N-(2-amino-3-methylbutyryl)amino, formyl, acetyl, propanoyl, benzoyl, methylsulfonyl, ethylsulfonyl, phenylsulfonyl, and the like.

The term “optionally substituted amide” means a primary (or unsubstituted) amide, a secondary (or monosubstituted) amide, or a tertiary (or disubstituted) amide.

The term “cycloalkyl” as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term “aryl” as used herein refers to a monocyclic or fused bicyclic ring system having the characteristics of aromaticity, wherein at least one ring contains a completely conjugated pi-electron system. Typically, aryl groups contain 6 to 14 carbon atoms (“C₆-C₁₄ aryl”) or preferably, 6 to 12 carbon atoms (“C₆-C₁₂ aryl”). Fused aryl groups may include an aryl ring (e.g., a phenyl ring) fused to another aryl ring, or fused to a saturated or partially unsaturated carbocyclic or heterocyclic ring. The point of attachment to the base molecule on such fused aryl ring systems may be a C atom of the aromatic portion or a C or N atom of the non-aromatic portion of the ring system. Examples, without limitation, of aryl groups include phenyl, biphenyl, naphthyl, anthracenyl, indanyl, indenyl, and tetrahydronaphthyl.

The term “cycloaryl” herein refers to a polycyclic group wherein an aryl group is fused to a 5- or 6-membered aliphatic ring. For example, “C₆-C₁₂ cycloaryl” means a C₆-C₁₂ aryl fused to a 5- or 6-membered aliphatic ring.

The term “heteroaryl” as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatoms, at least one ring is aromatic, each N is optionally in the form of an oxide, and each S in a ring which is not aromatic is optionally S(O) or S(O)₂. Typically, heteroaryl groups contain 5 to 14 ring atoms (“5-14 membered heteroaryl”), and preferably 5 to 12 ring atoms (“5- to 12-membered heteroaryl”). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1,2,3-triazolyl or 1,2,4-triazolyl), tetrazolyl, oxazolyl, isooxazolyl, oxadiazolyl (i.e., the 1,2,3-, 1,2,4-, 1,2,5-(furazanyl), or 1,3,4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1,2-a]pyridinyl, benzotriazolyl, indazolyl, indolinyl, and isoindolinyl.

The term “heteroaryloxy” or “heteroaryloxyl” as used herein refers to an —O— heteroaryl group.

The term “heterocycle,” “heterocyclyl,” or “heterocyclic” as used herein represents a stable 3- to 10-membered monocyclic, non-aromatic ring that is either saturated or unsaturated, and that consists of carbon atoms and from one to two heteroatoms selected from the group consisting of N, O, and S. Examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, and oxazepanyl.

The term “deuterium” as used herein refers to an isotope of hydrogen that has one proton and one neutron in its nucleus and that has twice the mass of ordinary hydrogen. Deuterium herein is represented by the symbol “D”.

The term “deuterated” by itself or used to modify a compound or group as used herein refers to the presence of at least one deuterium atom attached to carbon. For example, the term “deuterated compound” refers to a compound which contains one or more carbon-bound deuterium(s). In a deuterated compound of the present invention, when a particular position is designated as having deuterium, it is understood that the abundance of deuterium at that position is substantially greater than the natural abundance of deuterium, which is about 0.015%.

The term “undeuterated” or “non-deuterated” as used herein refers to the ratio of deuterium atoms of which is not more than the natural isotopic deuterium content, which is about 0.015%; in other words, all hydrogen are present at their natural isotopic percentages. Unless otherwise stated, when a position is designated specifically as “H” or “hydrogen,” the position is understood to have hydrogen at its natural abundance isotopic composition.

The term “isotopic enrichment factor” as used herein refers to the ratio between the isotope abundance and the natural abundance of a specified isotope.

The term “isotopologue” as used herein refers to a species in which the chemical structure differs from a specific compound of the invention only in the isotopic composition thereof.

The term “substantially free of other stereoisomers” as used herein means less than 10% of other stereoisomers, preferably less than 5% of other stereoisomers, more preferably less than 2% of other stereoisomers and most preferably less than 1% of other stereoisomers are present.

The term “pharmaceutically acceptable salt” as used herein refers to a salt that is not biologically or otherwise undesirable (e.g., not toxic or otherwise harmful). A salt of a compound of the invention is formed between an acid and a basic group of the compound, or a base and an acidic group of the compound. For example, when the compounds of the invention contain at least one basic group (i.e., groups that can be protonated), the invention includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the invention contain one or more acidic groups (e.g., a carboxylic acid), the invention includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be found in Stahl, P. H. et al. Pharmaceutical Salts: Properties, Selection, and Use, 2nd Revised Edition, Wiley, 2011.

The term “prodrug” as used herein refers to derivatives of compounds of the invention which may have reduced pharmacological activity, but can, when administered to a patient, be converted into the inventive compounds. Design and use of prodrugs may be found in “Pro-drugs as Novel Delivery Systems,” Vol. 14, ACS Symposium Series (T Higuchi and W Stella) and “Bioreversible Carriers in Drug Design,” Pergamon Press, 1987 (ed. E B Roche, American Pharmaceutical Association), the disclosures of which are incorporated herein by reference in their entireties. Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety. Some non-limiting examples of prodrugs in accordance with the invention include: (i) where the compound contains a carboxylic acid functionality —(COOH), an ester thereof, for example, replacement of the hydrogen with (C₁-C₆)alkyl; (ii) where the compound contains an alcohol functionality (—OH), an ether thereof, for example, replacement of the hydrogen with (C₁-C₆)alkanoyloxymethyl, or with a phosphate ether group; and (iii) where the compound contains a primary or secondary amino functionality (—NH₂ or —NHR, where R is not H), an amide thereof, for example, replacement of one or both hydrogens with C₁-C₆ alkanoyl. Further examples of replacement groups in accordance with the foregoing examples and examples of other prodrug types may be found in the aforementioned references.

The terms “treatment,” “treating,” and “treat” as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term “therapeutically effective amount” as used herein refers to that amount of compound of the invention that will elicit the biological or medical response of a tissue, system, animal, or human that is being sought by a researcher, veterinarian, medical doctor or other. As will be recognized by a person of ordinary skill in the art, a therapeutically effective amount of the compounds of the invention will vary and will depend on the diseases treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

### Compounds

In one embodiment, the present invention is directed to a compound, a prodrug thereof, or a pharmaceutically acceptable salt thereof represented by formula (1):

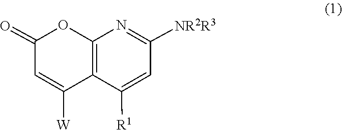

wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² and R³ are independently hydrogen, C₁-C₄ alkoxy, C₃-C₆ cycloalkyl, or C₁-C₄ alkyl optionally substituted with one or more substituents selected from the group consisting of deuterium, fluoro, C₁-C₄ alkoxy, cyano, C(O)OR⁴, and C(O)NR⁴R⁵;
or R² and R³ together with their connecting nitrogen form a 4- to 7-membered heterocyclic ring, a 7- to 12-membered spiro ring, or a 6- to 12-membered fused heterocyclic ring, wherein each ring optionally contains another heteroatom that is N, O, or S, and each ring is optionally substituted with one to four groups each independently selected from the group consisting of fluoro, chloro, C₁-C₄ alkoxy, C₁-C₄ alkyl-C₁-C₄ alkoxy, hydroxy, cyano, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁴ is hydrogen or C₁-C₃ alkyl; and
R⁵ is R⁴ or C₁-C₃ alkyl optionally substituted with C(O)OR⁴, C(O)NR⁴R⁴, or OR⁴.

In one embodiment, the present invention is directed to a compound, a prodrug thereof, or a pharmaceutically acceptable salt thereof represented by formula (2):

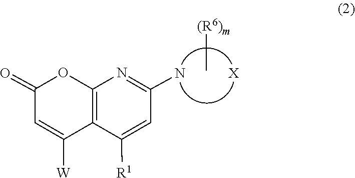

wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
X is C(R⁶)₂, NR⁷, O, or S;
R⁶ is selected from the group consisting of hydrogen, fluoro, C₁-C₄ alkoxy, C₁-C₄ alkyl-C₁-C₄ alkoxy, hydroxy, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁷ is selected from the group consisting of hydrogen, C₁-C₄ alkoxy, C₁-C₄ alkyl-C₁-C₄ alkoxy, hydroxy, acyl, and C₁-C₄ alkyl optionally substituted with C(O)OR⁴ or C(O)NR⁴R⁵;
R⁴ is hydrogen;
R⁵ is hydrogen;
m is 1-3; and
the ring represented by

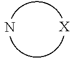

is a 4- to 7-membered heterocyclic ring.

In one embodiment, the present invention is directed to a compound, a prodrug thereof, or a pharmaceutically acceptable salt thereof represented by formula (3):

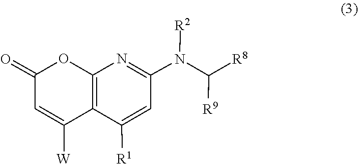

wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² is selected from the group consisting of CH₃ and CD₃;
R⁴ is hydrogen or C₁-C₃ alkyl;
R⁵ is R⁴ or C₁-C₃ alkyl substituted with C(O)OR⁴, C(O)NR⁴R⁴, or OR⁴;
R⁸ is C₁-C₄ alkyl optionally substituted with one or more substituents selected from the group consisting of fluoro, C₁-C₄ alkoxy, C(O)OR⁴, and C(O)NR⁴R⁵; and
R⁹ is hydrogen or CH₃.

In certain embodiments of formula (1), W is C₆-C₁₂ aryl which is optionally substituted with one or more groups, each independently selected from the group consisting of deuterium, fluoro, chloro, trifluoromethyl, difluoromethyl, cyano, hydroxyl, C₁-C₄ alkoxyl, and C₁-C₄ alkyl optionally substituted with OR⁴.

In certain embodiments of formula (1), W is 5-12 membered heteroaryl, either of which is optionally substituted with one or more groups, each independently selected from the group consisting of deuterium, fluoro, chloro, trifluoromethyl, difluoromethyl, cyano, hydroxyl, C₁-C₄ alkoxyl, and C₁-C₄ alkyl optionally substituted with OR⁴.

In certain embodiments of formula (1), W is ortho-tolyl.

In certain embodiments of formula (1), W is 2,6-dimethylphenyl.

In certain embodiments of formula (1), W is 2-chloro-4-fluorophenyl.

In certain embodiments of formula (1), R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines.

In certain embodiments of formula (1), R¹ is hydrogen.

In certain embodiments of formula (1), R¹ is deuterium.

In certain embodiments of formula (1), R¹ is hydroxyl.

In certain embodiments of formula (1), R¹ is cyano.

In certain embodiments of formula (1), R¹ is chlorine.

In certain embodiments of formula (1), R¹ is C₃-C₄ cycloalkyl.

In certain embodiments of formula (1), R¹ is C₁-C₃ alkyl optionally substituted with one or more fluorines.

In certain embodiments of formula (1), R¹ is C₁-C₃ alkoxyl optionally substituted with one or more fluorines.

In certain embodiments of formula (1), R² and R³ are independently hydrogen, C₁-C₄ alkoxy, C₃-C₆ cycloalkyl, or C₁-C₄ alkyl optionally substituted with one or more substituents selected from the group consisting of deuterium, fluoro, C₁-C₄ alkoxy, cyano, C(O)OR⁴, and C(O)NR⁴R⁵.

In certain embodiments of formula (1), R² is independently hydrogen.

In certain embodiments of formula (1), R² is independently C₁-C₄ alkoxy.

In certain embodiments of formula (1), R² is independently C₃-C₆ cycloalkyl.

In certain embodiments of formula (1), R² is independently C₁-C₄ alkyl optionally substituted with one or more substituents selected from the group consisting of deuterium, fluoro, C₁-C₄ alkoxy, cyano, C(O)OR⁴, and C(O)NR⁴R⁵.

In certain embodiments of formula (1), R² is independently C₁-C₄ alkyl optionally substituted with one or more deuterium.

In certain embodiments of formula (1), R² is independently C₁-C₄ alkyl optionally substituted with one or more fluoro.

In certain embodiments of formula (1), R² is independently C₁-C₄ alkyl optionally substituted with one or more C₁-C₄ alkoxy.

In certain embodiments of formula (1), R² is independently C₁-C₄ alkyl optionally substituted with one or more cyano.

In certain embodiments of formula (1), R² is independently C₁-C₄ alkyl optionally substituted with one or more C(O)OR⁴.

In certain embodiments of formula (1), R² is independently C₁-C₄ alkyl optionally substituted with one or more C(O)NR⁴R⁵.

In certain embodiments of formula (1), R³ is independently hydrogen.

In certain embodiments of formula (1), R³ is independently C₁-C₄ alkyl optionally substituted with one or more deuterium.

In certain embodiments of formula (1), R³ is independently C₁-C₄ alkyl optionally substituted with one or more fluoro.

In certain embodiments of formula (1), R³ is independently C₁-C₄ alkoxy.

In certain embodiments of formula (1), R³ is independently C₃-C₆ cycloalkyl.

In certain embodiments of formula (1), R² and R³ are each methyl.

In certain embodiments of formula (1), R² is methyl and R³ is methoxyethyl.

In certain embodiments of formula (1), R² and R³ are attached to the same nitrogen atom, and R² and R³ together with their connecting nitrogen form a 4- to 7-membered heterocyclic ring or 7- to 12-membered spiro ring, each optionally containing another heteroatom that is N, O, or S, and each ring is optionally substituted with one to four groups each independently selected from the group consisting of fluoro, chloro, C₁-C₄ alkyl, C₁-C₄ alkoxy, cyano, acyl, keto, carboxyl, optionally substituted amino, optionally substituted amide, and C₁-C₄ alkylcarboxylate.

In certain embodiments of formula (1), R² and R³ together with their connecting nitrogen form a morpholinyl ring.

In certain embodiments of formula (1), R² and R³ together with their connecting nitrogen form a morpholinyl ring substituted with a methyl group.

In certain embodiments of formula (1), R² and R³ together with their connecting nitrogen form a piperazinyl ring substituted with a methyl group.

In certain embodiments of formula (1), R² and R³ together with their connecting nitrogen form a piperazinyl ring substituted with a methyl group and a keto group.

In certain embodiments of formula (1), R² and R³ together with their connecting nitrogen form a piperazinyl ring substituted with an alkanoyl group.

In certain embodiments of formula (1), R² and R³ together with their connecting nitrogen form a piperazinyl ring substituted with an acetyl group.

In certain embodiments of formula (1), R² and R³ together with their connecting nitrogen form a 7-membered azaspiro ring substituted with an O atom.

In certain embodiments of formula (1), R² and R³ together with their connecting nitrogen form a pyrrolidinyl ring.

In certain embodiments of formula (1), R² and R³ together with their connecting nitrogen form a pyrrolidinyl ring substituted with alkoxy.

In certain embodiments of formula (1), R² and R³ together with their connecting nitrogen form a pyrrolidinyl ring substituted with methoxy.

In certain embodiments of formula (1), R² and R³ together with their connecting nitrogen form a pyrrolidinyl ring substituted with fluoro.

In certain embodiments of formula (1), R² and R³ together with their connecting nitrogen form a pyrrolidinyl ring substituted with difluoro.

In certain embodiments of formula (1), R² and R³ together with their connecting nitrogen form a pyrrolidinyl ring substituted with hydroxyl and methyl groups.

In certain embodiments of formula (1), R² and R³ together with their connecting nitrogen form a pyrrolidinyl ring substituted with an optionally substituted amino group.

In certain embodiments of formula (1), R² and R³ together with their connecting nitrogen form a pyrrolidinyl ring substituted with dimethyl amino.

In certain embodiments of formula (1), R² and R³ together with their connecting nitrogen form a pyrrolidinyl ring substituted with amide.

In certain embodiments of formula (1), R² and R³ together with their connecting nitrogen form a pyrrolidinyl ring substituted with C₁-C₄ alkoxy.

In certain embodiments of formula (1), R² and R³ together with their connecting nitrogen form a pyrrolidinyl ring substituted with methoxymethyl.

In certain embodiments of formula (1), R⁴ is hydrogen or C₁-C₃ alkyl.

In certain embodiments of formula (1), R⁴ is hydrogen.

In certain embodiments of formula (1), R⁴ is C₁-C₃ alkyl.

In certain embodiments of formula (1), R⁴ is CH₃.

In certain embodiments of formula (1), R⁵ is R⁴ or C₁-C₃ alkyl optionally substituted with C(O)OR⁴, C(O)NR⁴R⁴, or OR⁴.

In certain embodiments of formula (1), R⁵ is R⁴.

In certain embodiments of formula (1), R⁵ is C₁-C₃ alkyl.

In certain embodiments of formula (1), R⁵ is C₁-C₃ alkyl substituted with C(O)OR⁴, C(O)NR⁴R⁴, or OR⁴.

In certain embodiments of formula (1), R⁵ is C₁-C₃ alkyl substituted with C(O)OR⁴.

In certain embodiments of formula (1), R⁵ is C₁-C₃ alkyl substituted with C(O)NR⁴R⁴.

In certain embodiments of formula (1), R⁵ is C₁-C₃ alkyl substituted with OR⁴.

In certain embodiments, the compound is 2-(methyl(4-(2-(methyl-d₃)phenyl)-2-oxo-2H-pyrano[2,3-b]pyridin-7-yl)amino)acetamide, or a pharmaceutically acceptable salt thereof:

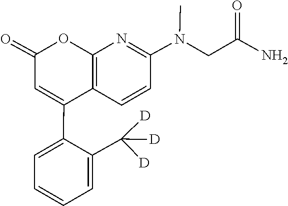 [SMILES: [2H]C([2H])([2H])c1ccccc1-c1cc(=O)oc2nc(N(C)CC(N)=O)ccc12]

In certain embodiments, the compound is an (R)- or (S)-enantiomer of 2-methyl-1-(2-oxo-4-(o-tolyl)-2H-pyrano[2,3-b]pyridin-7-yl)pyrrolidine-2-carboxylic acid, or a pharmaceutically acceptable salt thereof:

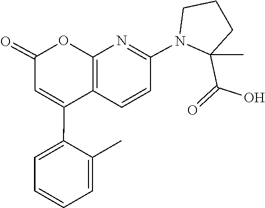 [SMILES: Cc1ccccc1-c1cc(=O)oc2nc(N3CCCC3(C)C(=O)O)ccc12]

In certain embodiments, the compound is an (R)- or (S)-enantiomer of (4-(2-(methyl-d₃)phenyl)-2-oxo-2H-pyrano[2,3-b]pyridin-7-yl)-L-proline, or a pharmaceutically acceptable salt thereof:

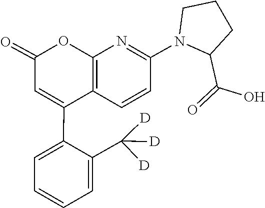 [SMILES: [2H]C([2H])([2H])c1ccccc1-c1cc(=O)oc2nc(N3CCCC3C(=O)O)ccc12]

In certain embodiments, the compound is an (R)- or (S)-enantiomer of 2-methyl-1-(4-(2-(methyl-d₃)phenyl)-2-oxo-2H-pyrano[2,3-b]pyridin-7-yl)pyrrolidine-2-carboxylic acid, or a pharmaceutically acceptable salt thereof:

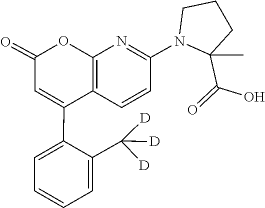 [SMILES: [2H]C([2H])([2H])c1ccccc1-c1cc(=O)oc2nc(N3CCCC3(C)C(=O)O)ccc12]

In certain embodiments, the compounds inhibits POLRMT.

In certain embodiments, the compounds promote POLRMT.

The compounds of the present invention may contain asymmetric carbon atoms (sometimes as the result of a deuterium atom) and thereby can exist as either individual stereoisomers or mixtures of the enantiomers or mixtures of diastereomers. Accordingly, a compound of the present invention may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. Carbon atoms labelled with * or ** refer to a compound that is chiral but the absolute stereochemistry has not been determined.

The compounds of the present invention may contain double bonds that may exist in more than one geometric isomer. Examples of such double bonds are carbon-carbon double bonds which form alkenes. In the case of carbon-carbon double bonds, the geometric isomers may be E or Z isomers.

Unless otherwise indicated, when a disclosed compound is named or depicted by a structure without specifying the geometric isomerism and has one or more possible geometric isomers, it is understood to represent all possible geometric isomers of the compound.

Certain compounds of the present invention may be able to exist as tautomers. All tautomeric forms of these compounds, whether isolated individually or in mixtures, are within the scope of the present invention. For example, in instances where an —OH substituent is permitted on a heteroaromatic ring and keto-enol tautomerism is possible, it is understood that the substituent might in fact be present, in whole or in part, in the oxo (=O) form.

Compounds of the present invention may exist in amorphous form and/or one or more crystalline forms. As such all amorphous and crystalline forms and mixtures thereof of the compounds of the invention are intended to be included within the scope of the present invention. In addition, some of the compounds of the present invention may form solvates with water (i.e., a hydrate) or common organic solvents. Such solvates and hydrates, particularly the pharmaceutically acceptable solvates and hydrates, of the compounds of this invention are likewise encompassed within the scope of the compounds of the invention and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the invention. See, e.g., Gannes, L Z et al., Comp. Biochem. Physiol. Mol. Integr. Physiol., 119, 725 (1998). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium. See Pirali et al, J. Med. Chem. 62, 5276-97 (2019). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances deuteration may have little to no impact on these properties, or may affect them in an undesirable manner. Whether and/or how such replacement will impact drug properties can only be determined if the drug is synthesized, evaluated, and compared to its non-deuterated counterpart. See Fukuto et al., J. Med. Chem. 34, 2871-76 (1991). Because some drugs have multiple sites of metabolism or more than one active sites for binding to a target, it is unpredictable as to which sites may benefit by deuterium replacement or to what extent isotope enrichment is necessary to produce a beneficial effect.

### Preparation of the Compounds

The starting materials and reagents used in each step in the preparation are known and can be readily prepared or purchased from commercial sources.

The compound obtained in each step can also be used for the next reaction as a reaction mixture thereof or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min. to 48 h., preferably 10 min. to 8 h.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters:ethyl acetate and the like; ketones: acetone, methyl ethyl ketone and the like; and water.

Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schutzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated POLRMT modulators of the present invention can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (“NMR”) can be used to determine a compound's structure while mass spectroscopy (“MS”) can be used to determine the amount of deuterium atom in the compound by comparison to its non-deuterated form.

### Compositions

The present invention further includes pharmaceutical compositions of the compounds, a pharmaceutically acceptable salt of said compounds, or prodrugs of said compounds. In addition to the compound of the invention, a salt thereof, or a prodrug thereof, the pharmaceutical compositions comprise one or more pharmaceutically acceptable excipients, such excipients being compatible with other ingredients in the composition and also being not toxic or otherwise harmful. Examples of excipients include carriers, lubricants, binders, disintegrants, solvents, solubilizing agents, suspending agents, isotonic agents, buffers, soothing agents, preservatives, antioxidants, colorants, taste-modifying agents, absorbents, and/or wetting agents.

The pharmaceutical compositions of the invention include those suitable for oral, rectal, nasal, topical, buccal, sublingual, vaginal or parenteral (including subcutaneous, intramuscular, intravenous and intradermal) administration. Such compositions may be prepared by any methods well known in the art of pharmaceutical formulations and pharmacy. See, e.g., “Remington: The Science and Practice of Pharmacy,” Elsevier Science, 23rd ed. (2020).

Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, hydrogels, gelatin, hydrogenated naphthalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, the compositions can be administered by oral ingestion. Compositions intended for oral use can be prepared in solid or liquid forms, according to any method known to a person of ordinary skill in the art for the manufacture of pharmaceutical compositions. Solid dosage forms for oral administration include capsules (both soft and hard gelatin capsules), tablets, powders, and granules. Generally, these pharmaceutical preparations contain active ingredients admixed with pharmaceutically acceptable excipients. These excipients include, for example, inert diluents, such as calcium carbonate, sodium carbonate, lactose, sucrose, glucose, mannitol, cellulose, starch, calcium phosphate, sodium phosphate, kaolin and the like; binding agents, buffering agents, and/or lubricating agents (e.g., magnesium stearate) may also be used. Tablets and capsules can additionally be prepared with release-controlling coatings such as enteric coatings. The compositions may optionally contain sweetening, flavoring, coloring, perfuming, and preserving agents in order to provide a more palatable preparation.

In another embodiment, a pharmaceutical composition of this invention further comprises a second therapeutic agent. The second therapeutic agent may be selected from any pharmaceutically active compound; preferably the second therapeutic agent is known to treat cancer, neurodegenerative disorders, or metabolic disorders. Alternatively, the compounds of the invention and second therapeutic agent may be administered together (within less than 24 hours of one another, consecutively or simultaneously) but in separate pharmaceutical compositions. In certain embodiments, the compounds of the invention and second therapeutic agent can be administered separately (e.g., more than 24 hours of one another). If the second therapeutic agent acts synergistically with the compounds of this invention, the therapeutically effective amount of such compounds and/or the second therapeutic agent may be less that such amount required when either is administered alone.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting.

### EXAMPLES

The examples and preparations provided below further illustrate and exemplify the compounds of the present invention and methods of preparing such compounds. It is to be understood that the scope of the present invention is not limited in any way by the scope of the following examples and preparations.

The structures of the compounds are confirmed by either mass spectrometry and/or NMR, where peaks assigned to the characteristic protons in the title compound are presented where appropriate. ¹H NMR shift (δ) are given in parts per million (ppm) down field from an internal reference standard.

The abbreviations used herein are known to a person of ordinary skill in the art. A partial list of abbreviations that may be used herein include: acetonitrile (MeCN), ammonium carbonate (NH₄)₂CO₃, ammonium chloride (NH₄Cl), aqueous (aq.), 1,1′-bis(diphenylphosphino)ferrocene (dppf), 1,3-bis(diphenylphosphino)propane (dppp), bis(pinacolato)diboron (B₂pin₂), N-bromosuccinimide (NBS), bromo-tris-pyrrolidino-phosphonium hexafluorophosphate (PyBroP), boron tribromide (BBr₃), butyl lithium (BuLi), calculated (Calcd.), cesium carbonate (Cs₂CO₃), dichloromethane (DCM, CH₂Cl₂), N,N-dicyclohexylcarbodiimide (DCC), dichloroethane (DCE), diethyl azodicarboxylate (DEAD), diisopropyl azodicarboxylate (DIAD), N,N-diisopropylethylamine (DIPEA), 4-dimethylaminopyridine (DMAP), dimethylformamide (DMF), dimethyl sulfoxide (DMSO), di-tert-butyl decarbonate (Boc₂O), 1-ethyl-3-(3-dimethylaminopropyl)carbodiimide (EDC), electrospray ionization (ESI), enantiomeric excess (ee), ethyl acetate (EtOAc), hour (h.), N-[(dimethylamino)-1H-1,2,3-triazolo-[4,5-b]pyridin-1-ylmethylene]-N-methylmethanaminium hexafluorophosphate N-oxide (HATU), high performance liquid chromatography (HPLC), hydroxybenzotriazole (HOBt), isopropyl alcohol (IPA), lithium hydroxide monohydrate (LiOH H₂O), methanol (MeOH), methyl iodide (Mel), minutes (min.), potassium carbonate (K₂CO₃), liquid chromatography-mass spectrometry (LCMS), phenyliodide(III) diacetate (PIDA), propylphosphonic anhydride (T₃P), reverse phase (RP), room/ambient temperature (rt, RT), silver oxide (Ag₂O), sodium hydride (NaH), sodium sulfate (Na₂SO₃), supercritical fluid chromatography (SFC), tetrahydrofuran (THF), triethylamine (Et₃N), thionyl chloride (SOCl₂), triphenylphosphine (PPh₃), dicyclohexyl[2′,4′,6′-tris(propan-2-yl)[1,1′-biphenyl]-2-yl]phosphane (XPhos).

The invention will now be described in reference to the following examples. These examples are not to be regarded as limiting the scope of the present invention, but shall only serve in an illustrative manner. Table 1 provides a listing of the example compounds of the present invention and IC₅₀ values.

### Example 1: Synthesis of 7-(dimethylamino)-4-(o-tolyl)-2H-pyrano[2,3-b]pyridin-2-one

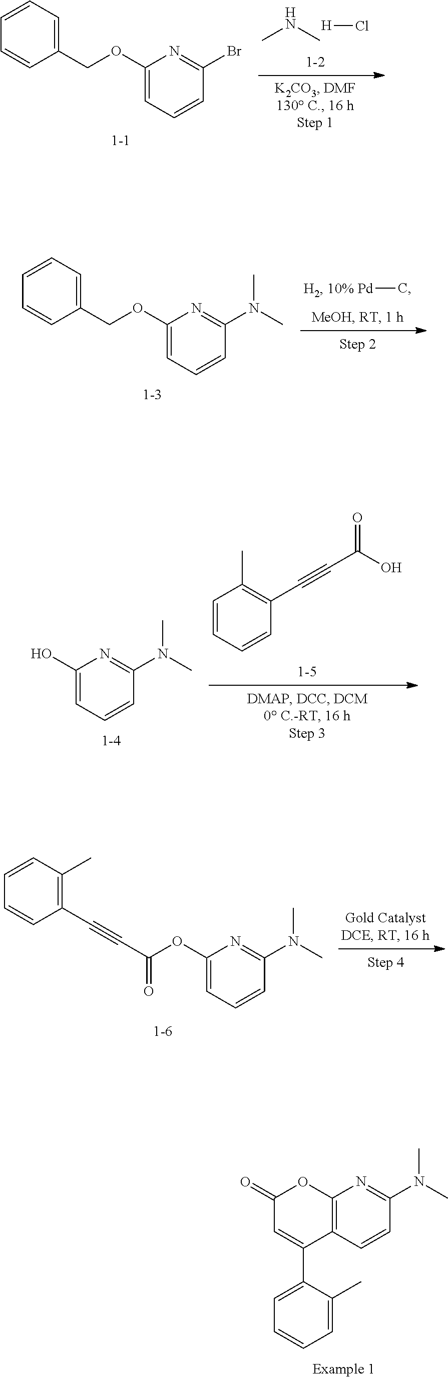

Synthesis of 6-(benzyloxy)-N,N-dimethylpyridin-2-amine, 1-3 [Step 1]: To stirred a solution of 2-(benzyloxy)-6-bromopyridine (1-1, 0.5 g, 1.9 mmol) in dimethylformamide (12.0 mL) was added potassium carbonate (1.0 g, 7.5 mmol) followed by dimethylamine hydrochloride (1-2, 465 mg, 5.6 mmol) at ambient temperature. The reaction mixture was gradually warmed to 130° C. and stirred for 16 h. The reaction mixture was cooled to ambient temperature and was partitioned between ethyl acetate and water. The organic layer was washed with cold water, brine, dried over anhydrous sodium sulfate, and concentrated under reduced pressure. The product was purified by flash column chromatography to obtain 6-(benzyloxy)-N,N-dimethylpyridin-2-amine (1-3, 300 mg). LCMS (ESI) Calcd. for C₁₄H₁₆N₂O: 228, found [M+H]⁺=229. ¹H NMR (400 MHz, DMSO-d₆) δ 7.46 (d, 2H), 7.38-7.24 (m, 5H), 6.05-6.00 (m, 2H), 5.34 (s, 1H), 3.03 (s, 6H).

Synthesis 6-(dimethylamino)pyridin-2-ol, 1-4 [Step 2]: To stirred a solution of 6-(benzyloxy)-N,N-dimethylpyridin-2-amine (1-3, 250 mg, 1.1 mmol) in methanol (10 mL) was added 10% palladium on carbon (75 mg) and the mixture was subjected to hydrogenolysis under a hydrogen balloon at ambient temperature for 1 hour. The reaction mixture was filtered through a celite bed, which was washed with methanol, and the filtrate was concentrated under reduced pressure to obtain 6-(dimethylamino)pyridin-2-ol (1-4, 100 mg). LCMS Calcd. for C₇H₁₀N₂O: 138, found [M+H]⁺=139. ¹H NMR (400 MHz, DMSO-d₆) δ 10.07 (br s, 1H), 7.28 (t, 1H), 5.84 (br s, 1H), 5.72 (d, 1H), 2.93 (s, 6H).

Synthesis of 6-(dimethylamino)pyridin-2-yl 3-(o-tolyl)propiolate, 1-6 [Step 3]: To a stirred solution 6-(dimethylamino)pyridine-2-ol (1-4, 100 mg, 0.7 mmol) and 3-(o-tolyl)prop-2-ynoic acid (1-5, 230 mg, 1.4 mmol) in dichloromethane (10 mL) was added a solution of N,N-dicyclohexylcarbodiimide (DCC) (149 mg, 0.7 mmol) in dichloromethane (3 mL) followed by 4-dimethylaminopyridine (DMAP) (90 mg, 0.7 mmol) at ambient temperature and stirred for 16 h. The reaction mixture was filtered, and the filtrate was washed with water, brine, dried over anhydrous sodium sulfate, and concentrated under reduced pressure. The product was purified by flash column chromatography to afford 6-(dimethylamino)pyridin-2-yl 3-(o-tolyl)propiolate (1-6, 50 mg). LCMS Calcd. for C₁₇H₁₆N₂O₂: 280, found [M+H]⁺=281. ¹H NMR (400 MHz, DMSO-d₆) δ 7.41-7.33 (m, 3H), 7.22 (d, 1H), 7.06 (d, 1H), 6.62 (d, 1H), 5.97 (s, 1H), 3.12 (s, 6H), 2.11 (s, 3H).

Synthesis of 7-(dimethylamino)-4-(o-tolyl)-2H-pyrano[2,3-b]pyridin-2-one, Example 1 [Step 4]: A solution of 6-(dimethylamino)pyridin-2-yl 3-(o-tolyl)propiolate (1-6, 100 mg, 0.4 mmol) in dichloroethane (10 mL) was degassed with nitrogen for 10 min. (Acetonitrile)[(2-biphenyl)di-tert-butylphosphine]gold(I) hexafluoroantimonate (Gold Catalyst) (30 mg, 0.03 mmol) was added to the reaction mixture, which was stirred at room temperature for 16 h. The reaction mixture was filtered through a celite bed, which was washed with dichloromethane (three times), and the filtrate was concentrated under reduced pressure. The product was purified by flash column chromatography and lyophilized to afford 7-(dimethylamino)-4-(o-tolyl)-2H-pyrano[2,3-b]pyridin-2-one (Example 1, 20 mg). LCMS Calcd. for C₁₇H₁₆N₂O₂: 280, found [M+H]⁺=281. ¹H NMR (400 MHz, DMSO-d₆) δ 7.43-7.37 (m, 2H), 7.33 (t, 1H), 7.22 (d, 1H), 7.06 (d, 1H), 6.62 (d, 1H), 5.96 (s, 1H), 3.12 (s, 6H), 2.11 (s, 3H).

### Example 2: Synthesis of 7-morpholino-4-(o-tolyl)-2H-pyrano[2,3-b]pyridin-2-one

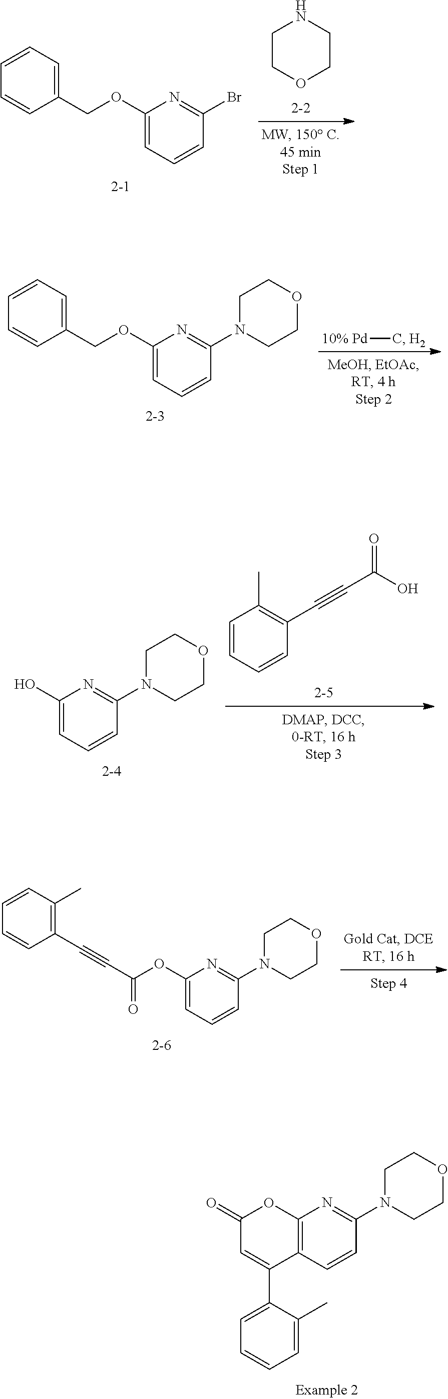

Synthesis of 4-(6-(benzyloxy)pyridin-2-yl)morpholine, 2-3 [Step 1]: A mixture of 2-(benzyloxy)-6-bromopyridine (2-1, 500 mg, 1.9 mmol) and morpholine (2-2, 1.1 mL, 13.3 mmol) was heated at 150° C. for 45 minutes using a microwave (MW) reactor. The reaction mixture was diluted with ethyl acetate and washed with water, brine, dried over anhydrous sodium sulfate, and concentrated under reduced pressure to yield the product. The product was purified by flash column chromatography to afford 4-(6-(benzyloxy)pyridin-2-yl)morpholine (2-3, 180 mg). LCMS (ESI) Calcd. for C₁₆H₁₈N₂O₂: 270, found [M+H]⁺=271. ¹H NMR (400 MHz, DMSO-d₆) δ 7.49 (t, 1H), 7.42 (d, 2H), 7.37 (t, 2H), 7.31 (t, 1H), 6.32 (d, 1H), 6.13 (d, 1H), 5.28 (s, 2H), 3.68 (br s, 4H), 3.40 (br s, 4H).

Synthesis of 6-morpholinopyridin-2-ol, 2-4 [Step 2]: To a solution of 4-(6-(benzyloxy)pyridin-2-yl)morpholine (2-3, 400 mg, 1.9 mmol) in ethanol (6 mL) was added 10% palladium on carbon (40 mg) and the mixture was subjected to hydrogenolysis under a hydrogen balloon for 16 h. The reaction mixture was filtered through a celite bed, and the filtrate was concentrated under reduced pressure to give 6-morpholinopyridin-2-ol (2-4, 230 mg). LCMS (ESI) Calcd. for C₉H₁₂N₂O₂: 180, found [M+H]⁺=181. ¹H NMR (400 MHz, DMSO-d₆) δ 12.36 (br s, 1H), 7.50 (s, 1H), 7.36 (t, 2H), 3.66 (br s, 4H), 3.54 (br s, 4H).

Synthesis of 6-morpholinopyridin-2-yl 3-(o-tolyl)propiolate, 2-6 [Step 3]: To an ice cooled mixture of 6-morpholinopyridin-2-ol (2-4, 230 mg, 1.3 mmol) and 3-(o-tolyl)propiolic acid (2-5, 204 mg, 1.3 mmol) in dichloromethane (5 mL) was added 4-dimethylaminopyridine (DMAP) (16 mg, 0.19 mmol) followed by a solution of N,N-dicyclohexylcarbodiimide (DCC) (420 mg, 2 mmol) in dichloromethane (1 mL). The reaction mixture was stirred and allowed to warm to ambient temperature over 16 h. The reaction mixture was filtered, and the filtrate was washed with water, brine, dried over anhydrous sodium sulfate, and concentrated under reduced pressure. The product was purified by flash column chromatography to afford 6-morpholinopyridin-2-yl 3-(o-tolyl)propiolate (2-6, 40 mg). LCMS(ESI) Calcd. for C₁₉H₁₈N₂O₃: 322, found [M+H]⁺=323.1. ¹H NMR (400 MHz, DMSO-d₆) δ 7.74 (t, 1H), 7.64 (d, 1H), 7.49 (t, 1H), 7.40 (d, 1H), 7.31 (t, 1H), 6.82 (d, 1H), 6.57 (d, 1H), 3.67 (br s, 4H), 3.44 (br s, 4H), 2.32 (s, 3H).

Synthesis of 7-morpholino-4-(o-tolyl)-2H-pyrano[2,3-b]pyridin-2-one, Example 2 [Step 4]: To a solution of 6-morpholinopyridin-2-yl 3-(o-tolyl)propiolate (2-6, 30 mg, 0.1 mmol) in dichloroethane (6 mL) was added (acetonitrile)[(2-biphenyl)di-tert-butylphosphine]gold(I) hexafluoroantimonate (Gold Catalyst) (7.2 mg, 0.01 mmol) under an inert atmosphere, and the reaction mixture was stirred for 16 h. at ambient temperature. The mixture was concentrated under reduced pressure and the product was purified by reverse-phase prep-HPLC to afford 7-morpholino-4-(o-tolyl)-2H-pyrano[2,3-b]pyridin-2-one (Example 2, 13 mg). LCMS Calcd. for C₁₉H₁₈N₂O₃: 322, found [M+H]⁺=323. ¹H NMR (400 MHz, DMSO-d₆) δ 7.42-7.34 (m, 3H), 7.23 (d, 1H), 7.10 (d, 1H), 6.79 (d, 1H), 6.03 (s, 1H), 3.69 (br s, 4H), 3.63 (br s, 4H), 2.11 (s, 3H).

### Example 3: Synthesis of 7-(pyrrolidin-1-yl)-4-(o-tolyl)-2H-pyrano[2,3-b]pyridin-2-one

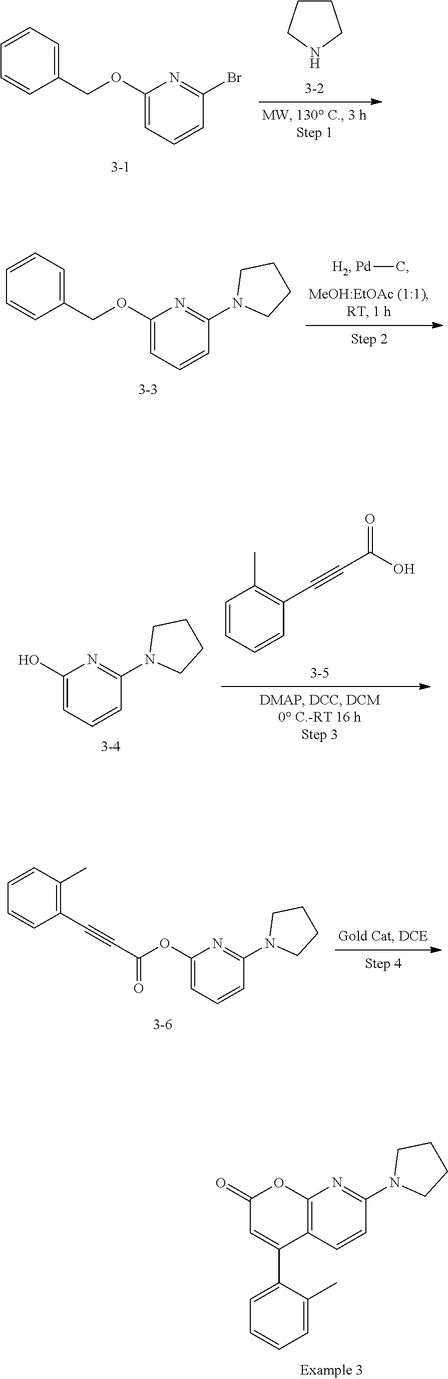

Synthesis of 2-(benzyloxy)-6-(pyrrolidin-1-yl)pyridine, 3-3 [Step 1]: A mixture of 2-(benzyloxy)-6-bromopyridine (3-1, 500 mg, 1.9 mmol) and pyrrolidine (3-2, 1.6 mL, 18.9 mmol) was heated at 130° C. for 3 h. using a microwave (MW) reactor. The product was diluted with ethyl acetate, washed with water (twice), and brine. The organic phase was concentrated under reduced pressure to yield 2-(benzyloxy)-6-(pyrrolidin-1-yl)pyridine (3-3, 400 mg). LCMS (ESI) Calcd. for C₁₆H₁₈N₂O: 254, found [M+H]⁺=255.

Synthesis of 6-(pyrrolidin-1-yl)pyridin-2-ol, 3-4 [Step 2]: To a stirred solution of 2-(benzyloxy)-6-(pyrrolidin-1-yl)pyridine (3-3, 390 mg, 1.5 mmol) in methanol (5 mL) and ethyl acetate (5 mL) was added palladium on carbon (10% wet) (60 mg, 1.5 mmol) at ambient temperature under an inert atmosphere. The reaction mixture was then subjected to hydrogenolysis in a Parr shaker under hydrogen gas at ambient temperature for 1 hour. The reaction mixture was filtered through a celite bed, which was washed with methanol. The filtrate was concentrated under reduced pressure to afford 6-(pyrrolidin-1-yl)pyridin-2-ol (3-4, 220 mg). LCMS (ESI) Calcd. for C₉H₁₂N₂O: 164, found [M+H]⁺=165.

Synthesis of 6-(pyrrolidin-1-yl)pyridin-2-yl 3-(o-tolyl)propiolate, 3-6 [Step 3]: To an ice-cold mixture of 6-(pyrrolidin-1-yl)pyridin-2-ol (3-4, 220 mg, 1.3 mmol) and 3-(o-tolyl)propiolic acid (3-5, 235 mg, 1.5 mmol) in dichloromethane (7 mL) was added a mixture of 4-dimethylaminoipyridine (DMAP) (35 mg, 0.26 mmol) and N,N-dicyclohexylcarbodiimide (DCC) (330 mg, 1.6 mmol) in dichloromethane (2 mL). The reaction mixture was stirred overnight and allowed to reach ambient temperature. The mixture was concentrated under reduced pressure and the product was purified by flash column chromatography to afford 6-(pyrrolidin-1-yl)pyridin-2-yl 3-(o-tolyl)propiolate (3-6, 170 mg). LCMS (ESI) Calcd. for C₁₉H₁₈N₂O₂: 306, found [M+H]⁺=307.

Synthesis of 7-(pyrrolidin-1-yl)-4-(o-tolyl)-2H-pyrano[2,3-b]pyridin-2-one, Example 3 [Step 4]: To a solution of 6-(pyrrolidin-1-yl)pyridin-2-yl 3-(o-tolyl)propiolate (3-6, 130 mg, 0.4 mmol) in dichloroethane (4 mL) was added (acetonitrile)[(2-biphenyl)di-tert-butylphosphine]gold(I) hexafluoroantimonate (Gold Catalyst) (35 mg, 0.04 mmol) under an inert atmosphere, and the reaction mixture was stirred for 16 h. at ambient temperature. The mixture was filtered through a celite bed, which was washed with dichloromethane (three times). The filtrate was concentrated under reduced pressure to give the product that was purified by reverse-phase prep-HPLC, and lyophilized to afford 7-(pyrrolidin-1-yl)-4-(o-tolyl)-2H-pyrano[2,3-b]pyridin-2-one (Example 3, 40 mg). LCMS (ESI) Calcd. for C₁₉H₁₈N₂O₂: 306, found [M+H]⁺=307. ¹H NMR (400 MHz, DMSO-d₆) 7.43-7.32 (m, 3H), 7.22-7.20 (m, 1H), 7.04-7.02 (m, 1H), 6.44-6.42 (m, 1H), 5.94 (s, 1H), 3.47 (br s, 4H), 2.11 (s, 3H), 1.96 (br s, 4H).

### Example 4: Synthesis of 7-((2-methoxyethyl)(methyl)amino)-4-(o-tolyl)-2H-pyrano[2,3-b]pyridin-2-one

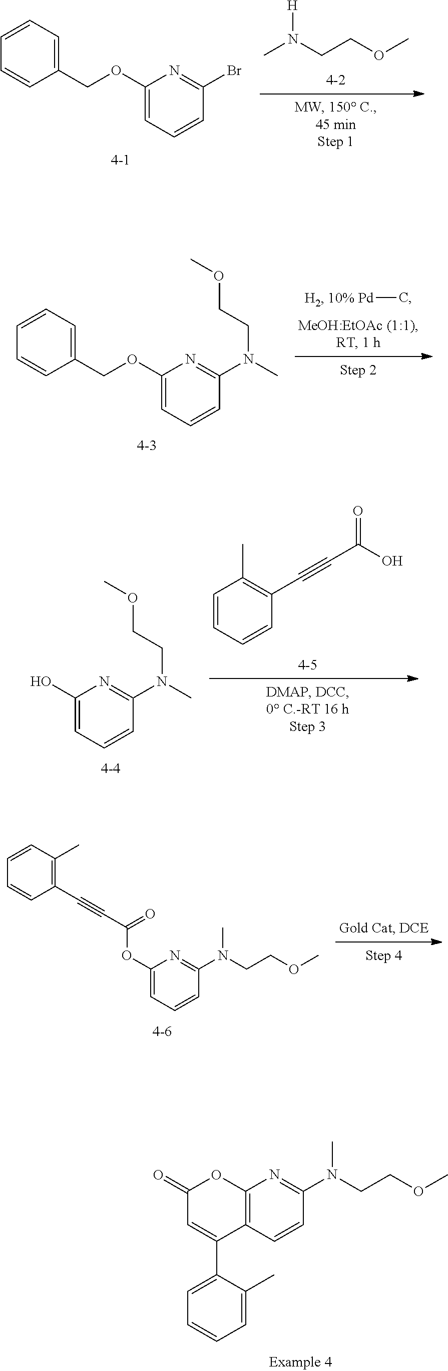

Synthesis of 6-(benzyloxy)-N-(2-methoxyethyl)-N-methylpyridin-2-amine, 4-3 [Step 1]: A mixture of 2-(benzyloxy)-6-bromopyridine (4-1, 500 mg, 1.9 mmol) and 2-methoxy-N-methylethan-1-amine (4-2, 2.1 mL, 18.9 mmol) was heated at 150° C. for 45 minutes using a microwave (MW) reactor. The reaction mixture was partitioned between ethyl acetate and water and the organic layer was collected, washed with brine, dried over anhydrous sodium sulfate, and concentrated under reduced pressure to give 6-(benzyloxy)-N-(2-methoxyethyl)-N-methylpyridin-2-amine (4-3, 390 mg). LCMS (ESI) Calcd. for C₁₆H₂₀N₂O₂: 272, found [M+H]⁺=273. ¹H NMR (400 MHz, DMSO-d₆) δ 7.42-7.26 (m, 6H), 6.09 (d, 1H), 6.00 (d, 1H), 5.29 (s, 2H), 3.62 (t, 2H), 3.40 (t, 2H), 3.21 (s, 3H), 2.96 (s, 3H).

Synthesis of 6-((2-methoxyethyl)(methyl)amino)pyridin-2-ol, 4-4 [Step 2]: To a stirred solution of 6-(benzyloxy)-N-(2-methoxyethyl)-N-methylpyridin-2-amine (4-3, 380 mg, 1.4 mmol) in methanol (4 mL) and ethyl acetate (4 mL) was added 10% palladium on carbon (60 mg) at ambient temperature under an inert atmosphere. The reaction mixture was subjected to hydrogenolysis under a hydrogen balloon at ambient temperature for 1 hour. The reaction mixture was filtered through a celite bed, which was washed with methanol. The filtrate was concentrated under reduced pressure to give 6-((2-methoxyethyl)(methyl)amino)pyridin-2-ol (4-4, 250 mg). LCMS (ESI) Calcd. for C₉H₁₄N₂O₂: 182, found [M+H]⁺=183. ¹H NMR (400 MHz, DMSO-d₆) δ 9.92 (br s, 1H), 7.28 (t, 1H), 5.87 (d, 1H), 5.73 (d, 1H), 3.60 (t, 2H), 3.46 (t, 2H), 3.24 (s, 3H), 2.93 (s, 3H).

Synthesis of 6-((2-methoxyethyl)(methyl)amino)pyridin-2-yl 3-(o-tolyl)propiolate, 4-6 [Step 3]: To an ice-cold mixture of 6-((2-methoxyethyl)(methyl)amino)pyridin-2-ol (4-4, 260 mg, 1.4 mmol) and 3-(o-tolyl)propiolic acid (4-5, 250 mg, 1.6 mmol) in DCM (4 mL) was added 4-dimethylaminopyridine (DMAP) (35 mg, 0.3 mmol) followed by N,N-dicyclohexylcarbodiimide (DCC) (350 mg, 1.7 mmol) in dichloromethane (2 mL) and the reaction mixture was stirred overnight and allowed to reach ambient temperature. The reaction mixture was concentrated under reduced pressure and purified by flash column chromatography to afford 6-((2-methoxyethyl)(methyl)amino)pyridin-2-yl 3-(o-tolyl)propiolate (4-6, 230 mg). LCMS (ESI) Calcd. for C₁₉H₂₀N₂O₃: 324, found [M+H]⁺=325. ¹H NMR (400 MHz, DMSO-d₆) δ 7.66-7.61 (m, 2H), 7.48 (t, 1H), 7.39 (d, 1H), 7.30 (t, 1H), 6.6 (d, 1H), 6.43 (d, 1H), 3.63 (t, 2H), 3.47 (t, 2H), 3.23 (s, 3H), 3.0 (s, 3H), 2.35 (s, 3H).

Synthesis of 7-((2-methoxyethyl)(methyl)amino)-4-(o-tolyl)-2H-pyrano[2,3-b]pyridin-2-one, Example 4 [Step 4]: A solution of 6-((2-methoxyethyl)(methyl)amino) pyridin-2-yl 3-(o-tolyl)propiolate (4-6, 160 mg, 0.5 mmol) in dichloroethane (3 mL) was degassed with nitrogen for 10 min. (Acetonitrile)[(2-biphenyl)di-tert-butylphosphine]gold(I) hexafluoroantimonate (Gold Catalyst) (40 mg, 0.04 mmol) was added to the reaction mixture, which was stirred at ambient temperature for 16 h. The reaction mixture was filtered through a celite bed, which was washed with dichloromethane (three times). The filtrate was concentrated under reduced pressure, purified by flash column chromatography, and lyophilized to afford 7-((2-methoxyethyl)(methyl)amino)-4-(o-tolyl)-2H-pyrano[2,3-b]pyridin-2-one (Example 4, 120 mg). LCMS (ESI) Calcd. for C₁₉H₂₀N₂O₃: 324, found [M+H]⁺=325. ¹H NMR (400 MHz, DMSO-d₆) 7.43-7.39 (m, 2H), 7.33 (t, 1H), 7.22 (d, 1H), 7.05 (d, 1H), 6.64 (d, 1H), 5.97 (s, 1H), 3.75 (m, 2H), 3.54 (t, 2H), 3.25 (s, 3H), 3.10 (s, 3H), 2.12 (s, 3H).

### Example 5: Synthesis of 7-(ethyl(methyl)amino)-4-(o-tolyl)-2H-pyrano[2,3-b]pyridin-2-one

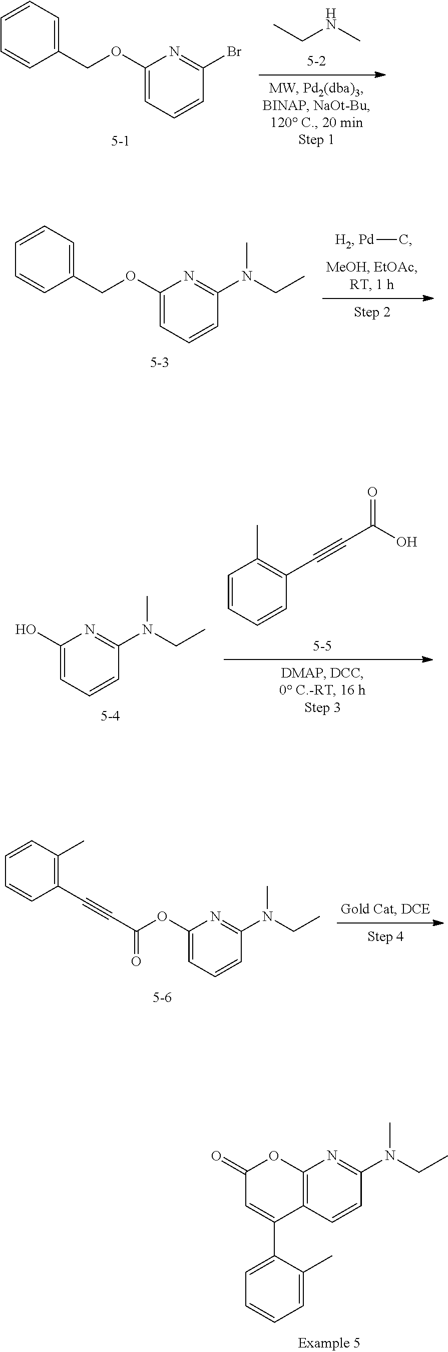

Synthesis of 6-(benzyloxy)-N-ethyl-N-methylpyridin-2-amine, 5-3 [Step 1]: A solution of 2-(benzyloxy)-6-bromopyridine (5-1, 500 mg, 1.9 mmol) and N-methylethanamine (5-2, 0.8 mL, 9.5 mmol) in toluene (3 mL) was kept under nitrogen gas. After 5 min., sodium tert-butoxide (275 mg, 2.8 mmol) was added and nitrogen purging was continued for 5 min. Then BINAP (1.2 g, 1.9 mmol) followed by Pd₂(dba)₃ (345 mg, 0.4 mmol) was added and the reaction mixture was heated to 120° C. in microwave for 20 minutes. The reaction mixture was concentrated under reduced pressure and the product was diluted with water, extracted with EtOAc (3×30 mL), and the organic phase was washed with brine, and dried over anhydrous Na₂SO₄. The product was purified by column chromatography over silica gel to afford 6-(benzyloxy)-N-ethyl-N-methylpyridin-2-amine (5-3, 250 mg). LCMS (ESI) Calcd. for C₁₅H₁₈N₂O: 242, found [M+H]⁺=243.

Synthesis of 6-(ethyl(methyl)amino)pyridin-2-ol, 5-4 [Step 2]: To a stirred solution of 6-(benzyloxy)-N-ethyl-N-methylpyridin-2-amine (5-3, 470 mg, 1.9 mmol) in a mixture of methanol (3 mL) and ethyl acetate (3 mL) was added 10% Pd—C(70 mg) at ambient temperature under an inert atmosphere. The reaction mixture was subjected to hydrogenolysis using a H₂ balloon at ambient temperature for 1 h. The reaction mixture was filtered through a bed of celite, which was washed with methanol. The filtrate was concentrated under reduced pressure to afford 6-(ethyl(methyl)amino)pyridin-2-ol (5-4, 250 mg). LCMS (ESI) Calcd. for C₈H₁₂N₂O: 152, found [M+H]⁺=153.

Synthesis 6-(ethyl(methyl)amino)pyridin-2-yl 3-(o-tolyl)propiolate, 5-6 [Step 3]: To a stirred solution of 6-(ethyl(methyl)amino)pyridin-2-ol (5-4, 240 mg, 1.6 mmol) and 3-(o-tolyl)prop-2-ynoic acid (5-5, 280 mg, 1.7 mmol) in DCM (4 mL) under a N₂ atmosphere was added DMAP (40 mg, 0.3 mmol) followed by a solution of DCC (390 mg, 1.9 mmol) in DCM (2 ml) under ice cold conditions. The reaction mixture was stirred at ambient temperature overnight. The product was purified by column chromatography using silica gel to afford 6-(ethyl(methyl)amino)pyridin-2-yl 3-(o-tolyl)propiolate (5-6, 120 mg). LCMS (ESI) Calcd. for C₁₈H₁₈N₂O₂: 294, found [M+H]⁺=296.

Synthesis of 7-(ethyl(methyl)amino)-4-(o-tolyl)-2H-pyrano[2,3-b]pyridin-2-one, Example 5 [Step 4]: A solution of 6-(ethyl(methyl)amino)pyridin-2-yl 3-(o-tolyl)propiolate (5-6, 110 mg, 0.4 mmol) in DCE (2 mL) was degassed with nitrogen for 10 min. Then (acetonitrile)[(2-biphenyl)di-tert-butylphosphine]gold(I) hexafluoroantimonate (Gold Cat.) (30 mg, 0.03 mmol) was added and the reaction mixture was stirred at ambient temperature for 16 h. The reaction mixture was filtered through a celite bed, which was washed with DCM (thrice). The combined filtrate was concentrated under reduced pressure. The product was purified by RP prep-HPLC and lyophilized to yield 7-(ethyl(methyl)amino)-4-(o-tolyl)-2H-pyrano[2,3-b]pyridin-2-one (Example 5, 60 mg). LCMS (ESI) Calcd. for C₁₈H₁₈N₂O₂: 294, found [M+H]⁺=295. ¹H NMR (400 MHz, DMSO-d₆) 7.43-7.37 (m, 2H), 7.35-7.31 (m, 1H), 7.22-7.20 (d, 1H), 7.04-7.02 (d, 1H), 6.61-6.59 (d, 1H), 5.95 (s, 1H), 3.63-3.58 (m, 2H), 3.07 (s, 3H), 2.11 (s, 3H), 1.09-1.03 (t, 3H).

### Example 6: Synthesis of 7-(4-acetylpiperazin-1-yl)-4-(o-tolyl)-2H-pyrano[2,3-b]pyridin-2-one

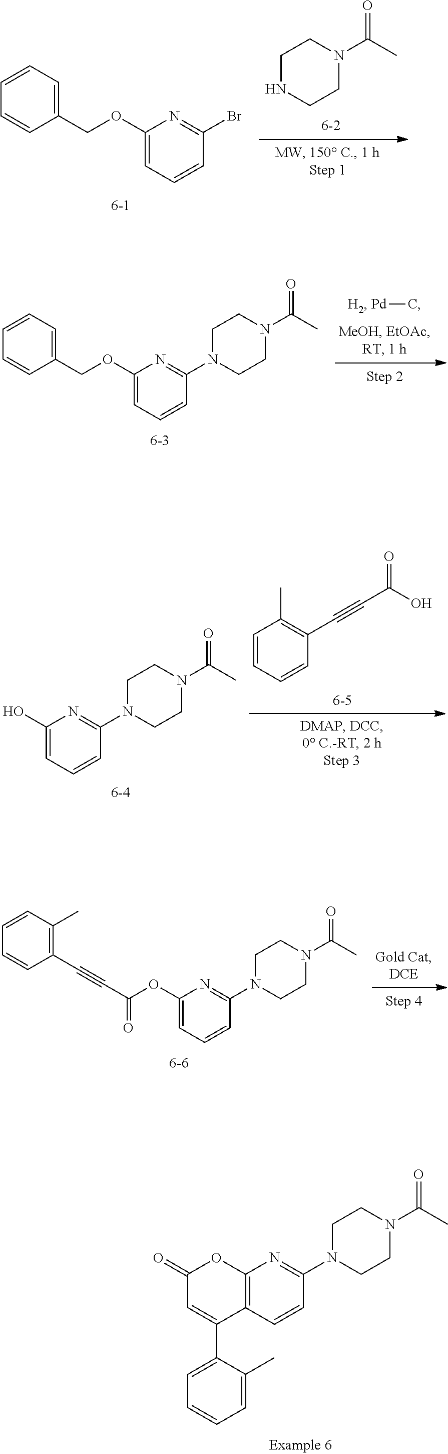

Synthesis of 1-(4-(6-(benzyloxy)pyridin-2-yl)piperazin-1-yl)ethan-1-one, 6-3 [Step 1]: In a dried microwave reactor was added 2-(benzyloxy)-6-bromopyridine (6-1, 500 mg, 1.9 mmol) and 1-(piperazin-1-yl)ethan-1-one (6-2, 2.4 g, 19 mmol), and the reaction mixture was heated using microwave (MW) at 150° C. for 10 min. The reaction mixture was diluted with EtOAc and washed with water (twice) and concentrated under reduced pressure. The product was purified by combiflash to afford 1-(4-(6-(benzyloxy)pyridin-2-yl)piperazin-1-yl)ethan-1-one (6-3, 400 mg). LCMS (ESI) Calcd. for C₁₈H₂₁N₃O₂: 311, found [M+H]⁺=312. ¹H NMR (400 MHz, DMSO-d₆): δ 7.49-7.47 (t, 1H), 7.45-7.39 (d, 2H), 7.36-7.29 (t, 2H), 7.25-7.23 (d, 1H), 6.34-6.32 (d, 1H), 6.12-6.10 (d, 1H), 5.30 (s, 2H), 3.50-3.41 (s, 6H), 3.33-3.29 (s, 2H), 2.01 (s, 3H).

Synthesis of 1-(4-(6-hydroxypyridin-2-yl)piperazin-1-yl)ethan-1-one, 6-4 [Step 2]: To a degassed solution of 1-(4-(6-(benzyloxy)pyridin-2-yl)piperazin-1-yl)ethan-1-one (6-3, 400 mg, 1.28 mmol) in a mixture of methanol (5 mL) and ethyl acetate (5 mL) was added Pd—C(10%) (40 mg) at ambient temperature. The reaction mixture was subjected to hydrogenolysis at ambient temperature under a hydrogen balloon pressure for 1 h. The reaction mixture was filtered through a celite bed, which was washed with methanol. The filtrate was concentrated under reduced pressure to afford 1-(4-(6-hydroxypyridin-2-yl)piperazin-1-yl)ethan-1-one (6-4, 220 mg). The product was used in the next step without further purification. LCMS (ESI) Calcd. for C₁₁H₁₅N₃O₂: 221, found [M+H]⁺=222. ¹H NMR (400 MHz, DMSO-d₆): δ 10.31-10.20 (br s, 1H), 7.38-7.34 (t, 1H), 6.15-6.11 (d, 1H), 5.88-5.86 (d, 1H), 3.50 (s, 4H), 3.43 (m, 2H), 3.22-3.30 (m, 2H), 2.03 (s, 3H).

Synthesis of 6-(4-acetylpiperazin-1-yl)pyridin-2-yl 3-(o-tolyl)propiolate, 6-6 [Step 3]: To a stirred solution of 1-(4-(6-hydroxypyridin-2-yl)piperazin-1-yl)ethan-1-one (6-4, 250 mg, 1.1 mmol) and 3-(o-tolyl)prop-2-ynoic acid (6-5, 200 mg, 1.2 mmol) in DCM (4 mL) was added DMAP (30 mg, 0.23 mmol) followed by a solution of DCC (280 mg, 1.4 mmol) in DCM (2 ml) under ice cold conditions. The reaction mixture was stirred at ambient temperature for 2 h and then filtered through a celite bed, which was washed with DCM. The filtrate was concentrated under reduced pressure. The product was purified by RP prep-HPLC afford 6-(4-acetylpiperazin-1-yl)pyridin-2-yl 3-(o-tolyl)propiolate (6-6, 130 mg). LCMS (ESI) Calcd. for C₂₁H₂₁N₃O₃: 363, found [M+H]⁺=364. ¹H NMR (400 MHz, DMSO-d₆): δ 7.74-7.70 (t, 1H), 7.64-7.62 (d, 1H), 7.51-7.47 (t, 1H), 7.40-7.33 (d, 1H), 7.31-7.29 (t, 1H), 6.84-6.81 (d, 1H), 6.55-6.54 (d, 1H), 3.53-3.48 (m, 8H), 2.32 (s, 3H), 2.07-2.03 (s, 3H).

Synthesis of 7-(4-acetylpiperazin-1-yl)-4-(o-tolyl)-2H-pyrano[2,3-b]pyridin-2-one, Example 6 [Step 4]: To a degassed solution of 6-(4-acetylpiperazin-1-yl)pyridin-2-yl 3-(o-tolyl)propiolate (6-6, 175 mg, 0.5 mmol) in DCE (5 mL) was added (acetonitrile)[(2-biphenyl)di-tert-butylphosphine]gold(I) hexafluoroantimonate (Gold Cat.) (35 mg, 0.05 mmol) and the reaction mixture stirred at 40° C. for 16 h. The reaction mixture was filtered through a celite bed, which was washed with DCM (thrice). The filtrate was concentrated under reduced pressure. The product was purified by prep-HPLC and lyophilized to afford 7-(4-acetylpiperazin-1-yl)-4-(o-tolyl)-2H-pyrano[2,3-b]pyridin-2-one (Example 6, 40 mg). LCMS (ESI) Calcd. for C₂₁H₂₁N₃O₃: 363, found [M+H]⁺=364. ¹H NMR (400 MHz, DMSO-d₆): δ 7.42-7.38 (m, 2H), 7.35-7.32 (m, 1H), 7.22-7.21 (d, 1H), 7.10-7.08 (d, 1H), 6.80-6.78 (d, 1H), 6.03 (s, 1H), 3.74-3.71 (m, 2H), 3.66-3.63 (m, 2H), 7.56 (m, 4H), 2.11 (s, 3H), 2.04 (s, 3H).

### Example 7: Synthesis of 7-(3-methoxypyrrolidin-1-yl)-4-(o-tolyl)-2H-pyrano[2,3-b]pyridin-2-one

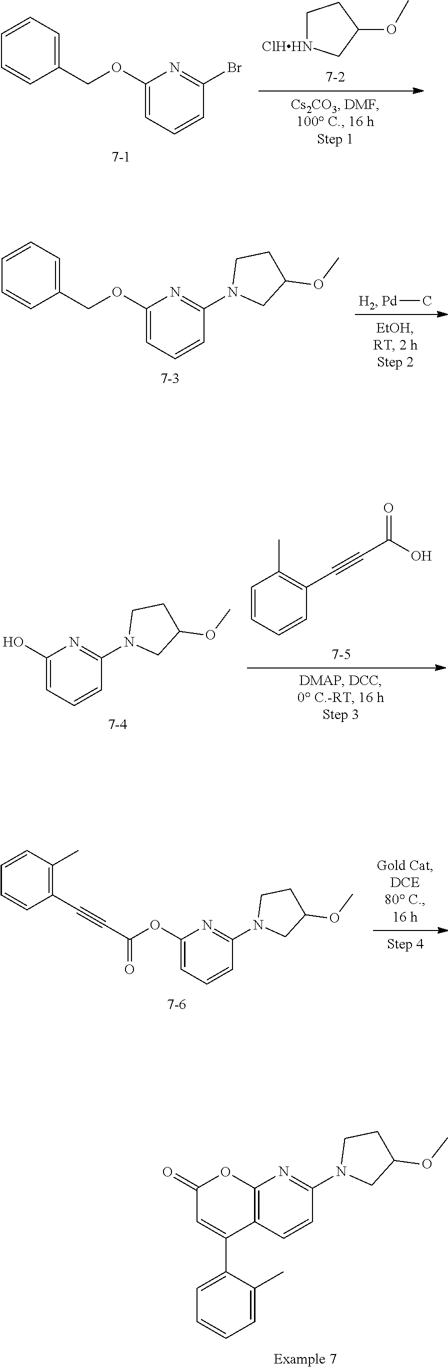

Synthesis of 2-(benzyloxy)-6-(3-methoxypyrrolidin-1-yl)pyridine, 7-3 [Step 1]: To a mixture of 2-(benzyloxy)-6-bromopyridine (7-1, 1.5 g, 5.6 mmol) and 3-methoxypyrrolidine hydrochloride (7-2, 1.0 g, 6.8 mmol) in DMF (10 mL), was added Cs₂CO₃ (5.6 g, 17 mmol) at ambient temperature and the reaction mixture was heated at 100° C. for 16 h. The reaction mixture was diluted with EtOAc, washed with water, brine, dried over anhydrous Na₂SO₄ and concentrated under reduced pressure. The product was purified by flash chromatography to afford 2-(benzyloxy)-6-(3-methoxypyrrolidin-1-yl)pyridine (7-3, 490 mg). LCMS (ESI) Calcd. for C₁₇H₂₀N₂O₂: 284, found [M+H]⁺=285. ¹H NMR (400 MHz, DMSO-d₆) δ 7.44 (d, 2H), 7.36 (m, 3H), 7.28 (t, 1H), 6.03 (d, 1H), 5.88 (d, 1H), 5.34 (s, 2H) 4.12 (s, 1H), 3.55 (s, 2H), 3.50 (m, 2H), 3.35 (s, 3H), 2.14 (m, 2H).

Synthesis of 6-(3-methoxypyrrolidin-1-yl)pyridin-2-ol, 7-4 [Step 2]: To a solution of 2-(benzyloxy)-6-(3-methoxypyrrolidin-1-yl)pyridine (7-3, 470 mg, 1.6 mmol) in EtOH (10 mL) was added 10% Pd—C(100 mg) and the reaction mixture was subjected to hydrogenolysis under a hydrogen balloon pressure for 2 h. at ambient temperature. The reaction mixture was filtered through celite bed and concentrated under reduced pressure to obtain 6-(3-methoxypyrrolidin-1-yl)pyridin-2-ol (7-4, 311 mg). LCMS (ESI) Calcd. for C₁₀H₁₄N₂O₂: 194, found [M+H]⁺=195. ¹H NMR (400 MHz, DMSO-d₆) δ 10.08 (br s, 1H), 7.27 (t, 1H), 5.64 (t, 2H), 4.03 (s, 1H), 3.60 (d, 1H), 3.41 (br s, 4H), 3.24 (s, 3H), 2.15 (t, 1H).

Synthesis of 6-(3-methoxypyrrolidin-1-yl)pyridin-2-yl 3-(o-tolyl)propiolate, 7-6 [Step 3]: To a stirred solution of 6-(3-methoxypyrrolidin-1-yl)pyridin-2-ol (7-4, 311 mg, 1.6 mmol) and 3-(o-tolyl)propiolic acid (7-5, 256 mg, 1.6 mmol) in DCM (5 mL) was added DMAP (32 mg, 0.24 mmol) followed by a solution of DCC (528 mg, 2.56 mmol) in DCM (1 mL), and the reaction mixture was stirred at ambient temperature for 16 h. The reaction mixture was filtered and the filtrate was washed with water, brine, dried over anhydrous Na₂SO₄, and concentrated under reduced pressure. The product was purified by flash chromatography to afford 6-(3-methoxypyrrolidin-1-yl)pyridin-2-yl 3-(o-tolyl)propiolate (7-6, 160 mg). ¹H NMR (400 MHz, DMSO-d₆) δ 7.66 (t, 2H), 7.48 (d, 1H), 7.39 (d, 1H), 7.32 (d, 1H), 6.44 (q, 2H), 4.05 (s, 1H), 3.45 (s, 1H), 3.29 (t, 2H), 2.37 (s, 3H), 2.04 (d, 2H), 1.2 (m, 1H).

Synthesis of 7-(3-methoxypyrrolidin-1-yl)-4-(o-tolyl)-2H-pyrano[2,3-b]pyridin-2-one, Example 7 [Step 4]: To a solution of 6-(3-methoxypyrrolidin-1-yl)pyridin-2-yl 3-(o-tolyl)propiolate (7-6, 130 mg, 0.38 mmol) in DCE (6 mL) was added (acetonitrile)[(2-biphenyl)di-tert-butylphosphine]gold(I) hexafluoroantimonate (Gold Cat.) (29 mg, 0.03 mmol) under an inert atmosphere and the reaction mixture was stirred for 16 h. at ambient temperature. The reaction mixture was concentrated under reduced pressure and purified by RP prep-HPLC to afford 7-(3-methoxypyrrolidin-1-yl)-4-(o-tolyl)-2H-pyrano[2,3-b]pyridin-2-one (Example 7, 107 mg). LCMS Calcd. for C₂₀H₂₀N₂O₃: 336, found [M+H]⁺=337. ¹H NMR (400 MHz, DMSO-d₆) δ 7.43 (m, 3H), 7.22 (d, 1H), 7.05 (d, 1H), 5.96 (s, 1H), 4.09 (s, 1H), 3.55 (d, 1H) 3.44 (s, 2H), 3.31 (d, 1H), 3.26 (s, 3H), 2.11 (s, 5H).

### Example 8: Synthesis of 7-[3-(dimethylamino)pyrrolidin-1-yl]-4-(o-tolyl)pyrano[2,3-b]pyridin-2-one

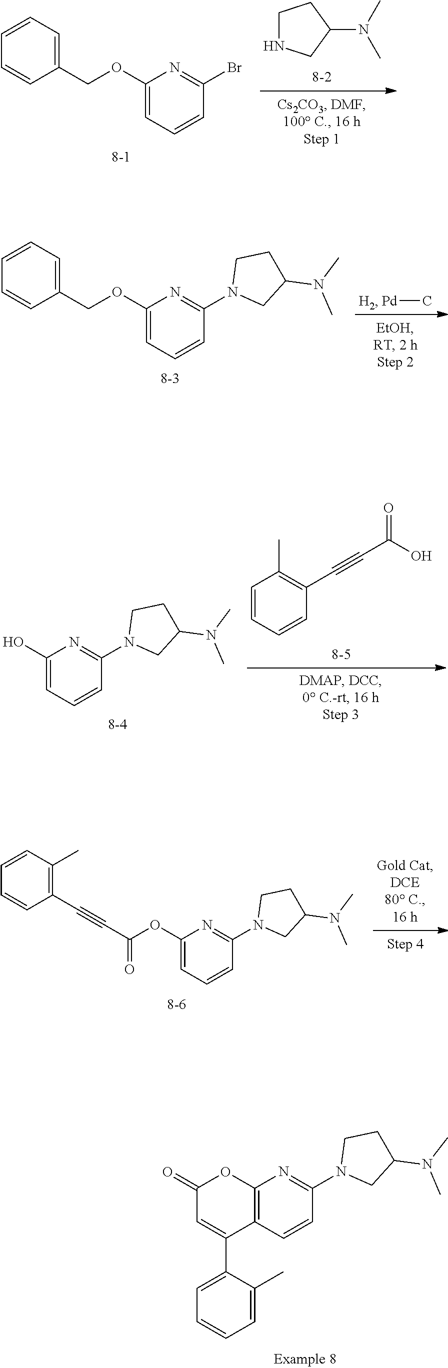

Synthesis of 1-(6-(benzyloxy)pyridin-2-yl)-N,N-dimethylpyrrolidin-3-amine, 8-3 [Step 1]: To a mixture of 2-(benzyloxy)-6-bromopyridine (8-1, 1.0 g, 3.79 mmol) and N,N-dimethylpyrrolidin-3-amine (8-2, 0.519 g, 4.54 mmol) in DMF (10 mL) was added CS₂CO₃ (3.7 g, 11.4 mmol) at ambient temperature and the reaction mixture was heated at 100° C. for 16 h. The reaction mixture was diluted with EtOAc, washed with water, brine, dried over anhydrous Na₂SO₄, and concentrated under reduced pressure. The product was purified by flash chromatography to afford 1-(6-(benzyloxy)pyridin-2-yl)-N,N-dimethylpyrrolidin-3-amine (8-3, 870 mg). LCMS (ESI) Calcd. for C₁₈H₂₃N₃O: 297, found [M+H]⁺=299. ¹H NMR (400 MHz, DMSO-d₆) δ 7.44 (d, 2H), 7.36 (m, 3H), 7.28 (m, 1H), 6.03 (d, 1H), 5.86 (d, 1H), 5.33 (s, 2H), 3.72-3.68 (m, 1H), 3.64-3.59 (m, 1H), 3.34-3.33 (m, 1H), 3.20 (t, 1H), 2.82 (m, 1H), 2.30 (s, 6H), 2.20 (m, 1H), 1.92 (m, 1H).

Synthesis of 6-(3-(dimethylamino)pyrrolidin-1-yl)pyridin-2-ol, 8-4 [Step 2]: To a solution of 1-(6-benzyloxy-2-pyridyl)-N,N-dimethyl-pyrrolidin-3-amine (8-3, 870 mg, 2.93 mmol) in EtOH (10 mL) was added 10% Pd—C(100 mg) under a nitrogen atmosphere and the reaction mixture was subjected to hydrogenolysis under a hydrogen balloon pressure for 2 h. at ambient temperature. The reaction mixture was filtered through a celite bed and concentrated under reduced pressure to obtain 6-[3-(dimethylamino)pyrrolidin-1-yl]pyridin-2-ol (8-4, 478 mg). The product was used directly in the next step without purification. ¹H NMR (400 MHz, DMSO-d₆) δ 10.13 (br s, 1H), 7.26 (t, 1H), 5.63 (m, 2H), 3.48 (t, 1H), 3.31 (m, 2H), 3.07 (t, 1H), 2.72 (m, 1H), 2.17 (br s, 6H), 2.08 (m, 1H), 1.77 (t, 1H).

Synthesis of [6-[3-(dimethylamino)pyrrolidin-1-yl]-2-pyridyl]3-(o-tolyl)prop-2-ynoate, 8-6 [Step 3]: To a stirred solution of 6-[3-(dimethylamino)pyrrolidin-1-yl]pyridin-2-ol (8-4, 478 mg, 2.31 mmol) and 3-(o-tolyl)prop-2-ynoic acid (8-5, 369 mg, 2.31 mmol) in DCM (5 mL) was added DMAP (42 mg, 0.34 mmol) followed by a solution of DCC (761 mg, 3.69 mmol) in DCM (1 mL) and the reaction mixture was stirred at ambient temperature for 16 h. The reaction mixture was filtered and the filtrate was washed with water, brine, dried over anhydrous Na₂SO₄, and concentrated under reduced pressure. The product was purified by flash chromatography to afford [6-[3-(dimethylamino)pyrrolidin-1-yl]-2-pyridyl]3-(o-tolyl)prop-2-ynoate (8-6, 311 mg). LCMS Calcd. for C₂₁H₂₃N₃O₂: 349, found [M+H]⁺=350. ¹H NMR (400 MHz, DMSO-d₆) δ 7.66 (t, 2H), 7.50 (t, 1H), 7.40 (d, 1H), 7.32 (t, 1H), 6.44 (t, 2H), 4.03 (m, 1H), 3.65 (t, 1H), 3.56 (t, 1H), 3.11 (t, 1H), 2.79 (s, 1H), 2.37 (s, 3H), 2.20 (s, 6H), 1.98 (s, 1H), 1.82 (m, 1H).

Synthesis of 7-[3-(dimethylamino)pyrrolidin-1-yl]-4-(o-tolyl)pyrano[2,3-b]pyridin-2-one, Example 8 [Step 4]: To a solution of 6-[3-(dimethylamino)pyrrolidin-1-yl]-2-pyridyl]3-(o-tolyl)prop-2-ynoate (8-6, 200 mg, 0.57 mmol) in DCE (8 mL) was added (acetonitrile)[(2-biphenyl)di-tert-butylphosphine]gold(I) hexafluoroantimonate (Gold Cat.) (44 mg, 0.05 mmol) under an inert atmosphere and the reaction mixture was stirred for 16 h. at ambient temperature. The reaction mixture was concentrated under reduced pressure and purified by RP prep-HPLC to afford 7-[3-(dimethylamino)pyrrolidin-1-yl]-4-(o-tolyl)pyrano[2,3-b]pyridin-2-one (Example 8, 33 mg). LCMS Calcd. for C₂₁H₂₃N₃O₂: 349, found [M+H]⁺=350. ¹H NMR-VT (400 MHz, DMSO-d₆, 100° C.) δ 7.41 (m, 3H), 7.21 (d, 1H), 7.06 (d, 1H), 6.44 (d, 1H), 5.90 (s, 1H), 3.75 (m, 2H) 3.48 (m, 1H), 3.31 (t, 1H), 2.95-2.90 (m, 1H), 2.24 (m, 6H), 2.14 (m, 4H), 1.92 (m, 1H).

### Example 9: Synthesis of 7-(4-methylpiperazin-1-yl)-4-(o-tolyl)pyrano[2,3-b]pyridin-2-one

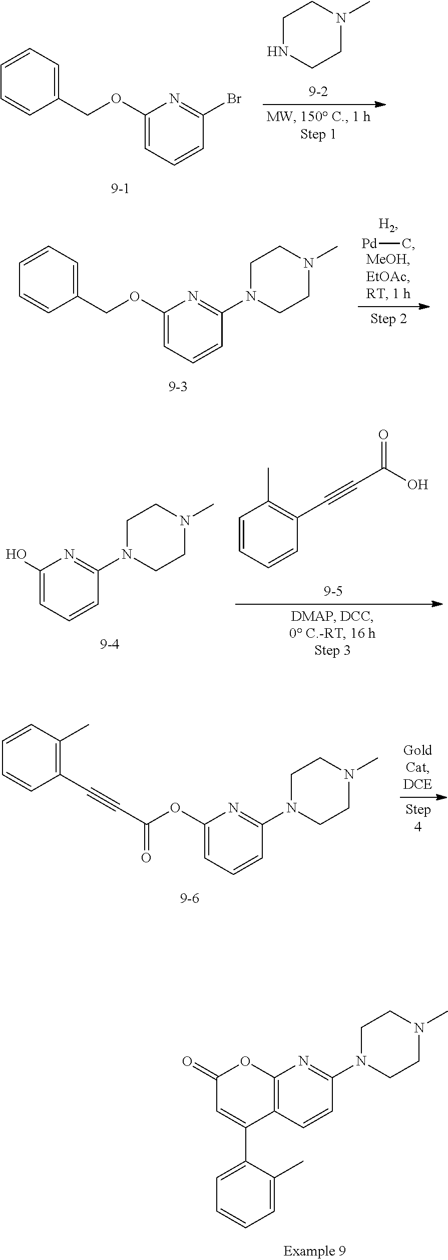

Synthesis of 1-(6-(benzyloxy)pyridin-2-yl)-4-methylpiperazine, 9-3 [Step 1]: In a dried microwave tube, 2-benzyloxy-6-bromo-pyridine (9-1, 500 mg, 1.9 mmol) and 1-methyl-piperazine (9-2, 2.1 mL, 18.9 mmol) was charged and the mixture was heated at 150° C. for 50 min. in a microwave reactor. The reaction mixture was diluted with water and extracted with EtOAc. The organic layer was washed with brine, dried over anhydrous sodium sulfate, and concentrated under reduced pressure. The product was purified by flash chromatography on silica gel to afford 1-(6-benzyloxy-2-pyridyl)-4-methyl-piperazine (9-3, 400 mg). ¹H NMR (400 MHz, DMSO-d₆) δ 7.45-7.25 (m, 6H), 6.30 (d, 1H), 6.07 (d, 1H), 5.26 (s, 2H), 3.43 (s, 4H), 3.37 (s, 4H), 2.20 (s, 3H). LC/MS: Calcd. for C₁₇H₂₁N₃O: 283, found [M+H]⁺=284.

Synthesis of 6-(4-methylpiperazin-1-yl)pyridin-2-ol, 9-4 [Step 2]: To a degassed solution of 1-(6-benzyloxy-2-pyridyl)-4-methyl-piperazine (9-3, 100 mg, 0.3 mmol) in a mixture of methanol (2 mL) and ethyl acetate (2 mL) was added Pd—C(10%, 15 mg, 0.3 mmol). The reaction mixture was purged with Argon for 5 min. and subjected to hydrogenolysis using a hydrogen balloon for 16 h. The reaction mixture was filtered through a celite bed and the filtrate was concentrated under reduced pressure to obtain 6-(4-methylpiperazin-1-yl)pyridin-2-ol (9-4, 50 mg). ¹H NMR (400 MHz, DMSO-d₆) δ 10.12 (s, 1H), 7.32 (t, 1H), 6.03 (d, 1H), 5.82 (d, 1H), 3.36-3.31 (m, 4H), 2.36-2.29 (s, 4H), 2.19 (s, 3H). LC/MS: Calcd. for C₁₀H₁₅N₃O: 193, found [M+H]⁺=194.

Synthesis of 6-(4-methylpiperazin-1-yl)pyridin-2-yl 3-(o-tolyl)propiolate, 9-6 [Step 3]: To a stirred solution of 6-(4-methylpiperazin-1-yl)pyridin-2-ol (9-4, 200 mg, 1.0 mmol) and 3-(o-tolyl)prop-2-ynoic acid (9-5, 182 mg, 1.1 mmol) in DCM (4 mL) was added DMAP (25 mg, 0.2 mmol) followed by the dropwise addition of a solution of DCC (260 mg, 1.2 mmol) in DCM (2 ml) at 0° C. The reaction mixture was stirred at ambient temperature for 2 h., and then diluted with water and extracted with EtOAc. The organic layer was washed with brine, dried over anhydrous Na₂SO₄, and concentrated under reduced pressure. The product was purified by flash column chromatography by using silica gel to afford [6-(4-methylpiperazin-1-yl)-2-pyridyl]3-(o-tolyl)prop-2-ynoate (9-6, 100 mg). ¹H NMR (400 MHz, DMSO-d₆) δ 7.69 (t, 1H), 7.62 (d, 1H), 7.49 (t, 1H), 7.39 (d, 1H), 7.29 (t, 1H), 6.80 (d, 1H), 6.51 (d, 1H), 3.46 (m, 4H), 2.37 (m, 7H), 2.20 (s, 3H). LC/MS: Calcd. for C₂₀H₂₁N₃O₂: 335, found [M+H]⁺=336.

Synthesis of 7-(4-methylpiperazin-1-yl)-4-(o-tolyl)-2H-pyrano[2,3-b]pyridin-2-one, Example 9 [Step 4]: To a degassed solution of [6-(4-methylpiperazin-1-yl)-2-pyridyl]3-(o-tolyl)prop-2-ynoate (9-6, 80 mg, 0.2 mmol) in DCE (4 mL) was added (acetonitrile)[(2-biphenyl)di-tert-butylphosphine]gold(I) hexafluoroantimonate (Gold Cat.) (19 mg, 0.03 mmol) and the reaction mixture was stirred at 40° C. for 16 h. The reaction mixture was filtered through celite bed, which was washed with DCM (thrice). The organic layer was washed with brine, dried over anhydrous Na₂SO₄ and concentrated under reduced pressure. The product was purified by prep-HPLC and lyophilized to afford 7-(4-methylpiperazin-1-yl)-4-(o-tolyl)pyrano[2,3-b]pyridin-2-one (Example 9, 12 mg). ¹H NMR (400 MHz, DMSO-d₆) δ 7.44-7.37 (m, 2H), 7.34 (t, 1H), 7.21 (d, 1H), 7.05 (d, 1H), 6.79 (d, 1H), 6.00 (s, 1H), 3.65 (t, 4H), 2.38 (t, 4H), 2.32 (s, 3H), 2.11 (s, 3H). LCMS: Calcd. for C₂₀H₂₁N₃O₂: 335, found [M+H]⁺=336.

### Example 10: Synthesis of 7-(2-oxa-6-azaspiro[3.3]heptan-6-yl)-4-(o-tolyl)-2H-pyrano[2,3-b]pyridin-2-one

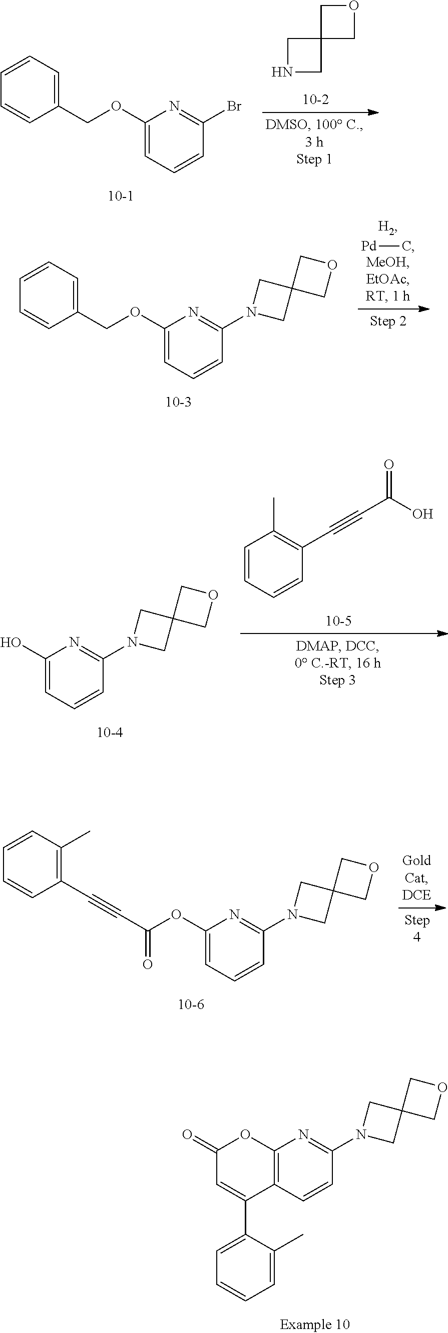

Synthesis of 6-(6-(benzyloxy)pyridin-2-yl)-2-oxa-6-azaspiro[3.3]heptane, 10-3 [Step 1]: 2-(benzyloxy)-6-bromopyridine (10-1, 600 mg, 2.27 mmol) and 2-oxa-6-azaspiro[3.3]heptane (10-2, 2.25 g, 22.7 mmol) were dissolved in DMF (3 mL) and the reaction mixture was stirred at 110° C. for 3 h. The reaction mixture was diluted with EtOAc and washed with water (twice). The organic layer was concentrated under reduced pressure to obtain the product that was purified by combiflash to afford 6-(6-(benzyloxy)pyridin-2-yl)-2-oxa-6-azaspiro[3.3]heptane (10-3, 500 mg). LCMS (ESI) Calcd. for C₁₇H₁₈N₂O₂: 282, found [M+H]⁺=283. ¹H NMR (400 MHz, DMSO-d₆): δ 7.43-7.29 (m, 6H), 6.07-6.05 (d, 1H), 5.90-5.88 (d, 1H), 5.26 (s, 2H), 4.70 (s, 4H), 4.06 (s, 4H).

Synthesis of 6-(2-oxa-6-azaspiro[3.3]heptan-6-yl)pyridin-2-ol, 10-4 [Step 2]: To a de-gassed solution of 6-(6-(benzyloxy)pyridin-2-yl)-2-oxa-6-azaspiro[3.3]heptane (10-3, 350 mg, 1.24 mmol) in a mixture of methanol (5 mL) and ethyl acetate (5 mL) was added Pd—C(10%, 40 mg) at ambient temperature and the reaction mixture was allowed to stir under a H₂ balloon for 1 h. The reaction mixture was filtered through a celite bed, which was washed with MeOH. The solvent was concentrated under reduced pressure to obtain 6-(2-oxa-6-azaspiro[3.3]heptan-6-yl)pyridin-2-ol (10-4, 200 mg). The product was used in the next step without further purification. LCMS (ESI) Calcd. for C₁₀H₁₂N₂O₂: 192, found [M+H]⁺=193. ¹H NMR (400 MHz, DMSO-d₆): δ 10.48 (br s, 1H), 7.27-7.23 (t, 1H), 5.69-5.67 (d, 1H), 5.44-5.43 (d, 1H), 4.68 (s, 4H), 4.04 (s, 4H).

Synthesis of 6-(2-oxa-6-azaspiro[3.3]heptan-6-yl)pyridin-2-yl 3-(o-tolyl)propiolate, 10-6 [Step 3]: To a stirred solution of 6-(2-oxa-6-azaspiro[3.3]heptan-6-yl)pyridin-2-ol (10-4, 220 mg, 1.1 mmol) and 3-(o-tolyl)propiolic acid (10-5, 202 mg, 1.3 mmol) in DCM (4 mL) was added DMAP (28 mg, 0.23 mmol) followed by a solution of DCC (283 mg, 1.4 mmol) in DCM (2 ml) under ice cold conditions, and the reaction mixture was stirred at ambient temperature for 2 h. The reaction mixture was filtered through a celite bed, which was washed with DCM. The filtrate was concentrated under reduced pressure to obtain the product that was purified by prep-HPLC chromatography. The product was concentrated under reduced pressure to afford 6-(2-oxa-6-azaspiro[3.3]heptan-6-yl)pyridin-2-yl 3-(o-tolyl)propiolate (10-6, 110 mg). LCMS (ESI) Calcd. for C₂₀H₁₈N₂O₃: 334, found [M+H]⁺=335. ¹H NMR (400 MHz, DMSO-d₆): δ 7.68-7.62 (m, 2H), 7.52-7.47 (m, 1H), 7.40 (d, 1H), 7.33-7.31 (d, 1H), 6.52-6.50 (d, 1H), 6.36-6.34 (d, 1H), 4.70 (s, 4H), 4.11 (s, 4H), 2.33-2.32 (s, 3H).

Synthesis of 7-(2-oxa-6-azaspiro[3.3]heptan-6-yl)-4-(o-tolyl)-2H-pyrano[2,3-b]pyridin-2-one, Example 10 [Step 4]: To a degassed solution of 6-(2-oxa-6-azaspiro[3.3]heptan-6-yl)pyridin-2-yl 3-(o-tolyl)propiolate (10-6, 150 mg, 0.5 mmol) in DCE (5 mL) was added (acetonitrile)[(2-biphenyl)di-tert-butylphosphine]gold(I) hexafluoroantimonate (Gold Cat.) (35 mg. 0.05 mmol) and the reaction mixture was stirred at 40° C. for 16 h. The reaction mixture was filtered through a celite bed, which was washed with DCM (thrice). The filtrate was concentrated under a reduced pressure and the product was purified by prep-HPLC and lyophilized to afford 7-(2-oxa-6-azaspiro[3.3]heptan-6-yl)-4-(o-tolyl)-21J-pyrano[2,3-b]pyridin-2-one (Example 10, 65 mg). LCMS (ESI) Calcd. for C₂₀H₁₈N₂O₃: 334, found [M+H]^{t}=335. ¹H NMR (400 MHz. DMSO-d₆): δ 7.41-7.31 (m, 3H), 7.21-7.19 (d, 1H), 7.05-7.03 (d, 1H), 6.30-6.27 (d, 1H), 5.99 (s, 1H), 4.72 (s, 4H), 4.25 (s, 4H), 2.09 (s, 3H).

### Example 11: Synthesis of 7-(4-methyl-3-oxopiperazin-1-yl)-4-(o-tolyl)-2H-pyrano[2,3-b]pyridin-2-one

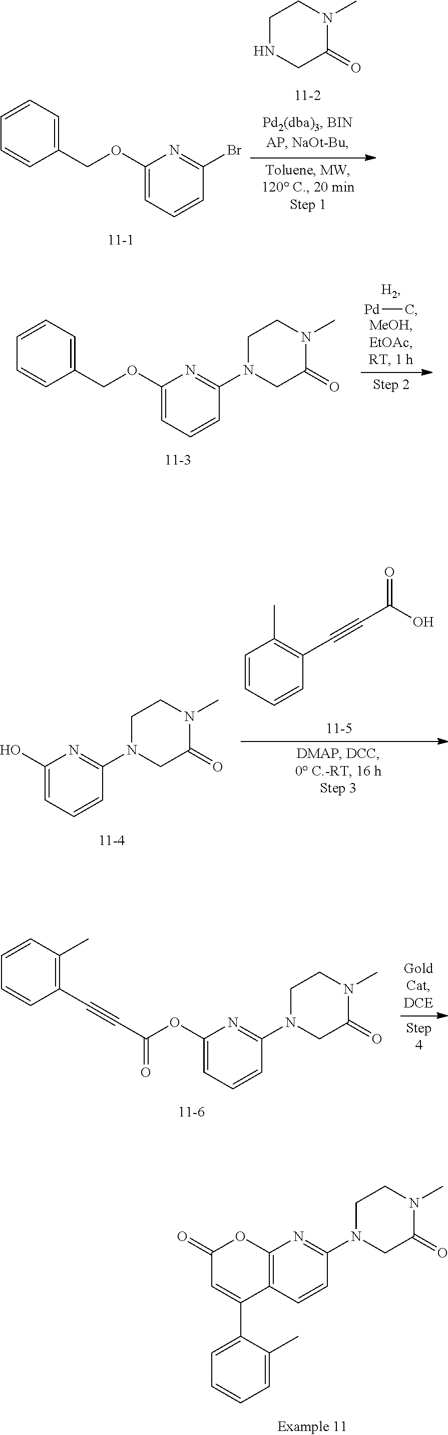

Synthesis of 4-(6-(benzyloxy)pyridin-2-yl)-1-methylpiperazin-2-one, 11-3 [Step 1]: To a stirred solution of 2-benzyloxy-6-bromo-pyridine (11-1, 250 mg, 0.9 mmol) and 1-methylpiperazin-2-one (11-2, 108 mg, 0.9 mmol) in toluene (2 mL) under an inert atmosphere was added NaOt-Bu (182 mg, 1.9 mmol) and BINAP (0.2 eq, 118 mg, 0.189 mmol) under an inert atmosphere. The reaction mixture was stirred for 5 min. and Pd₂(dba)₃ (87 mg, 0.09 mmol) was added and the reaction mixture was heated to 120° C. in a microwave for 20 min. The reaction mixture was filtered through a celite bed, which was washed with ethyl acetate. The organic layer was washed with water and dried over anhydrous Na₂SO₄, concentrated under reduced pressure, and purified by column chromatography to obtain 4-(6-(benzyloxy)pyridin-2-yl)-1-methylpiperazin-2-one (11-3, 200 mg). ¹H NMR (400 MHz, DMSO-d₆) δ 7.5-7.46 (m, 1H), 7.43 (d, 2H), 7.37 (t, 2H), 7.31 (d, 1H), 6.34 (d, 1H), 6.12 (d, 1H), 5.29 (s, 2H), 4.00 (d, 2H), 3.77 (d, 2H), 3.38 (d, 2H), 2.88 (s, 3H). LC/MS: Calcd. for C₁₇H₁₉N₃O₂: 297, found [M+H]⁺=298.

Synthesis of 4-(6-hydroxypyridin-2-yl)-1-methylpiperazin-2-one, 11-4 [Step 2]: A solution of 4-(6-(benzyloxy)pyridin-2-yl)-1-methylpiperazin-2-one (11-3, 200 mg, 0.7 mmol) in methanol (2 mL) and ethyl acetate (2 mL) was degassed with N₂ followed by the addition of 10% Pd/C (40 mg). The reaction mixture was stirred for 4 h. under H₂ gas. The reaction mixture was filtered through a celite bed, which was washed with methanol, concentrated under reduced pressure and purified by column chromatography to obtain 4-(6-hydroxypyridin-2-yl)-1-methylpiperazin-2-one (11-4, 100 mg). LC/MS: Calcd. for C₁₀H₁₃N₃O₂: 207, found [M+H]⁺=208.

Synthesis of 6-(4-methyl-3-oxopiperazin-1-yl)pyridin-2-yl 3-(o-tolyl)propiolate, 11-6 [Step 3]: A solution of 4-(6-hydroxypyridin-2-yl)-1-methylpiperazin-2-one (11-4, 160 mg, 0.8 mmol) and 3-(o-tolyl)prop-2-ynoic acid (11-5, 136 mg, 0.8 mmol) in DCM (4 mL) was placed under an inert atmosphere. To the mixture was added DMAP (19 mg, 0.15 mmol) followed by DCC (191 mg, 0.9 mmol) in DCM (2 mL) under ice cold conditions. The reaction was allowed to warn to ambient temperature and stirred for 1 h. The reaction mixture was concentrated under reduced pressure, and the product was purified by column chromatography to afford 6-(4-methyl-3-oxopiperazin-1-yl)pyridin-2-yl 3-(o-tolyl)propiolate (11-6, 60 mg). ¹H NMR (400 MHz, DMSO-d₆) δ 7.76 (t, 1H), 7.64 (d, 1H), 7.49 (d, 1H), 7.40 (d, 1H), 7.33 (t, 1H), 6.83 (d, 1H), 6.58 (s, 1H), 4.03 (s, 2H), 3.80 (t, 2H), 3.40 (d, 2H), 2.87 (s, 3H), 2.38 (s, 3H). LC/MS: Calcd. for C₂₀H₁₉N₃O₃: 349, found [M+H]⁺=350.

Synthesis of 7-(4-methyl-3-oxopiperazin-1-yl)-4-(o-tolyl)-2H-pyrano[2,3-b]pyridin-2-one, Example 11 [Step 4]: A solution of 6-(4-methyl-3-oxopiperazin-1-yl)pyridin-2-yl 3-(o-tolyl)propiolate (11-6, 60 mg, 0.2 mmol) in DCE (3 mL) was degassed with nitrogen for 10 min. To the mixture was added (acetonitrile)[(2-biphenyl)di-tert-butylphosphine]gold(I) hexafluoroantimonate (Gold Cat.) (13 mg, 0.02 mmol) and the reaction mixture was stirred at 40° C. for 16 h. The reaction mixture was filtered through a celite bed, which was washed with DCM (thrice). The filtrate was concentrated under reduced pressure and the product was purified by Prep-HPLC and lyophilized to obtain 7-(4-methyl-3-oxopiperazin-1-yl)-4-(o-tolyl)-2H-pyrano[2,3-b]pyridin-2-one (Example 11, 30 mg). ¹H NMR (400 MHz, DMSO-d₆) δ 7.44-7.32 (m, 3H), 7.22 (d, 1H), 7.14 (d, 1H), 6.75 (d, 1H), 6.00 (s, 1H), 4.18 (s, 2H), 3.94 (t, 2H), 3.49 (t, 2H), 2.92 (s, 3H), 2.14 (s, 3H). LC/MS: Calcd. for C₂₀H₁₉N₃O₃: 349, found [M+H]⁺=350.

### Example 12: Synthesis of 7-(3-(methoxymethyl)pyrrolidin-1-yl)-4-(o-tolyl)-2H-pyrano[2,3-b]pyridin-2-one

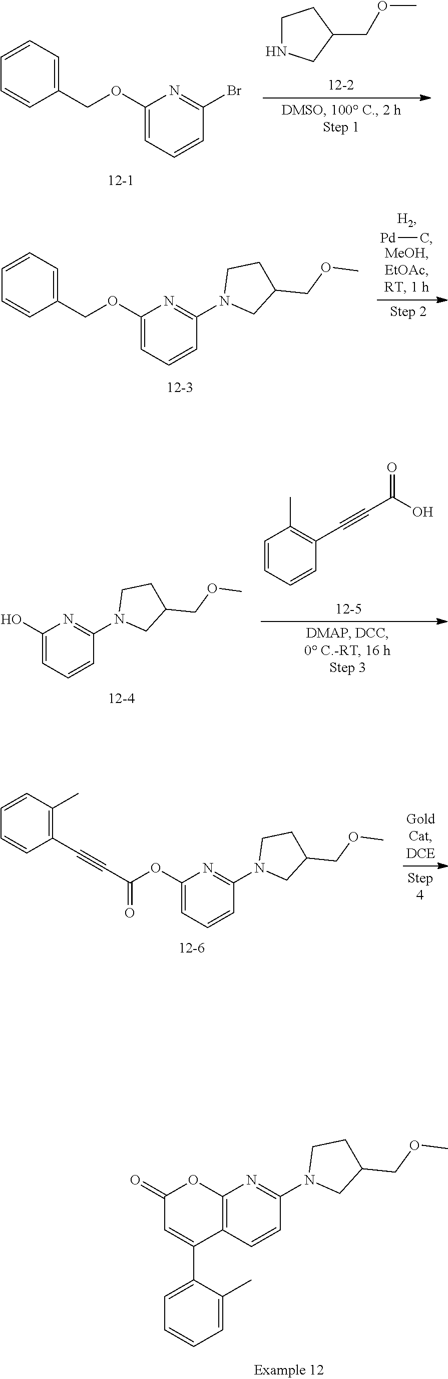

Synthesis of 2-(benzyloxy)-6-(3-(methoxymethyl)pyrrolidin-1-yl)pyridine, 12-3 [Step 1]: A solution of 2-benzyloxy-6-bromo-pyridine (12-1, 400 mg, 1.5 mmol) and 3-(methoxymethyl)pyrrolidine (12-2, 327 mg, 2.8 mmol) in DMSO (1 mL) was placed under an inert atmosphere and the reaction mixture was heated and stirred at 100° C. for 2 h. The reaction mixture was diluted with ethyl acetate, washed with cold water, and concentrated under reduced pressure. The product was purified by column chromatography to obtain 2-(benzyloxy)-6-(3-(methoxymethyl) pyrrolidin-1-yl) pyridine (12-3, 200 mg). ¹H NMR (400 MHz, DMSO-d₆) δ 7.42 (t, 3H), 7.37-7.28 (m, 3H), 5.97-5.75 (m, 2H), 5.29 (s, 2H), 3.50 (t, 2H), 3.36-3.30 (m, 2H), 3.26 (s, 3H), 3.11-3.07 (m, 2H), 2.52 (d, 1H), 2.02 (m, 1H), 1.40 (m, 1H). LC/MS: Calcd. for C₁₈H₂₂N₂O₂: 298, found [M+H]⁺=299.

Synthesis of 6-(3-(methoxymethyl)pyrrolidin-1-yl)pyridin-2-ol, 12-4 [Step 2]: A solution of 2-(benzyloxy)-6-(3-(methoxymethyl) pyrrolidin-1-yl)pyridine (12-3, 350 mg, 1.2 mmol) in methanol (5 mL) and ethyl acetate (5 mL) was degassed with N₂ gas and Pd—C(30 mg, 0.3 mmol) was added to the mixture, which was stirred for 2 h under a H₂ gas balloon. Then reaction mixture was filtered through a celite bed, which washed with methanol, concentrated under reduced pressure, and purified by column chromatography to afford 6-(3-(methoxymethyl)pyrrolidin-1-yl)pyridin-2-ol (12-4, 180 mg). LC/MS: Calcd. for C₁₁H₁₆N₂O₂: 208, found [M+H]⁺=209. ¹H NMR (400 MHz, DMSO-d₆) δ 10.06 (s, 1H), 7.25 (t, 1H), 5.62 (d, 2H), 3.45-3.25 (m, 5H), 3.09-3.03 (m, 3H), 2.01 (t, 2H), 1.69 (t, 2H).

Synthesis of 6-(3-(methoxymethyl)pyrrolidin-1-yl)pyridin-2-yl 3-(o-tolyl)propiolate, 12-6 [Step 3]: To stirred solution of 3-(o-tolyl)prop-2-ynoic acid (12-5, 152 mg, 0.9 mmol) and 6-(3-(methoxymethyl)pyrrolidin-1-yl)pyridin-2-ol (12-4, 180 mg, 0.9 mmol) in DCM (4 mL) under a N₂ atmosphere was added DMAP (21 mg, 0.173 mmol) followed by DCC (214 mg, 1 mmol) in DCM (2 ml) under ice cold conditions. The reaction mixture was allowed to warm to ambient temperature and stirred for 1 h. The reaction mixture was filtered through sintered glass funnel, washed with DCM (thrice), and concentrated under reduced pressure. The product was purified by column chromatography using silica gel to afford 6-(3-(methoxymethyl) pyrrolidin-1-yl) pyridin-2-yl 3-(o-tolyl)propiolate (12-6, 60 mg). LC/MS: Calcd. for C₂₁H₂₂N₂O₃: 350, found [M+H]⁺=351.

Synthesis of 7-(3-(methoxymethyl)pyrrolidin-1-yl)-4-(o-tolyl)-2H-pyrano[2,3-b]pyridin-2-one, Example 12 [Step 4]: A solution of 6-(3-(methoxymethyl)pyrrolidin-1-yl)pyridin-2-yl 3-(o-tolyl)propiolate (12-6, 60 mg, 0.2 mmol) in DCE (2 mL) was degassed with nitrogen for 10 min. To the solution was added (acetonitrile)[(2-biphenyl)di-tert-butylphosphine]gold(I) hexafluoroantimonate (Gold Cat.) (13 mg, 0.02 mmol) and the reaction mixture was stirred at 40° C. for 16 h. The reaction mixture was filtered through a celite bed, which was washed with DCM (thrice). The filtrate was concentrated under reduced pressure and the product was purified by prep-HPLC and lyophilized to obtain 7-(3-(methoxymethyl)pyrrolidin-1-yl)-4-(o-tolyl)-2H-pyrano[2,3-b]pyridin-2-one (Example 12, 15 mg). LC/MS: Calcd. for C₂₁H₂₂N₂O₃: 350, found [M+H]⁺=351. ¹H NMR (400 MHz, DMSO-d₆) δ 7.41-7.31 (m, 3H), 7.22 (d, 1H), 7.03 (d, 1H), 6.45 (d, 1H), 5.94 (s, 1H), 3.59 (s, 2H), 3.44-3.33 (m, 4H), 3.27 (s, 3H), 2.58 (s, 1H), 2.10 (s, 3H), 2.08 (d, 1H), 1.75 (s, 1H).

### Example 13: Synthesis of 7-(3-hydroxy-3-methylpyrrolidin-1-yl)-4-(o-tolyl)-2H-pyrano[2,3-b]pyridin-2-one

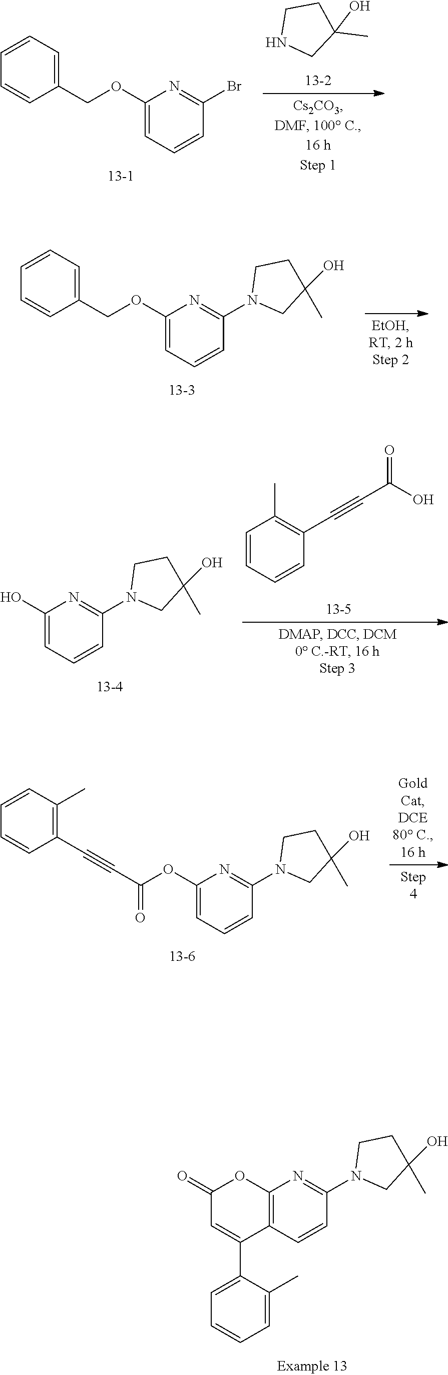

Synthesis of 1-(6-(benzyloxy)pyridin-2-yl)-3-methylpyrrolidin-3-ol, 13-3 [Step 1]: To a mixture of 2-(benzyloxy)-6-bromopyridine (13-1, 1.0 g, 3.79 mmol) and 3-methylpyrrolidin-3-ol (13-2, 0.46 g, 4.54 mmol) in DMF (15 mL) was added Cs₂CO₃ (3.7 g, 11.4 mmol) at ambient temperature and the reaction mixture was heated at 100° C. for 16 h. The reaction mixture was diluted with EtOAc, washed with water, brine, dried over anhydrous Na₂SO₄ and concentrated under reduced pressure. The product was purified by flash chromatography to afford 1-(6-(benzyloxy)pyridin-2-yl)-3-methylpyrrolidin-3-ol (13-3, 800 mg). LCMS (ESI) Calcd. for C₁₇H₂₀N₂O₂: 284, found [M+H]⁺=285. ¹H NMR (400 MHz, DMSO-d₆) δ 7.43 (m, 2H), 7.35 (m, 3H), 7.33 (m, 2H), 5.99 (d, 1H), 5.89 (d, 1H), 5.29 (s, 2H), 4.76 (s, 1H), 3.51 (t, 2H), 3.23 (m, 1H), 1.90 (m, 2H), 1.33 (s, 3H).

Synthesis of 6-(3-hydroxy-3-methylpyrrolidin-1-yl)pyridin-2-ol, 13-4 [Step 2]: To a solution of 1-(6-(benzyloxy)pyridin-2-yl)-3-methylpyrrolidin-3-ol (13-3, 500 mg, 1.76 mmol) in EtOH (20 mL) was added 10% Pd—C(140 mg) under a nitrogen atmosphere and the reaction mixture was subjected to hydrogenolysis under a hydrogen balloon pressure for 1 h at ambient temperature. The reaction mixture was filtered through a celite bed and concentrated under reduced pressure to obtain 6-(3-hydroxy-3-methyl-pyrrolidin-1-yl)pyridin-2-ol (13-4, 266 mg). The product was used directly in the next step without purification. LCMS (ESI) Calcd. for C₁₀H₁₄N₂O₂: 194, found [M+H]⁺=195. ¹H NMR (400 MHz, DMSO-d₆) δ 10.07 (br s, 1H), 7.30 (t, 1H), 5.62 (m, 2H), 4.83 (s, 1H), 3.39 (m, 2H), 3.19 (m, 2H), 1.86 (m, 2H), 1.31 (s, 3H).

Synthesis of 6-(3-hydroxy-3-methylpyrrolidin-1-yl)pyridin-2-yl 3-(o-tolyl)propiolate, 13-6 [Step 3]: To a stirred solution of 6-(3-hydroxy-3-methyl-pyrrolidin-1-yl)pyridin-2-ol (13-4 266 mg, 1.37 mmol) and 3-(o-tolyl)prop-2-ynoic acid (13-5, 219 mg, 1.37 mmol) in DCM (10 mL) was added DMAP (25 mg, 0.205 mmol) followed by a solution of DCC (450 mg, 2.19 mmol) in DCM (2 mL) at ambient temperature and stirring was continued for 16 h. The reaction mixture was filtered and the filtrate was washed with water, brine, dried over anhydrous Na₂SO₄, and concentrated under reduced pressure. The product was purified by flash chromatography to afford 6-(3-hydroxy-3-methylpyrrolidin-1-yl)pyridin-2-yl 3-(o-tolyl)propiolate (13-6, 130 mg). LCMS (ESI) Calcd. for C₂₀H₂₀N₂O₃: 336, found [M+H]⁺=337.

Synthesis of 7-(3-hydroxy-3-methylpyrrolidin-1-yl)-4-(o-tolyl)-2H-pyrano[2,3-b]pyridin-2-one, Example 13 [Step 4]: To a solution of 6-(3-hydroxy-3-methylpyrrolidin-1-yl)pyridin-2-yl 3-(o-tolyl)propiolate (13-6, 100 mg, 0.29 mmol) in DCE (2 mL) was added (acetonitrile)[(2-biphenyl)di-tert-butylphosphine]gold(I)hexafluoroantimonate (Gold Cat.) (23 mg, 0.02 mmol) under an inert atmosphere and the reaction mixture was stirred for 16 h. at ambient temperature. The reaction mixture was concentrated under reduced pressure and purified by RP prep-HPLC to afford 7-(3-hydroxy-3-methylpyrrolidin-1-yl)-4-(o-tolyl)-2H-pyrano[2,3-b]pyridin-2-one (Example 13, 15 mg). LCMS Calcd. for C₂₀H₂N₂O₃: 336, found [M+H]⁺=337. ¹H NMR (400 MHz, DMSO-d₆) δ 7.43 (m, 3H), 7.35 (m, 1H), 7.22 (d, 1H), 7.03 (d, 1H), 6.41 (s, 1H), 5.94 (s, 1H), 4.88 (s, 1H), 3.62 (m, 3H), 2.11 (s, 3H), 1.93 (br s, 2H), 1.35 (s, 3H).

### Example 14-15: Synthesis of chiral isomers of 7-(3-methylmorpholino)-4-(o-tolyl)-2H-pyrano[2,3-b]pyridin-2-one

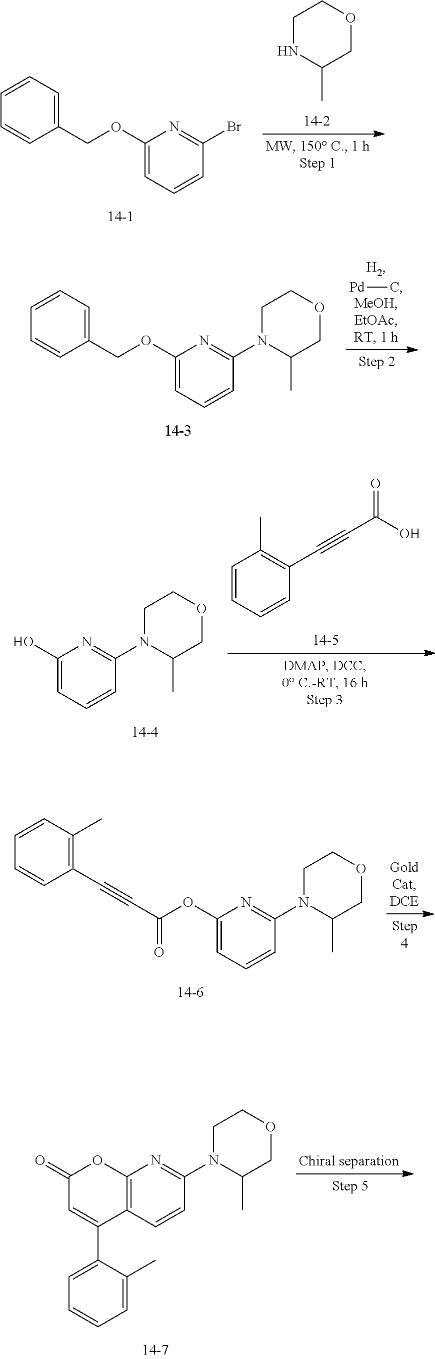

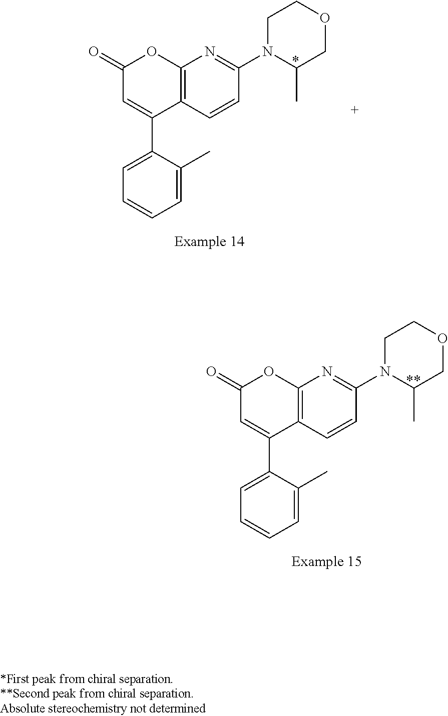 [SMILES: Cc1ccccc1-c1cc(=O)oc2nc(N3CCOCC3C)ccc12 ; Cc1ccccc1-c1cc(=O)oc2nc(N3CCOCC3C)ccc12]

Synthesis of 4-(6-(benzyloxy)pyridin-2-yl)-3-methylmorpholine, 14-3 [Step 1]: In a dried microwave tube was added 2-benzyloxy-6-bromo-pyridine (14-1, 500 mg, 1.9 mmol) and 3-methylmorpholine (14-2, 1 g, 9.5 mmol) and the reaction mixture was heated at 150° C. for 1 h. in a microwave reactor. The reaction mixture was diluted with water and extracted with EtOAc. The organic layer was washed with brine, dried over anhydrous sodium sulfate, and concentrated under reduced pressure. The product was purified by flash chromatography on silica gel to afford 4-(6-(benzyloxy)pyridin-2-yl)-3-methylmorpholine (14-3, 300 mg). LC/MS: Calcd. for C₁₇H₂₀N₂O₂: 284, found [M+H]⁺=285. ¹H NMR (400 MHz, DMSO-d₆) δ 7.45 (t, 1H), 7.41-7.28 (m, 5H), 6.24 (d, 1H), 6.08 (d, 1H), 5.27 (q, 2H), 4.25 (s, 1H), 3.92 (d, 1H), 3.89-3.67 (m, 2H), 3.59 (s, 1H), 3.44 (t, 1H), 3.00 (t, 1H), 1.04 (d, 3H).

Synthesis of 6-(3-methylmorpholino)pyridin-2-ol, 14-4 [Step 2]: To a degassed solution of 4-(6-(benzyloxy)pyridin-2-yl)-3-methylmorpholine (14-3, 300 mg, 1.0 mmol) in methanol (2 mL) and ethyl acetate (2 mL) was added 10% Pd/C (30 mg, 1.0 mmol). The reaction mixture was purged with argon for 5 min. and subjected to hydrogenolysis using a hydrogen balloon for 16 h. The reaction mixture was filtered through a celite bed and the filtrate was concentrated to obtain 6-(3-methylmorpholino)pyridin-2-ol (14-4, 170 mg). LC/MS: Calcd. for C₁₀H₁₄N₂O₂: 194, found [M+H]⁺=195. ¹H NMR (400 MHz, DMSO-d₆) δ 10.09 (s, 1H), 7.34 (t, 1H), 6.01 (s, 1H), 5.83 (d, 1H), 4.18 (s, 1H), 3.89 (d, 1H), 3.69-3.66 (m, 2H), 3.60-3.57 (m, 1H), 3.43 (t, 1H), 2.99 (t, 1H), 1.07 (d, 3H).

Synthesis of 6-(3-methylmorpholino)pyridin-2-yl 3-(o-tolyl)propiolate, 14-6 [Step 3]: To a stirred solution of 6-(3-methylmorpholino)pyridin-2-ol (14-4, 170 mg, 0.8 mmol) and 3-(o-tolyl)prop-2-ynoic acid (14-5, 155 mg, 0.9 mmol) in DCM (4 mL) was added DMAP (25 mg, 0.2 mmol) followed by a solution of DCC (220 mg, 1.0 mmol) in DCM (2 mL) dropwise at 0° C. and the reaction mixture was stirred at ambient temperature for 2 h. The reaction mixture was diluted with water and extracted with EtOAc. The organic layer was washed with brine, dried over anhydrous Na₂SO₄, and concentrated under reduced pressure. The product was purified by flash column chromatography by using silica gel to afford 6-(3-methylmorpholino)pyridin-2-yl-3-(o-tolyl)propiolate (14-6, 100 mg). LC/MS: Calcd. for C₂₀H₂₀N₂O₃: 336, found [M+H]⁺=337. ¹H NMR (400 MHz, DMSO-d₆) δ 7.71 (t, 1H), 7.62 (d, 1H), 7.49 (t, 1H), 7.39 (d, 1H), 7.31 (t, 1H), 6.73 (d, 1H), 6.52 (d, 1H), 4.25 (d, 1H), 3.90 (d, 1H), 3.80 (d, 1H), 3.41-3.68 (m, 1H), 3.60-3.57 (m, 1H), 3.43 (t, 1H), 3.06 (t, 1H), 2.37 (s, 3H), 1.11 (d, 3H).

Synthesis of 7-(3-methylmorpholino)-4-(o-tolyl)-2H-pyrano[2,3-b]pyridin-2-one, 14-7 [Step 4]: To a degassed solution of 6-(3-methylmorpholino)pyridin-2-yl 3-(o-tolyl)propiolate (14-6, 140 mg, 0.41 mmol) in DCE (4 mL) was added (acetonitrile)[(2-biphenyl)di-tert-butylphosphine]gold(I)hexafluoroantimonate (Gold Cat.) (19 mg, 0.03 mmol) and the reaction mixture was stirred at 40° C. for 16 h. The reaction mixture was filtered through a celite bed, which was washed with DCM (thrice). The organic layer was washed with brine, dried over anhydrous Na₂SO₄, and concentrated under reduced pressure. The product was purified by flash chromatography to afford 7-(3-methylmorpholino)-4-(o-tolyl)-2H-pyrano[2,3-b]pyridin-2-one (14-7, 120 mg).

Synthesis of chiral isomers of 7-(3-methylmorpholino)-4-(o-tolyl)-2H-pyrano[2,3-b]pyridin-2-one, Example 14 and Example 15 [Step 5]: 7-(3-methylmorpholino)-4-(o-tolyl)-2H-pyrano[2,3-b]pyridin-2-one (14-7, 120 mg) was purified by chiral prep-HPLC separation and the first product isolated was 7-(3-methylmorpholino)-4-(o-tolyl)-2H-pyrano[2,3-b]pyridin-2-one (Example 14, 34 mg) as Peak 1, and the second peak as 7-(3-methylmorpholino)-4-(o-tolyl)-2H-pyrano[2,3-b]pyridin-2-one (Example 15, 36 mg) as Peak 2. The absolute stereochemistry of these compounds were not determined.

Example 14: 7-(3-methylmorpholino)-4-(o-tolyl)-2H-pyrano[2,3-b]pyridin-2-one, Peak 1: LCMS (ESI) Calcd. for C₂₀H₂₀N₂O₃: 336, found [M+H]⁺=337. ¹H NMR (400 MHz, DMSO-d₆) δ 7.42-7.37 (m, 2H), 7.35 (t, 1H), 7.21 (d, 1H), 7.09 (d, 1H), 6.75-6.73 (m, 1H), 6.02 (s, 1H), 4.40 (s, 1H), 4.00 (t, 1H), 3.93 (d, 1H), 3.74 (d, 1H), 3.62 (d, 1H) 3.58 (t, 1H), 3.29 (t, 1H), 2.12 (t, 3H), 1.19 (d, 3H).

Example 15: 7-(3-methylmorpholino)-4-(o-tolyl)-2H-pyrano[2,3-b]pyridin-2-one, Peak 2: LCMS (ESI): Calcd. for C₂₀H₂₀N₂O₃: 336, found [M+H]⁺=337. ¹H NMR (400 MHz, DMSO-d₆) δ 7.42-7.37 (m, 2H), 7.35 (t, 1H), 7.21 (d, 1H), 7.09 (d, 1H), 6.75-6.73 (m, 1H), 6.02 (s, 1H), 4.40 (s, 1H), 4.00 (t, 1H), 3.93 (d, 1H), 3.74 (d, 1H), 3.62 (d, 1H) 3.58 (t, 1H), 3.29 (t, 1H), 2.12 (t, 3H), 1.19 (d, 3H).

Chiral prep-HPLC: Chiral separation was performed on an Agilent 1200 series instrument. Column: CHIRALPAK AS-H (250×20 mm) 5μ, operating at 35° C. with a flowrate of 50 g/min. Mobile Phase: 70% CO₂+30% (0.3% isopropyl amine in methanol) at 100 bar. Diluent: methanol, acetonitrile, isopropyl amine. Sample concentration:15.7 mg/ml and loading: 17.2 mg/6.1 min. Detection at 364 nm wavelength.

### Example 16: Synthesis of 1-(2-oxo-4-(o-tolyl)-2H-pyrano[2,3-b]pyridin-7-yl)pyrrolidine-3-carboxamide

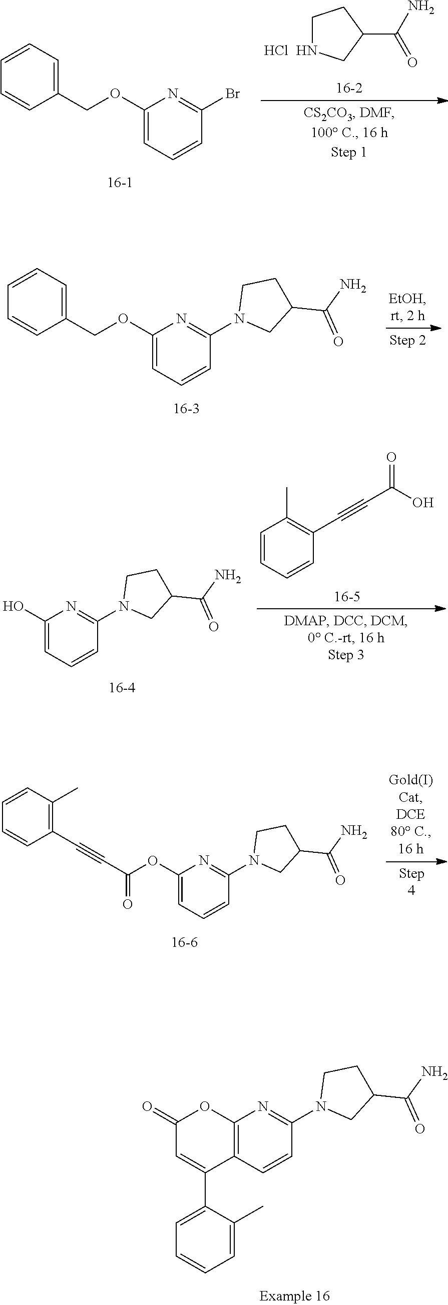

Synthesis of 1-(6-(benzyloxy)pyridin-2-yl)pyrrolidine-3-carboxamide, 16-3 [Step 1]: To a mixture of 2-(benzyloxy)-6-bromopyridine (16-1, 1.2 g, 4.54 mmol) and pyrrolidine-3-carboxamide hydrochloride (16-2, 1.03 g, 6.82 mmol) in DMF (20 mL), Cs₂CO₃ (4.4 g, 13.6 mmol) was added at rt and then heated at 100° C. for 16 h. Reaction mixture was diluted with EtOAc and washed with water, brine, dried over Na₂SO₄ and concentrated to yield a crude residue. The crude was purified by flash chromatography to afford 1-(6-benzyloxy-2-pyridyl)pyrrolidine-3-carboxamide (16-3, 650 mg). LCMS (ESI) Calcd. for C₁₇H₁₉N₃O₂: 297, found [M+H]⁺=298. ¹H NMR (400 MHz, DMSO-d₆) δ 7.41-7.30 (m, 6H), 6.95 (s, 2H), 5.98 (d, 1H), 5.94 (d, 1H), 5.30 (s, 2H), 3.57 (t, 1H), 3.42 (m, 1H), 3.40 (m, 1H), 3.32 (br s, 1H), 3.00 (m, 1H), 2.2-1.9 (m, 2H).

Synthesis of 1-(6-hydroxypyridin-2-yl)pyrrolidine-3-carboxamide, 16-4 [Step 2]: To a solution of 1-(6-benzyloxy-2-pyridyl)pyrrolidine-3-carboxamide (16-3, 300 mg, 1.01 mmol) in EtOH (5 mL) was added 10% Pd—C(125 mg) under nitrogen atmosphere and then hydrogenated under hydrogen balloon pressure for 1 h at rt. The reaction mixture was filtered through celite bed and evaporated to get 1-(6-hydroxy-2-pyridyl)pyrrolidine-3-carboxamide (16-4, 200 mg) as crude material and directly forwarded to next step without purification. LCMS (ESI) Calcd. for C₁₀H₁₃N₃O₂: 207, found [M+H]⁺=208. ¹H NMR (400 MHz, DMSO-d₆) δ 10.1 (s, 1H), 7.46 (s, 1H), 7.27 (t, 1H), 6.94 (s, 1H), 5.64 (d, 2H), 3.50-3.27 (m, 4H), 3.00 (m, 1H), 2.13-2.02 (m, 2H).

Synthesis of 6-(3-carbamoylpyrrolidin-1-yl)pyridin-2-yl 3-(o-tolyl)propiolate, 16-6 [Step 3]: To a stirred solution of 1-(6-hydroxy-2-pyridyl)pyrrolidine-3-carboxamide (16-4, 229 mg, 1.11 mmol) and 3-(o-tolyl)prop-2-ynoic acid (16-5, 177 mg, 1.11 mmol) in DCM (10 mL) was added DMAP (20 mg, 0.166 mmol) followed by a solution of DCC (365 mg, 1.77 mmol) in DCM (2 mL) and stirred at RT for 16 h. Reaction mixture was filtered and the filtrate liquid was washed with water, brine, dried over Na₂SO₄ and concentrated. The crude was purified by flash chromatography to afford 6-(3-carbamoylpyrrolidin-1-yl)pyridin-2-yl 3-(o-tolyl)propiolate (16-6, 130 mg). LCMS (ESI) Calcd. for C₂₀H₁₉N₃O₃: 349, found [M+H]⁺=350. ¹H NMR (400 MHz, DMSO-d₆) δ 7.68 (m, 2H), 7.50 (m, 2H), 7.39 (m, 1H), 7.30 (m, 1H), 6.95 (s, 1H), 6.42 (d, 2H), 4.03-3.31 (m, 4H), 3.02 (m, 1H), 2.35 (s, 3H), 1.19-1.04 (m, 2H).

Synthesis of 1-(2-oxo-4-(o-tolyl)-2H-pyrano[2,3-b]pyridin-7-yl)pyrrolidine-3-carboxamide, Example 16 [Step 4]: To a solution of 6-(3-hydroxy-3-methylpyrrolidin-1-yl)pyridin-2-yl 3-(o-tolyl)propiolate (16-6, 100 mg, 0.28 mmol) in DCE (2 mL) was added (acetonitrile)[(2-biphenyl)di-tert-butylphosphine]gold(I) hexafluoroantimonate (22 mg, 0.02 mmol) under inert atmosphere and stirred for 16 h. at RT. Volatiles were removed under reduced pressure and purified by RP prep-HPLC purification to afford 1-(2-oxo-4-(o-tolyl)-2H-pyrano[2,3-b]pyridin-7-yl)pyrrolidine-3-carboxamide (Example 16, 35 mg). LCMS: Calcd. for C₂₀H₁₉N₃O₃: 349, found [M+H]⁺=350. ¹H NMR (400 MHz, DMSO-d₆) δ 7.41-7.30 (m, 3H), 7.20-7.14 (m, 2H), 6.45 (d, 1H), 5.94 (s, 1H), 3.68-3.34 (m, 4H), 3.21-3.17 (m, 1H), 2.33-3.23 (m, 2H), 2.15 (s, 3H).

### Example 17: Synthesis of 7-(3,3-difluoropyrrolidin-1-yl)-4-(o-tolyl)-2H-pyrano[2,3-b]pyridin-2-one

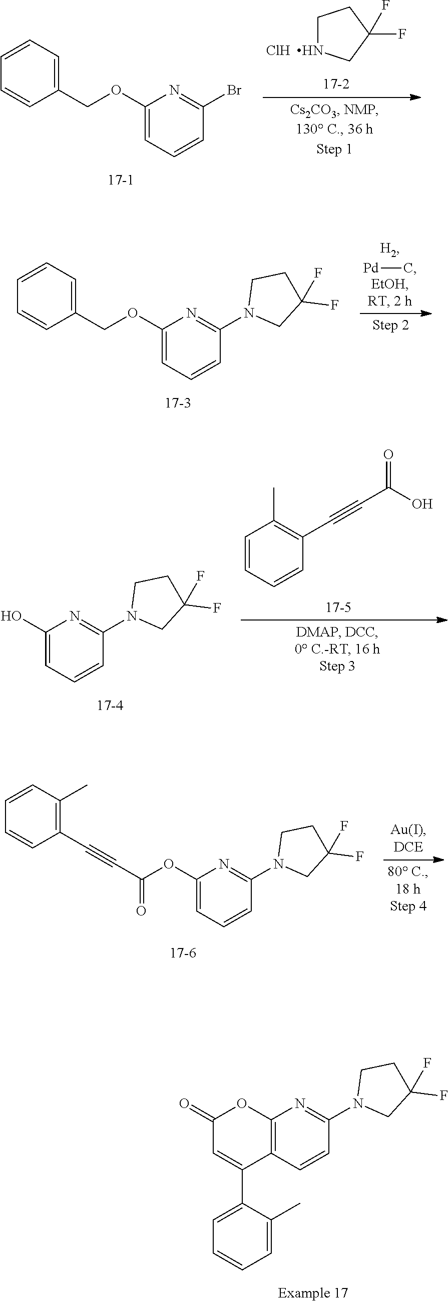

Synthesis of 2-(benzyloxy)-6-(3,3-difluoropyrrolidin-1-yl)pyridine, 17-3 [Step 1]: To a stirred solution of 2-benzyloxy-6-bromo-pyridine (17-1, 1.0 g, 3.79 mmol) and 3,3-difluoropyrrolidine hydrochloride (17-2, 0.87 g, 6.06 mmol) in NMP was added Cs₂CO₃ (4.93 g, 15.1 mmol), and the mixture was heated at 130° C. for 36 h. Then the mixture was diluted with water and the aqueous layer was extracted with ethyl acetate. The organic part was dried over Na₂SO₄, filtered, and concentrated in rotary evaporator. The crude was purified through combi flash chromatography to afford 2-benzyloxy-6-(3,3-difluoropyrrolidin-1-yl)pyridine (17-3, 470 mg). LCMS (ESI): Calcd. for C₁₆H₁₆F₂N₂O: 290, found [M+H]⁺=291.

Synthesis of 6-(3,3-difluoropyrrolidin-1-yl)pyridin-2-ol, 17-4 [Step 2]: To a stirred solution of 2-benzyloxy-6-(3,3-difluoropyrrolidin-1-yl)pyridine (17-3, 430 mg, 1.48 mmol) in ethanol was purged with argon for 5 min. Pd/C (80 mg, 0.148 mmol) was added and the mixture was hydrogenated using H₂ balloon for 2 h. The reaction mixture was filtered through a celite bed and concentrated to afford 6-(3,3-difluoropyrrolidin-1-yl)pyridin-2-ol as a crude material (17-4, 220 mg). The crude was used for the next step. LCMS (ESI): Calcd. for C₉H₁₀F₂N₂O: 200, found [M+H]⁺=201.

Synthesis of 6-(3,3-difluoropyrrolidin-1-yl)pyridin-2-yl 3-(o-tolyl)propiolate, 17-6 [Step 3]: To a stirred solution of 3-(o-tolyl)prop-2-ynoic acid (17-5, 176 mg, 1.10 mmol) and 6-(3,3-difluoropyrrolidin-1-yl)pyridin-2-ol (17-4, 200 mg, 0.999 mmol) in DCM was added DMAP (24 mg, 0.200 mmol). The mixture was cooled to 0° C. and slowly DCC (247 mg, 1.20 mmol) in DCM was added to the mixture and stirred at room temperature for 16 h. The reaction was diluted with DCM filtered, washed with water and brine. Organic layer was dried over Na₂SO₄, filtered and concentrated. The crude was purified through column chromatography to afford [6-(3,3-difluoropyrrolidin-1-yl)-2-pyridyl]3-(o-tolyl)prop-2-ynoate (17-6, 211 mg). LCMS (ESI): Calcd. for C₁₉H₁₆F₂N₂O₂: 342, found [M+H]⁺=343.

Synthesis of 7-(3,3-difluoropyrrolidin-1-yl)-4-(o-tolyl)-2H-pyrano[2,3-b]pyridin-2-one, Example 17 [Step 4]: A stirred solution of [6-(3,3-difluoropyrrolidin-1-yl)-2-pyridyl]3-(o-tolyl)prop-2-ynoate (17-6, 100 mg, 0.292 mmol) in DCE (2 mL) was degassed with argon for 5 min. Then Gold Catalyst (23 mg, 0.0292 mmol) was added and the mixture was stirred for 18 h. at 80° C. Then the mixture was diluted with 50 mL DCM and passed through celite bed, concentrated and submitted for RP prep-HPLC purification to afford 7-(3,3-difluoropyrrolidin-1-yl)-4-(o-tolyl)pyrano[2,3-b]pyridin-2-one (Example 17, 71 mg). LCMS (ESI) Calcd. for C₁₉H₁₆F₂N₂O₂: 342, found [M+H]⁺=343. ¹H NMR (400 MHz, DMSO-d₆) δ 7.37 (t, 1H), 7.32-7.25 (m, 2H), 7.18-7.13 (m, 2H), 6.19 (d, 1H), 6.05 (s, 1H), 3.92 (t, 2H), 3.78 (t, 2H), 3.57-3.46 (m, 2H), 2.14 (s, 3H).

### Example 18: Synthesis of 7-(3-fluoropyrrolidin-1-yl)-4-(o-tolyl)-2H-pyrano[2,3-b]pyridin-2-one

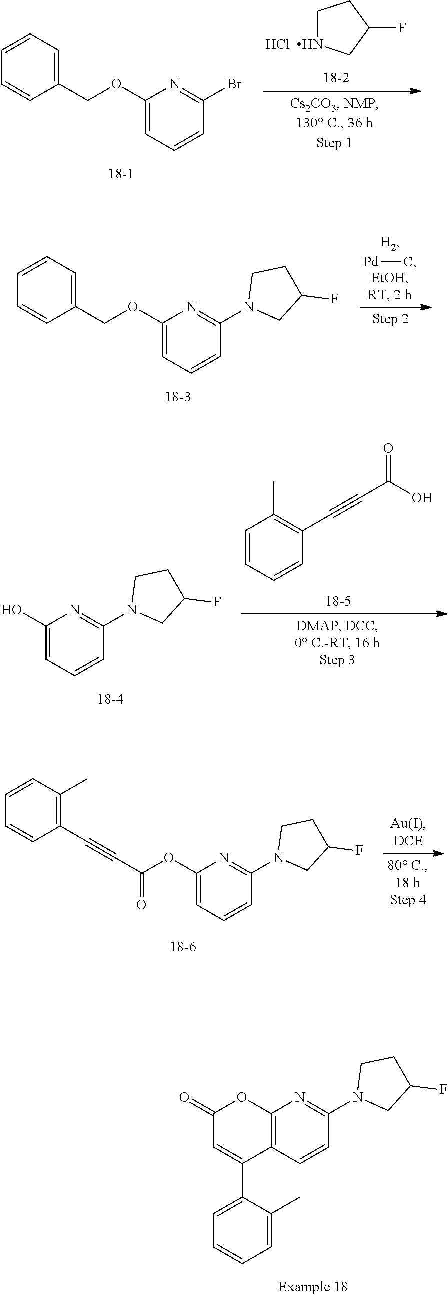

Synthesis of 2-(benzyloxy)-6-(3-fluoropyrrolidin-1-yl)pyridine, 18-3 [Step 1]: To a stirred solution of 3-fluoropyrrolidine hydrochloride (18-2, 456 mg, 3.63 mmol) and 2-benzyloxy-6-bromo-pyridine (18-1, 600 mg, 2.27 mmol) in NMP was added cesium carbonate (2961 mg, 9.09 mmol) and the mixture was heated at 130° C. for 36 h. Then the mixture was diluted with water and the aqueous layer was extracted with ethyl acetate. The organic part was dried over Na₂SO₄, filtered and concentrated in rotary evaporator. The crude was purified through combiflash chromatography to afford 2-benzyloxy-6-(3-fluoropyrrolidin-1-yl)pyridine (18-3, 440 mg). LCMS (ESI): Calcd. for C₁₆H₁₇FN₂O: 272, found [M+H]⁺=273. ¹H NMR (400 MHz, CDCl₃) δ 7.44 (d, 2H), 7.39-7.30 (m, 4H), 6.09 (d, 1H), 5.92 (d, 1H), 5.43-5.29 (m, 3H), 3.88-3.78 (m, 1H), 3.71-3.61 (m, 2H), 3.59-3.52 (m, 1H), 2.42-2.32 (m, 1H), 2.21-2.06 (m, 1H).

Synthesis of 6-(3-fluoropyrrolidin-1-yl)pyridin-2-ol, 18-4 [Step 2]: To a stirred solution of 2-benzyloxy-6-(3-fluoropyrrolidin-1-yl)pyridine (18-3, 390 mg, 1.43 mmol) in ethanol (15 mL) was purged with argon for 5 min. Then in to this solution Pd/C (80 mg, 0.143 mmol) was added and the mixture was hydrogenated using a H₂ balloon for 2 h. at RT. Reaction mixture was filtered through a celite bed and concentrated to afford 6-(3-fluoropyrrolidin-1-yl)pyridin-2-ol as a crude material (18-4, 250 mg). The crude was used for next step. LCMS (ESI): Calcd. for C₉H₁₁FN₂O: 182, found [M+H]⁺=183.

Synthesis of 6-(3-fluoropyrrolidin-1-yl)pyridin-2-yl 3-(o-tolyl)propiolate, 18-6 [Step 3]: To a stirred solution of 3-(o-tolyl)prop-2-ynoic acid (18-5, 264 mg, 1.65 mmol) and 6-(3-fluoropyrrolidin-1-yl)pyridin-2-ol (18-4, 250 mg, 1.37 mmol) in DCM was added DMAP (34 mg, 0.274 mmol). The mixture was cooled to 0° C. and slowly DCC (396 mg, 1.92 mmol) in 2 mL DCM was added to the mixture and stirred at room temperature for 16 h. The reaction was diluted with DCM, filtered, washed with water and brine. Organic layer was dried over Na₂SO₄, filtered and concentrated. The crude was purified by column chromatography to afford [6-(3-fluoropyrrolidin-1-yl)-2-pyridyl]3-(o-tolyl)prop-2-ynoate (18-6, 340 mg). LCMS (ESI): Calcd. for C₁₉H₁₇FN₂O₂: 324, found [M+H]⁺=325. ¹H NMR (400 MHz, CDCl₃) δ 7.57-7.51 (m, 2H), 7.35 (t, 1H), 7.24-7.12 (m, 2H), 6.39 (d, 1H), 6.28 (d, 1H), 5.35 (d, 1H), 3.87-3.77 (m, 1H), 3.69-3.52 (m, 3H), 2.47 (s, 3H), 2.41-2.32 (m, 1H), 2.21-2.03 (m, 1H).

Synthesis of 7-(3-fluoropyrrolidin-1-yl)-4-(o-tolyl)-2H-pyrano[2,3-b]pyridin-2-one, Example 18 [Step 4]: A stirred solution of [6-(3-fluoropyrrolidin-1-yl)-2-pyridyl]3-(o-tolyl)prop-2-ynoate (18-6, 110 mg, 0.339 mmol) in DCE (2 mL) was degassed with argon for 5 min. Then Gold Catalyst (26 mg, 0.0339 mmol) was added and the mixture was stirred for 18 h. at 80° C. Then the mixture was diluted with 50 ml DCM and passed through celite bed, concentrated and submitted for RP prep-HPLC purification to afford 7-(3-fluoropyrrolidin-1-yl)-4-(o-tolyl)pyrano[2,3-b]pyridin-2-one (Example 18, 90 mg). LCMS (ESI) Calcd. for C₁₉H₁₇FN₂O₂: 324, found [M+H]⁺=325. ¹H NMR (400 MHz, DMSO-d₆) δ 7.44-7.38 (m, 2H), 7.34 (t, 1H), 7.22 (d, 1H), 7.07 (d, 1H), 6.49 (d, 1H), 5.99 (s, 1H), 5.47 (d, 1H), 3.74-3.62 (m, 3H), 3.51-3.47 (m, 1H), 2.30-2.25 (m, 2H), 2.11 (s, 3H).

### Example 19: Synthesis of 7-(azetidin-1-yl)-4-(o-tolyl)-2H-pyrano[2,3-b]pyridin-2-one

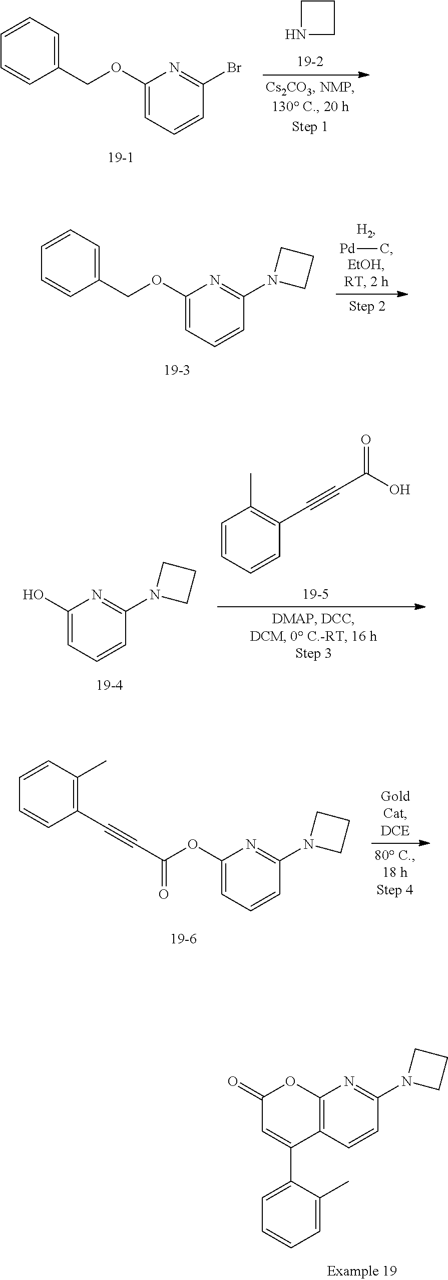

Synthesis of 2-(azetidin-1-yl)-6-(benzyloxy)pyridine, 19-3 [Step 1]: To a stirred solution of 2-benzyloxy-6-bromo-pyridine (19-1, 600 mg, 2.3 mmol) and azetidine (19-2, 210 mg, 3.6 mmol) in NMP was added cesium carbonate (3.0 g, 9.1 mmol) and the mixture was heated at 130° C. for 20 h. Then the mixture was diluted with water and the aqueous layer was extracted with ethyl acetate. The organic part was dried over Na₂SO₄, filtered and concentrated to yield crude residue. The crude was purified through combiflash chromatography to afford 2-(azetidin-1-yl)-6-benzyloxy-pyridine (19-3, 425 mg). LCMS (ESI): Calcd. for C₁₅H₁₆N₂O: 240, found [M+H]⁺=241. ¹H NMR (400 MHz, CDCl₃) δ 7.45 (d, 2H), 7.36-7.32 (m, 3H), 7.28 (d, 1H), 6.07 (d, 1H), 5.80 (d, 1H), 5.32 (s, 2H), 3.98 (t, 4H), 3.34 (p, 2H).

Synthesis of 6-(azetidin-1-yl)pyridin-2-ol, 19-4 [Step 2]: To a stirred solution of 2-(azetidin-1-yl)-6-benzyloxy-pyridine (19-3, 420 mg, 1.8 mmol) in ethanol (15 mL) was purged with argon gas for 5 min. Then in to this solution Pd/C (80 mg, 0.2 mmol) was added and the mixture was hydrogenated using a H₂ balloon for 2 h. at RT. The reaction mixture was filtered through a celite bed and concentrated to afford 6-(azetidin-1-yl)pyridin-2-ol (19-4, 245 mg). The crude was used for next step. LCMS (ESI): Calcd. for C₈H₁₀N₂O:150, found [M+H]⁺=151.

Synthesis of 6-(azetidin-1-yl)pyridin-2-yl 3-(o-tolyl)propiolate, 19-6 [Step 3]: To a stirred solution of 6-(azetidin-1-yl)pyridin-2-ol (19-4, 250 mg, 1.7 mmol) and 3-(o-tolyl)prop-2-ynoic acid (19-5, 320 mg, 2.0 mmol) in DCM was added DMAP (40 mg, 0.3 mmol). The mixture was cooled to 0° C. and slowly DCC (515 mg, 2.5 mmol) in 2 mL DCM was added to the mixture and stirred at room temperature for overnight. The reaction was diluted with DCM, filtered, washed with water and brine. The organic layer was dried over Na₂SO₄, filtered and concentrated. The crude was purified by column chromatography to afford 6-(azetidin-1-yl)pyridin-2-yl 3-(o-tolyl)propiolate (19-6, 325 mg). LCMS (ESI) Calcd. for C₁₈H₁₆N₂O₂: 292, found [M+H]⁺=293.

Synthesis of 7-(azetidin-1-yl)-4-(o-tolyl)-2H-pyrano[2,3-b]pyridin-2-one, Example 19 [Step 4]: A stirred solution of 6-(azetidin-1-yl)pyridin-2-yl 3-(o-tolyl)propiolate (19-6, 100 mg, 0.3 mmol) in DCE (2 mL) was degassed with argon gas for 5 min. Then (acetonitrile)[(2-biphenyl)di-tert-butylphosphine]gold(I) hexafluoroantimonate (25 mg, 0.03 mmol) was added and the mixture was stirred for 18 h. at 80° C. Then the mixture was diluted with DCM and passed through celite bed, concentrated and the residue was submitted for RP prep-HPLC purification to afford 7-(azetidin-1-yl)-4-(o-tolyl)pyrano[2,3-b]pyridin-2-one (Example 19, 86 mg). LCMS (ESI) Calcd. for C₁₈H₁₆N₂O₂: 292, found [M+H]⁺=293. ¹H NMR (400 MHz, DMSO-d₆) δ 7.43-7.31 (m, 3H), 7.20 (d, 1H), 7.03 (d, 1H), 6.25 (d, 1H), 5.96 (s, 1H), 4.08 (t, 4H), 2.37 (p, 2H), 2.10 (br s, 3H).

### Example 20: Synthesis of 7-(3-methoxyazetidin-1-yl)-4-(o-tolyl)-2H-pyrano[2,3-b]pyridin-2-one

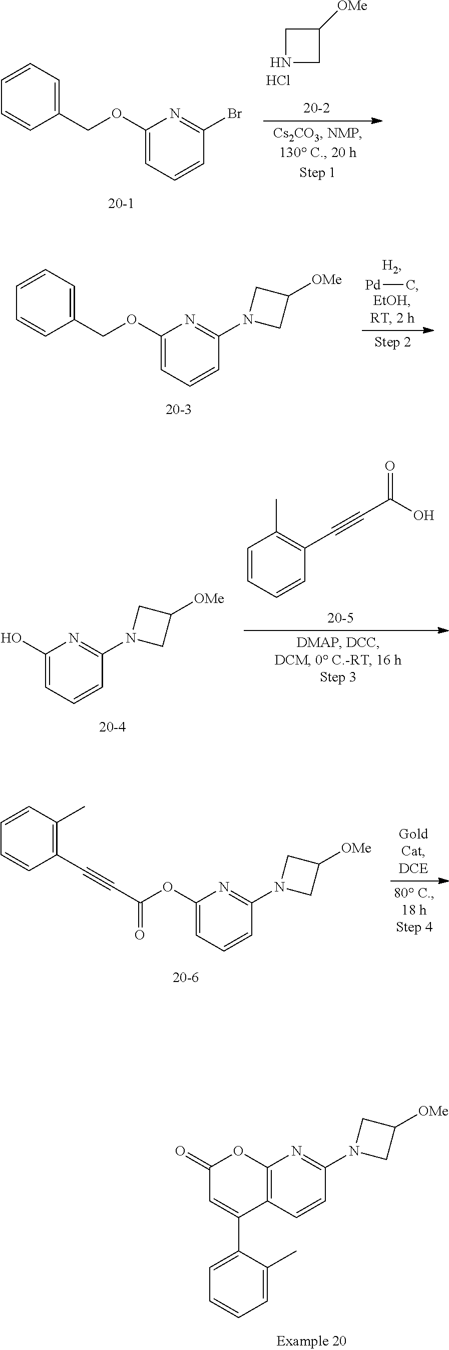

Synthesis of 2-(benzyloxy)-6-(3-methoxyazetidin-1-yl)pyridine, 20-3 [Step 1]: To a stirred solution of 2-benzyloxy-6-bromo-pyridine (20-1, 500 mg, 1.9 mmol) and 3-methoxyazetidine hydrochloride (20-2, 370 mg, 3.0 mmol) in NMP was added cesium carbonate (2.5 g, 7.6 mmol) and the mixture was heated at 130° C. for 20 h. Then the mixture was diluted with water and the aqueous layer was extracted with ethyl acetate. The organic part was dried over Na₂SO₄, filtered and concentrated under reduced pressure. The crude was purified through combiflash chromatography to afford 2-benzyloxy-6-(3-methoxyazetidin-1-yl)pyridine (20-3, 460 mg). LCMS (ESI): Calcd. for C₁₆H₁₈N₂O₂: 270, found [M+H]⁺=271. ¹H NMR (400 MHz, CDCl₃) δ 7.44 (d, 2H), 7.37-7.32 (m, 3H), 7.28 (d, 1H), 6.09 (d, 1H), 5.84 (d, 1H), 5.32 (s, 2H), 4.34-4.29 (m, 1H), 4.16 (t, 2H), 3.85-3.82 (m, 2H), 3.33 (s, 3H).

Synthesis of 6-(3-methoxyazetidin-1-yl)pyridin-2-ol, 20-4 [Step 2]: To a stirred solution of 2-benzyloxy-6-(3-methoxyazetidin-1-yl)pyridine (20-3, 450 mg, 1.7 mmol) in ethanol (15 mL), was purged argon gas for 5 min. Then in to this solution 10% Pd/C (80 mg, 0.2 mmol) was added and the mixture was hydrogenated using a H₂ balloon for 2 h. at RT. The reaction mixture was filtered through a celite bed and concentrated to afford 6-(3-methoxyazetidin-1-yl)pyridin-2-ol (20-4, 280 mg). LCMS (ESI): Calcd. for C₉H₁₂N₂O₂: 180, found [M+H]⁺=181.

Synthesis of 6-(3-methoxyazetidin-1-yl)pyridin-2-yl 3-(o-tolyl)propiolate, 20-6 [Step 3]: To a stirred solution of 6-(3-methoxyazetidin-1-yl)pyridin-2-ol (20-4, 280 mg, 1.6 mmol) and 3-(o-tolyl)propiolic acid (20-5, 300 mg, 1.9 mmol) in DCM was added DMAP (40 mg, 0.3 mmol). The mixture was cooled to 0° C. and DCC (480 mg, 2.3 mmol) in 2 mL DCM was added to the mixture and stirred at room temperature for overnight. The reaction was diluted with DCM, filtered, washed with water and brine. The organic layer was dried over Na₂SO₄, filtered and concentrated. The crude was purified by column chromatography to afford 6-(3-methoxyazetidin-1-yl)pyridin-2-yl 3-(o-tolyl)propiolate (20-6, 390 mg). LCMS (ESI) Calcd. for C₁₉H₁₈N₂O₃: 322, found [M+H]⁺=323. ¹H NMR (400 MHz, CDCl₃) δ 7.56-7.51 (m, 2H), 7.35 (t, 1H), 7.24 (d, 1H), 7.19 (t, 1H), 6.42 (d, 1H), 6.19 (d, 1H), 4.32-4.29 (m, 1H), 4.21 (t, 2H), 3.92-3.89 (m, 2H), 3.32 (s, 3H), 2.48 (s, 3H).

Synthesis of 7-(3-methoxyazetidin-1-yl)-4-(o-tolyl)-2H-pyrano[2,3-b]pyridin-2-one, Example 20 [Step 4]: A stirred solution of 6-(3-methoxyazetidin-1-yl)pyridin-2-yl 3-(o-tolyl)propiolate (20-6, 120 mg, 0.4 mmol) in DCE (2 mL) was degassed with argon gas for 5 min. Then (acetonitrile)[(2-biphenyl)di-tert-butylphosphine]gold(I) hexafluoroantimonate (30 mg, 0.04 mmol) was added and the mixture was stirred for 18 h. at 80° C. Then the mixture was diluted with DCM and passed through celite bed and concentrated to yield a sticky gum. The crude was purified by RP prep-HPLC purification and lyophilized to afford 7-(3-methoxy azetidin-1-yl)-4-(o-tolyl)-2H-pyrano[2,3-b]pyridin-2-one (Example 20, 91 mg). LCMS (ESI) Calcd. for C₁₉H₁₈N₂O₃: 322, found [M+H]⁺=323. ¹H NMR (400 MHz, DMSO-d₆) δ 7.43-7.31 (m, 3H), 7.21 (d, 1H), 7.05 (d, 1H), 6.30 (d, 1H), 5.99 (s, 1H), 4.35-4.33 (m, 1H), 4.26 (t, 2H), 3.91-3.88 (m, 2H), 3.26 (s, 3H), 2.10 (s, 3H).

### Example 21: Synthesis of 4-(2-chloro-4-fluorophenyl)-7-morpholino-2H-pyrano[2,3-b]pyridin-2-one

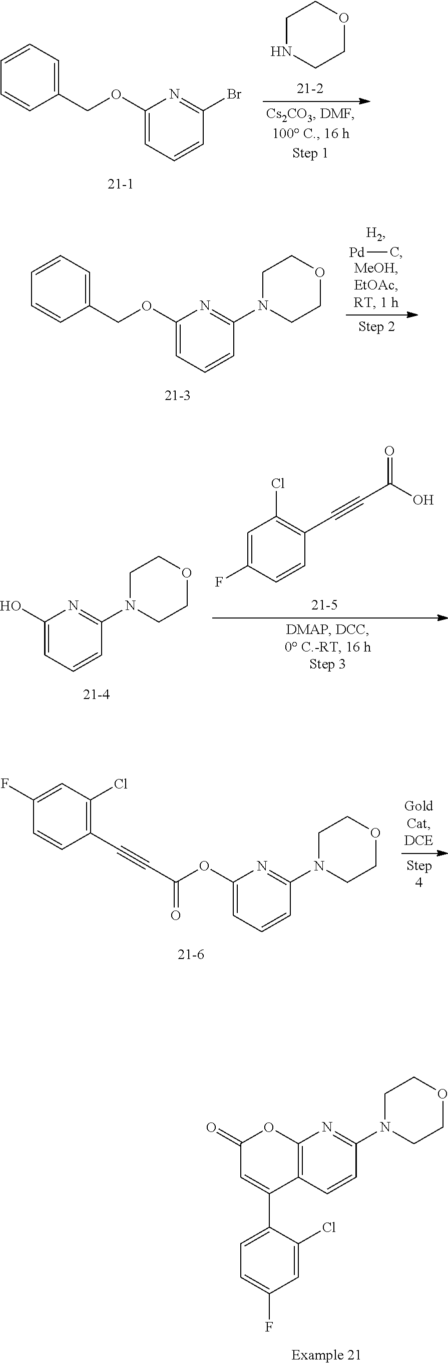

Synthesis of 4-(6-(benzyloxy)pyridin-2-yl)morpholine, 21-3 [Step 1]: To a stirred solution of 2-(benzyloxy)-6-bromopyridine (21-1, 800 mg, 3.1 mmol) in DMF (8 mL) was added Cs₂CO₃ (3.0 g, 9.1 mmol) followed by morpholine (21-2, 1.3 mL, 15.1 mmol) and stirred at 100° C. for 16 h. The reaction mixture was quenched with water and extracted with ethyl acetate. The organic layer was collected, washed with brine, dried over anhydrous sodium sulfate and concentrated under reduced pressure to get the crude. It was purified by flash column chromatography to get 4-(6-(benzyloxy)pyridin-2-yl)morpholine (21-3, 500 mg). LCMS (ESI) Calcd. for C₁₆H₁₈N₂O₂: 270; [M+H]⁺=271. ¹H NMR (400 MHz, DMSO-d₆) δ 7.49 (t, 1H), 7.42 (d, 2H), 7.37 (t, 2H), 7.30 (d, 1H), 6.32 (d, 1H), 6.13 (d, 1H), 5.30 (s, 2H), 3.68 (t, 4H), 3.41 (t, 4H).

Synthesis of 6-morpholinopyridin-2-ol, 21-4 [Step 2]: To a stirred solution of 4-(6-(benzyloxy)pyridin-2-yl)morpholine (21-3, 200 mg, 0.8 mmol) in methanol (2 mL): ethyl acetate (2 mL) mixture was purged with argon gas for 5 min. Then into this purged solution was added Pd/C (10%) (40 mg, 0.03 mmol) and was hydrogenated using hydrogen balloon at RT for 1 h. The reaction mixture was filtered through celite bed and washed with EtOAc. The filtrate was concentrated to get 6-morpholinopyridin-2-ol (21-4, 120 mg). LCMS (ESI) Calcd. for C₉H₁₂N₂O₂: 180; [M+H]⁺=181.

Synthesis of 6-morpholinopyridin-2-yl 3-(2-chloro-4-fluorophenyl)propiolate, 21-6 [Step 3]: To a stirred solution of 6-morpholinopyridin-2-ol (21-4, 200 mg, 1.1 mmol) and 3-(2-chloro-4-fluorophenyl)propiolic acid (21-5, 220 mg, 1.1 mmol) in DCM (1 mL) was added DMAP (20 mg, 0.2 mmol) followed by DCC (365 mg, 1.8 mmol) portion wise at 0° C. and stirred at RT for 16 h. The reaction mixture was filtered and the filtrate was evaporated under reduced pressure to get the crude residue. The crude was purified by flash column chromatography to afford 6-morpholinopyridin-2-yl 3-(2-chloro-4-fluorophenyl)propiolate (21-6, 60 mg). LCMS (ESI) Calcd. for C₁₈H₁₄ClFN₂O₃: 361; [M+H]⁺=361. ¹H NMR (400 MHz, DMSO-d₆) δ 7.95 (t, 1H), 7.73 (d, 2H), 7.40 (s, 1H), 6.82 (d, 1H), 6.57 (d, 1H), 3.67 (br s, 4H), 3.42 (br s, 4H).

Synthesis of 4-(2-chloro-4-fluorophenyl)-7-morpholino-2H-pyrano[2,3-b]pyridin-2-one, Example 21 [Step 4]: To a stirred solution of 6-morpholinopyridin-2-yl 3-(2-chloro-4-fluorophenyl)propiolate (21-6, 60 mg, 0.2 mmol) in DCE (1.5 mL) was degassed with argon for 10 min. Then (acetonitrile)[(2-biphenyl)di-tert-butylphosphine]gold(I) hexafluoroantimonate (15 mg, 0.02 mmol) was added and the mixture was stirred at 80° C. for 18 h. The reaction mixture was filtered through celite bed and the filtrate was concentrated under reduced pressure to get the crude. The crude was purified by RP prep-HPLC and isolated compound was lyophilized to afford 4-(2-chloro-4-fluorophenyl)-7-morpholino-2H-pyrano[2,3-b]pyridin-2-one (Example 21, 12 mg). LCMS (ESI) Calcd. for C₁₈H₁₄ClFN₂O₃: 361, found [M+H]⁺=362. ¹H NMR (400 MHz, DMSO-d₆) δ 7.70 (dd, 1H), 7.55-7.52 (m, 1H), 7.44-7.40 (m, 1H), 7.20 (d, 1H), 6.81 (d, 1H), 6.14 (s, 1H), 3.69 (d, 4H), 3.65 (d, 4H).

### Example 22: Synthesis of N-(1-(2-oxo-4-(o-tolyl)-2H-pyrano[2,3-b]pyridin-7-yl)pyrrolidin-3-yl)acetamide

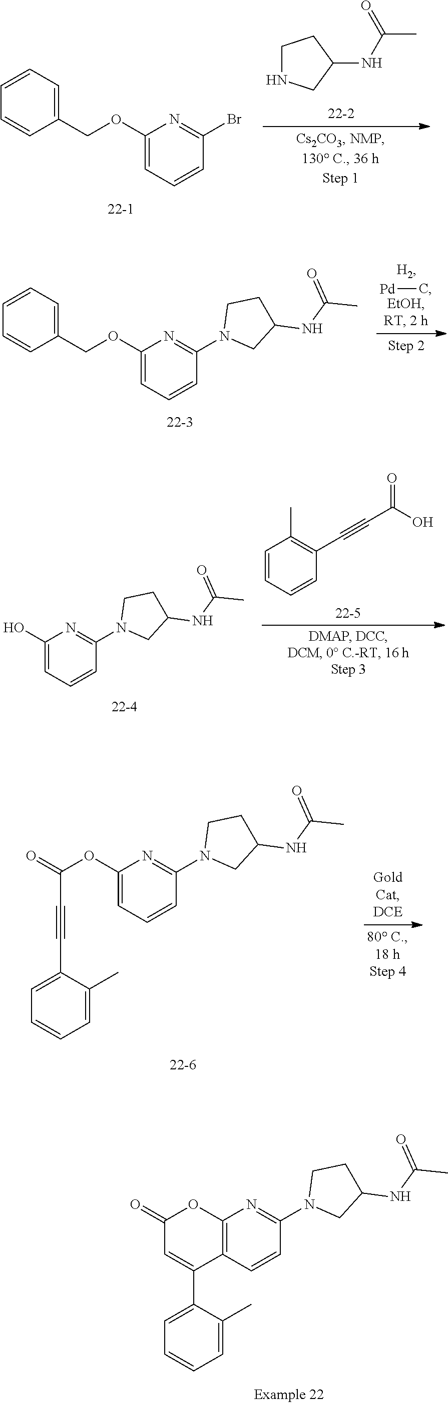

Synthesis of N-(1-(6-(benzyloxy)pyridin-2-yl)pyrrolidin-3-yl)acetamide, 22-3 [Step 1]: To a stirred solution of 2-benzyloxy-6-bromo-pyridine (22-1, 600 mg, 2.3 mmol) and N-(pyrrolidin-3-yl)acetamide (22-2, 437 mg, 3.4 mmol) in NMP was added cesium carbonate (2.9 g, 9.1 mmol). Then the mixture was heated at 130° C. for 36 h. Then the mixture was diluted with water and the aqueous layer was extracted with ethyl acetate. The organic part was dried over Na₂SO₄, filtered and concentrated in rotary evaporator. The crude was purified through combiflash chromatography to afford N-(1-(6-(benzyloxy)pyridin-2-yl)pyrrolidin-3-yl)acetamide (22-3, 442 mg). LCMS (ESI): Calcd. for C₁₈H₂₁N₃O₂: 311, found [M+H]⁺=312.

Synthesis of N-(1-(6-hydroxypyridin-2-yl)pyrrolidin-3-yl)acetamide, 22-4 [Step 2]: To a stirred solution of N-[1-(6-benzyloxy-2-pyridyl)pyrrolidin-3-yl]acetamide (22-3, 390 mg, 1.3 mmol) in ethanol (15 mL) was purged with argon for 5 min. Then in to this solution Pd/C (75 mg, 0.13 mmol) was added and the mixture was hydrogenated using a H₂ balloon at RT for 2 h. Reaction mixture was filtered through a celite bed and concentrated to afford N-(1-(6-hydroxypyridin-2-yl)pyrrolidin-3-yl)acetamide (22-4, 255 mg) as a crude material. The crude was used for next step. LCMS (ESI): Calcd. for C₁₁H₁₅N₃O₂: 221, found [M+H]⁺=222.

Synthesis of 6-(3-acetamidopyrrolidin-1-yl)pyridin-2-yl 3-(o-tolyl)propiolate, 22-6 [Step 3]: To a stirred solution of N-(1-(6-hydroxypyridin-2-yl)pyrrolidin-3-yl)acetamide (22-4, 200 mg, 0.9 mmol) and 3-(o-tolyl)prop-2-ynoic acid (22-5, 174 mg, 1.1 mmol) in DCM was added DMAP (26 mg, 0.2 mmol). The mixture was cooled to 0° C. and slowly DCC (280 mg, 1.4 mmol) in 2 mL DCM was added into the mixture and stirred at room temperature for 16 h. The reaction mixture was diluted with DCM, filtered, and washed with water and brine. Organic layer was dried over Na₂SO₄, filtered, and concentrated. The crude was purified by column chromatography to afford 6-(3-acetamidopyrrolidin-1-yl)pyridin-2-yl 3-(o-tolyl)propiolate (22-6, 225 mg). LCMS (ESI) Calcd. for C₂₁H₂₁N₃O₃: 363, found [M+H]⁺=364.

Synthesis of N-(1-(2-oxo-4-(o-tolyl)-2H-pyrano[2,3-b]pyridin-7-yl)pyrrolidin-3-yl)acetamide, Example 22 [Step 4]: A stirred solution of 6-(3-acetamidopyrrolidin-1-yl)pyridin-2-yl 3-(o-tolyl)propiolate (22-6, 100 mg, 0.3 mmol) in DCE (2 mL) was degassed with argon for 5 min. Then (acetonitrile)[(2-biphenyl)di-tert-butylphosphine]gold(I) hexafluoroantimonate (21 mg, 0.03 mmol) was added and the mixture was stirred at 80° C. for 18 h. Then the mixture was diluted with DCM and passed through celite bed, concentrated and submitted for RP prep-HPLC purification to afford N-(1-(2-oxo-4-(o-tolyl)-2H-pyrano[2,3-b]pyridin-7-yl)pyrrolidin-3-yl)acetamide (Example 22, 79 mg). LCMS (ESI) Calcd. C₂₁H₂₁N₃O₃: 363, found [M+H]⁺=364. ¹H NMR (400 MHz, DMSO-d₆) δ 8.15 (d, 1H), 7.44-7.32 (m, 3H), 7.21 (d, 1H), 7.04 (d, 1H), 6.45 (d, 1H), 5.96 (s, 1H), 4.39 (bs, 1H), 3.73 (t, 1H), 3.64 (d, 1H), 3.57 (bs, 1H), 3.37 (d, 1H), 2.21 (bs, 1H), 2.14 (s, 3H), 1.95 (bs, 1H), 1.84 (s, 3H).

### Example 23-24: Synthesis of chiral isomers of 7-(3-methoxypyrrolidin-1-yl)-4-(o-tolyl)-2H-pyrano[2,3-b]pyridin-2-one

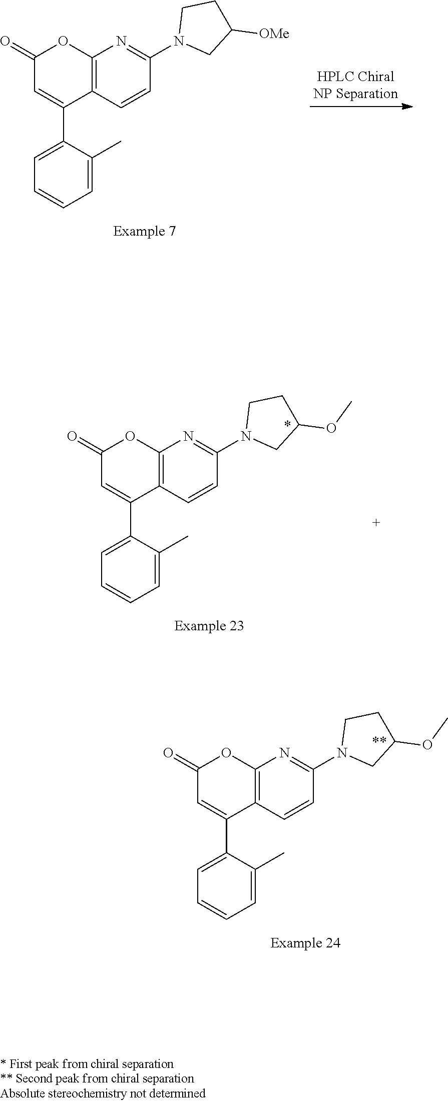 [SMILES: COC1CCN(c2ccc3c(-c4ccccc4C)cc(=O)oc3n2)C1 ; COC1CCN(c2ccc3c(-c4ccccc4C)cc(=O)oc3n2)C1 ; COC1CCN(c2ccc3c(-c4ccccc4C)cc(=O)oc3n2)C1]

Racemic 7-(3-methoxypyrrolidin-1-yl)-4-(o-tolyl)-2H-pyrano[2,3-b]pyridin-2-one (Example 7, 125 mg, 0.4 mmol) was used for HPLC chiral (NP) separation to afford 7-(3-methoxypyrrolidin-1-yl)-4-(o-tolyl)-2H-pyrano[2,3-b]pyridin-2-one (Example 23, 60 mg) as Peak 1, and 7-(3-methoxypyrrolidin-1-yl)-4-(o-tolyl)-2H-pyrano[2,3-b]pyridin-2-one (Example 24, 47 mg) as Peak 2. The absolute stereochemistries were not determined.

PREP HPLC CHIRAL METHOD: Chiral separation was done on Agilent 1200 series instrument. Column name: CHIRALPAK IG (250×21 mm) 5u. Operating at ambient temperature and flow rate is 21.0 mL/min. Mobile phase was mixture of 70% hexane, 15% DCM, and 15% ethanol, held this isocratic mixture run up to 20 min. with wavelength of 366 nm.

Example 23: 7-(3-methoxypyrrolidin-1-yl)-4-(o-tolyl)-2H-pyrano[2,3-b]pyridin-2-one, Peak 1: LCMS (ESI) Calcd. for C₂₀H₂₀N₂O₃: 336, found [M+H]⁺=337. ¹H NMR (400 MHz, DMSO-d₆) δ 4.44-7.32 (m, 3H), 7.21 (d, 1H), 7.04 (d, 1H), 6.45 (d, 1H), 5.96 (s, 1H), 4.09 (bs, 1H), 3.64-3.53 (m, 3H), 3.49 (t, 1H), 3.30 (s, 3H), 2.14-2.09 (m, 5H).

Example 24: 7-(3-methoxypyrrolidin-1-yl)-4-(o-tolyl)-2H-pyrano[2,3-b]pyridin-2-one, Peak 2: LCMS (ESI) Calcd. for C₂₀H₂₀N₂O₃: 336, found [M+H]⁺=337. ¹H NMR (400 MHz, DMSO-d₆) δ 4.44-7.32 (m, 3H), 7.22 (d, 1H), 7.04 (d, 1H), 6.45 (d, 1H), 5.96 (s, 1H), 4.09 (bs, 1H), 3.64-3.47 (m, 4H), 3.30 (s, 3H), 2.14-2.09 (m, 5H). The absolute stereochemistry was not determined.

### Example 25-26: Synthesis of chiral isomers of 4-(2-chloro-4-fluorophenyl)-7-(3-methoxypyrrolidin-1-yl)-2H-pyrano[2,3-b]pyridin-2-one

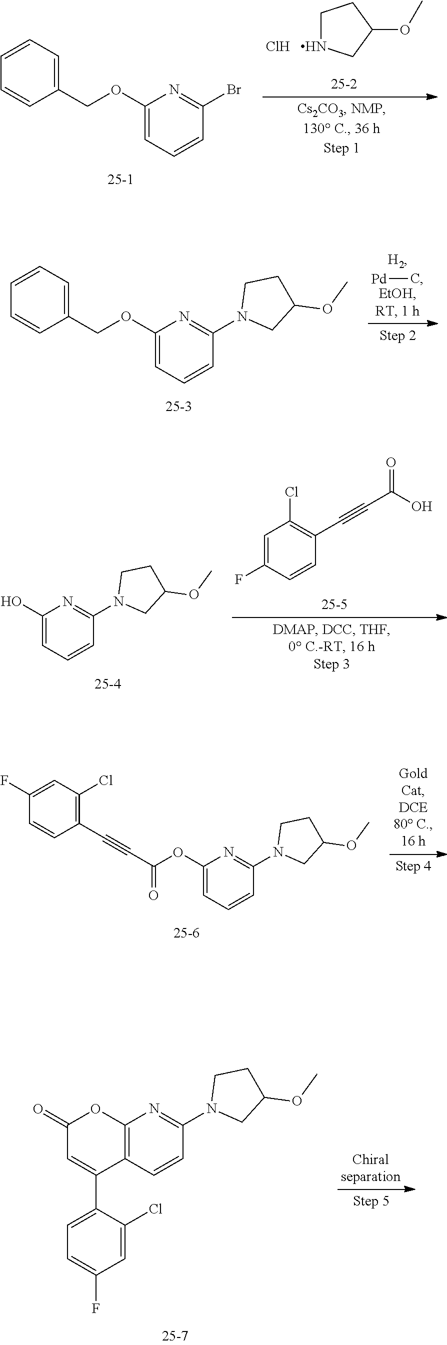

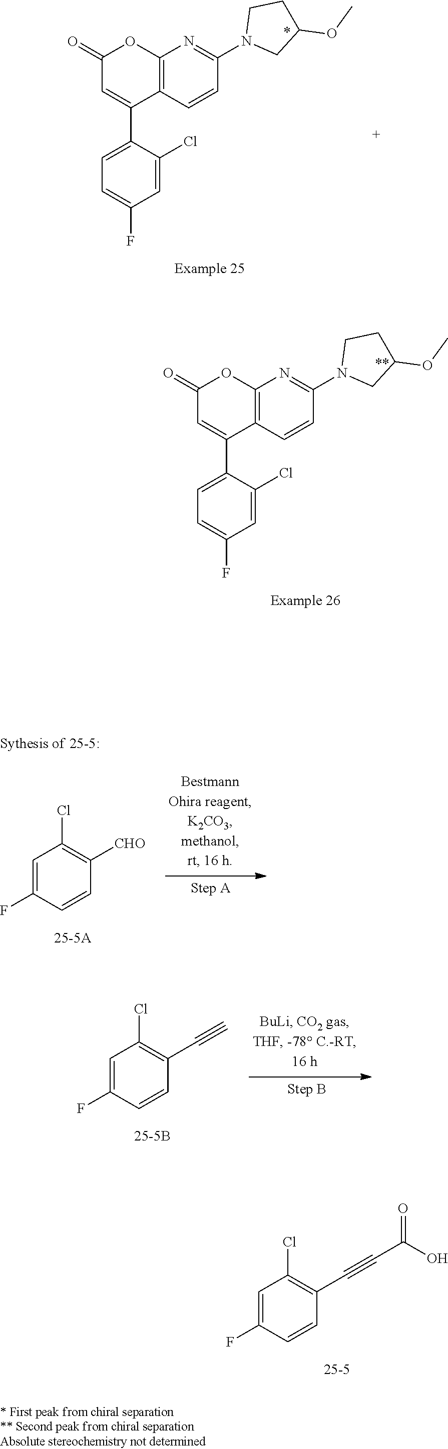

Synthesis of 2-benzyloxy-6-(3-methoxypyrrolidin-1-yl)pyridine, 25-3 [Step 1]: To a stirred solution of 2-benzyloxy-6-bromo-pyridine (25-1, 1.3 g, 4.9 mmol) and 3-methoxypyrrolidine hydrochloride (25-2, 1.0 g, 7.4 mmol) in NMP (15 mL) was added cesium carbonate (6.4 g, 19.7 mmol) and the mixture was heated at 130° C. for 36 h. The reaction mixture was diluted with water and extracted with ethyl acetate (3×50 mL). The organic layer was collected, washed with brine, dried over Na₂SO₄ and concentrated to get the crude residue. The crude was purified by flash column chromatography to afford 2-benzyloxy-6-(3-methoxypyrrolidin-1-yl)pyridine (25-3, 1.2 g). LCMS (ESI) Calcd. for C₁₇H₂₀N₂O₂: 284, found [M+H]⁺=285. ¹H NMR (400 MHz, CDCl₃) δ 7.36 (d, 2H), 7.34-7.32 (m, 3H), 7.28-7.25 (m, 1H), 6.04 (d, 1H), 5.88 (d, 1H), 5.35 (s, 2H), 4.07-4.03 (m, 1H), 3.55 (m, 2H), 3.51 (m, 2H), 3.35 (s, 3H), 2.14 (m, 2H).

Synthesis of 6-(3-methoxypyrrolidin-1-yl)pyridin-2-ol, 25-4 [Step 2]: To a stirred solution of 2-benzyloxy-6-(3-methoxypyrrolidin-1-yl)pyridine (25-3, 500 mg, 1.7 mmol) in ethanol (15 mL), Pd—C(10%) (250 mg, 2.4 mmol) was added at RT under nitrogen atmosphere. The mixture was stirred for 1.5 h at rt under hydrogen atmosphere. The reaction mixture was filtered through celite bed and washed the bed with ethanol. The filtrate was evaporated under reduced pressure to afford 6-(3-methoxypyrrolidin-1-yl)pyridin-2-ol (25-4, 340 mg). LCMS (ESI) Calcd. for C₁₀H₁₄N₂O₂: 194, found [M+H]⁺=195.

Synthesis of 6-(3-methoxypyrrolidin-1-yl)pyridin-2-yl 3-(2-chloro-4-fluorophenyl)propiolate, 25-6 [Step 3]: To a stirred solution of 6-(3-methoxypyrrolidin-1-yl)pyridin-2-ol (25-4, 340 mg, 1.74 mmol), 3-(2-chloro-4-fluoro-phenyl)prop-2-ynoic acid (25-5, 350 mg, 1.7 mmol) in THF (15 mL), was added DCC (575 mg, 2.8 mmol) and DMAP (0.04 mL, 0.3 mmol) at 0° C. and then stirred at rt for 16 h. The reaction mixture was filtered through celite bed and washed the bed with ethyl acetate. The filtrate was evaporated under reduced pressure. The crude material was purified by flash chromatography using 24% ethyl acetate in hexanes as eluent to afford 6-(3-methoxypyrrolidin-1-yl)pyridin-2-yl 3-(2-chloro-4-fluorophenyl)propiolate (25-6, 170 mg). LCMS (ESI): Calcd. for C₁₉H₁₆ClFN₂O₃: 374, found [M+H]⁺=375.

Synthesis of 4-(2-chloro-4-fluorophenyl)-7-(3-methoxypyrrolidin-1-yl)-2H-pyrano[2,3-b]pyridin-2-one, 25-7 [Step 4]: To a stirred solution of 6-(3-methoxypyrrolidin-1-yl)pyridin-2-yl 3-(2-chloro-4-fluorophenyl)propiolate (25-6, 170 mg, 0.5 mmol) in DCE (3 mL), nitrogen was bubbled for 10 min. Then (acetonitrile)[(2-biphenyl)di-tert-butylphosphine]gold(I) hexafluoroantimonate (35 mg, 0.05 mmol) was added and the mixture was heated at 80° C. for 16 h. The reaction mixture was quenched with water and extracted with DCM. The organic phase was dried over sodium sulfate, filtered and evaporated under reduced pressure to afford 4-(2-chloro-4-fluorophenyl)-7-(3-methoxypyrrolidin-1-yl)-2H-pyrano[2,3-b]pyridin-2-one (25-7, 94 mg). LCMS (ESI): Calcd. for C₁₉H₁₆ClFN₂O₃: 374, found [M+H]⁺=375.

Synthesis of chiral isomers of 4-(2-chloro-4-fluorophenyl)-7-(3-methoxypyrrolidin-1-yl)-2H-pyrano[2,3-b]pyridin-2-one, Example 25 and Example 26 [Step 5]: Racemic 4-(2-chloro-4-fluorophenyl)-7-(3-methoxypyrrolidin-1-yl)-2H-pyrano[2,3-b]pyridin-2-one (25-7, 94 mg) was submitted for chiral prep SFC purification. The fractions obtained are lyophilized to yield 4-(2-chloro-4-fluorophenyl)-7-(3-methoxypyrrolidin-1-yl)-2H-pyrano[2,3-b]pyridin-2-one (Example 25, 12 mg) as Peak 1, and 4-(2-chloro-4-fluorophenyl)-7-(3-methoxypyrrolidin-1-yl)-2H-pyrano[2,3-b]pyridin-2-one (Example 26, 9.6 mg) as Peak 2. The absolute stereochemistries were not determined.

Example 25: 4-(2-chloro-4-fluorophenyl)-7-(3-methoxypyrrolidin-1-yl)-2H-pyrano[2,3-b]pyridin-2-one, Peak 1: LCMS (ESI): Calcd. for C₁₉H₁₆ClFN₂O₃: 374, found [M+H]⁺=375. ¹H NMR (400 MHz, DMSO-d₆) δ 7.70-7.67 (m, 1H), 7.55-7.51 (m, 1H), 7.44-7.39 (m, 1H), 7.14 (d, 1H), 6.48 (d, 1H), 6.06 (s, 1H), 4.09 (s, 1H), 3.56-3.28 (m, 6H), 3.26 (m, 1H), 2.09-2.07 (br s, 2H).

Example 26: 4-(2-chloro-4-fluorophenyl)-7-(3-methoxypyrrolidin-1-yl)-2H-pyrano[2,3-b]pyridin-2-one, Peak 2: LCMS (ESI): Calcd. for C₁₉H₁₆ClFN₂O₃: 374, found [M+H]⁺=375. ¹H NMR (400 MHz, DMSO-d₆) δ 7.70-7.67 (m, 1H), 7.55-7.51 (m, 1H), 7.44-7.41 (m, 1H), 7.14 (d, 1H), 6.48 (d, 1H), 6.06 (s, 1H), 4.09 (s, 1H), 3.56-3.28 (m, 4H), 3.26 (m, 3H), 2.09-2.07 (br s, 2H).

Prep SFC method: Column: C-AMYLOSE-A (30 mm×250 mm), 5μ, Flow: 60 g/min, Mobile Phase: 60% CO₂₊₄₀% (0.5% IPAMINE in IPA), ABPR: 100 bar, Temp: 35° C., UV: 370 nm, DILUENT: MeOH, Sample concentration: 22 mg/ml, loading: 6.6 mg/5 min.

Synthesis of 2-chloro-1-ethynyl-4-fluorobenzene, 25-5B [Step A]: To a stirred solution of 2-chloro-4-fluoro-benzaldehyde (25-5A, 8.6 g, 54.2 mmol) in methanol (30 mL), was added potassium carbonate (15.0 g, 108 mmol) and Bestmann-Ohira reagent (12.5 g, 65.1 mmol) at rt and stirred for 16 h. The reaction mixture was evaporated under reduced pressure and then diluted with ethyl acetate. The organic layer was washed with water and brine solution, then dried over sodium sulfate and evaporated under reduced pressure. The crude material was purified with flash chromatography to afford 2-chloro-1-ethynyl-4-fluoro-benzene (25-5B, 1.60 g). ¹H NMR (400 MHz, CDCl₃) δ 7.69 (m, 1H), 7.24 (d, 1H), 7.25 (m, 1H), 6.97 (m, 1H), 3.32 (s, 1H).

Synthesis of 3-(2-chloro-4-fluorophenyl)propiolic acid, 25-5 [Step B]: To a stirred solution of 2-chloro-1-ethynyl-4-fluoro-benzene (25-5B, 1.6 g, 10.4 mmol) in THF (10 mL), was added n-BuLi (2.6 M) (3.7 mL, 10.4 mmol) at −78° C. and stirred for 1 h. at same temperature. After that reaction mixture was stirred under carbon dioxide gas environment at −78° C. to rt for 16 h. The reaction mixture was quenched with water and washed the aqueous layer with ethyl acetate. Then aqueous layer was acidified with 1N HCl solution and extracted with ethyl acetate (3×50 mL). The organic layer was dried over sodium sulfate and evaporated under reduced pressure to afford 3-(2-chloro-4-fluorophenyl)propiolic acid (25-5, 1.40 g). ¹H NMR (400 MHz, DMSO-d₆) δ 14.0 (s, 1H), 7.85-7.82 (m, 1H), 7.71-7.68 (br s, 1H), 7.38-7.33 (m, 1H).

### Example 27-28: Synthesis of chiral isomers of 7-(3-hydroxy-3-methylpyrrolidin-1-yl)-4-(o-tolyl)-2H-pyrano[2,3-b]pyridin-2-one

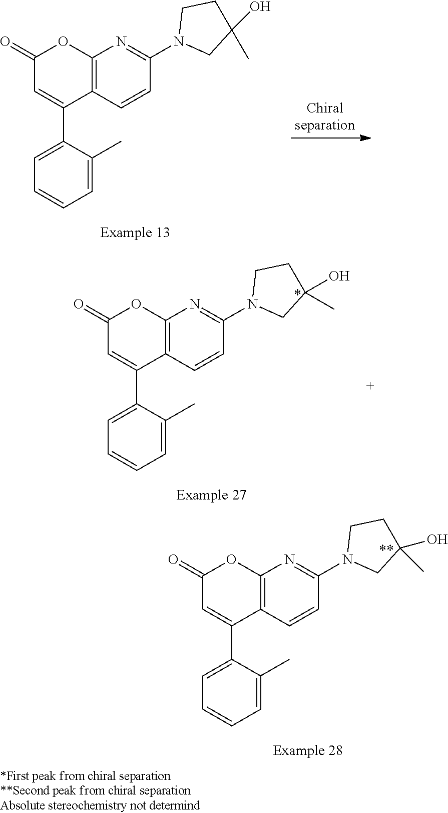 [SMILES: Cc1ccccc1-c1cc(=O)oc2nc(N3CCC(C)(O)C3)ccc12 ; Cc1ccccc1-c1cc(=O)oc2nc(N3CCC(C)(O)C3)ccc12 ; Cc1ccccc1-c1cc(=O)oc2nc(N3CCC(C)(O)C3)ccc12]

Racemic 7-(3-hydroxy-3-methylpyrrolidin-1-yl)-4-(o-tolyl)-2H-pyrano[2,3-b]pyridin-2-one (Example 13, 55 mg, 0.2 mmol) was separated by SFC HPLC chiral purification and lyophilized to afford the first product as 7-(3-hydroxy-3-methylpyrrolidin-1-yl)-4-(o-tolyl)-2H-pyrano[2,3-b]pyridin-2-one (Example 27, 15 mg) as Peak 1, and the second product as 7-(3-hydroxy-3-methylpyrrolidin-1-yl)-4-(o-tolyl)-2H-pyrano[2,3-b]pyridin-2-one (Example 28, 16 mg) as Peak 2. The absolute stereochemistries of these compounds were not determined.

Example 27: 7-(3-hydroxy-3-methylpyrrolidin-1-yl)-4-(o-tolyl)-2H-pyrano[2,3-b]pyridin-2-one, Peak 1: LCMS (ESI) Calcd. for C₂₀H₂₀N₂O₃: 336, found [M+H]⁺=337. ¹H NMR (400 MHz, DMSO-d₆) δ 7.39-7.31 (m, 3H), 7.22 (d, 1H), 7.03 (d, 1H), 6.42 (br s, 1H), 5.94 (s, 1H), 4.87 (br s, 1H), 3.59-3.46 (m, 4H), 2.11 (s, 3H), 1.94 (br s, 2H), 1.33 (s, 3H).

Example 28: 7-(3-hydroxy-3-methylpyrrolidin-1-yl)-4-(o-tolyl)-2H-pyrano[2,3-b]pyridin-2-one, Peak 2: LCMS (ESI) Calcd. for C₂₀H₂₀N₂O₃: 336, found [M+H]⁺=337. ¹H NMR (400 MHz, DMSO-d₆) δ 7.44-7.36 (m, 3H), 7.24 (d, 1H), 7.05 (d, 1H), 6.44 (br s, 1H), 5.96 (s, 1H), 4.90 (br s, 1H), 3.69-3.55 (m, 4H), 2.13 (s, 3H), 2.01 (br s, 2H), 1.38 (s, 3H).

SFC chiral HPLC method: Chiral separation was performed on Thar SFC-80 series instrument. Column: CHIRALCEL OX-H column (250×21 mm), 5μ, operating at a temperature of 35° C. with a flow rate of 60 gm/min. Mobile phase: 70% CO₂, 30% methanol, held isocratic and isobaric (100 bar) up to 13 min. with detection at a wavelength of 370 nm.

### Example 29-30: Synthesis of chiral isomers of 1-(2-oxo-4-(o-tolyl)-2H-pyrano[2,3-b]pyridin-7-yl)pyrrolidine-3-carboxamide

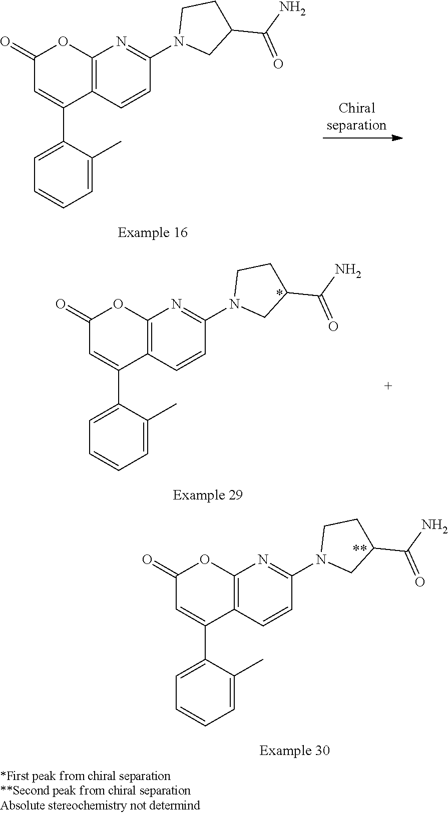 [SMILES: Cc1ccccc1-c1cc(=O)oc2nc(N3CCC(C(N)=O)C3)ccc12 ; Cc1ccccc1-c1cc(=O)oc2nc(N3CCC(C(N)=O)C3)ccc12 ; Cc1ccccc1-c1cc(=O)oc2nc(N3CCC(C(N)=O)C3)ccc12]

Racemic 1-(2-oxo-4-(o-tolyl)-2H-pyrano[2,3-b]pyridin-7-yl)pyrrolidine-3-carboxamide (Example 16, 120 mg) was submitted for chiral HPLC (NP) separation to afford the first product as 1-(2-oxo-4-(o-tolyl)-2H-pyrano[2,3-b]pyridin-7-yl)pyrrolidine-3-carboxamide (Example 29, 50 mg) as Peak 1, and the second product as 1-(2-oxo-4-(o-tolyl)-2H-pyrano[2,3-b]pyridin-7-yl)pyrrolidine-3-carboxamide (Example 30, 52 mg) as Peak 2. The absolute stereochemistries of these compounds were not determined.

Example 29: 1-(2-oxo-4-(o-tolyl)-2H-pyrano[2,3-b]pyridin-7-yl)pyrrolidine-3-carboxamide, Peak 1: LCMS (ESI) Calcd. for C₂₀H₁₉N₃O₃: 349, found [M+H]⁺=350. ¹H NMR (400 MHz, DMSO-d₆) δ 7.52 (br s, 1H), 7.44-7.37 (m, 2H), 7.34 (t, 1H), 7.21 (d, 1H), 7.04 (d, 1H), 6.99 (br s, 1H), 6.44 (d, 1H), 5.96 (s, 1H), 3.71 (t, 1H), 3.62-3.58 (m, 2H), 3.51 (t, 1H), 3.11 (t, 1H), 2.23-2.18 (m, 1H), 2.16-2.11 (m, 4H).

Example 30: 1-(2-oxo-4-(o-tolyl)-2H-pyrano[2,3-b]pyridin-7-yl)pyrrolidine-3-carboxamide, Peak 2: LCMS (ESI) Calcd. for C₂₀H₁₉N₃O₃: 349, found [M+H]⁺=350. ¹H NMR (400 MHz, DMSO-d₆) δ 7.52 (br s, 1H), 7.44-7.37 (m, 2H), 7.34 (t, 1H), 7.21 (d, 1H), 7.04 (d, 1H), 6.99 (br s, 1H), 6.44 (d, 1H), 5.96 (s, 1H), 3.71 (t, 1H), 3.62-3.58 (m, 2H), 3.50 (t, 1H), 3.10 (t, 1H), 2.23-2.19 (m, 1H), 2.18-2.14 (m, 4H).

PREP HPLC CHIRAL METHOD: Chiral separation was done on Agilent 1200 series instrument. Column name: CHIRALPAK IG (250×21 mm) 5u. operating at ambient temperature and flow rate is 21.0 ml/min. Mobile phase was the mixture of 50% hexane, 25% DCM, 25% EtOH, held this isocratic mixture run up to 18 min. with wavelength of 236 nm.

### Example 31-32: Synthesis of chiral isomers of 7-(3-(methoxymethyl)pyrrolidin-1-yl)-4-(o-tolyl)-2H-pyrano[2,3-b]pyridin-2-one

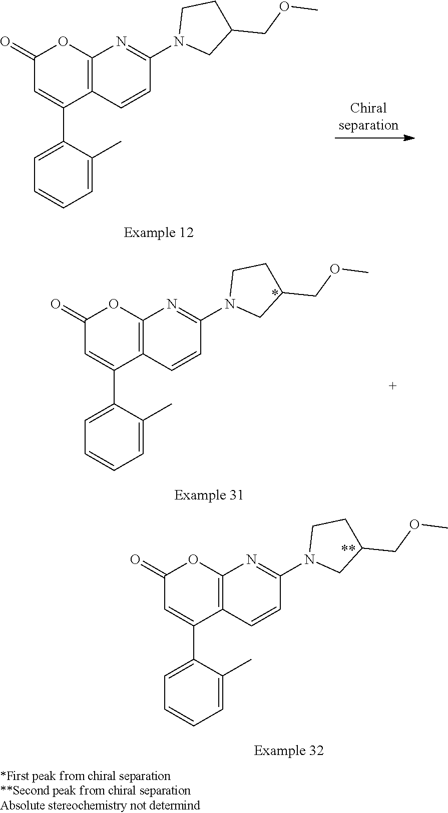 [SMILES: COCC1CCN(c2ccc3c(-c4ccccc4C)cc(=O)oc3n2)C1 ; COCC1CCN(c2ccc3c(-c4ccccc4C)cc(=O)oc3n2)C1 ; COCC1CCN(c2ccc3c(-c4ccccc4C)cc(=O)oc3n2)C1]

Racemic 7-(3-(methoxymethyl)pyrrolidin-1-yl)-4-(o-tolyl)-2H-pyrano[2,3-b]pyridin-2-one (Example 12, 270 mg) was separated by SFC chiral HPLC purification and lyophilized to afford the first product as 7-(3-(methoxymethyl)pyrrolidin-1-yl)-4-(o-tolyl)-2H-pyrano[2,3-b]pyridin-2-one (Example 31, 6 mg) as Peak 1, and the second product as 7-(3-(methoxymethyl)pyrrolidin-1-yl)-4-(o-tolyl)-2H-pyrano[2,3-b]pyridin-2-one (Example 32, 6 mg) as Peak 2. The absolute stereochemistries of these compounds were not determined.

Example 31: 7-(3-(methoxymethyl)pyrrolidin-1-yl)-4-(o-tolyl)-2H-pyrano[2,3-b]pyridin-2-one, Peak 1: LCMS (ESI) Calcd. for C₂₁H₂₂N₂O₃: 350, found [M+H]⁺=351. ¹H NMR (400 MHz, DMSO-d₆) δ 7.43 (br s, 3H), 7.35 (d, 1H), 7.03 (d, 1H), 6.45 (d, 1H), 5.95 (s, 1H), 3.59 (br s, 3H), 3.35 (br s, 6H), 2.50 (br s, 1H), 2.10 (d, 4H), 1.74 (s, 1H).

Example 32: 7-(3-(methoxymethyl)pyrrolidin-1-yl)-4-(o-tolyl)-2H-pyrano[2,3-b]pyridin-2-one, Peak 2: LCMS (ESI) Calcd. for C₂₁H₂₂N₂O₃: 350, found [M+H]⁺=351. ¹H NMR (400 MHz, DMSO-d₆) δ 7.43 (br s, 3H), 7.35 (d, 1H), 7.03 (d, 1H), 6.45 (d, 1H), 5.95 (s, 1H), 3.59 (br s, 3H), 3.35 (br s, 6H), 2.50 (br s, 1H), 2.10 (d, 4H), 1.74 (s, 1H).

SFC chiral HPLC method: Chiral separation was performed on a chiral Pak IE (250×21 mm), 5μ, operating at a temperature of 35° C. with a flow rate of 30 ml/min. Mobile phase: 30% methanol with of 0.3% ipamine, held isocratic and isobaric (100 bar) up to 11 min., with detection at a wavelength of 250 nm.

### Example 33-34: Synthesis of chiral isomers of 1-(2-oxo-4-(o-tolyl)-2H-pyrano[2,3-b]pyridin-7-yl)pyrrolidine-3-carboxylic acid

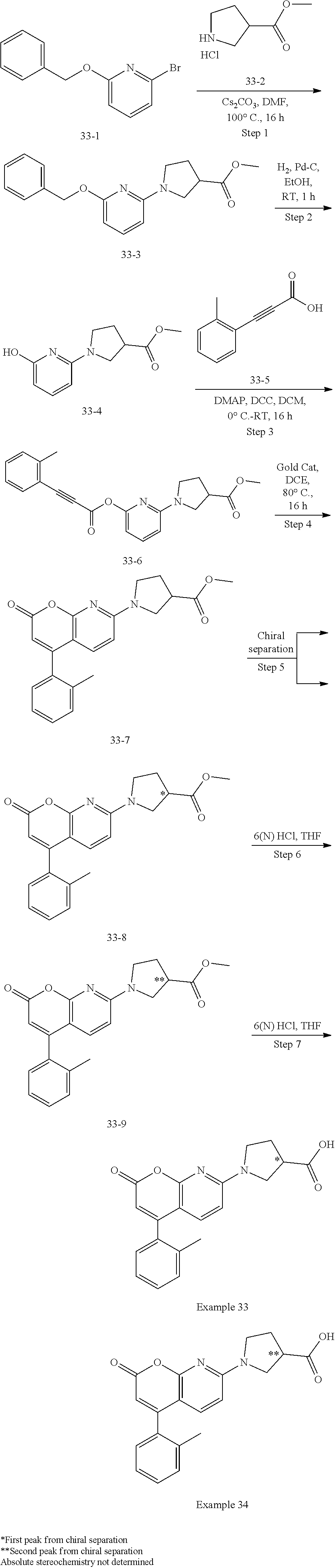

Synthesis of methyl 1-(6-(benzyloxy)pyridin-2-yl)pyrrolidine-3-carboxylate, 33-3 [Step 1]: To a stirred solution of methyl pyrrolidine-3-carboxylate hydrochloride (33-2, 470 mg, 3.0 mmol) and 2-(benzyloxy)-6-bromopyridine (33-1, 500 mg, 2 mmol) in NMP (5 mL) was added cesium carbonate (2.5 g, 7.0 mmol) and the mixture was heated at 100° C. for 16 h. The mixture was cooled to ambient temperature and water was added and extracted with ethyl acetate (twice). The organic part was dried over anhydrous Na₂SO₄ and concentrated under reduced pressure. The product was purified by flash chromatography to afford methyl 1-(6-(benzyloxy)pyridin-2-yl)pyrrolidine-3-carboxylate (33-3, 400 mg). LCMS (ESI) Calcd. for C₁₈H₂₀N₂O₃: 312, found [M+H]⁺=313.

Synthesis of methyl 1-(6-hydroxypyridin-2-yl)pyrrolidine-3-carboxylate, 33-4 [Step 2]: A stirred solution of methyl 1-(6-(benzyloxy)pyridin-2-yl)pyrrolidine-3-carboxylate (33-3, 300 mg, 0.9 mmol) in ethanol (10 mL) was degassed by bubbling with argon gas for 10 min. In to this solution was added 10% Pd/C (80 mg, 0.1 mmol) and the mixture was hydrogenated using hydrogen balloon at ambient temperature for 2 h. The mixture was filtered through celite bed and concentrated under reduced pressure to afford methyl 1-(6-hydroxypyridin-2-yl)pyrrolidine-3-carboxylate (33-4, 200 mg). LCMS (ESI) Calcd. for C₁₁H₁₄N₂O₃: 222, found [M+H]⁺=223.

Synthesis of methyl 1-(6-((3-(o-tolyl)propioloyl)oxy)pyridin-2-yl)pyrrolidine-3-carboxylate, 33-6 [Step 3]: In a 50 mL two neck round bottom flask methyl 1-(6-hydroxypyridin-2-yl)pyrrolidine-3-carboxylate (33-4, 200 mg, 0.9 mmol) and 3-(o-tolyl)propiolic acid (33-5, 175 mg, 1.0 mmol) were taken in DCM (5 mL) and DMAP (19 mg, 0.2 mmol) was added. The mixture was cooled to 0° C. and DCC (280 mg, 1.3 mmol) in DCM (2 mL) was added slowly to the mixture and stirred at ambient temperature for 16 h. The reaction mixture was diluted with DCM and washed with water and brine. The organic layer was dried over anhydrous Na₂SO₄, filtered and concentrated under reduced pressure. The product was purified by column chromatography to yield methyl 1-(6-((3-(o-tolyl)propioloyl)oxy)pyridin-2-yl)pyrrolidine-3-carboxylate (33-6, 130 mg). LCMS (ESI) Calcd. for C₂₁H₂₀N₂O₄: 364, found [M+H]⁺=365.

Synthesis of methyl 1-(2-oxo-4-(o-tolyl)-2H-pyrano[2,3-b]pyridin-7-yl)pyrrolidine-3-carboxylate, 33-7 [Step 4]: A stirred solution of methyl 1-(6-((3-(o-tolyl)propioloyl)oxy)pyridin-2-yl)pyrrolidine-3-carboxylate (33-6, 200 mg, 0.6 mmol) in DCE (2 mL) was degassed with argon gas for 5 min. Into the mixture was added (acetonitrile)[(2-biphenyl)di-tert-butylphosphine]gold(I) hexafluoroantimonate (40 mg, 0.06 mmol) and the mixture was stirred at 80° C. for 16 h. The mixture was diluted with DCM and filtered through celite bed and concentrated under reduced pressure. The product was purified by flash chromatography to afford methyl 1-(2-oxo-4-(o-tolyl)-2H-pyrano[2,3-b]pyridin-7-yl)pyrrolidine-3-carboxylate (33-7, 160 mg). LCMS (ESI) Calcd. for C₂₁H₂₀N₂O₄: 364, found [M+H]⁺=365.

Synthesis of chiral isomers of methyl 1-(2-oxo-4-(o-tolyl)-2H-pyrano[2,3-b]pyridin-7-yl)pyrrolidine-3-carboxylate, 33-8 and 33-9 [Step 5]: Racemic methyl 1-(2-oxo-4-(o-tolyl)-2H-pyrano[2,3-b]pyridin-7-yl)pyrrolidine-3-carboxylate (33-7, 150 mg) was separated by chiral NP prep purification and lyophilized to afford the first product as methyl 1-(2-oxo-4-(o-tolyl)-2H-pyrano[2,3-b]pyridin-7-yl)pyrrolidine-3-carboxylate (33-8, 50 mg) as Peak 1, and the second product as methyl 1-(2-oxo-4-(o-tolyl)-2H-pyrano[2,3-b]pyridin-7-yl)pyrrolidine-3-carboxylate (33-9, 50 mg) as Peak 2. The absolute stereochemistries of these compounds were not determined.

33-8: Methyl 1-(2-oxo-4-(o-tolyl)-2H-pyrano[2,3-b]pyridin-7-yl)pyrrolidine-3-carboxylate, Peak 1: LCMS (ESI) Calcd. for C₂₁H₂₀N₂O₄: 364, found [M+H]⁺=365.

33-9: Methyl 1-(2-oxo-4-(o-tolyl)-2H-pyrano[2,3-b]pyridin-7-yl)pyrrolidine-3-carboxylate, Peak 2: LCMS (ESI) Calcd. for C₂₁H₂₀N₂O₄: 364, found [M+H]⁺=365.

NP chiral HPLC method: Chiral separation was performed on a Agilent 1200 series instrument. Column: CHIRALPAK IG (250×20 mm) 5u, operating at a temperature of 35° C. with a flow rate of 21 mL/min. Mobile phase: mixture of 60% hexane, 20% DCM, 20% ethanol, held this isocratic mixture run up to 20 min. with wavelength of 364 nm.

Synthesis of chiral 1-(2-oxo-4-(o-tolyl)-2H-pyrano[2,3-b]pyridin-7-yl)pyrrolidine-3-carboxylic acid, Example 33 [Step 6]: To a stirred solution of methyl 1-(2-oxo-4-(o-tolyl)-2H-pyrano[2,3-b]pyridin-7-yl)pyrrolidine-3-carboxylate (33-8, 50 mg, 0.1 mmol) in THF (2 mL) was added HCl (0.5 mL, 2.7 mmol, 6 N). The reaction mixture was stirred at ambient temperature for 18 h. The resulting mixture was concentrated under reduced pressure. The product which was purified by RP prep-HPLC purification and lyophilized to afford 1-(2-oxo-4-(o-tolyl)-2H-pyrano[2,3-b]pyridin-7-yl)pyrrolidine-3-carboxylic acid (Example 33, 10 mg). LCMS (ESI) Calcd. for C₂₀H₁₈N₂O₄: 350, found [M+H]⁺=351. ¹H NMR (400 MHz, CDCl₃) δ 7.38-7.34 (m, 1H), 7.31-7.28 (m, 2H), 7.14-7.10 (m, 2H), 6.20 (d, 1H), 6.01 (s, 1H), 3.86 (br s, 2H), 3.76-3.71 (m, 1H), 3.60 (br s, 1H), 3.29 (t, 1H), 2.36 (q, 2H), 2.14 (s, 3H). The absolute stereochemistry of this compound was not determined.

Synthesis of chiral 1-(2-oxo-4-(o-tolyl)-2H-pyrano[2,3-b]pyridin-7-yl)pyrrolidine-3-carboxylic acid, Example 34 [Step 7]: To a stirred solution of methyl 1-(2-oxo-4-(o-tolyl)-2H-pyrano[2,3-b]pyridin-7-yl)pyrrolidine-3-carboxylate (33-9, 50 mg, 0.14 mmol) in THF (2 mL) was added HCl (0.5 ml, 2.7 mmol, 6N). The reaction mixture was stirred at ambient temperature for 18 h. The resulting mixture was concentrated under reduced pressure. The product was purified by RP prep-HPLC purification and lyophilized to afford 1-(2-oxo-4-(o-tolyl)-2H-pyrano[2,3-b]pyridin-7-yl)pyrrolidine-3-carboxylic acid (Example 34, 10 mg). ¹H NMR (400 MHz, CDCl₃) δ 7.38-7.34 (m, 1H), 7.31-7.28 (m, 2H), 7.14-7.10 (m, 2H), 6.20 (d, 1H), 6.00 (s, 1H), 3.85 (br s, 2H), 3.78-3.70 (m, 1H), 3.64-3.60 (m, 1H), 3.28 (t, 1H), 2.35 (q, 2H), 2.13 (s, 3H). LCMS (ESI) Calcd. for C₂₀H₁₈N₂O₄: 350, found [M+H]⁺=351. The absolute stereochemistry of this compound was not determined.

### Example 35: Synthesis of methyl N-methyl-N-(2-oxo-4-(o-tolyl)-2H-pyrano[2,3-b]pyridin-7-yl)glycinate

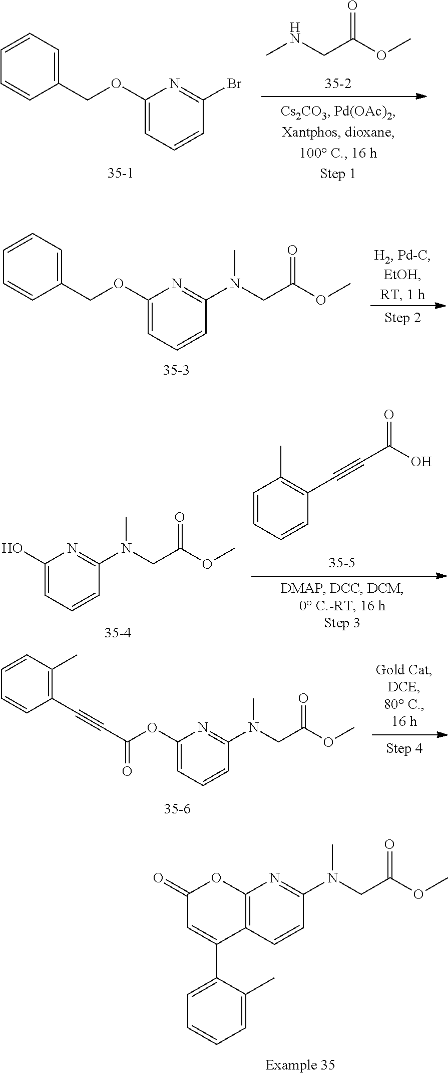

Synthesis of methyl N-(6-(benzyloxy)pyridin-2-yl)-N-methylglycinate, 35-3 [Step 1]: To a stirred solution of methyl methylglycinate (35-2, 585 mg, 6.0 mmol) and 2-(benzyloxy)-6-bromopyridine (35-1, 1.0 g, 4.0 mmol) in 1,4-dioxane (15 mL) was added Cs₂CO₃ (3.7 g, 11.4 mmol). The mixture was purged with argon gas and Pd(OAc)₂ (85 mg, 0.4 mmol) and Xantphos (440 mg, 0.75 mmol) were added. The mixture was heated at 100° C. for 16 h. The mixture was cooled to ambient temperature and water was added and extracted with ethyl acetate (twice). The organic part was dried over anhydrous Na₂SO₄, filtered and concentrated under reduced pressure. The product was purified by combi flash chromatography to get methyl N-(6-(benzyloxy)pyridin-2-yl)-N-methylglycinate (35-3, 500 mg). LCMS (ESI) Calcd. for C₁₆H₁₈N₂O₃: 286, found [M+H]⁺=287.

Synthesis of methyl N-(6-hydroxypyridin-2-yl)-N-methylglycinate, 35-4 [Step 2]: To a stirred solution of methyl N-(6-(benzyloxy)pyridin-2-yl)-N-methylglycinate (35-3, 500 mg, 1.7 mmol) in ethanol (10 mL) was purged with argon gas for 5 min. Then 10% Pd/C (100 mg, 0.1 mmol) was added to the mixture and hydrogenated using a H₂ balloon at ambient temperature for 2 h. The mixture was filtered through celite bed and concentrated under reduced pressure to afford methyl N-(6-hydroxypyridin-2-yl)-N-methylglycinate (35-4, 340 mg). ¹H NMR (400 MHz, DMSO-d₆) δ 9.97 (br s, 1H), 7.35 (t, 1H), 6.01 (d, 1H), 5.84 (d, 1H), 4.33 (s, 2H), 3.62 (s, 3H), 2.97 (s, 3H).

Synthesis of 6-((2-methoxy-2-oxoethyl)(methyl)amino)pyridin-2-yl 3-(o-tolyl)propiolate, 35-6 [Step 3]: In a 50 mL two neck round bottom flask methyl N-(6-hydroxypyridin-2-yl)-N-methylglycinate (35-4, 340 mg, 1.7 mmol) and 3-(o-tolyl)propiolic acid (35-5, 335 mg, 2.1 mmol) were taken. To the mixture DCM (10 mL) was added followed by DMAP (0.05 mL, 0.3 mmol). The mixture was stirred at 0° C. and DCC (570 mg, 2.8 mmol) in DCM (2 mL) was added to the mixture and stirred at ambient temperature for 16 h. The reaction mixture was diluted with DCM and washed with water and brine. The organic layer was dried over anhydrous Na₂SO₄, filtered and concentrated under reduced pressure. The product was purified by column chromatography to yield 6-((2-methoxy-2-oxoethyl)(methyl)amino)pyridin-2-yl 3-(o-tolyl)propiolate (35-6, 500 mg). LCMS (ESI) Calcd. for C₁₉H₁₈N₂O₄: 338, found [M+H]⁺=339.

Synthesis of methyl N-methyl-N-(2-oxo-4-(o-tolyl)-2H-pyrano[2,3-b]pyridin-7-yl)glycinate, Example 35 [Step 4]: A stirred solution of 6-((2-methoxy-2-oxoethyl)(methyl)amino)pyridin-2-yl 3-(o-tolyl)propiolate (35-6, 100 mg, 0.3 mmol) in DCE (2 mL) was degassed with argon gas for 5 min. Then (acetonitrile)[(2-biphenyl)di-tert-butylphosphine]gold(I) hexafluoroantimonate (25 mg, 0.03 mmol) was added and the mixture was heated at 80° C. for 18 h. After completion, the mixture was diluted with DCM, filtered through celite bed and concentrated under reduced pressure. The product was purified by RP prep-HPLC purification and lyophilized to afford methyl N-methyl-N-(2-oxo-4-(o-tolyl)-2H-pyrano[2,3-b]pyridin-7-yl)glycinate (Example 35, 80 mg). LCMS (ESI) Calcd. for C₁₉H₁₈N₂O₄: 338, found [M+H]⁺=339. ¹H NMR (400 MHz, DMSO-d₆) δ 7.42-7.40 (m, 2H), 7.34 (t, 1H), 7.22 (d, 1H), 7.12 (d, 1H), 6.68 (d, 1H), 6.03 (s, 1H), 4.46 (s, 2H), 3.67 (s, 3H), 3.14 (s, 3H), 2.12 (s, 3H).

### Example 36: Synthesis of N-methyl-N-(2-oxo-4-(o-tolyl)-2H-pyrano[2,3-b]pyridin-7-yl)glycine

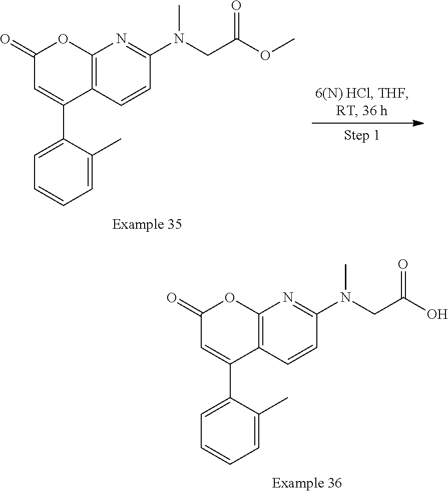 [SMILES: COC(=O)CN(C)c1ccc2c(-c3ccccc3C)cc(=O)oc2n1 ; Cc1ccccc1-c1cc(=O)oc2nc(N(C)CC(=O)O)ccc12]

Synthesis of N-methyl-N-(2-oxo-4-(o-tolyl)-2H-pyrano[2,3-b]pyridin-7-yl)glycine, Example 36 [Step 1]: To a stirred solution of methyl N-methyl-N-(2-oxo-4-(o-tolyl)-2H-pyrano[2,3-b]pyridin-7-yl)glycinate (Example 35, 150 mg, 0.4 mmol) in THF (1 mL) was added solution of HCl (2.0 ml, 9.0 mmol, 6 N) dropwise at 0° C. The reaction mixture was stirred for 36 h. at ambient temperature. The resulting mixture was concentrated under reduced pressure to afford product which was purified by prep HPLC purification and lyophilized to afford N-methyl-N-(2-oxo-4-(o-tolyl)-2H-pyrano[2,3-b]pyridin-7-yl)glycine (Example 36, 120 mg). LCMS: Calcd. for C₁₈H₁₆N₂O₄: 324, found [M+H]⁺=325. ¹H NMR (400 MHz, CDCl₃) δ 7.34-7.32 (m, 2H), 7.12-7.07 (m, 2H), 6.38 (d, 1H), 5.94 (s, 1H), 4.29-4.28 (m, 2H), 3.14 (s, 3H), 2.10 (s, 3H).

### Example 37-38: Synthesis of chiral isomers of 1-(2-oxo-4-(o-tolyl)-2H-pyrano[2,3-b]pyridin-7-yl)pyrrolidine-2-carboxylic acid

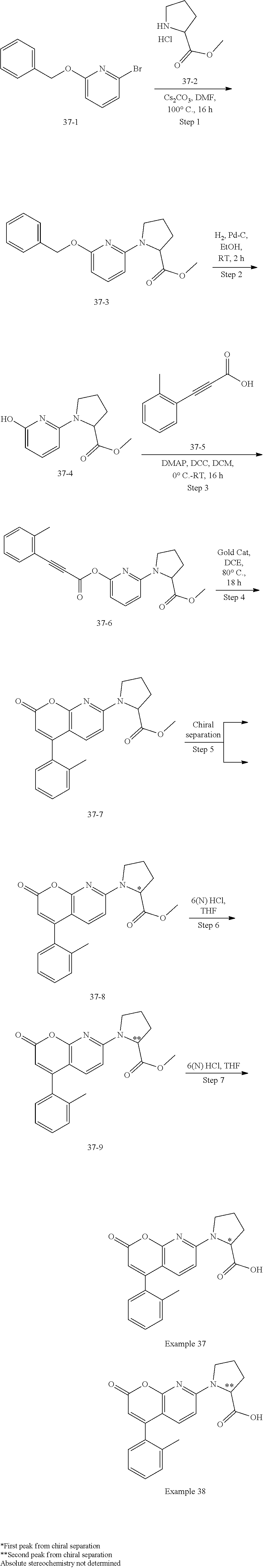

Synthesis of methyl 1-(6-(benzyloxy)pyridin-2-yl)pyrrolidine-2-carboxylate, 37-3 [Step 1]: To a stirred solution of methyl pyrrolidine-2-carboxylate hydrochloride (37-2, 480 mg, 3 mmol) and 2-benzyloxy-6-bromo-pyridine (37-1, 500 mg, 2 mmol) in 1,4-dioxane (5 mL) was added Cs₂CO₃ (2.0 g, 6.0 mmol) and purged under argon atmosphere for 5 min. Then Pd(OAc)₂ (45 mg, 0.2 mmol) and Xantphos (220 mg, 0.4 mmol) were added and the mixture was heated at 100° C. for 16 h. The mixture was cooled to ambient temperature, water was added and extracted with ethyl acetate. The organic part was dried over anhydrous Na₂SO₄, filtered and concentrated under reduced pressure. The product was purified through combiflash chromatography to afford and methyl 1-(6-benzyloxy-2-pyridyl)pyrrolidine-2-carboxylate (37-3, 400 mg). LCMS (ESI) Calcd. for C₁₈H₂₀N₂O₃: 312, found [M+H]⁺=313.

Synthesis of methyl 1-(6-hydroxypyridin-2-yl)pyrrolidine-2-carboxylate, 37-4 [Step 2]: To a stirred solution of methyl 1-(6-(benzyloxy)pyridin-2-yl)pyrrolidine-3-carboxylate (37-3, 400 mg, 1.3 mmol) in ethanol (10 mL) was degassed by bubbling with argon gas for 10 min. followed by the addition of 10% Pd/C (100 mg, 0.1 mmol) and the mixture was hydrogenated using H₂ balloon at ambient temperature for 2 h. The mixture was filtered through celite bed and concentrated under reduced pressure to afford methyl 1-(6-hydroxypyridin-2-yl)pyrrolidine-2-carboxylate (37-4, 290 mg). ¹H NMR (400 MHz, DMSO-d₆) δ 9.92 (s, 1H), 7.31 (t, 1H), 5.79 (d, 2H), 4.46 (d, 1H), 3.61 (s, 3H), 3.52-3.45 (m, 1H), 2.22-2.15 (m, 1H), 1.95 (br s, 2H).

Synthesis of methyl 1-(6-((3-(o-tolyl)propioloyl)oxy)pyridin-2-yl)pyrrolidine-2-carboxylate, 37-6 [Step 3]: To a stirred solution of methyl 1-(6-hydroxypyridin-2-yl)pyrrolidine-3-carboxylate (37-4, 290 mg, 1.3 mmol) and 3-(o-tolyl)propiolic acid (37-5, 250 mg, 1.5 mmol) in DCM (5 mL) was added DMAP (25 mg, 0.3 mmol). The mixture was stirred at 0° C. and slowly DCC (430 mg, 2.1 mmol) in DCM (2 mL) was added to the mixture and stirred at ambient temperature for 16 h. The reaction mixture was diluted with DCM and washed with water and brine. The organic layer was dried over anhydrous Na₂SO₄, filtered and concentrated under reduced pressure. The product was purified by column chromatography over silica gel to afford methyl 1-(6-((3-(o-tolyl)propioloyl)oxy)pyridin-2-yl)pyrrolidine-2-carboxylate (37-6, 300 mg). LCMS (ESI) Calcd. for C₂₁H₂₀N₂O₄: 364, found [M+H]⁺=365.

Synthesis of methyl 1-(2-oxo-4-(o-tolyl)-2H-pyrano[2,3-b]pyridin-7-yl)pyrrolidine-2-carboxylate, 37-7 [Step 4]: To a stirred solution of methyl 1-(6-((3-(o-tolyl)propioloyl)oxy)pyridin-2-yl)pyrrolidine-2-carboxylate (37-6, 300 mg, 0.8 mmol) in DCE (4 mL) was degassed with argon gas for 5 min. (Acetonitrile)[(2-biphenyl)di-tert-butylphosphine]gold(I) hexafluoroantimonate (65 mg, 0.1 mmol) was added to the mixture and the mixture was heated at 80° C. for 18 h. The mixture was diluted with DCM, filtered through celite bed and concentrated under reduced pressure. The product was purified by flash chromatography to afford methyl 1-(2-oxo-4-(o-tolyl)-2H-pyrano[2,3-b]pyridin-7-yl)pyrrolidine-2-carboxylate (37-7, 300 mg). LCMS (ESI) Calcd. for C₂₁H₂₀N₂O₄: 364, found [M+H]⁺=365.

Synthesis of chiral isomers of methyl 1-(2-oxo-4-(o-tolyl)-2H-pyrano[2,3-b]pyridin-7-yl)pyrrolidine-2-carboxylate, 37-8 and 37-9 [Step 5]: Racemic methyl 1-(2-oxo-4-(o-tolyl)-2H-pyrano[2,3-b]pyridin-7-yl)pyrrolidine-2-carboxylate (37-7, 300 mg) was separated by chiral NP prep purification and lyophilized to afford the first product as methyl 1-(2-oxo-4-(o-tolyl)-2H-pyrano[2,3-b]pyridin-7-yl)pyrrolidine-2-carboxylate (37-8, 120 mg) as Peak 1, and the second product as methyl 1-(2-oxo-4-(o-tolyl)-2H-pyrano[2,3-b]pyridin-7-yl)pyrrolidine-2-carboxylate (37-9, 120 mg) as Peak 2. The absolute stereochemistries of these compounds were not determined.

37-8: Methyl 1-(2-oxo-4-(o-tolyl)-2H-pyrano[2,3-b]pyridin-7-yl)pyrrolidine-2-carboxylate, Peak 1: LCMS (ESI) Calcd. for C₂₁H₂₀N₂O₄: 364, found [M+H]⁺=365.

37-9: Methyl 1-(2-oxo-4-(o-tolyl)-2H-pyrano[2,3-b]pyridin-7-yl)pyrrolidine-2-carboxylate, Peak 2: LCMS (ESI) Calcd. for C₂₁H₂₀N₂O₄: 364, found [M+H]⁺=365.

SFC chiral HPLC method: Chiral separation was performed on a Agilent 1200 series instrument. Column: CHIRALPAK IG (250×20 mm) 5u, operating at a temperature of 35° C. with a flow rate of 21 mL/min. Mobile phase: mixture of 60% hexane, 20% DCM, 20% ethanol, held this isocratic mixture run up to 20 min. with wavelength of 364 nm.

Synthesis of chiral 1-(2-oxo-4-(o-tolyl)-2H-pyrano[2,3-b]pyridin-7-yl)pyrrolidine-2-carboxylic acid, Example 37 [Step 6]: To a stirred solution of methyl 1-(2-oxo-4-(o-tolyl)-2H-pyrano[2,3-b]pyridin-7-yl)pyrrolidine-2-carboxylate as Peak 1 (37-8, 120 mg, 0.3 mmol) in THF (4 mL) was added HCl (2 ml, 7 mmol, 6 N). The reaction mixture was stirred at ambient temperature for 18 h. The resulting mixture was concentrated under reduced pressure and the product was purified by RP prep-HPLC purification and lyophilized to afford chiral 1-(2-oxo-4-(o-tolyl)-2H-pyrano[2,3-b]pyridin-7-yl)pyrrolidine-2-carboxylic acid (Example 37, 70 mg). LCMS (ESI) Calcd. for C₂₀H₁₈N₂O₄: 350, found [M+H]⁺=351. ¹H NMR (400 MHz, CDCl₃) δ 7.40-7.36 (m, 1H), 7.32-7.27 (m, 2H), 7.25-7.20 (m, 1H), 7.13 (t, 1H), 6.32 (d, 1H), 6.07 (s, 1H), 4.74 (d, 1H), 3.59-3.55 (m, 1H), 3.43-3.37 (m, 1H), 2.58-2.55 (m, 1H), 2.20-2.14 (m, 4H), 2.10-2.07 (m, 2H). The absolute stereochemistry of this compound was not determined.

Synthesis of chiral 1-(2-oxo-4-(o-tolyl)-2H-pyrano[2,3-b]pyridin-7-yl)pyrrolidine-2-carboxylic acid, Example 38 [Step 7]: To a stirred solution of methyl 1-(2-oxo-4-(o-tolyl)-2H-pyrano[2,3-b]pyridin-7-yl)pyrrolidine-2-carboxylate (37-9, 120 mg, 0.3 mmol) in THF (2 mL) was added HCl (2 ml, 7 mmol, 6 N). The reaction mixture was stirred at ambient temperature for 18 h. The resulting mixture was concentrated under reduced pressure and the product was purified by RP prep-HPLC purification and lyophilized to afford chiral 1-(2-oxo-4-(o-tolyl)-2H-pyrano[2,3-b]pyridin-7-yl)pyrrolidine-2-carboxylic acid (Example 38, 90 mg). ¹H NMR (400 MHz, DMSO-d₆) (100° C.) δ 7.43-7.37 (m, 2H), 7.35-7.31 (m, 1H), 7.20 (d, 1H), 7.08 (d, 1H), 6.43 (d, 1H), 5.92 (s, 1H), 4.48 (br s, 1H), 3.58 (d, 2H), 2.32-2.27 (m, 1H), 2.14 (s, 3H), 2.12-2.07 (m, 1H), 2.04-2.01 (m, 2H). LCMS (ESI) Calcd. for C₂₀H₁₈N₂O₄: 350, found [M+H]⁺=351. The absolute stereochemistry of this compound was not determined.

### Example 39: Synthesis of N-methyl-2-[methyl-[4-(o-tolyl)-2-oxo-pyrano[2,3-b]pyridin-7-yl]amino]acetamide

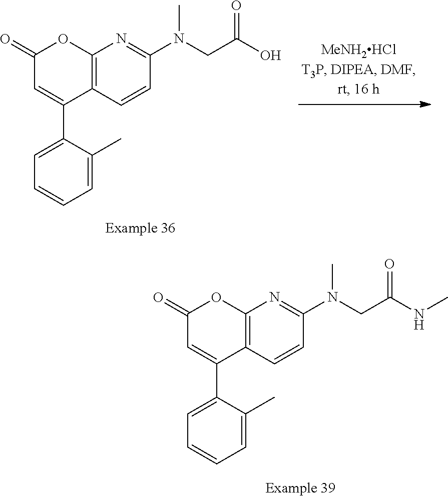 [SMILES: Cc1ccccc1-c1cc(=O)oc2nc(N(C)CC(=O)O)ccc12 ; CNC(=O)CN(C)c1ccc2c(-c3ccccc3C)cc(=O)oc2n1]

To a stirred solution of 2-[methyl-[4-(o-tolyl)-2-oxo-pyrano[2,3-b]pyridin-7-yl]amino]acetic acid (Example 36, 80 mg, 0.247 mmol) in dry DMF (1 mL), T3P (0.13 mL, 0.444 mmol) and DIPEA (0.15 mL, 0.863 mmol) were added. The reaction mixture was stirred at 0° C. for 15 min. and methanamine hydrochloride (25 mg, 0.370 mmol) was added. The resulting reaction mixture and stirred at ambient temperature for additional 16 h. The reaction mixture was diluted with ethyl acetate, washed with ice-cold water, dried over anhydrous sodium sulphate, filtered and concentrated under reduced pressure. The product was purified by prep-HPLC to afford N-methyl-2-[methyl-[4-(o-tolyl)-2-oxo-pyrano[2,3-b]pyridin-7-yl]amino]acetamide (Example 39, 10 mg). LCMS (ESI) Calcd. for C₁₉H₁₉N₃O₃: 337, found [M+H]⁺=338. ¹H NMR (400 MHz, DMSO-d₆): δ 7.91 (d, 1H), 7.44-7.40 (m, 2H), 7.38-7.32 (m, 1H), 7.22-7.20 (m, 1H), 7.09-7.07 (m, 1H), 6.62 (d, 1H), 6.00 (s, 1H), 4.21 (br s, 2H), 3.11 (s, 3H), 2.60 (d, 3H), 2.12 (s, 3H).

### Example 40: Synthesis of 2-[methyl-[4-(o-tolyl)-2-oxo-pyrano[2,3-b]pyridin-7-yl]amino]acetamide

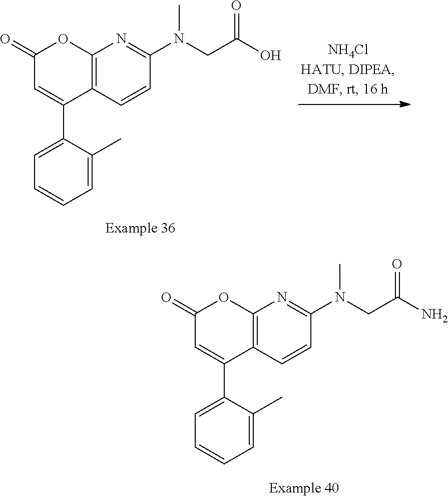 [SMILES: Cc1ccccc1-c1cc(=O)oc2nc(N(C)CC(=O)O)ccc12 ; Cc1ccccc1-c1cc(=O)oc2nc(N(C)CC(N)=O)ccc12]

To a stirred solution of 2-[methyl-[4-(o-tolyl)-2-oxo-pyrano[2,3-b]pyridin-7-yl]amino]acetic acid (Example 36, 80 mg, 0.247 mmol) in dry DMF (1 mL), HATU (141 mg, 0.370 mmol) and DIPEA (0.17 mL, 0.987 mmol) were added. The reaction mixture was stirred at 25° C. for 15 min. and ammonium chloride (40 mg, 0.740 mmol) was added. The resulting reaction mixture was stirred at ambient temperature for additional 16 h., diluted with ethyl acetate, and washed with ice-cold water. The combined organic extracts were dried over anhydrous sodium sulphate, filtered and concentrated under reduced pressure. The product was purified by prep-HPLC to afford 2-[methyl-[4-(o-tolyl)-2-oxo-pyrano[2,3-b]pyridin-7-yl]amino]acetamide (Example 40, 23 mg). LCMS (ESI) Calcd. for C₁₈H₁₇N₃O₃: 323, found [M+H]⁺=324. ¹H NMR (400 MHz, DMSO-d₆): δ 7.46-7.38 (m, 3H), 7.36-7.32 (m, 1H), 7.21 (d, 1H), 7.09-7.07 (m, 2H), 6.61-6.59 (m, 1H), 6.00 (s, 1H), 4.19 (s, 2H), 3.11 (s, 3H), 2.12 (s, 3H).

### Example 41-43: Synthesis of 3-[methyl-[4-(o-tolyl)-2-oxo-pyrano[2,3-b]pyridin-7-yl]amino]propanoic acid, 3-[methyl-[4-(o-tolyl)-2-oxo-pyrano[2,3-b]pyridin-7-yl]amino]propenamide, and N-methyl-3-[methyl-[4-(o-tolyl)-2-oxo-pyrano[2,3-b]pyridin-7-yl]amino]propenamide

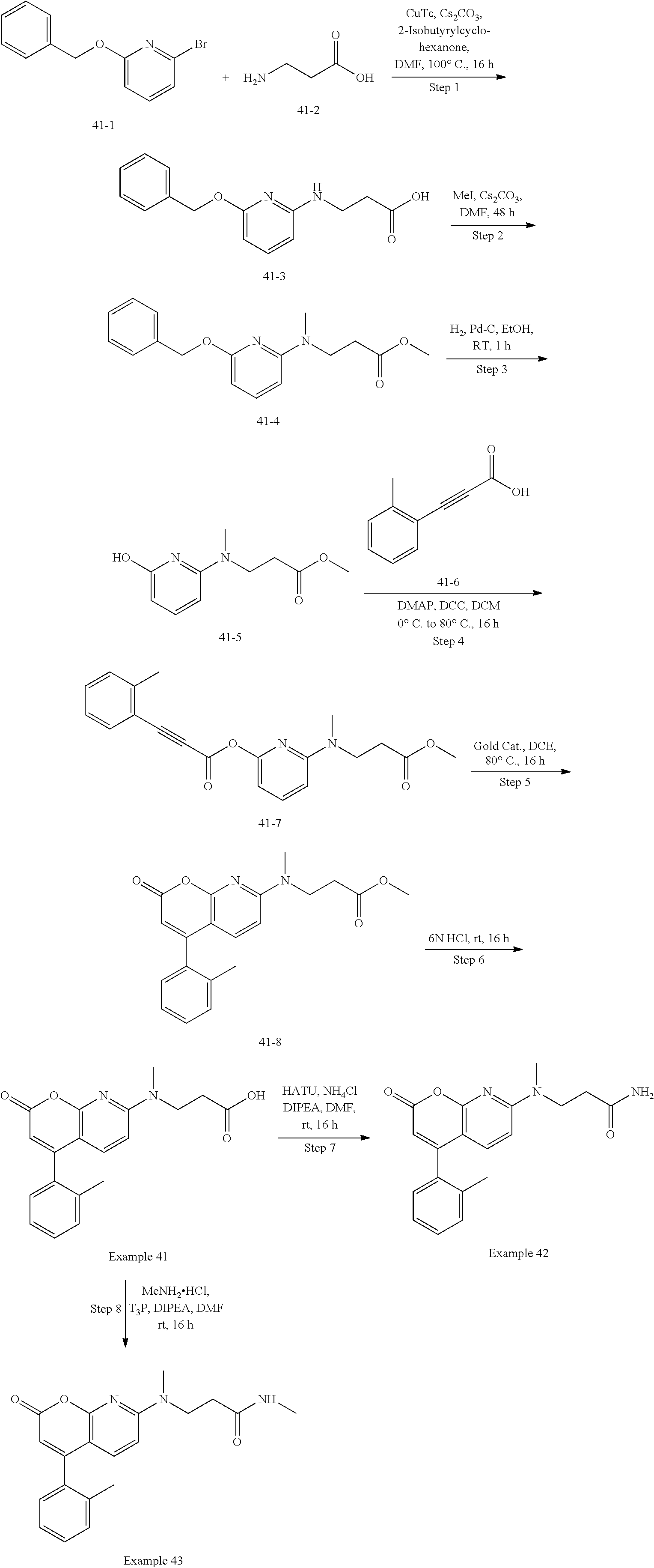

Synthesis of 3-[(6-benzyloxy-2-pyridyl)amino]propanoic acid, 41-3 [Step 1]: In a sealed tube, a mixture of 2-benzyloxy-6-bromo-pyridine (41-1, 3.00 g, 11.4 mmol), cesium carbonate (7.40 g, 22.7 mmol) and 3-amino propionic acid (41-2, 1.21 g, 13.6 mmol) in N,N-dimethylformamide (20 mL) was purged with argon for 15 min. Copper thiophene-2-carboxylate (433 mg, 2.27 mmol) and 2-isobutyrylcyclohexanone (1.53 g, 9.09 mmol) were added. The resulting mixture was heated at 100° C. for 16 h. The reaction mixture was filtered through a celite pad and washed with ethyl acetate. The combined filtrate was concentrated under reduced pressure. The residue was re-dissolved in water, washed with ethyl acetate. The aqueous layer was acidified to pH 4 with TN aqueous solution of hydrochloric acid and extracted with ethyl acetate. The combined extracts were washed with brine, dried over anhydrous sodium sulphate and concentrated under reduced pressure to afford 3-[(6-benzyloxy-2-pyridyl)amino]propanoic acid (41-3, 3.00 g). The product was carried to next step without further purification. LCMS (ESI) Calcd. for C₁₅H₁₆N₂O₃: 272, found [M+H]⁺=273.

Synthesis of methyl 3-[(6-benzyloxy-2-pyridyl)-methyl-amino]propanoate, 41-4 [Step 2]: To a stirred solution of 3-[(6-benzyloxy-2-pyridyl)amino]propanoic acid (41-3, 3.0 g, 11.0 mmol) in N,N-dimethylformamide (30 mL), cesium carbonate (10.77 g, 33.1 mmol) and iodomethane (2.1 mL, 33.1 mmol) were added. The reaction mixture was stirred at ambient temperature for 48 h. The reaction mixture diluted with ethyl acetate, filtered through sintered funnel and washed with ice-cold water followed by brine wash. The combined organic extract was dried over anhydrous sodium sulphate and concentrated under reduced pressure. The product was purified by flash chromatography to afford methyl methyl 3-[(6-benzyloxy-2-pyridyl)-methyl-amino]propanoate (41-4, 830 mg). LCMS (ESI) Calcd. for C₁₇H₂₀N₂O₃: 300, found [M+H]⁺=301. ¹H NMR (400 MHz, DMSO-d₆): δ 7.44-7.32 (m, 5H), 7.30-7.28 (m, 1H), 6.09 (d, 1H), 6.02 (d, 1H), 5.29 (s, 2H), 3.72 (t, 2H), 3.57 (s, 3H), 2.92 (s, 3H), 2.50-2.45 (m, 2H).

Synthesis of methyl 3-[(6-hydroxy-2-pyridyl)-methyl-amino]propanoate, 41-5 [Step 3]: To a stirred solution of methyl 2-[(6-benzyloxy-2-pyridyl)-methyl-amino]acetate (41-4, 400 mg, 1.38 mmol) in dry methanol (5 mL), 10% Pd/C (w/w) (120 mg) was added. The reaction mixture was hydrogenated using hydrogen balloon at ambient temperature for 1 h. The reaction was filtered through a pad of celite and washed with methanol. The combined filtrate was concentrated under reduced pressure to afford methyl 3-[(6-hydroxy-2-pyridyl)-methyl-amino]propanoate (41-5, 400 mg). The product was carried to the next step without further purification. LCMS (ESI) Calcd. for C₁₀H₁₄N₂O₃: 210, found [M+H]⁺=211.

Synthesis of methyl 3-[methyl-[6-[3-(o-tolyl)prop-2-ynoyloxy]-2-pyridyl]amino]propanoate, 41-7 [Step 4]: To a solution of 3-[(6-hydroxy-2-pyridyl)-methyl-amino]propanoate (41-5, 400 mg, 1.99 mmol), 3-(o-tolyl)prop-2-ynoic acid (41-6, 352 mg, 2.19 mmol) and DMAP (48 mg, 0.399 mmol) in dichloromethane (6 mL) at 0° C., a solution of N,N-dicyclohexylcarbodiimide (659 mg, 3.19 mmol) in dichloromethane (1 mL) was added dropwise. The resulting reaction mixture was allowed to stir at ambient temperature for 16 h., diluted with dichloromethane and washed with water followed by brine. The combined organic extracts were dried over anhydrous sodium sulphate and concentrated under reduced pressure. The product was purified by combiflash chromatography to afford methyl 3-[methyl-6-[3-(o-tolyl)prop-2-ynoyloxy-2-pyridyl]amino]propanoate (41-7, 150 mg). LCMS (ESI) Calcd. for C₂₀H₂₀N₂O₄: 352, found [M+H]⁺=353. ¹H NMR (400 MHz, DMSO-d₆): δ 7.69-7.62 (m, 2H), 7.47 (t, 1H), 7.39 (d, 1H), 7.31 (t, 1H), 6.60 (d, 1H), 6.45 (d, 1H), 3.79-3.72 (m, 2H), 3.61 (s, 3H), 2.98 (s, 3H), 2.66-2.55 (m, 2H), 2.35 (s, 3H).

Synthesis of methyl 3-[methyl-[4-(o-tolyl)-2-oxo-pyrano[2,3-b]pyridin-7-yl]amino]propanoate, 41-8 [Step 5]: A solution 3-[methyl-[6-[3-(o-tolyl)prop-2-ynoyloxy]-2-pyridyl]amino]propanoate (41-7, 150 mg, 0.381 mmol) in dry dichloroethane (2 mL) was purged with argon for 10 min. and (acetonitrile)[(2-biphenyl)di-tert-butylphosphine]gold(I) hexafluoroantimonate (32 mg, 0.042 mmol) was added. The reaction mixture was heated at 70° C. for 4 h. The reaction mixture was filtered through a pad of celite, washed with ethyl acetate and the combined filtrates were concentrated under reduced pressure. The product was purified by flash chromatography to afford methyl 3-[methyl-[4-(o-tolyl)-2-oxo-pyrano[2,3-b]pyridin-7-yl]amino]propanoate (41-8, 80 mg, 0.208 mmol). LCMS (ESI) Calcd. for C₂₀H₂₀N₂O₄: 352, found [M+H]⁺=353. ¹H NMR (400 MHz, DMSO-d₆): δ 7.42-7.33 (m, 3H), 7.21 (d, 1H), 7.06 (d, 1H), 6.62 (d, 1H), 5.99 (s, 1H), 3.85-3.84 (m, 2H), 3.60 (s, 3H), 3.08 (s, 3H), 2.65 (t, 2H), 2.12 (s, 3H).

Synthesis of 3-[methyl-[4-(o-tolyl)-2-oxo-pyrano[2,3-b]pyridin-7-yl]amino]propanoic acid, Example 41 [Step 6]: A solution of methyl 3-[methyl-[4-(o-tolyl)-2-oxo-pyrano[2,3-b]pyridin-7-yl]amino]propanoate (41-8) aqueous 6N HCl (4 mL) was stirred at ambient temperature for 16 h. The volatile was evaporated under reduced pressure. The residue was triturated with n-pentane and lyophilized to afford 3-[methyl-[4-(o-tolyl)-2-oxo-pyrano[2,3-b]pyridin-7-yl]amino]propanoic acid (Example 41, 27 mg). LCMS (ESI) Calcd. C₁₉H₁₈N₂O₄ for 338, found [M+H]⁺=339. ¹H NMR (400 MHz, DMSO-d₆): δ 7.42-7.32 (m, 3H), 7.21 (d, 1H), 7.05 (d, 1H), 6.62 (d, 1H), 5.99 (s, 1H), 3.79 (t, 2H), 3.09 (s, 3H), 2.56 (t, 2H), 2.12 (s, 3H).

Synthesis of 3-[methyl-[4-(o-tolyl)-2-oxo-pyrano[2,3-b]pyridin-7-yl]amino]propenamide, Example 42 [Step 7]: To a solution of 3-[methyl-[4-(o-tolyl)-2-oxo-pyrano[2,3-b]pyridin-7-yl]amino]propanoic acid (Example 41, 55 mg, 0.163 mmol) in N,N-dimethylformamide (3 mL), DIPEA (0.11 mL, 0.650 mmol) and HATU (93 mg, 0.244 mmol) were added. The reaction mixture was stirred at ambient temperature for 10 min. and ammonium chloride (26 mg, 0.488 mmol) was added. The resulting reaction mixture was allowed to stir at ambient temperature for additional 16 h. and concentrated under reduced pressure. The product was purified by prep-HPLC to afford 3-[methyl-[4-(o-tolyl)-2-oxo-pyrano[2,3-b]pyridin-7-yl]amino]propenamide (Example 42, 18 mg). LCMS (ESI) Calcd. for C₁₉H₁₉N₃O₃: 337, found [M+H]⁺=338. ¹H NMR (400 MHz, DMSO-d₆): δ 7.43-7.38 (m, 3H), 7.34 (t, 1H), 7.21 (d, 1H), 7.05 (d, 1H), 6.88 (br s, 1H), 6.62 (d, 1H), 5.97 (s, 1H), 3.77 (t, 2H), 3.08 (s, 3H), 2.38 (t, 2H), 2.11 (s, 3H).

Synthesis of N-methyl-3-[methyl-[4-(o-tolyl)-2-oxo-pyrano[2,3-b]pyridin-7-yl]amino]propenamide, Example 43 [Step 8]: To a solution of 3-[methyl-[4-(o-tolyl)-2-oxo-pyrano[2,3-b]pyridin-7-yl]amino]propanoic acid (Example 41, 55 mg, 0.163 mmol) in N,N-dimethylformamide (3 mL), DIPEA (0.11 mL, 0.650 mmol) and propylphosphonic anhydride (0.19 mL, 0.325 mmol) were added. The reaction mixture was stirred at ambient temperature for 10 min. and methyl amine hydrochloride (16 mg, 0.244 mmol) was added. The reaction mixture was allowed to stir at ambient temperature additional 16 h. and concentrated under reduced pressure. The product was purified by prep-HPLC to afford N-methyl-3-[methyl-[4-(o-tolyl)-2-oxo-pyrano[2,3-b]pyridin-7-yl]amino]propenamide (Example 43, 14 mg). LCMS (ESI) Calcd. for C₂₀H₂₁N₃O₃: 351, found [M+H]⁺=352. ¹H NMR (400 MHz, DMSO-d₆): δ 7.85 (br s, 1H), 7.42-7.34 (m, 3H), 7.21 (d, 1H), 7.05 (d, 1H), 6.60 (d, 1H), 5.98 (s, 1H), 3.78 (t, 2H), 3.06 (s, 3H), 2.55 (s, 3H), 2.39 (t, 2H), 2.12 (s, 3H).

### Example 44-46: N-(4-(2-chloro-4-fluorophenyl)-2-oxo-2H-pyrano[2,3-b]pyridin-7-yl)-N-methylglycine, 2-((4-(2-chloro-4-fluorophenyl)-2-oxo-2H-pyrano[2,3-b]pyridin-7-yl)(methyl)amino)-N-methylacetamide, and 2-((4-(2-chloro-4-fluorophenyl)-2-oxo-2H-pyrano[2,3-b]pyridin-7-yl)(methyl)amino)acetamide

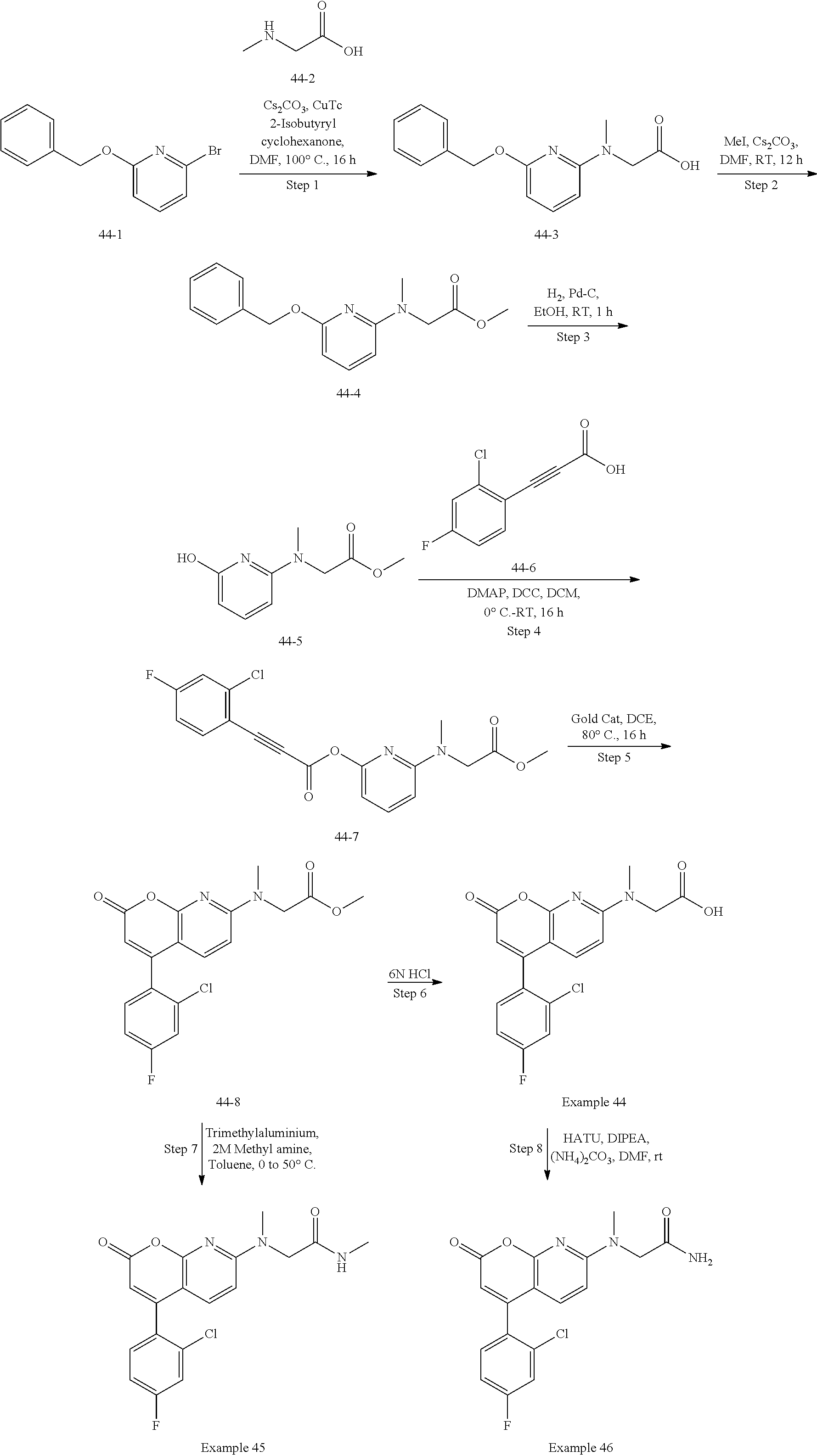

Synthesis of N-(6-(benzyloxy)pyridin-2-yl)-N-methylglycine, 44-3 [Step 1]: A solution of 2-benzyloxy-6-bromo-pyridine (44-1, 1.0 g, 3.8 mmol), 2-(methylamino)acetic acid (44-2, 335 mg, 3.8 mmol), Cs₂CO₃ (2.5 g, 7.6 mmol) in dry DMF (7 mL) taken in a sealed tube was degassed with argon gas for 15 min. Copper thiophene 2-carboxylate (145 mg, 0.8 mmol) and 2-isobutyrylcyclohexanone (0.5 mL, 3.0 mmol) were added to the reaction mixture and it was stirred at 100° C. for 16 h. The reaction mass was cooled, diluted with water and ethyl acetate and aqueous layer was separated. The aqueous fraction was acidified to ˜pH 4-5 using aqueous HCl (2N) and the mixture was extracted with a mixture of methanol in dichloromethane (1:9). The combined organic extracts were dried over anhydrous sodium sulphate, filtered and concentrated under reduced pressure to afford N-(6-(benzyloxy)pyridin-2-yl)-N-methylglycine (44-3, 900 mg). LCMS (ESI) Calcd. for C₁₅H₁₆N₂O₃: 272, found [M+H]⁺=273.

Synthesis of methyl N-(6-(benzyloxy)pyridin-2-yl)-N-methylglycinate 44-4 [Step 2]: A mixture of N-(6-(benzyloxy)pyridin-2-yl)-N-methylglycine (44-3, 1.0 g, 3.7 mmol) and Cs₂CO₃ (2.4 g, 7.3 mmol) in dry dimethylformamide (5 mL) was allowed to cool at 0° C. for 10 min. Iodomethane (0.5 mL, 7.3 mmol) was added to the resulting mixture dropwise. The reaction mixture was allowed to stir at ambient temperature for 16 h. The reaction was diluted with ethyl acetate and washed with ice-water and cold brine. The organic extract was dried over anhydrous sodium sulphate, filtered and concentrated under reduced pressure. The product was purified by column-chromatography to afford methyl N-(6-(benzyloxy)pyridin-2-yl)-N-methylglycinate (44-4, 1.0 g). LCMS (ESI) Calcd. for C₁₆H₁₈N₂O₃: 286, found [M+H]⁺=287. ¹H NMR (400 MHz, DMSO-d₆) δ 7.45 (d, 1H), 7.38-7.27 (m, 5H), 6.18 (d, 1H), 6.07 (d, 1H), 5.21 (s, 2H), 4.27 (s, 2H), 3.58 (d, 3H), 3.02 (s, 3H).

Synthesis of methyl N-(6-hydroxypyridin-2-yl)-N-methylglycinate, 44-5 [Step 3]: To a stirred solution of methyl N-(6-(benzyloxy)pyridin-2-yl)-N-methylglycinate (44-4, 700 mg, 2.4 mmol) in dry methanol (5 mL), 10% Pd/C (w/w) (350 mg, 3.3 mmol) and (0.2 mL) HCl solution were added under inert atmosphere and allowed to stir at ambient temperature for 1 h. under H₂ balloon. The reaction was filtered over celite bed and washed with methanol, concentrated under reduced pressure to afford methyl N-(6-hydroxypyridin-2-yl)-N-methylglycinate (44-5, 450 mg). LCMS (ESI) Calcd. for C₉H₁₂N₂O₃: 196, found [M+H]⁺=197.

Synthesis of 6-((2-methoxy-2-oxoethyl)(methyl)amino)pyridin-2-yl 3-(2-chloro-4-fluorophenyl)propiolate, 44-7 [Step 4]: To a stirred solution of methyl N-(6-hydroxypyridin-2-yl)-N-methylglycinate (44-5, 150 mg, 0.8 mmol)) in dichloromethane (20 mL), 3-(2-chloro-4-fluorophenyl)propiolic acid (44-6, 150 mg, 0.8 mmol) and DMAP (45 mg, 0.4 mmol) were added. Into this mixture a solution of DCC (205 mg, 1.0 mmol) in dichloromethane (2 mL) was added dropwise at 0° C. The reaction mixture was stirred at ambient temperature for 16 h. After completion, the reaction mixture was filtered through celite bed and washed with dichloromethane and concentrated under reduced pressure. The product was purified using column chromatography to afford 6-((2-methoxy-2-oxoethyl)(methyl)amino)pyridin-2-yl 3-(2-chloro-4-fluorophenyl)propiolate (44-7, 90 mg). LCMS (ESI) Calcd. for C₁₈H₁₄ClFN₂O₄: 377, found [M+H]⁺=377.

Synthesis of methyl N-(4-(2-chloro-4-fluorophenyl)-2-oxo-2H-pyrano[2,3-b]pyridin-7-yl)-N-methylglycinate, 44-8 [Step 5]: To a stirred solution of 6-((2-methoxy-2-oxoethyl)(methyl)amino)pyridin-2-yl 3-(2-chloro-4-fluorophenyl)propiolate (44-7, 90 mg, 0.2 mmol) in dichloroethane (2 mL) (Acetonitrile)[(2-biphenyl)di-tert-butylphosphine]gold(I) hexafluoroantimonate (20 mg, 0.02 mmol) was added under inert atmosphere and the reaction mixture was stirred at 80° C. for 18 h. The mixture was diluted with dichloromethane, filtered through celite bed and concentrated under reduced pressure. The product was purified by column chromatography to afford methyl N-(4-(2-chloro-4-fluorophenyl)-2-oxo-2H-pyrano[2,3-b]pyridin-7-yl)-N-methylglycinate (44-8, 50 mg). LCMS (ESI) Calcd. for C₁₈H₁₄ClFN₂O₄: 377, found [M+H]⁺=377. ¹H NMR (400 MHz, DMSO-d₆) δ 7.70 (dd, 1H), 7.56-7.52 (m, 1H), 7.44-7.40 (m, 1H), 7.22 (d, 1H), 6.71 (d, 1H), 6.14 (s, 1H), 4.46 (s, 2H), 3.67 (s, 3H), 3.14 (s, 3H).

Synthesis of N-(4-(2-chloro-4-fluorophenyl)-2-oxo-2H-pyrano[2,3-b]pyridin-7-yl)-N-methylglycine, Example 44 [Step 6]: To a stirred solution of methyl N-(4-(2-chloro-4-fluorophenyl)-2-oxo-2H-pyrano[2,3-b]pyridin-7-yl)-N-methylglycinate (44-8, 55 mg, 0.1 mmol) in THF (2 mL), 6 N HCl solution (2 mL) was added to the reaction mixture and it was stirred at ambient temperature for 48 h. The volatiles were concentrated under reduced pressure and the product was purified by reverse phase PREP HPLC purification and lyophilised to afford N-(4-(2-chloro-4-fluorophenyl)-2-oxo-2H-pyrano[2,3-b]pyridin-7-yl)-N-methylglycine (Example 44, 18 mg). LCMS (ESI) Calcd. for C₁₇H₁₂ClFN₂O₄: 363, found [M+H]⁺=363. ¹H NMR (400 MHz, DMSO-d₆) δ 7.69 (d, 1H), 7.54 (t, 1H), 7.43 (t, 1H), 7.12 (s, 1H), 6.61 (s, 1H), 6.06 (s, 1H), 4.18 (s, 2H), 3.10 (s, 3H).

Synthesis of 2-((4-(2-chloro-4-fluorophenyl)-2-oxo-2H-pyrano[2,3-b]pyridin-7-yl)(methyl)amino)-N-methylacetamide, Example 45 [Step 7]: A stirred solution methyl N-(4-(2-chloro-4-fluorophenyl)-2-oxo-2H-pyrano[2,3-b]pyridin-7-yl)-N-methylglycinate (44-8, 75 mg, 0.2 mmol) in toluene (2 mL) was cooled to 0° C. and 2 M methyl amine in THF (0.5 mL, 0.4 mmol) was added dropwise followed by 2 M trimethyl aluminium (0.5 mL, 0.4 mmol) at same temperature and the reaction mixture was heated at 50° C. for 12 h. After completion, the reaction mixture was quenched with ice water and extracted with ethyl acetate. The organic extract was washed with brine, dried over anhydrous Na₂SO₄, concentrated under reduced pressure and purified by reverse phase PREP HPLC and lyophilised to afford 2-((4-(2-chloro-4-fluorophenyl)-2-oxo-2H-pyrano[2,3-b]pyridin-7-yl)(methyl)amino)-N-methylacetamide (Example 45, 16 mg) LCMS (ESI) Calcd. for C₁₈H₁₅ClFN₃O₃:376, found [M+H]⁺=376. ¹H NMR (400 MHz, DMSO-d₆) δ 7.92 (d, 1H), 7.70 (dd, 1H), 7.54-7.51 (m, 1H), 7.42 (d, 1H), 7.18 (d, 1H), 6.64 (s, 1H), 6.11 (s, 1H), 4.22 (d, 2H), 3.11 (s, 3H), 2.60 (d, 3H).

Synthesis of 2-((4-(2-chloro-4-fluorophenyl)-2-oxo-2H-pyrano[2,3-b]pyridin-7-yl)(methyl)amino)acetamide, Example 46 [Step 8]: To a stirred solution of N-(4-(2-chloro-4-fluorophenyl)-2-oxo-2H-pyrano[2,3-b]pyridin-7-yl)-N-methylglycine (Example 44, 80 mg, 0.2 mmol) in DMF (3 mL), DIPEA (0.26 mL, 1.5 mmol) and HATU (90 mg, 0.2 mmol) were added. The reaction mixture was allowed to stir for 5 min. and ammonium carbonate (145 mg, 1.5 mmol) was added. The reaction mixture was stirred at ambient temperature for 16 h. After completion the mixture was diluted with ethyl acetate and washed with ice-cold water. The organic extract was dried over anhydrous Na₂SO₄, filtered and concentrated under reduced pressure. The product was purified by reverse phase prep HPLC and lyophilised to afford 2-((4-(2-chloro-4-fluorophenyl)-2-oxo-2H-pyrano[2,3-b]pyridin-7-yl)(methyl)amino)acetamide (Example 46, 26 mg). LCMS (ESI) Calcd. for C₁₇H₁₃ClFN₃O₃: 362, found [M+H]⁺: 362. ¹H NMR (400 MHz, DMSO-d₆) δ 7.69 (t, 1H), 7.55 (t, 1H), 7.44 (t, 2H), 7.17 (d, 1H), 7.08 (s, 1H), 6.62 (s, 1H), 6.10 (s, 1H), 4.20 (s, 2H), 3.14 (s, 3H).

### Example 47: Synthesis of 3-((4-(2-chloro-4-fluorophenyl)-2-oxo-2H-pyrano[2,3-b]pyridin-7-yl)(methyl)amino)-N-methylpropanamide

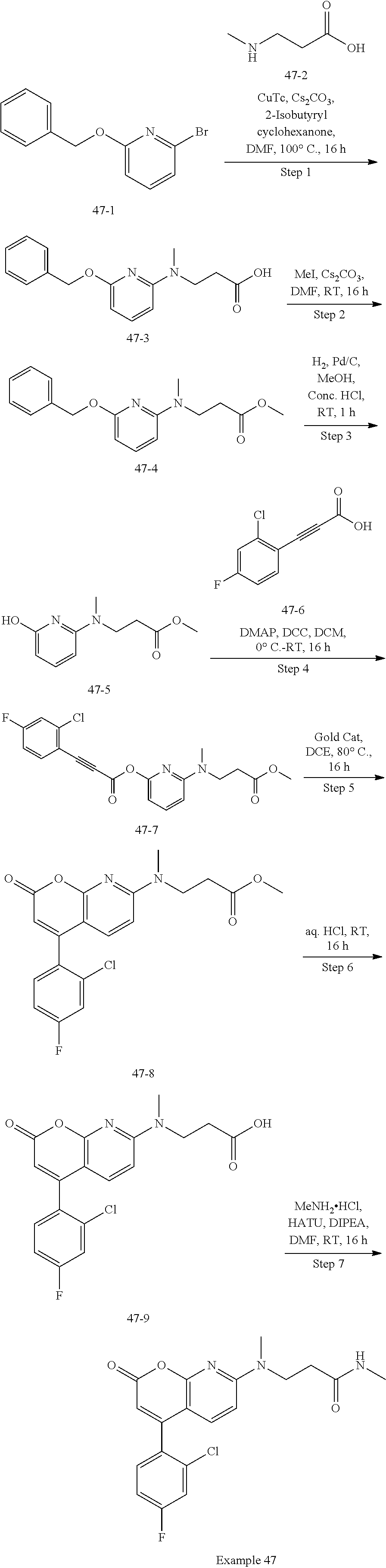

Synthesis of 3-((6-(benzyloxy)pyridin-2-yl)(methyl)amino)propanoic acid, 47-3 [Step 1]: In a sealed tube, a mixture of 2-benzyloxy-6-bromo-pyridine (47-1, 2.0 g, 7.57 mmol), Cs₂CO₃ (4.9 g, 15.1 mmol), 3-(methylamino)propanoic acid (47-2, 935 mg, 9.1 mmol) and 2-isobutyrylcyclohexanone (1.0 mL, 6.1 mmol) in dry DMF (5 mL) was purged with argon gas for 15 min. Copper thiophene-2-carboxylate (290 mg, 1.5 mmol) was added. The resulting mixture was heated at 100° C. for 16 h. After cooling, the reaction mixture diluted with ethyl acetate, washed with water. The aqueous fraction was acidified to pH=5 by aqueous HCl (2N) and extracted with ethyl acetate (thrice). The combined organic extracts were dried over anhydrous sodium sulphate, filtered and concentrated under reduced pressure to give 3-((6-(benzyloxy)pyridin-2-yl)(methyl)amino)propanoic acid (47-3, 1.0 g). LCMS (ESI) Calcd. for C₁₆H₁₈N₂O₃: 286, found [M+H]⁺=287.

Synthesis of methyl 3-((6-(benzyloxy)pyridin-2-yl)(methyl)amino)propanoate, 47-4 [Step 2]: To a stirred solution of 3-((6-(benzyloxy)pyridin-2-yl)(methyl)amino)propanoic acid (47-3, 1.0 g, 3.5 mmol) in DMF (5 mL) at 0° C., Cs₂CO₃ (3.4 g, 10.5 mmol) was added. Methyl iodide (0.7 mL, 14.0 mmol) was added and the reaction mixture was stirred at ambient temperature for 16 h. After completion, the reaction mixture was diluted with ethyl acetate and washed with brine solution. The combined organic extracts were dried over anhydrous sodium sulphate, filtered and concentrated under reduced pressure. The product was purified by flash chromatography to give methyl 3-((6-(benzyloxy)pyridin-2-yl)(methyl)amino)propanoate (47-4, 450 mg). LCMS (ESI) Calcd. for C₁₇H₂₀N₂O₃: 300, found [M+H]⁺=301, ¹H NMR (400 MHz, DMSO-d₆) δ 7.44-7.27 (m, 6H), 6.09 (d, 1H), 6.02 (d, 1H), 5.29 (s, 2H), 3.72 (t, 2H), 3.57 (s, 3H), 3.31-3.29 (m, 2H), 2.92 (s, 3H).

Synthesis of methyl 3-((6-hydroxypyridin-2-yl)(methyl)amino)propanoate, 47-5 [Step 3]: To a stirred solution of methyl 3-((6-(benzyloxy)pyridin-2-yl)(methyl)amino)propanoate (47-4, 200 mg, 0.7 mmol) in dry methanol (5 mL), nitrogen was purged for 5 min. Pd/C (10% w/w) (85 mg, 0.8 mmol) and conc. hydrochloric acid (1.2 mL) were added to the mixture. The reaction mixture was hydrogenated using hydrogen balloon at ambient temperature for 1 h. After completion, the reaction was filtered through a pad of celite and washed with methanol. The combined filtrate was concentrated under reduced pressure to afford methyl 3-((6-hydroxypyridin-2-yl)(methyl)amino)propanoate (47-5, 110 mg). LCMS (ESI) Calcd. for C₁₀H₁₄N₂O₃: 210, found [M+H]⁺=211.

Synthesis of 6-((3-methoxy-3-oxopropyl)(methyl)amino)pyridin-2-yl 3-(2-chloro-4-fluorophenyl)propiolate, 47-7 [Step 4]: To a solution of 3-(2-chloro-4-fluorophenyl)propiolic acid (47-6, 105 mg, 0.5 mmol), methyl 3-((6-hydroxypyridin-2-yl)(methyl)amino)propanoate (47-5, 100 mg, 0.5 mmol) and DMAP (25 mg, 0.2 mmol) in dichloromethane (4 mL) at 0° C., a solution of DCC (155 mg, 0.8 mmol) in dichloromethane (1 mL) was added dropwise. The resulting reaction mixture was allowed to stir at ambient temperature for 16 h. After completion, the reaction mixture was diluted with dichloromethane and filtered. The filtrate was dried over anhydrous sodium sulphate and concentrated under reduced pressure. The product was purified by flash chromatography to afford 6-((3-methoxy-3-oxopropyl)(methyl)amino)pyridin-2-yl 3-(2-chloro-4-fluorophenyl)propiolate (47-7, 70 mg). LCMS (ESI) Calcd. for C₁₉H₁₆ClFN₂O₄: 390, found [M+H]⁺=391. ¹H NMR (400 MHz, DMSO-d₆) δ 7.95-7.91 (m, 1H), 7.75-7.73 (m, 1H), 7.67 (t, 1H), 7.42-7.38 (m, 1H), 6.60 (d, 1H), 6.46 (d, 1H), 3.74-3.69 (m, 2H), 3.56 (s, 3H), 2.99 (s, 3H), 2.59-2.57 (m, 2H).

Synthesis of methyl 3-((4-(2-chloro-4-fluorophenyl)-2-oxo-2H-pyrano[2,3-b]pyridin-7-yl)amino)propanoate, 47-8 [Step 5]: A solution of 6-((3-methoxy-3-oxopropyl)(methyl)amino)pyridin-2-yl 3-(2-chloro-4-fluorophenyl)propiolate (47-7, 80 mg, 0.2 mmol) in dry dichloroethane (2 mL) was purged with argon gas for 10 min. and (acetonitrile)[(2-biphenyl)di-tert-butylphosphine]gold(I) hexafluoroantimonate (16 mg, 0.21 mmol) was added. The reaction mixture was heated at 80° C. for 16 h. After cooling, the reaction mixture was filtered through a pad of celite, washed with ethyl acetate and concentrated under reduced pressure to afford methyl 3-((4-(2-chloro-4-fluorophenyl)-2-oxo-2H-pyrano[2,3-b]pyridin-7-yl)amino)propanoate (47-8, 70 mg). LCMS (ESI) Calcd. for C₁₈H₁₆ClFN₂O₄: 390, found [M+H]⁺=391.

Synthesis of 3-((4-(2-chloro-4-fluorophenyl)-2-oxo-2H-pyrano[2,3-b]pyridin-7-yl)(methyl)amino)propanoic acid, 47-9 [Step 6]: A mixture of methyl 3-((4-(2-chloro-4-fluorophenyl)-2-oxo-2H-pyrano[2,3-b]pyridin-7-yl)amino)propanoate (47-8, 70 mg, 0.1 mmol) and aqueous hydrochloric acid (6N) (2 mL) was stirred at ambient temperature for 16 h. After completion, the volatiles was evaporated under reduced pressure to afford 3-((4-(2-chloro-4-fluorophenyl)-2-oxo-2H-pyrano[2,3-b]pyridin-7-yl)(methyl)amino)propanoic acid (47-9, 60 mg). LCMS (ESI) Calcd. for C₁₈H₁₄ClFN₂O₄: 376, found [M+H]⁺=377.

Synthesis of 3-((4-(2-chloro-4-fluorophenyl)-2-oxo-2H-pyrano[2,3-b]pyridin-7-yl)(methyl)amino)-N-methylpropanamide, Example 47 [Step 7]: To a stirred solution of 3-((4-(2-chloro-4-fluorophenyl)-2-oxo-2H-pyrano[2,3-b]pyridin-7-yl)(methyl)amino)propanoic acid (47-9, 60 mg, 0.2 mmol) in dry DMF (1 mL), HATU (120 mg, 0.3 mmol) and DIPEA (0.1 mL, 0.6 mmol) were added. The reaction mixture was stirred at ambient temperature for 15 min. and methylamine hydrochloride (45 mg, 0.6 mmol) was added. The resulting reaction mixture was stirred at ambient temperature for additional 16 h. The reaction mixture was diluted with ethyl acetate, washed with ice-cold water, dried over anhydrous sodium sulphate, filtered and concentrated under reduced pressure. The product was purified by reverse phase prep-HPLC to afford 3-((4-(2-chloro-4-fluorophenyl)-2-oxo-2H-pyrano[2,3-b]pyridin-7-yl)(methyl)amino)-N-methylpropanamide (Example 47, 4.2 mg). LCMS (ESI) Calcd. for C₁₉H₁₇ClFN₃O₃: 389, found [M+H]⁺=390. ¹H NMR (400 MHz, DMSO-d₆) δ 7.87-7.86 (m, 1H), 7.71-7.68 (m, 1H), 7.55-7.51 (m, 1H), 7.43 (t, 1H), 7.14 (d, 1H), 6.63 (d, 1H), 6.09 (s, 1H), 3.78 (br s, 2H), 3.07 (s, 3H), 2.55 (d, 3H), 2.38 (t, 2H).

### Example 48-49: Synthesis of chiral isomers of 2-(methyl(2-oxo-4-(o-tolyl)-2H-pyrano[2,3-b]pyridin-7-yl)amino)propanamide

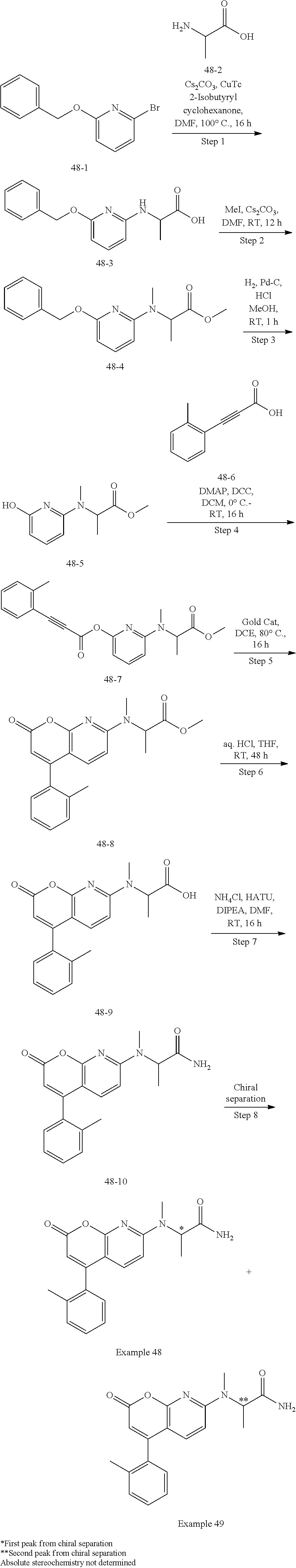

Synthesis of (6-(benzyloxy)pyridin-2-yl)alanine, 48-3 [Step 1]: In a sealed tube, a mixture of 2-(benzyloxy)-6-bromopyridine (48-1, 2.0 g, 7.6 mmol), Cs₂CO₃ (4.9 g, 15.1 mmol) and alanine (48-2, 810 mg, 9.08 mmol) in DMF (15 mL) was purged with argon gas for 15 min. 2-Isobutyrylcyclohexanone (1.0 g, 1.1 mmol) and copper thiophene-2-carboxylate (280 mg, 1.5 mmol) were added. The resulting mixture was heated at 110° C. for 16 h. After cooling, the reaction mixture was filtered through a pad of celite and washed with ethyl acetate. The combined filtrate was dried over anhydrous sodium sulphate and concentrated under reduced pressure to give (6-(benzyloxy)pyridin-2-yl)alanine (48-3, 1.2 g). LCMS (ESI) Calcd. for C₁₅H₁₆N₂O₃: 272, found [M+H]⁺=273.

Synthesis of methyl N-(6-(benzyloxy)pyridin-2-yl)-N-methylalaninate, 48-4 [Step 2]: To a stirred solution of (6-(benzyloxy)pyridin-2-yl)alanine (48-3, 1.2 g, 4.4 mmol) in DMF (10 mL), Cs₂CO₃ (2.9 g, 8.8 mmol) and methyl iodide (2.5 g, 17.6 mmol) were added. The resulting mixture was allowed to stir at ambient temperature for 16 h. The reaction was filtered through a pad of celite and washed with methanol. The combined filtrate was concentrated under reduced pressure to afford methyl N-(6-(benzyloxy)pyridin-2-yl)-N-methylalaninate (48-4, 600 mg). LCMS (ESI) Calcd. for C₁₇H₂₀N₂O₃: 300, found [M+H]⁺=301.

Synthesis of methyl N-(6-hydroxypyridin-2-yl)-N-methylalaninate, 48-5 [Step 3]: To a stirred solution of methyl N-(6-(benzyloxy)pyridin-2-yl)-N-methylalaninate (48-4, 600 mg, 0.9 mmol) in dry methanol (15 mL) Pd/C (w/w) (180 mg, 1.7 mmol, 10%) was added followed by aqueous hydrochloric acid (6 N) (1.2 mL). The reaction mixture was hydrogenated using hydrogen balloon at ambient temperature for 3 h. The reaction was filtered through a pad of celite and washed with methanol. The combined filtrate was concentrated under reduced pressure to afford methyl N-(6-hydroxypyridin-2-yl)-N-methylalaninate (48-5, 310 mg). LCMS (ESI) Calcd. for C₁₀H₁₄N₂O₃: 210, found [M+H]⁺=211.

Synthesis of 6-((1-methoxy-1-oxopropan-2-yl)(methyl)amino)pyridin-2-yl 3-(o-tolyl)propiolate, 48-7 [Step 4]: To a solution of methyl N-(6-hydroxypyridin-2-yl)-N-methylalaninate (48-5, 310 mg, 1.5 mmol), 3-(o-tolyl)propiolic acid (48-6, 285 mg, 1.8 mmol) and DMAP (30 mg, 0.3 mmol) in dichloromethane (15 mL) at 0° C., a solution of DCC (485 mg, 2.4 mmol) in dichloromethane (2 mL) was added dropwise. The resulting reaction mixture was allowed to stir at ambient temperature for 16 h. After completion, the reaction mixture was diluted with dichloromethane and washed with water and brine. The organic extract was dried over anhydrous sodium sulphate and concentrated under reduced pressure. The product was purified by flash chromatography to afford 6-((1-methoxy-1-oxopropan-2-yl)(methyl)amino)pyridin-2-yl 3-(o-tolyl)propiolate (48-7, 150 mg). LCMS (ESI) Calcd. for C₂₀H₂₀N₂O₄: 352, found [M+H]⁺=353.

Synthesis of methyl N-methyl-N-(2-oxo-4-(o-tolyl)-2H-pyrano[2,3-b]pyridin-7-yl)alaninate, 48-8 [Step 5]: A solution of 6-((1-methoxy-1-oxopropan-2-yl)(methyl)amino)pyridin-2-yl 3-(o-tolyl)propiolate (48-7, 150 mg, 0.3 mmol) in dry dichloroethane (3 mL) was purged with argon gas for 10 min. and (acetonitrile)[(2-biphenyl)di-tert-butylphosphine]gold(I) hexafluoroantimonate (49.3 mg, 0.07 mmol) was added. The reaction mixture was heated at 70° C. for 16 h. After cooling, the reaction mixture was filtered through a pad of celite, washed with ethyl acetate and concentrated under reduced pressure to afford methyl N-methyl-N-(2-oxo-4-(o-tolyl)-2H-pyrano[2,3-b]pyridin-7-yl)alaninate (48-8, 88 mg). LCMS (ESI) Calcd. for C₂₀H₂₀N₂O₄: 352, found [M+H]⁺=353.

Synthesis of N-methyl-N-(2-oxo-4-(o-tolyl)-2H-pyrano[2,3-b]pyridin-7-yl)alanine, 48-9 [Step 6]: A solution of methyl N-methyl-N-(2-oxo-4-(o-tolyl)-2H-pyrano[2,3-b]pyridin-7-yl)alaninate (48-8, 86.0 mg) in tetrahydrofuran (1 mL), aqueous hydrochloric acid (6 N) (2 mL) was added and was stirred at ambient temperature for 48 h. The volatiles was evaporated under reduced pressure to afford N-methyl-N-(2-oxo-4-(o-tolyl)-2H-pyrano[2,3-b]pyridin-7-yl)alanine (48-9, 80 mg). LCMS (ESI) Calcd. for C₁₉H₁₈N₂O₄: 338, found [M+H]⁺=339.

Synthesis of 2-(methyl(2-oxo-4-(o-tolyl)-2H-pyrano[2,3-b]pyridin-7-yl)amino)propanamide, 10 [Step 7]: To a stirred solution of N-methyl-N-(2-oxo-4-(o-tolyl)-2H-pyrano[2,3-b]pyridin-7-yl)alanine (48-9, 80.0 mg, 0.2 mmol) in DMF (5 mL), DIPEA (91.5 mg, 0.7 mmol) and HATU (224.7 mg, 0.59 mmol) were added. The reaction mixture was stirred at 25° C. for 5 min. and ammonium chloride (40 mg, 0.7 mmol) was added. The reaction mixture was allowed to stir at ambient temperature for 16 h. The reaction was diluted with ethyl acetate and washed with ice-cold water. The organic extract was dried over anhydrous sodium sulphate, filtered and concentrated under reduced pressure. The product was purified by reverse phase prep-HPLC to afford 2-(methyl(2-oxo-4-(o-tolyl)-2H-pyrano[2,3-b]pyridin-7-yl)amino)propanamide (48-10, 40 mg). LCMS (ESI) Calcd. for C₁₉H₁₉N₃O₃: 337, found [M+H]⁺=338.

Synthesis of chiral isomers of 2-(methyl(2-oxo-4-(o-tolyl)-2H-pyrano[2,3-b]pyridin-7-yl)amino)propanamide, Examples 48 and 49 [Step 8]: Racemic 2-(methyl(2-oxo-4-(o-tolyl)-2H-pyrano[2,3-b]pyridin-7-yl)amino)propanamide (48-10, 40 mg. 0.1 mmol) was separated by SFC HPLC chiral purification and lyophilized to afford the first product as chiral 2-(methyl(2-oxo-4-(o-tolyl)-2H-pyrano[2,3-b]pyridin-7-yl)amino)propanamide (Example 48, 11 mg) as Peak 1, and the second product as chiral 2-(methyl(2-oxo-4-(o-tolyl)-2H-pyrano[2,3-b]pyridin-7-yl)amino)propanamide (Example 49, 10 mg) as Peak 2. The absolute stereochemistries of these compounds were not determined.

Example 48: Chiral 2-(methyl(2-oxo-4-(o-tolyl)-2H-pyrano[2,3-b]pyridin-7-yl)amino)propanamide (Peak-1): LCMS (ESI) Calcd. for C₁₉H₁₉N₃O₃: 337, found [M+H]⁺=338. ¹H NMR (400 MHz, DMSO-d₆) δ 7.44-7.36 (m, 3H), 7.34-7.32 (m, 1H), 7.22-7.21 (m, 1H), 7.09 (d, 2H), 6.65 (d, 1H), 6.01 (s, 1H), 5.19 (s, 1H), 2.95 (s, 3H), 2.12 (s, 3H), 1.36 (s, 3H).

Example 49: Chiral 2-(methyl(2-oxo-4-(o-tolyl)-2H-pyrano[2,3-b]pyridin-7-yl)amino)propanamide (Peak-2): LCMS (ESI) Calcd. for C₁₉H₁₉N₃O₃: 337, found [M+H]⁺=338. ¹H NMR (400 MHz, DMSO-d₆) δ 7.44-7.36 (m, 3H), 7.34-7.32 (m, 1H), 7.22-7.21 (m, 1H), 7.09 (d, 2H), 6.65 (d, 1H), 6.01 (s, 1H), 5.19 (s, 1H), 2.95 (s, 3H), 2.12 (s, 3H), 1.36 (s, 3H).

SFC chiral HPLC method: Chiral separation was performed on Thar SFC-80 series instrument. The column used was Chiralpak IG (21 mm×250 mm), 5μ which was operating at a temperature of 35° C. with a flow rate of 60 gm/min. The mobile phase used is 50% CO₂ at super critical state, 50% methanol, held isocratic and isobaric (100 bar) up to 20 min. with detection at a wavelength of 370 nm.

### Example 50-51: Synthesis of chiral isomers of (4-(2,6-dimethylphenyl)-2-oxo-2H-pyrano[2,3-b]pyridin-7-yl)-D-proline

### Synthesis of 50-5

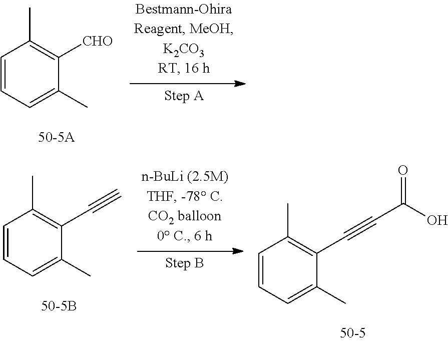

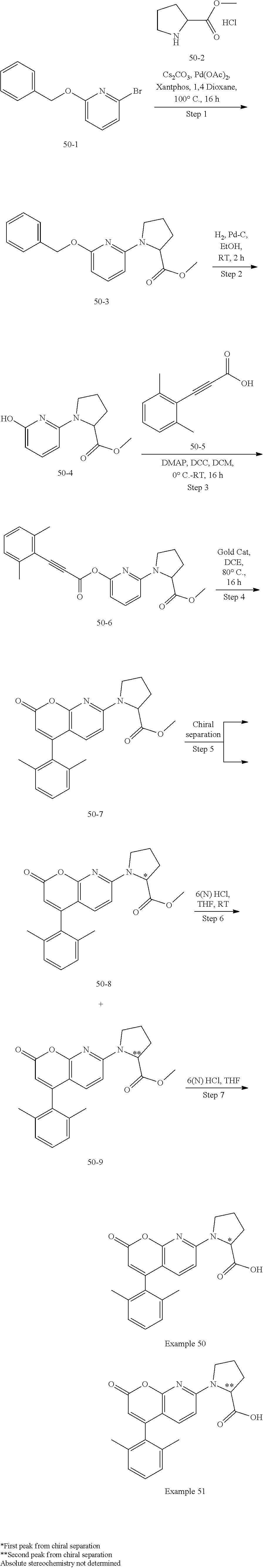

Synthesis of 2-ethynyl-1,3-dimethylbenzene, 50-5B [Step A]: To a stirred solution of 2,6-dimethylbenzaldehyde (50-5A, 2.0 g, 14.9 mmol) in methanol (10 mL), K₂CO₃ (4.1 g, 29.8 mmol) and Bestmann-Ohira reagent (14.6 mL, 19.4 mmol) were added and the resulting suspension was stirred at ambient temperature for 16 h under inert atmosphere. After completion, the reaction mixture was quenched with water and concentrated under reduced pressure. The residue was diluted with ethyl acetate and washed with water. The organic extracts were dried over anhydrous Na₂SO₄, filtered and concentrated under reduced pressure to afford 2-ethynyl-1,3-dimethylbenzene (50-5B, 800 mg). ¹H NMR (400 MHz, DMSO-d₆) δ 7.17 (t, 1H), 7.09 (d, 2H), 4.58 (s, 1H), 2.36 (s, 6H).

Synthesis of 3-(2,6-dimethylphenyl)propiolic acid, 50-5C [Step B]: To a stirred solution of 2-ethynyl-1,3-dimethylbenzene (50-5B, 500 mg, 3.2 mmol) in THF (10 mL), n-BuLi (2.6 M) (3.9 mL, 10.1 mmol) was added at −78° C. and stirred at same temperature for 1 h. Then carbon dioxide gas (using balloon) was purged into the reaction mixture and the reaction mixture was allowed to warm up to ambient temperature over 16 h. After completion, the reaction mixture was quenched with water and the aqueous phase was washed with ethyl acetate. The aqueous extract was acidified with 1N HCl solution to pH=3-4 and extracted with ethyl acetate (thrice). The combined organic layer was dried over anhydrous sodium sulphate and concentrated under reduced pressure to afford 3-(2,6-dimethylphenyl)propiolic acid (50-5C, 300 mg). ¹H NMR (400 MHz, DMSO-d₆) δ 13.72 (s, 1H), 7.30 (t, 1H), 7.16 (d, 2H), 2.40 (s, 6H).

Synthesis of methyl (6-(benzyloxy)pyridin-2-yl)prolinate, 50-3 [Step 1]: To a stirred solution of 2-benzyloxy-6-bromo-pyridine (50-1, 700 mg, 2.7 mmol) and methyl pyrrolidine-2-carboxylate hydrochloride (50-2, 660 mg, 3.9 mmol) in 1,4-dioxane (8 mL), Cs₂CO₃ (2.6 g, 7.9 mmol) was added and purged under argon atmosphere. Into the mixture, Pd(OAc)₂ (60 mg, 0.3 mmol) and Xantphos (305 mg, 0.5 mmol) were added and the mixture was heated at 100° C. for 16 h. The mixture was cooled to ambient temperature, water was added and extracted with ethyl acetate (twice). The combined organic extract was dried over anhydrous Na₂SO₄, filtered and concentrated under reduced pressure. The product was purified through combi flash chromatography to afford methyl (6-(benzyloxy)pyridin-2-yl)prolinate (50-3, 510 mg). LCMS (ESI) Calcd. for C₁₈H₂₀N₂O₃: 312, found [M+H]⁺=313. ¹H NMR (400 MHz, DMSO-d₆) δ 7.45-7.27 (m, 6H), 6.04-6.00 (in, 2H), 5.26 (t, 1H), 5.14 (t, 1H), 4.43-4.40 (m, 1H), 3.63-3.46 (m, 3H), 3.45 (t, 1H), 3.36-3.30 (m, 1H), 2.26-2.23 (m, 1H), 2.21-1.91 (m, 3H).

Synthesis of methyl (6-hydroxypyridin-2-yl)prolinate, 50-4 [Step 2]: To a stirred solution of methyl (6-(benzyloxy)pyridin-2-yl)prolinate (50-3, 640 mg, 2.1 mmol) in ethanol (10 mL) and catalytic amount of conc. HCl (0.3 mL) was added, then argon gas was purged for 5 min. In to the solution Pd—C(100 mg, 0.03 mmol, 10%) was added and the mixture was hydrogenated using a hydrogen balloon at ambient temperature for 2 h. After completion, the mixture was passed through celite bed, and the filtrate was concentrated under reduced pressure to afford methyl (6-hydroxypyridin-2-yl)prolinate (50-4, 445 mg). LCMS (ESI) Calcd. for C₁₁H₁₄N₂O₃: 222, found [M+H]⁺=223.

Synthesis of methyl (6-((3-(2,6-dimethylphenyl)propioloyl)oxy)pyridin-2-yl)prolinate, 50-6 [Step 3]: To a stirred solution of methyl (6-hydroxypyridin-2-yl)prolinate (50-4, 225 mg, 1.0 mmol) and 3-(2,6-dimethylphenyl)propiolic acid (50-5C, 195 mg, 1.2 mmol) in dichloromethane (4 mL), DMAP (20 mg, 0.2 mmol) was added. The mixture was cooled at 0° C. and DCC (334 mg, 1.6 mmol) in dichloromethane (1 mL) was slowly added to the mixture and stirred at ambient temperature for 16 h. After completion, the reaction mixture was diluted with dichloromethane and the mixture was filtered through celite bed. The filtrate was concentrated under reduced pressure and the product was purified by flash column chromatography and to afford methyl (6-((3-(2,6-dimethylphenyl)propioloyl)oxy)pyridin-2-yl)prolinate (50-6, 124 mg). LCMS (ESI) Calcd. for C₂₂H₂₂N₂O₄: 378, found [M+H]⁺=379. ¹H NMR (400 MHz, DMSO-d₆) δ 7.68 (t, 1H), 7.35 (t, 1H), 7.18 (d, 2H), 6.48 (t, 2H), 4.43-4.40 (m, 1H), 3.57-3.43 (m, 4H), 3.29 (d, 1H), 2.34 (s, 6H), 1.98 (s, 4H).

Synthesis of methyl (4-(2,6-dimethylphenyl)-2-oxo-2H-pyrano[2,3-b]pyridin-7-yl)prolinate, 50-7 [Step 4]: A stirred solution of methyl (6-((3-(2,6-dimethylphenyl)propioloyl)oxy)pyridin-2-yl)prolinate (50-6, 60 mg, 0.2 mmol) in dichloroethane (3 mL) was degassed with argon gas for 10 min. Then (Acetonitrile)[(2-biphenyl)di-tert-butylphosphine]gold(I) hexafluoroantimonate (12 mg, 0.02 mmol) was added under inert atmosphere and was heated at 90° C. for 18 h. The mixture was diluted with dichloromethane and filtered through celite bed. The filtrate was concentrated under reduced pressure and purified by column chromatography to afford methyl (4-(2,6-dimethylphenyl)-2-oxo-2H-pyrano[2,3-b]pyridin-7-yl)prolinate (50-7, 50 mg). LCMS (ESI) Calcd. For C₂₂H₂₂N₂O₄: 378, found [M+H]⁺=379. ¹H NMR (400 MHz, DMSO-d₆) δ 7.29 (t, 1H), 7.20 (d, 2H), 6.95 (d, 1H), 5.99 (s, 1H), 4.61 (d, 1H), 4.02 (d, 1H), 3.68 (s, 3H), 3.56 (s, 1H), 3.49 (s, 1H), 2.36 (s, 1H), 2.02 (br s, 7H), 1.25 (d, 1H), 1.17 (br s, 1H).

Synthesis of chiral isomers of methyl (4-(2,6-dimethylphenyl)-2-oxo-2H-pyrano[2,3-b]pyridin-7-yl)prolinate, 50-8 and 50-9 [Step 5]: Racemic methyl (4-(2,6-dimethylphenyl)-2-oxo-2H-pyrano[2,3-b]pyridin-7-yl)prolinate (50-7, 90 mg) was submitted to chiral HPLC SFC for chiral separation. The fractions obtained were lyophilized to afford the first product as chiral methyl (4-(2,6-dimethylphenyl)-2-oxo-2H-pyrano[2,3-b]pyridin-7-yl)prolinate (50-8, 40 mg) as Peak 1, and the second product as chiral methyl (4-(2,6-dimethylphenyl)-2-oxo-2H-pyrano[2,3-b]pyridin-7-yl)prolinate (50-9, 30 mg) as Peak 2. The absolute stereochemistries were not determined.

50-8: Chiral methyl (4-(2,6-dimethylphenyl)-2-oxo-2H-pyrano[2,3-b]pyridin-7-yl)prolinate, Peak 1: LCMS (ESI) Calcd. for C₂₂H₂₂N₂O₄: 378, found [M+H]⁺=379. ¹H NMR (400 MHz, DMSO-d₆) δ 7.29 (t, 1H), 7.20 (d, 2H), 6.95 (d, 1H), 5.99 (s, 1H), 4.61 (d, 1H), 4.02 (d, 1H), 3.68 (s, 3H), 3.56 (s, 1H), 3.49 (s, 1H), 2.36 (s, 1H), 2.02 (br s, 7H), 1.25 (d, 1H), 1.17 (br s, 1H).

50-9: Chiral methyl (4-(2,6-dimethylphenyl)-2-oxo-2H-pyrano[2,3-b]pyridin-7-yl)prolinate, Peak 2: LCMS (ESI) Calcd. for C₂₂H₂₂N₂O₄: 378, found [M+H]⁺=379. ¹H NMR (400 MHz, DMSO-d₆) δ 7.29 (t, 1H), 7.20 (d, 2H), 6.95 (d, 1H), 5.99 (s, 1H), 4.61 (d, 1H), 4.02 (d, 1H), 3.68 (s, 3H), 3.56 (s, 1H), 3.49 (s, 1H), 2.36 (s, 1H), 2.02 (br s, 7H), 1.25 (d, 1H), 1.17 (br s, 1H).

Prep SFC method: SFC Prep purification was performed using REFLECT (R,R) WHELK-01 (21.1 mm×250 mm), 5μ operating at 35° C. temperature, maintaining flow rate of 60 gm/min, using 65% CO₂ in super critical state & 35% of (0.5% Ipamine in IPA) as mobile phase. Run this isocratic mixture up to 20 min. and also maintained the isobaric condition of 100 bar at 220 nm wavelength.

Synthesis of (4-(2,6-dimethylphenyl)-2-oxo-2H-pyrano[2,3-b]pyridin-7-yl)-L-proline, Example 50 [Step 6]: To a stirred solution of chiral methyl (4-(2,6-dimethylphenyl)-2-oxo-2H-pyrano[2,3-b]pyridin-7-yl)prolinate (50-8, 40 mg, 0.1 mmol) in THF (2 mL), HCl (0.90 mL, 5.3 mmol, 6 N) was added. The reaction mixture was stirred for two days at ambient temperature. After completion, the resulting mixture was concentrated under reduced pressure. The product was purified by reverse phase prep HPLC purification and lyophilized to afford (4-(2,6-dimethylphenyl)-2-oxo-2H-pyrano[2,3-b]pyridin-7-yl)-L-proline (Example 50, 11 mg). LCMS (ESI) Calcd. For C₂₁H₂₀N₂O₄: 364, found [M+H]⁺=365. ¹H NMR (400 MHz, DMSO-d₆) δ 7.28 (t, 1H), 7.19 (d, 2H), 6.92 (s, 1H), 6.69 (s, 1H), 6.50 (s, 1H), 5.95 (s, 1H), 5.90 (br s, 1H), 4.54 (s, 1H), 3.34-3.16 (m, 2H), 2.07 (s, 1H), 2.02-1.98 (s, 9H).

Synthesis of (4-(2,6-dimethylphenyl)-2-oxo-2H-pyrano[2,3-b]pyridin-7-yl)-D-proline, Example 51 [Step 7]: To a stirred solution of chiral methyl (4-(2,6-dimethylphenyl)-2-oxo-2H-pyrano[2,3-b]pyridin-7-yl)prolinate (50-9, 30 mg, 0.1 mmol) in THF (2 mL), HCl (0.6 mL, 3.9 mmol, 6 N) was added. The reaction mixture was stirred for two days at ambient temperature. After completion, the resulting mixture was concentrated under reduced pressure. The product was purified by reverse phase prep HPLC purification and lyophilized to afford (4-(2,6-dimethylphenyl)-2-oxo-2H-pyrano[2,3-b]pyridin-7-yl)-D-proline (Example 51, 15 mg). LCMS (ESI) Calcd. for C₂₁H₂₀N₂O₄: 364, found [M+H]⁺=365. ¹H NMR (400 MHz, DMSO-d₆) δ 7.28 (t, 1H), 7.19 (d, 2H), 6.92 (s, 1H), 6.69 (s, 1H), 6.50 (s, 1H), 5.95 (s, 1H), 5.90 (br s, 1H), 4.54 (s, 1H), 3.34-3.16 (m, 2H), 2.07 (s, 1H), 2.02-1.98 (s, 9H).

### Example 52: Synthesis of 3-((4-(2-chloro-4-fluorophenyl)-2-oxo-2H-pyrano[2,3-b]pyridin-7-yl)(methyl)amino)propanamide

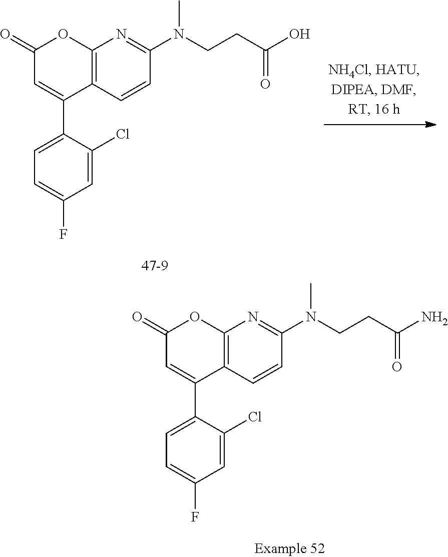 [SMILES: CN(CCC(=O)O)c1ccc2c(-c3ccc(F)cc3Cl)cc(=O)oc2n1 ; CN(CCC(N)=O)c1ccc2c(-c3ccc(F)cc3Cl)cc(=O)oc2n1]

To a stirred solution of 3-((4-(2-chloro-4-fluorophenyl)-2-oxo-2H-pyrano[2,3-b]pyridin-7-yl)(methyl)amino)propanoic acid (47-9, 70 mg, 0.2 mmol) in dry DMF (1 mL), HATU (105 mg, 0.3 mmol) and DIPEA (0.1 mL, 0.7 mmol) were added. The reaction mixture was stirred at ambient temperature for 15 min. and ammonium chloride (30 mg, 0.6 mmol) was added. The reaction mixture was stirred at ambient temperature for additional 16 h. The reaction mixture was diluted with ethyl acetate, washed with ice-cold water, dried over anhydrous sodium sulphate, filtered and concentrated under reduced pressure. The product was purified by reverse phase prep-HPLC to afford 3-((4-(2-chloro-4-fluorophenyl)-2-oxo-2H-pyrano[2,3-b]pyridin-7-yl)(methyl)amino)propanamide (Example 52, 14 mg). LCMS (ESI) Calcd. for C₁₈H₁₅ClFN₃O₃: 375, found [M+H]⁺=376. ¹H NMR (400 MHz, DMSO-d₆) δ 7.70-7.68 (m, 1H), 7.55-7.51 (m, 1H), 7.45-7.40 (m, 2H), 7.14 (d, 1H), 6.89 (br s, 1H), 6.64 (d, 1H), 6.09 (s, 1H), 3.78 (t, 2H), 3.09 (s, 3H), 2.39 (t, 2H).

### Example 53-54: Synthesis of chiral isomers of methyl N-(4-(2-chloro-4-fluorophenyl)-2-oxo-2H-pyrano[2,3-b]pyridin-7-yl)-N-methyl-L-alaninate

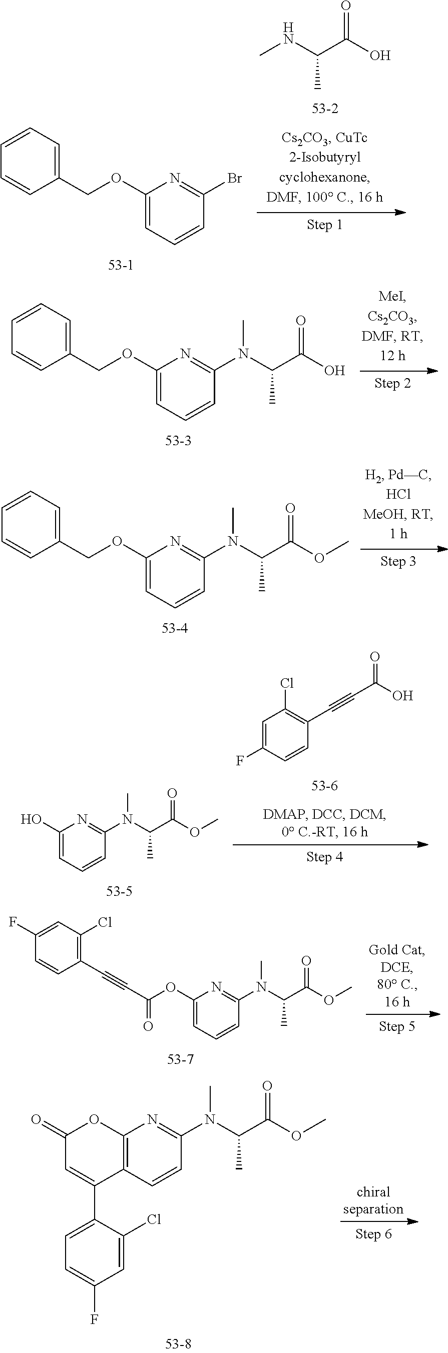

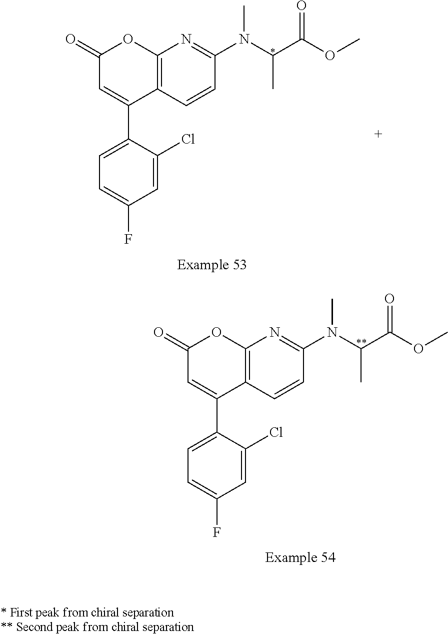 [SMILES: COC(=O)C(C)N(C)c1ccc2c(-c3ccc(F)cc3Cl)cc(=O)oc2n1 ; COC(=O)C(C)N(C)c1ccc2c(-c3ccc(F)cc3Cl)cc(=O)oc2n1]

Synthesis of N-(6-(benzyloxy)pyridin-2-yl)-N-methyl-L-alanine [Step 1]: A stirred solution of 2-(benzyloxy)-6-bromopyridine (53-1, 1.0 g, 3.8 mmol), methyl-L-alanine (53-2, 430 mg, 4.2 mmol), and cesium carbonate (2.5 g, 7.6 mmol) in DMF (6 mL) was allowed to degass with Argon gas at ambient temperature for 15 min. Copper(I) thiophene 2-carboxylate (145 mg, 0.8 mmol) and 2-isobutyrylcyclohexanone (510 mg, 3.0 mmol) were added to the reaction mixture and heated at 100° C. for 16 h. After completion, the reaction mixture was cooled, diluted with water and ethyl acetate. The aqueous layer was acidified to ˜pH 4-5 using aqueous HCl (2N). The mixture was extracted with 10% methanol in dichloromethane (thrice). The combined organic extracts were dried over anhydrous sodium sulfate, filtered, and concentrated under reduced pressure to afford N-(6-(benzyloxy)pyridin-2-yl)-N-methyl-L-alanine (53-3, 1.2 g). LCMS (ESI) Calcd. for C₁₆H₁₈N₂O₃: 286, found [M+H]⁺=287.

Synthesis of methyl N-(6-(benzyloxy)pyridin-2-yl)-N-methyl-L-alaninate, 53-4 [Step 2]: A stirred solution of N-(6-(benzyloxy)pyridin-2-yl)-N-methyl-L-alanine (53-3, 1.2 g, 3.0 mmol) and cesium carbonate (1.9 g, 6.0 mmol) in dry DMF (7 mL) was cooled to 0° C. Methyl iodide (1.0 mL, 15.1 mmol) was added dropwise to the cold mixture. The reaction mixture was allowed to stir at ambient temperature for 12 h. After completion, the reaction mixture was diluted water and was extracted with ethyl acetate (twice). The organic extract was dried over anhydrous Na₂SO₄, filtered, and concentrated under reduced pressure. The product was purified by flash-chromatography to afford methyl N-(6-(benzyloxy)pyridin-2-yl)-N-methyl-L-alaninate (53-4, 700 mg). LCMS (ESI) Calcd. for C₁₇H₂₀N₂O₃: 300, found [M+H]⁺=301. ¹H NMR (400 MHz, CDCl₃) δ 7.43-7.40 (m, 3H), 7.33 (t, 2H), 7.27 (d, 1H), 6.11 (d, 1H), 6.06 (d, 1H), 5.26 (s, 2H), 5.21 (q, 1H), 3.65 (s, 3H), 2.93 (s, 3H), 1.43 (d, 3H).

Synthesis of methyl N-(6-hydroxypyridin-2-yl)-N-methyl-L-alaninate hydrochloride, 53-5 [Step 3]: A stirred solution of methyl N-(6-(benzyloxy)pyridin-2-yl)-N-methyl-L-alaninate (53-4, 500 mg, 1.7 mmol) and 12 (N) HCl (0.5 mL) in methanol (30 mL) was purged with Argon gas for 5 min. To this solution Pd/C (200 mg, 0.2 mmol, 10%) was added and the mixture was hydrogenated using a hydrogen balloon at ambient temperature for 1 h. After completion, the reaction mixture was filtered through celite bed and concentrated under reduced pressure to afford methyl N-(6-hydroxypyridin-2-yl)-N-methyl-L-alaninate hydrochloride (53-5, 350 mg). LCMS (ESI) Calcd. for C₁₀H₁₄N₂O₃: 210, found [M+H]⁺=211.

Synthesis of (S)-6-((1-methoxy-1-oxopropan-2-yl)(methyl)amino)pyridin-2-yl 3-(2-chloro-4-fluorophenyl)propiolate, 53-7 [Step 4]: To a stirred solution of methyl N-(6-hydroxypyridin-2-yl)-N-methyl-L-alaninate hydrochloride (53-5, 350 mg, 1.7 mmol) and 3-(2-chloro-4-fluorophenyl)propiolic acid (53-6, 400 mg, 2.0 mmol) in dichloromethane (20 mL), DMAP (35 mg, 0.3 mmol) was added and the mixture was cooled to 0° C. Then DCC (515 mg, 2.5 mmol) in 2 mL dichloromethane was added slowly in to the mixture and stirred at ambient temperature for 16 h. The reaction mixture was diluted with dichloromethane, filtered through celite bed and concentrated under reduced pressure. The product was purified by column chromatography to afford (S)-6-((1-methoxy-1-oxopropan-2-yl)(methyl)amino)pyridin-2-yl 3-(2-chloro-4-fluorophenyl)propiolate (53-7, 180 mg). LCMS (ESI) Calcd. for C₁₉H₁₆ClFN₂O₄: 390, found [M+H]⁺=391. ¹H NMR (400 MHz, CDCl₃) δ 7.64-7.56 (m, 2H), 7.19 (d, 1H), 7.04 (t, 1H), 6.46-6.39 (m, 2H), 5.44-5.40 (m, 1H), 3.69 (s, 3H), 2.94 (s, 3H), 1.45 (d, 3H).

Synthesis of methyl N-(4-(2-chloro-4-fluorophenyl)-2-oxo-2H-pyrano[2,3-b]pyridin-7-yl)-N-methyl-L-alaninate, 53-8 [Step 5]: A stirred solution of (S)-6-((1-methoxy-1-oxopropan-2-yl)(methyl)amino)pyridin-2-yl 3-(2-chloro-4-fluorophenyl)propiolate (53-7, 180 mg, 0.5 mmol) in dichloroethane (2 mL) was degassed with argon for 5 min. Then (acetonitrile)[(2-biphenyl)di-tert-butylphosphine]gold(I) hexafluoroantimonate (40 mg, 0.05 mmol) was added and the mixture was heated at 80° C. for 16 h. After completion, the mixture was diluted with dichloromethane and filtered through celite bed and concentrated under reduced pressure. The product was purified by column chromatography to afford methyl N-(4-(2-chloro-4-fluorophenyl)-2-oxo-2H-pyrano[2,3-b]pyridin-7-yl)-N-methyl-L-alaninate (53-8, 110 mg). LCMS (ESI) Calcd. for C₁₉H₁₆ClFN₂O₄=390, found [M+H]⁺=391. ¹H NMR (400 MHz, DMSO-d₆) δ 7.69 (d, 1H), 7.56-7.52 (m, 1H), 7.43 (t, 1H), 7.21 (d, 1H), 6.70 (d, 1H), 6.15 (s, 1H), 5.36 (br s, 1H), 3.65 (s, 3H), 2.99 (s, 3H), 1.45 (d, 3H). The product was characterized as a mixture of enantiomers in a ratio of about 8:2 (L:D).

Separation of chiral methyl N-(4-(2-chloro-4-fluorophenyl)-2-oxo-2H-pyrano[2,3-b]pyridin-7-yl)-N-methyl-L-alaninate, Example 53, and methyl N-(4-(2-chloro-4-fluorophenyl)-2-oxo-2H-pyrano[2,3-b]pyridin-7-yl)-N-methyl-D-alaninate, Example 54 [Step 6]: A mixture of methyl N-(4-(2-chloro-4-fluorophenyl)-2-oxo-2H-pyrano[2,3-b]pyridin-7-yl)-N-methyl-alaninate (53-8, 110 mg, 0.4 mmol) was separated by SFC HPLC prep purification to afford methyl N-(4-(2-chloro-4-fluorophenyl)-2-oxo-2H-pyrano[2,3-b]pyridin-7-yl)-N-methyl-L-alaninate (Example 53, 55 mg) as Peak 1 (major isomer), and methyl N-(4-(2-chloro-4-fluorophenyl)-2-oxo-2H-pyrano[2,3-b]pyridin-7-yl)-N-methyl-D-alaninate (Example 54, 15 mg) as Peak 2 (minor isomer). 0346 Example 53: Methyl N-(4-(2-chloro-4-fluorophenyl)-2-oxo-2H-pyrano[2,3-b]pyridin-7-yl)-N-methyl-L-alaninate, Peak 1: LCMS (ESI) Calcd. for C₁₉H₁₆ClFN₂O₄: 390, found [M+H]⁺=391. ¹H NMR (400 MHz, DMSO-d₆) δ 7.70 (d, 1H), 7.56-7.52 (m, 1H), 7.43 (t, 1H), 7.22 (d, 1H), 6.70 (d, 1H), 6.15 (s, 1H), 5.36 (br s, 1H), 3.65 (s, 3H), 2.99 (s, 3H), 1.45 (d, 3H).

Example 54: Methyl N-(4-(2-chloro-4-fluorophenyl)-2-oxo-2H-pyrano[2,3-b]pyridin-7-yl)-N-methyl-D-alaninate, Peak 2: LCMS (ESI) Calcd. for C₁₉H₁₆ClFN₂O₄: 390, found [M+H]⁺=391. ¹H NMR (400 MHz, DMSO-d₆) δ 7.70 (d, 1H), 7.56-7.52 (m, 1H), 7.43 (t, 1H), 7.22 (d, 1H), 6.70 (d, 1H), 6.15 (s, 1H), 5.35 (br s, 1H), 3.65 (s, 3H), 2.99 (s, 3H), 1.45 (d, 3H).

Chiral HPLC prep-Method (SFC): Chiral separation was performed on a THAR SFC-80 instrument using (R,R) Whelk-01 (21.0 mm×250 mm), 5μ, operating at 35° C. temperature, maintaining flow rate of 60 gm/min, using 60% CO₂ in super critical state & 40% of (100% MeOH) as mobile phase. Run this isocratic mixture up to 8.0 minutes and also maintained the isobaric condition of 100 bar at 220 nm wavelength.

### Example 55: Synthesis of 3-((4-(2-chloro-4-fluorophenyl)-2-oxo-2H-pyrano[2,3-b]pyridin-7-yl)(methyl)amino)propanoic acid

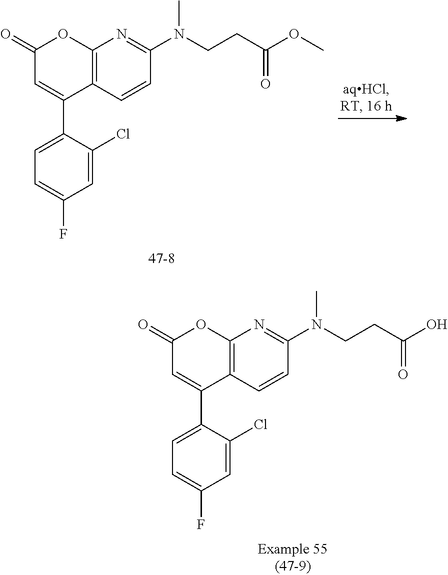 [SMILES: COC(=O)CCN(C)c1ccc2c(-c3ccc(F)cc3Cl)cc(=O)oc2n1 ; CN(CCC(=O)O)c1ccc2c(-c3ccc(F)cc3Cl)cc(=O)oc2n1]

A solution of methyl 3-((4-(2-chloro-4-fluorophenyl)-2-oxo-2H-pyrano[2,3-b]pyridin-7-yl)amino)propanoate (47-8, 30 mg, 0.08 mmol) in aqueous solution of hydrochloric acid (6N) (2 mL) was stirred at ambient temperature for 16 h. The volatiles were evaporated under reduced pressure to afford 3-((4-(2-chloro-4-fluorophenyl)-2-oxo-2H-pyrano[2,3-b]pyridin-7-yl)(methyl)amino)propanoic acid (Example 55 (47-9), 5.67 mg). LCMS (ESI) Calcd. for C₁₈H₁₄ClFN₂O₄: 376, found [M+H]⁺=377. ¹H NMR (400 MHz, DMSO-d₆) (12.36 (br s, 1H), 7.70-7.68 (m, 1H), 7.53-7.51 (m, 1H), 7.42 (m, 1H), 7.14 (d, 1H), 6.64 (d, 1H), 6.09 (s, 1H), 3.79 (br s, 2H), 3.09 (s, 3H), 2.33 (m, 2H).

### Example 56: Synthesis of 2-((4-(2,6-dimethylphenyl)-2-oxo-2H-pyrano[2,3-b]pyridin-7-yl)(methyl)amino)acetamide

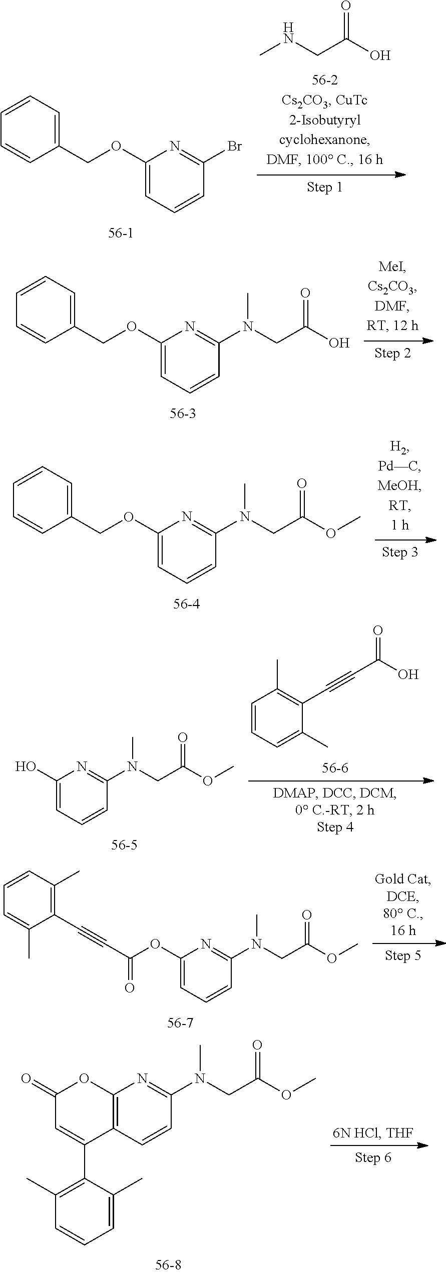

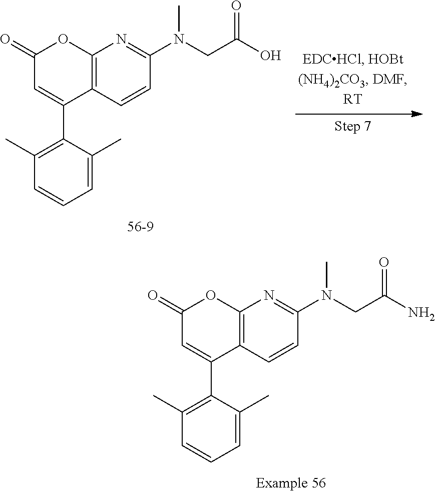 [SMILES: Cc1cccc(C)c1-c1cc(=O)oc2nc(N(C)CC(=O)O)ccc12 ; Cc1cccc(C)c1-c1cc(=O)oc2nc(N(C)CC(N)=O)ccc12]

Synthesis of N-(6-(benzyloxy)pyridin-2-yl)-N-methylglycine, 56-3 [Step 1]: A solution of 2-benzyloxy-6-bromo-pyridine (56-1, 1.0 g, 3.8 mmol) and 2-(methylamino)acetic acid (56-2, 0.4 g, 4.5 mmol) in dry DMF (15 mL), Cs₂CO₃ (2.5 g, 7.6 mmol) was added in a sealed tube and degassed with Argon gas for 5 min. 2-isobutylcyclohexanone (0.5 g, 3.0 mmol), and Copper thiophene-2-carboxylate (0.14 g, 0.76 mmol) were added to the reaction mixture and it was heated at 100° C. for 16 h. The reaction mass was cooled, diluted with water and ethyl acetate and aqueous layer was separated. The aqueous fraction was acidified to ˜pH 4-5 using aqueous HCl (2N) and the mixture was extracted with ethyl acetate. The combined organic extracts were dried over anhydrous sodium sulphate, filtered, and concentrated under reduced pressure to afford N-(6-(benzyloxy)pyridin-2-yl)-N-methylglycine (56-3, 1.1 g). LCMS (ESI) Calcd. for C₁₅H₁₆N₂O₃: 272, found [M+H]⁺=273.

Synthesis of methyl N-(6-(benzyloxy)pyridin-2-yl)-N-methylglycinate 56-4 [Step 2]: A mixture of N-(6-(benzyloxy)pyridin-2-yl)-N-methylglycine (56-3, 1.0 g, 3.7 mmol) and Cs₂CO₃ (2.4 g, 7.3 mmol) in dry dimethylformamide (7 mL) was allowed to cool at 0° C. and iodomethane (0.5 mL, 7.3 mmol) was added to the resulting mixture dropwise and stirred at ambient temperature for 16 h. The reaction was diluted with ethyl acetate and washed with ice-water and cold brine. The organic extract was dried over anhydrous sodium sulphate, filtered and concentrated under reduced pressure. The product was purified by flash column-chromatography to afford methyl N-(6-(benzyloxy)pyridin-2-yl)-N-methylglycinate (56-4, 700 mg). LCMS (ESI) Calcd. for C₁₆H₁₈N₂O₃: 286, found [M+H]⁺=287. ¹H NMR (400 MHz, DMSO-d₆) δ 7.46 (t, 1H), 7.38-7.29 (m, 5H), 6.17 (d, 1H), 6.06 (d, 1H), 5.21 (s, 2H), 4.27 (s, 2H), 3.58 (s, 3H), 3.02 (S, 3H).

Synthesis of methyl N-(6-hydroxypyridin-2-yl)-N-methylglycinate, 56-5 [Step 3]: To a stirred solution of methyl N-(6-(benzyloxy)pyridin-2-yl)-N-methylglycinate (56-4, 700 mg, 2.4 mmol) in dry methanol (5 mL), Pd/C (350 mg, 3.3 mmol, 10% w/w) and (0.2 mL) HCl solution were added under inert atmosphere and stirred at ambient temperature for 1 h under H₂ balloon. The reaction was filtered over celite bed, washed with methanol and concentrated under reduced pressure to afford methyl N-(6-hydroxypyridin-2-yl)-N-methylglycinate (56-5, 400 mg). LCMS (ESI) Calcd. for C₉H₁₂N₂O₃: 196, found [M+H]⁺=197.

Synthesis of 6-((2-methoxy-2-oxoethyl)(methyl)amino)pyridin-2-yl 3-(2,6-dimethylphenyl)propiolate, 56-7 [Step 4]: To a stirred solution of methyl N-(6-hydroxypyridin-2-yl)-N-methylglycinate (56-5, 300 mg, 1.5 mmol), 3-(2,6-dimethylphenyl)prop-2-ynoic acid (56-6, 320 mg, 1.8 mmol) in dichloromethane (20 mL), DMAP (30 mg, 0.3 mmol) was added followed by DCC (630 mg, 3.1 mmol) in dichloromethane (2 mL) dropwise at 0° C. and stirred for 1 h. After completion, the reaction mixture was filtered through celite bed and washed with dichloromethane and concentrated under reduced pressure. The product was purified by column chromatography to afford 6-((2-methoxy-2-oxoethyl)(methyl)amino)pyridin-2-yl 3-(2,6-dimethylphenyl)propiolate (56-7, 150 mg). LCMS (ESI) Calcd. for C₂₀H₂₀N₂O₄: 352, found [M+H]⁺=353. ¹H NMR (400 MHz, DMSO-d₆) δ 7.70 (t, 1H), 7.35 (t, 1H), 7.19 (d, 2H), 6.65 (d, 1H), 6.52 (d, 1H), 4.31 (s, 2H), 3.58 (s, 3H), 3.05 (s, 3H), 2.35 (s, 6H).

Synthesis of methyl N-(4-(2,6-dimethylphenyl)-2-oxo-2H-pyrano[2,3-b]pyridin-7-yl)-N-methylglycinate, 56-8 [Step 5]: To a stirred solution of 6-((2-methoxy-2-oxoethyl)(methyl)amino)pyridin-2-yl 3-(2,6-dimethylphenyl)propiolate (56-7, 100 mg, 0.3 mmol) in dichloroethane (3 mL), (Acetonitrile)[(2-biphenyl)di-tert-butylphosphine]gold(I) hexafluoroantimonate (20 mg, 0.02 mmol) was added under inert atmosphere and the reaction mixture was stirred at 80° C. for 18 h. The mixture was diluted with dichloromethane, filtered through celite bed and concentrated under reduced pressure. The product was purified by column chromatography to afford methyl N-(4-(2,6-dimethylphenyl)-2-oxo-2H-pyrano[2,3-b]pyridin-7-yl)-N-methylglycinate (56-8, 90 mg). LCMS (ESI) Calcd. for C₂₀H₂₀N₂O₄: 352, found [M+H]⁺=353. ¹H NMR (400 MHz, DMSO-d₆) δ 7.28 (d, 1H), 7.20 (d, 2H), 6.96 (d, 1H), 6.66 (d, 1H), 6.02 (s, 1H), 4.46 (s, 2H), 3.67 (s, 3H), 3.13 (s, 3H), 2.03 (s, 6H).

Synthesis of N-(4-(2,6-dimethylphenyl)-2-oxo-2H-pyrano[2,3-b]pyridin-7-yl)-N-methylglycine, 56-9 [Step 6]: To a stirred solution of methyl N-(4-(2,6-dimethylphenyl)-2-oxo-2H-pyrano[2,3-b]pyridin-7-yl)-N-methylglycinate (56-8, 50 mg, 0.1 mmol) in THF (2 mL), 6N HCl solution (6 mL) was added to the reaction mixture and it was stirred at ambient temperature for 16 h. After completion, the reaction mixture was diluted with water and extracted with ethyl acetate. The combined organic extract was dried over anhydrous sodium sulphate and concentrated under reduced pressure to afford N-(4-(2,6-dimethylphenyl)-2-oxo-2H-pyrano[2,3-b]pyridin-7-yl)-N-methylglycine (56-9, 45 mg). LCMS (ESI) Calcd. for C₁₉H₁₈N₂O₄: 338, found [M+H]⁺=339.

Synthesis of 2-((4-(2,6-dimethylphenyl)-2-oxo-2H-pyrano[2,3-b]pyridin-7-yl)(methyl)amino)acetamide, Example 56 [Step 7]: To a stirred solution of N-(4-(2,6-dimethylphenyl)-2-oxo-2H-pyrano[2,3-b]pyridin-7-yl)-N-methylglycine (56-9, 250 mg, 0.74 mmol) and (NH₄)₂CO₃ (284 mg, 2.9 mmol) in DMF (5 mL), HOBt (150 mg, 1.1 mmol), EDC·HCl (425 mg, 2.2 mmol), DIPEA (0.5 mL, 3.7 mmol) were added subsequently at 0° C. and stirred at ambient temperature for 16 h. The reaction mixture was diluted with ethyl acetate, washed with ice-cold water, dried over anhydrous sodium sulphate, filtered, and concentrated under reduced pressure. The product was purified by reverse phase prep-HPLC to afford 2-((4-(2,6-dimethylphenyl)-2-oxo-2H-pyrano[2,3-b]pyridin-7-yl)(methyl)amino)acetamide (Example 56, 52 mg). LCMS (ESI) Calcd. for C₁₉H₁₉N₃O₃: 337, found [M+H]⁺: 338. ¹H NMR (400 MHz, DMSO-d₆) δ 7.47 (s, 1H), 7.29 (t, 1H), 7.20-7.19 (m, 2H), 7.09 (s, 1H), 6.93 (d, 1H), 6.58 (d, 1H), 5.97 (s, 1H), 4.19 (s, 2H), 3.10 (s, 3H), 2.01 (s, 6H).

### Example 57-58: Synthesis of chiral isomers of 2-((4-(2,6-dimethylphenyl)-2-oxo-2H-pyrano[2,3-b]pyridin-7-yl)(methyl)amino)propanamide

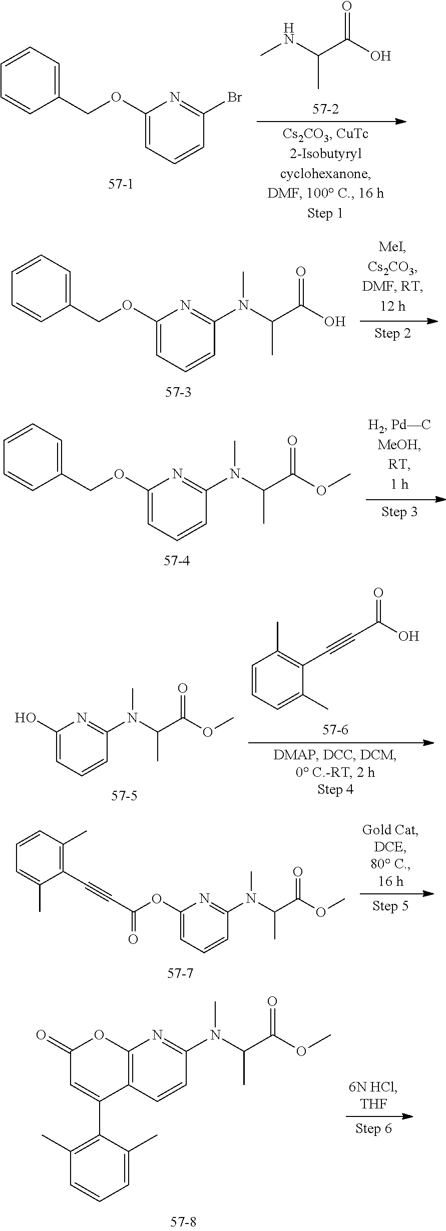

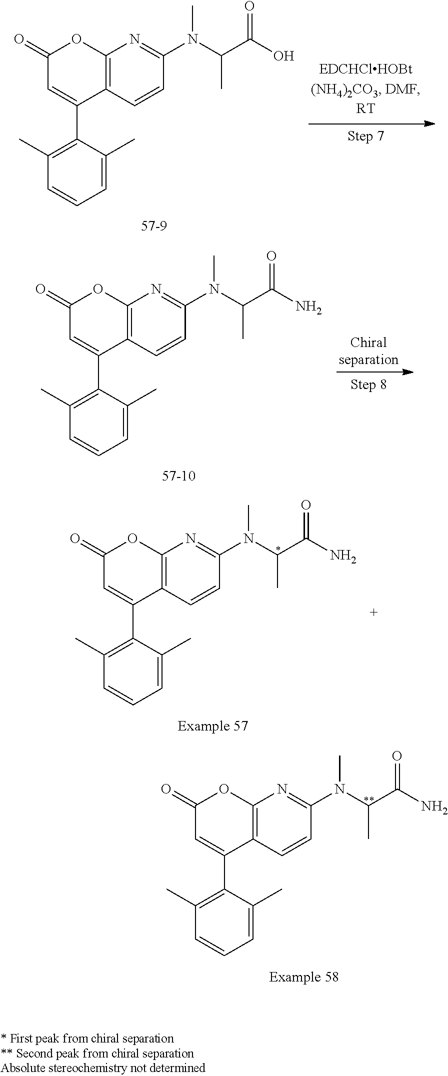 [SMILES: Cc1cccc(C)c1-c1cc(=O)oc2nc(N(C)C(C)C(=O)O)ccc12 ; Cc1cccc(C)c1-c1cc(=O)oc2nc(N(C)C(C)C(N)=O)ccc12 ; Cc1cccc(C)c1-c1cc(=O)oc2nc(N(C)C(C)C(N)=O)ccc12 ; Cc1cccc(C)c1-c1cc(=O)oc2nc(N(C)C(C)C(N)=O)ccc12]

Synthesis of N-(6-(benzyloxy)pyridin-2-yl)-N-methylalanine, 57-3 [Step 1]: A stirred solution of 2-(methylamino)propanoic acid (57-2, 0.5 g, 4.5 mmol) and 2-benzyloxy-6-bromo-pyridine (57-1, 1 g, 3.8 mmol) in dry DMF (15 mL), Cs₂CO₃ (2.5 g, 7.6 mmol) was added in a sealed tube and degassed with Argon gas for 5 min. 2-Isobutylcyclohexanone (0.5 g, 3.0 mmol) and copper thiophene-2-carboxylate (0.15 g, 0.8 mmol) were added to the reaction mixture and it was heated at 100° C. for 16 h. The reaction mass was cooled, diluted with water and ethyl acetate and aqueous layer was separated. The aqueous fraction was acidified to ˜pH 4-5 using aqueous HCl (2N) and the mixture was extracted with ethyl acetate (thrice). The combined organic extracts were dried over anhydrous sodium sulphate, filtered, and concentrated under reduced pressure to afford N-(6-(benzyloxy)pyridin-2-yl)-N-methylalanine (57-3, 1.5 g). LCMS (ESI) Calcd. for C₁₆H₁₈N₂O₃: 286, found [M+H]⁺=287.

Synthesis of methyl N-(6-(benzyloxy)pyridin-2-yl)-N-methylalaninate, 57-4 [Step 2]: A mixture of N-(6-(benzyloxy)pyridin-2-yl)-N-methylalanine (57-3, 1.5 g, 5.5 mmol) and Cs₂CO₃ (3.5 g, 11 mmol) in dry dimethylformamide (7 mL) was allowed to cool at 0° C. and iodomethane (0.5 mL, 7.6 mmol) was added to the resulting mixture dropwise and stirred at ambient temperature for 16 h. The reaction was diluted with ethyl acetate and washed with ice-water and cold brine. The organic extract was dried over anhydrous sodium sulphate, filtered, and concentrated under reduced pressure. The product was purified by flash column-chromatography to afford methyl N-(6-(benzyloxy)pyridin-2-yl)-N-methylalaninate (57-4, 700 mg). LCMS (ESI) Calcd. for C₁₇H₂₀N₂O₃: 300, found [M+H]⁺=301. ¹H NMR (400 MHz, DMSO-d₆) δ 7.45 (t, 1H), 7.39-7.26 (m, 5H), 6.16 (d, 1H), 6.08 (d, 1H), 5.24-5.16 (m, 2H), 5.01 (q, 1H), 3.56 (s, 3H), 2.89 (s, 3H), 1.35 (d, 3H).

Synthesis of methyl N-(6-hydroxypyridin-2-yl)-N-methylalaninate, 57-5 [Step 3]: To a stirred solution of methyl N-(6-(benzyloxy)pyridin-2-yl)-N-methylalaninate (57-4, 1 g, 3.3 mmol) in dry methanol (30 mL), 10% Pd/C (w/w) (200 mg, 3.3 mmol) and (0.5 mL) HCl solution were added under inert atmosphere and stirred at ambient temperature for 1 h under H₂ balloon. After completion, the reaction was filtered over celite bed and washed with methanol, concentrated under reduced pressure to afford methyl N-(6-hydroxypyridin-2-yl)-N-methylalaninate (57-5, 600 mg). LCMS (ESI) Calcd. for C₁₀H₁₄N₂O₃: 210, found [M+H]⁺=211.

Synthesis of 6-((1-methoxy-1-oxopropan-2-yl)(methyl)amino)pyridin-2-yl 3-(2,6-dimethylphenyl)propiolate, 57-7 [Step 4]: To a stirred solution of methyl N-(6-hydroxypyridin-2-yl)-N-methylalaninate (57-5, 0.6 g, 2.4 mmol), 3-(2,6-dimethylphenyl)prop-2-ynoic acid (57-6, 0.5 g, 2.9 mmol) in dichloromethane (20 mL), DMAP (50 mg, 0.5 mmol) followed by DCC (652 mg, 3.1 mmol) in dichloromethane (2 mL) was added dropwise at 0° C. and stirred for 1 h. After completion, the reaction mixture was filtered through celite bed and washed with dichloromethane and concentrated under reduced pressure. The product was purified using column chromatography to afford 6-((1-methoxy-1-oxopropan-2-yl)(methyl)amino)pyridin-2-yl 3-(2,6-dimethylphenyl)propiolate (57-7, 250 mg). ¹H NMR (400 MHz, DMSO-d₆) δ 7.71 (t, 1H), 7.35 (t, 1H), 7.18 (d, 2H), 6.66 (d, 1H), 6.52 (d, 1H), 5.15-5.10 (m, 1H), 3.59 (s, 3H), 2.90 (s, 3H), 2.42 (s, 6H), 1.50 (d, 3H).

Synthesis of methyl N-(4-(2,6-dimethylphenyl)-2-oxo-2H-pyrano[2,3-b]pyridin-7-yl)-N-methylalaninate, 57-8 [Step 5]: To a stirred solution of 6-((1-methoxy-1-oxopropan-2-yl)(methyl)amino)pyridin-2-yl 3-(2,6-dimethylphenyl)propiolate (57-7, 100 mg, 0.3 mmol) in dichloroethane (3 mL), (acetonitrile)[(2-biphenyl)di-tert-butylphosphine]gold(I) hexafluoroantimonate (20 mg, 0.02 mmol) was added under inert atmosphere and the reaction mixture was heated at 80° C. for 18 h. After completion, the mixture was diluted with dichloromethane, filtered through celite bed, and concentrated under reduced pressure. The product was purified by column chromatography to afford methyl N-(4-(2,6-dimethylphenyl)-2-oxo-2H-pyrano[2,3-b]pyridin-7-yl)-N-methylalaninate (57-8, 90 mg). LCMS (ESI) Calcd. for C₂₁H₂₂N₂O₄ 366, found [M+H]⁺=367. ¹H NMR (400 MHz, DMSO-d₆) δ 7.28 (d, 1H), 7.20 (d, 2H), 6.97 (d, 1H), 6.66 (d, 1H), 6.01 (s, 1H), 5.40 (br s, 1H), 3.65 (s, 3H), 2.97 (s, 3H), 2.03 (s, 6H), 1.45 (d, 3H).

Synthesis of N-(4-(2,6-dimethylphenyl)-2-oxo-2H-pyrano[2,3-b]pyridin-7-yl)-N-methylalanine, 57-9 [Step 6]: To a stirred solution of methyl N-(4-(2,6-dimethylphenyl)-2-oxo-2H-pyrano[2,3-b]pyridin-7-yl)-N-methylalaninate (57-8, 50 mg, 0.1 mmol) in THF (2 mL), 6 N HCl solution (6 mL) was added to the reaction mixture and it was stirred at ambient temperature for 16 h. After completion, the reaction mixture was diluted with water and extracted with ethyl acetate (thrice). The combined organic extract was dried over anhydrous sodium sulphate and concentrated under reduced pressure to afford of N-(4-(2,6-dimethylphenyl)-2-oxo-2H-pyrano[2,3-b]pyridin-7-yl)-N-methylalanine (57-9, 45 mg). LCMS (ESI) Calcd. for C₂₀H₂₀N₂O₄: 352, found [M+H]⁺=353.

Synthesis of 2-((4-(2,6-dimethylphenyl)-2-oxo-2H-pyrano[2,3-b]pyridin-7-yl)(methyl)amino)propanamide, 57-10 [Step 7]: To a stirred solution of N-(4-(2,6-dimethylphenyl)-2-oxo-2H-pyrano[2,3-b]pyridin-7-yl)-N-methylalanine (57-9, 250 mg, 0.7 mmol) and (NH₄)₂CO₃ (273 mg, 2.9 mmol) in DMF (5 mL), HOBt (144 mg, 1.1 mmol), EDC·HCl (408 mg, 2.2 mmol), DIPEA (0.6 ml, 3.7 mmol) were added subsequently at 0° C. and stirred at ambient temperature for 16 h. The reaction mixture was diluted with ethyl acetate, washed with ice-cold water, dried over anhydrous sodium sulphate, filtered, and concentrated under reduced pressure. The product was purified by flash column chromatography to afford 2-((4-(2,6-dimethylphenyl)-2-oxo-2H-pyrano[2,3-b]pyridin-7-yl)(methyl)amino)propanamide (57-10, 200 mg). LCMS (ESI) Calcd. for C₂₀H₂₁N₃O₃: 351, found [M+H]⁺=352.

Synthesis of chiral isomers of 2-((4-(2,6-dimethylphenyl)-2-oxo-2H-pyrano[2,3-b]pyridin-7-yl)(methyl)amino)propanamide, Example 57 and 58 [Step 8]: Racemic 2-((4-(2,6-dimethylphenyl)-2-oxo-2H-pyrano[2,3-b]pyridin-7-yl)(methyl)amino)propanamide (57-10, 80 mg. 0.1 mmol) was separated by SFC HPLC chiral purification and lyophilized to afford the first product as chiral 2-((4-(2,6-dimethylphenyl)-2-oxo-2H-pyrano[2,3-b]pyridin-7-yl)(methyl)amino)propanamide (Example 57, 5 mg) as Peak 1, and the second product as chiral 2-((4-(2,6-dimethylphenyl)-2-oxo-2H-pyrano[2,3-b]pyridin-7-yl)(methyl)amino)propanamide (Example 58, 10 mg) as Peak 2. The absolute stereochemistry of these compounds were not determined.

Example 57: Chiral 2-((4-(2,6-dimethylphenyl)-2-oxo-2H-pyrano[2,3-b]pyridin-7-yl)(methyl)amino)propanamide, Peak 1: LCMS (ESI) Calcd. for C₂₀H₂₁N₃O₃: 351, found [M+H]⁺=352. ¹H NMR (400 MHz, DMSO-d₆) δ 7.39 (s, 1H), 7.29 (d, 1H), 7.21-7.19 (m, 2H), 7.10 (s, 1H), 6.94 (d, 1H), 6.63 (d, 1H), 5.98 (s, 1H), 5.22 (br s, 1H), 2.94 (s, 3H), 2.03 (s, 6H), 1.35 (d, 3H).

Example 58: Chiral 2-((4-(2,6-dimethylphenyl)-2-oxo-2H-pyrano[2,3-b]pyridin-7-yl)(methyl)amino)propanamide, Peak 2: LCMS (ESI) Calcd. for C₂₀H₂₁N₃O₃: 351, found [M+H]⁺=352. ¹H NMR (400 MHz, DMSO-d₆) δ 7.39 (s, 1H), 7.29 (d, 1H), 7.21-7.19 (m, 2H), 7.10 (s, 1H), 6.94 (d, 1H), 6.63 (d, 1H), 5.98 (s, 1H), 5.22 (br s, 1H), 2.94 (s, 3H), 2.03 (s, 6H), 1.35 (d, 3H).

### Example 59: Synthesis of 2-methyl-2-(2-(methyl(2-oxo-4-(o-tolyl)-2H-pyrano[2,3-b]pyridin-7-yl)amino)acetamido)propanoic acid

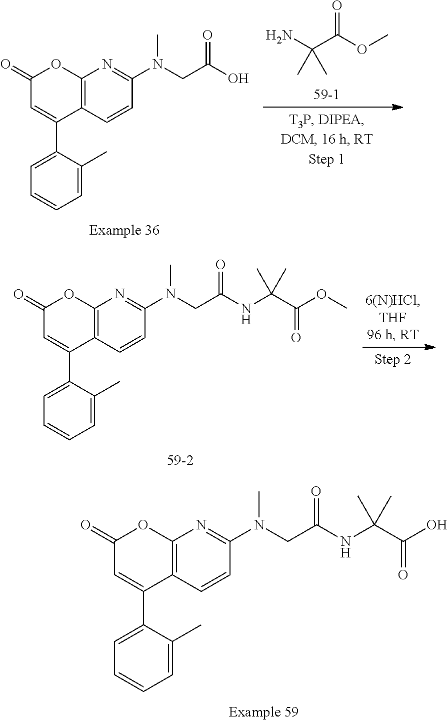

Synthesis of methyl 2-methyl-2-(2-(methyl(2-oxo-4-(o-tolyl)-2H-pyrano[2,3-b]pyridin-7-yl)amino)acetamido)propanoate, 59-2 [Step 1]: A stirred solution of N-methyl-N-(2-oxo-4-(o-tolyl)-2H-pyrano[2,3-b]pyridin-7-yl)glycine (Example 36, 120 mg, 0.4 mmol) and methyl 2-amino-2-methylpropanoate (59-1, 52 mg, 0.4 mmol) in dichloromethane (4 mL) was cooled to 0° C. and DIPEA (0.2 mL, 1.1 mmol) was added. Into this cold reaction mixture, T3P (0.3 mL, 0.6 mmol, 50% in ethyl acetate) was added dropwise and the reaction mixture was allowed to warm up to ambient temperature and stirred for 16 h. After completion, the reaction mixture was quenched with ice cold water and extracted with dichloromethane (twice). The combined organic extract was washed with brine, dried over anhydrous Na₂SO₄, and concentrated under reduced pressure. The product was purified by flash chromatography to afford methyl 2-methyl-2-(2-(methyl(2-oxo-4-(o-tolyl)-2H-pyrano[2,3-b]pyridin-7-yl)amino)acetamido)propanoate (59-2, 110 mg). LCMS (ESI) Calcd. for C₂₃H₂₅N₃O₅: 423, found [M+H]⁺=424.

Synthesis of 2-methyl-2-(2-(methyl(2-oxo-4-(o-tolyl)-2H-pyrano[2,3-b]pyridin-7-yl)amino)acetamido)propanoic acid, Example 59 [Step 2]: A solution of methyl 2-methyl-2-(2-(methyl(2-oxo-4-(o-tolyl)-2H-pyrano[2,3-b]pyridin-7-yl)amino)acetamido)propanoate (59-2, 110 mg, 0.3 mmol) in THF (2 mL) was cooled to 0° C. and 6(N) HCl (6 mL) was added dropwise and stirred at ambient temperature for 96 h. After completion, the volatiles were removed under reduced pressure and purified by reverse phase prep HPLC purification method and lyophilized to afford 2-methyl-2-(2-(methyl(2-oxo-4-(o-tolyl)-2H-pyrano[2,3-b]pyridin-7-yl)amino)acetamido)propanoic acid (Example 59, 10 mg). LCMS (ESI) Calcd. for C₂₂H₂₃N₃O₅: 409, found [M+H]⁺=410. ¹H NMR (400 MHz, DMSO-d₆) δ 8.25 (br s, 1H), 7.42-7.33 (m, 3H), 7.22-7.20 (m, 1H), 7.07-7.05 (m, 1H), 6.60 (m, 1H), 5.99 (s, 1H), 4.21 (s, 2H), 3.08 (s, 3H), 2.12 (s, 3H), 1.34-1.23 (s, 6H).

### Example 60: Synthesis of N-methyl-N-(2-oxo-4-(o-tolyl)-2H-pyrano[2,3-b]pyridin-7-yl)glycylglycine

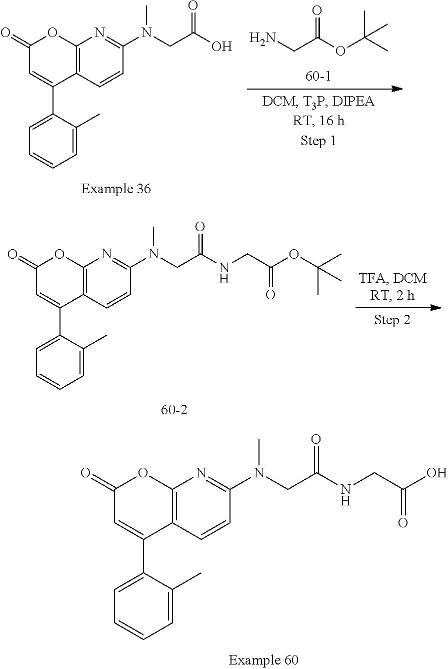

Synthesis of tert-butyl N-methyl-N-(2-oxo-4-(o-tolyl)-2H-pyrano[2,3-b]pyridin-7-yl)glycylglycinate, 60-2 [Step 1]: A stirred solution of N-methyl-N-(2-oxo-4-(o-tolyl)-2H-pyrano[2,3-b]pyridin-7-yl)glycine (Example 36, 120 mg, 0.4 mmol) and tert-butyl glycinate (60-1, 60 mg, 0.4 mmol) in dichloromethane (4 mL) was cooled to 0° C. and DIPEA (0.2 mL, 1.1 mmol) was added. In to this cold reaction mixture, T3P (0.3 mL, 0.6 mmol, 50% in ethyl acetate) was added drop wise and the reaction mixture was allowed to warm up to ambient temperature and stirred for 16 h. After completion, the reaction mixture was quenched with ice cold water and extracted with dichloromethane (twice). The combined organic extract was washed with brine, dried over anhydrous Na₂SO₄ and concentrated under reduced pressure. The product was purified by flash chromatography to afford tert-butyl N-methyl-N-(2-oxo-4-(o-tolyl)-2H-pyrano[2,3-b]pyridin-7-yl)glycylglycinate (60-2, 140 mg). LCMS (ESI) Calcd. for C₂₄H₂₇N₃O₅: 437, found [M+H−56]⁺=382. ¹H NMR (400 MHz, DMSO-d₆) δ 8.36-8.33 (m, 1H), 7.42-7.32 (m, 3H), 7.21-7.20 (m, 1H), 7.09-7.06 (m, 1H), 6.64-6.61 (m, 1H), 6.01 (m, 1H), 4.30-4.29 (s, 2H), 3.74-3.73 (s, 2H), 3.11 (s, 3H), 2.11 (s, 3H), 1.38 (s, 9H).

Synthesis of N-methyl-N-(2-oxo-4-(o-tolyl)-2H-pyrano[2,3-b]pyridin-7-yl)glycylglycine, Example 60 [Step 2]: A solution of tert-butyl N-methyl-N-(2-oxo-4-(o-tolyl)-2H-pyrano[2,3-b]pyridin-7-yl)glycylglycinate (60-2, 140 mg, 0.3 mmol) in dichloromethane (4 mL) was cooled to 0° C. and into the reaction mixture trifluoroacetic acid (1.5 mL, 20.1 mmol) was added dropwise and continued to stir at ambient temperature for 2 h. After completion, the volatiles were removed under reduced pressure. The product was purified by reverse phase prep HPLC purification method and lyophilized to afford N-methyl-N-(2-oxo-4-(o-tolyl)-2H-pyrano[2,3-b]pyridin-7-yl)glycylglycine (Example 60, 30 mg). LCMS (ESI) Calcd. for C₂₀H₁₉N₃O₅: 381, found [M+H]⁺=382. ¹H NMR (400 MHz, DMSO-d₆) δ 8.25 (br s, 1H), 7.42-7.32 (m, 3H), 7.22-7.20 (m, 1H), 7.08-7.06 (m, 1H), 6.63-6.61 (m, 1H), 6.07 (br s, 1H), 4.30 (s, 2H), 3.72 (br s, 2H), 3.10 (s, 3H), 2.11-2.07 (m, 3H).

### Example 61: Synthesis of 2-(1-(2-oxo-4-(o-tolyl)-2H-pyrano[2,3-b]pyridin-7-yl)pyrrolidin-2-yl)acetic acid

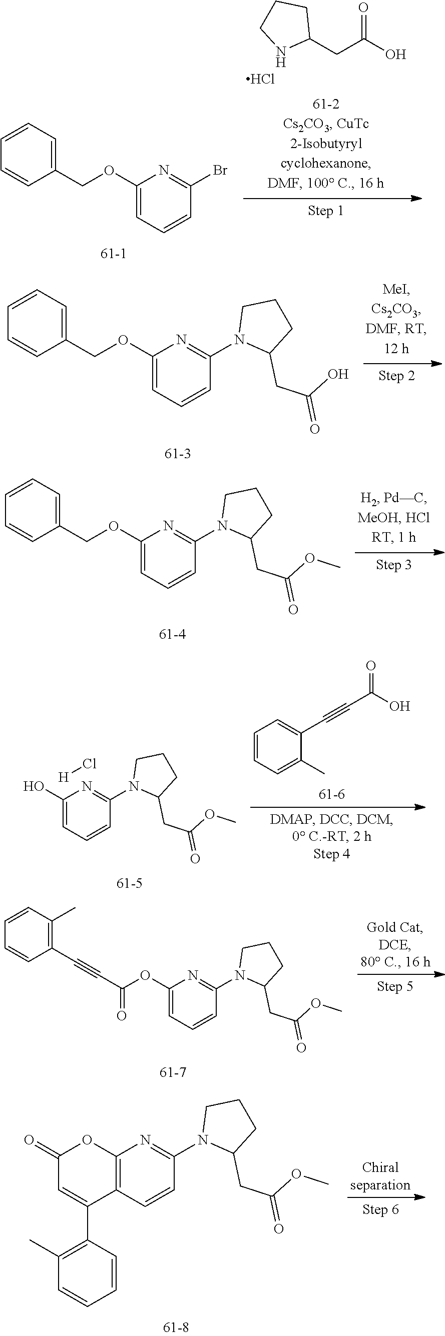

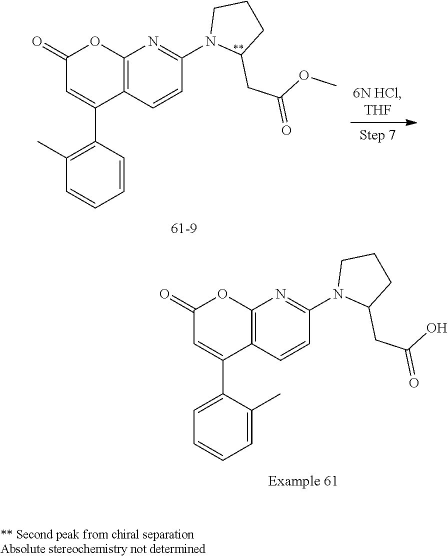 [SMILES: COC(=O)CC1CCCN1c1ccc2c(-c3ccccc3C)cc(=O)oc2n1 ; Cc1ccccc1-c1cc(=O)oc2nc(N3CCCC3CC(=O)O)ccc12]

Synthesis of 2-(1-(6-(benzyloxy)pyridin-2-yl)pyrrolidin-2-yl)acetic acid, 61-3 [Step 1]: A stirred solution of 2-(pyrrolidin-2-yl)acetic acid (61-2, 0.75 g, 4.5 mmol) and 2-benzyloxy-6-bromo-pyridine (61-1, 1 g, 3.8 mmol) in dry DMF (15 mL), Cs₂CO₃ (2.5 g, 7.6 mmol) was added in a sealed tube and degassed with Argon gas for 5 min. 2-Isobutylcyclohexanone (0.5 g, 3.0 mmol) and copper thiophene-2-carboxylate (0.15 g, 0.8 mmol) were added to the reaction mixture and it was heated at 100° C. for 16 h. The reaction mass was cooled, diluted with water and washed with ethyl acetate. The aqueous extract was collected and acidified to ˜pH 4-5 using aqueous HCl (2N) and the mixture was extracted with ethyl acetate. The combined organic extracts were dried over anhydrous sodium sulphate, filtered, and concentrated under reduced pressure to afford 2-(1-(6-(benzyloxy)pyridin-2-yl)pyrrolidin-2-yl)acetic acid (61-3, 1 g). LCMS (ESI) Calcd. for C₁₈H₂₀N₂O₃: 312, found [M+H]⁺=313.

Synthesis of methyl 2-(1-(6-(benzyloxy)pyridin-2-yl)pyrrolidin-2-yl)acetate, 61-4 [Step 2]: A mixture of 2-(1-(6-(benzyloxy)pyridin-2-yl)pyrrolidin-2-yl)acetic acid (61-3, 1.1 g, 5.5 mmol) and Cs₂CO₃ (2.5 g, 7.7 mmol) in dry dimethylformamide (7 mL) was allowed to cool at 0° C. and iodomethane (0.5 mL, 7.6 mmol) was added to the resulting mixture dropwise and stirred at ambient temperature for 16 h. The reaction was diluted with ethyl acetate and washed with ice-water and cold brine. The organic extract was dried over anhydrous sodium sulphate, filtered and concentrated under reduced pressure. The product was purified by flash column-chromatography to afford methyl 2-(1-(6-(benzyloxy)pyridin-2-yl)pyrrolidin-2-yl)acetate (61-4, 900 mg). LCMS (ESI) Calcd. for C₁₉H₂₂N₂O₃: 326, found [M+H]⁺=327.

Synthesis of methyl 2-(1-(6-hydroxypyridin-2-yl)pyrrolidin-2-yl)acetate hydrochloride, 61-5 [Step 3]: To a stirred solution of methyl 2-(1-(6-(benzyloxy)pyridin-2-yl)pyrrolidin-2-yl)acetate (61-4, 800 mg, 2.4 mmol) in dry methanol (30 mL), Pd/C (80 mg, 0.7 mmol, 10%) and (0.5 mL) 12 N HCl solution were added under inert atmosphere and stirred at ambient temperature for 1 h. under H₂ balloon. The reaction was filtered over celite bed and washed with methanol. The volatiles were concentrated under reduced pressure to afford methyl 2-(1-(6-hydroxypyridin-2-yl)pyrrolidin-2-yl)acetate hydrochloride (61-5, 500 mg). LCMS (ESI) Calcd. for C₁₀H₁₅ClN₂O₃: 236, found [M+H]⁺=237.

Synthesis of 6-(2-(2-methoxy-2-oxoethyl)pyrrolidin-1-yl)pyridin-2-yl 3-(o-tolyl)propiolate, 61-7 [Step 4]: To a stirred solution of methyl 2-(1-(6-hydroxypyridin-2-yl)pyrrolidin-2-yl)acetate hydrochloride (61-6, 1.1 gm, 4.0 mmol) and 3-(o-tolyl)prop-2-ynoic acid (775 mg, 4.8 mmol) in dichloromethane (20 mL), DMAP (100 mg, 0.8 mmol) followed by DCC (1.2 g, 6.0 mmol) in dichloromethane (4 mL) was added dropwise at 0° C. and stirred for 1 h. After completion, the reaction mixture was filtered through celite bed and washed with dichloromethane and concentrated under reduced pressure. The product was purified using column chromatography to afford 6-(2-(2-methoxy-2-oxoethyl)pyrrolidin-1-yl)pyridin-2-yl 3-(o-tolyl)propiolate (61-7, 900 mg). LCMS (ESI) Calcd. for C₂₂H₂₂N₂O₄: 378, found [M+H]⁺=379.

Synthesis of methyl 2-(1-(2-oxo-4-(o-tolyl)-2H-pyrano[2,3-b]pyridin-7-yl)pyrrolidin-2-yl)acetate, 61-8 [Step 5]: To a stirred solution of 6-(2-(2-methoxy-2-oxoethyl)pyrrolidin-1-yl)pyridin-2-yl 3-(o-tolyl)propiolate (61-7, 900 mg, 2.4 mmol) in dichloroethane (15 mL), (acetonitrile)[(2-biphenyl)di-tert-butylphosphine]gold(I) hexafluoroantimonate (185 mg, 0.2 mmol) was added under inert atmosphere and the reaction mixture was heated at 80° C. for 18 h. The mixture was diluted with dichloromethane, filtered through celite bed and concentrated under reduced pressure. The product was purified by column chromatography to afford methyl 2-(1-(2-oxo-4-(o-tolyl)-2H-pyrano[2,3-b]pyridin-7-yl)pyrrolidin-2-yl)acetate (61-8, 830 mg). LCMS (ESI) Calcd. for C₂₂H₂₂N₂O₄: 378, found [M+H]⁺=379.

Synthesis of chiral methyl 2-(1-(2-oxo-4-(o-tolyl)-2H-pyrano[2,3-b]pyridin-7-yl)pyrrolidin-2-yl)acetate, 61-9 [Step 6]: Racemic methyl 2-(1-(2-oxo-4-(o-tolyl)-2H-pyrano[2,3-b]pyridin-7-yl)pyrrolidin-2-yl)acetate (61-8, 190 mg) was used for HPLC chiral SFC separation to afford the second product as chiral methyl 2-(1-(2-oxo-4-(o-tolyl)-2H-pyrano[2,3-b]pyridin-7-yl)pyrrolidin-2-yl)acetate (61-9, 75 mg) as Peak 2.

Chiral HPLC prep-Method (SFC): SFC prep purification of the racemate (190 mg) has been completed on PIC SOLUTION-175 instrument by using Chiralpak IC (21 mm×250 mm), 5 min. operating at 35° C. temperature, maintaining flow rate of 70 ml/min, using 60% CO₂ in super critical state & 40% of (100% MeOH) as mobile phase. Run this isocratic mixture up to 20 minutes and also maintained the isobaric condition of 100 bar at 371 nm wavelength. The absolute stereochemistry was not determined.

Synthesis of 2-(1-(2-oxo-4-(o-tolyl)-2H-pyrano[2,3-b]pyridin-7-yl)pyrrolidin-2-yl)acetic acid, Example 61 [Step 7]: To a stirred solution of chiral methyl 2-(1-(2-oxo-4-(o-tolyl)-2H-pyrano[2,3-b]pyridin-7-yl)pyrrolidin-2-yl)acetate (61-9, 50 mg, 0.1 mmol) in tetrahydrofuran (2 mL) and H₂O (2 mL) mixture, 12 N HCl (4 mL) was added and it was stirred at ambient temperature for 16 h. After completion, the reaction mixture was diluted with water and extracted with ethyl acetate. The combined organic extract was dried over anhydrous sodium sulphate and concentrated under reduced pressure. The product was purified by reverse phase prep HPLC to afford 2-(1-(2-oxo-4-(o-tolyl)-2H-pyrano[2,3-b]pyridin-7-yl)pyrrolidin-2-yl)acetic acid (Example 61, 45 mg). LCMS (ESI) Calcd. for C₂₁H₂₀N₂O₄: 364, found [M+H]⁺=365. ¹H NMR (400 MHz, DMSO-d₆ at 100° C.) δ 6 7.41-7.37 (m, 2H), 7.35-7.31 (m, 1H), 7.20 (d, 1H), 7.06 (d, 1H), 6.44 (d, 1H), 5.92 (s, 1H), 4.45 (br s, 1H), 3.54-3.52 (m, 1H), 3.46-3.42 (m, 1H), 2.80 (m, 1H), 2.36-2.32 (m, 1H), 2.14 (s, 3H), 2.09-2.05 (m, 2H), 1.98-1.92 (m, 2H). The compound racemized during hydrolysis.

### Example 62: Synthesis of 1-(2-oxo-4-(o-tolyl)-2H-pyrano[2,3-b]pyridin-7-yl)piperidine-2-carboxylic acid

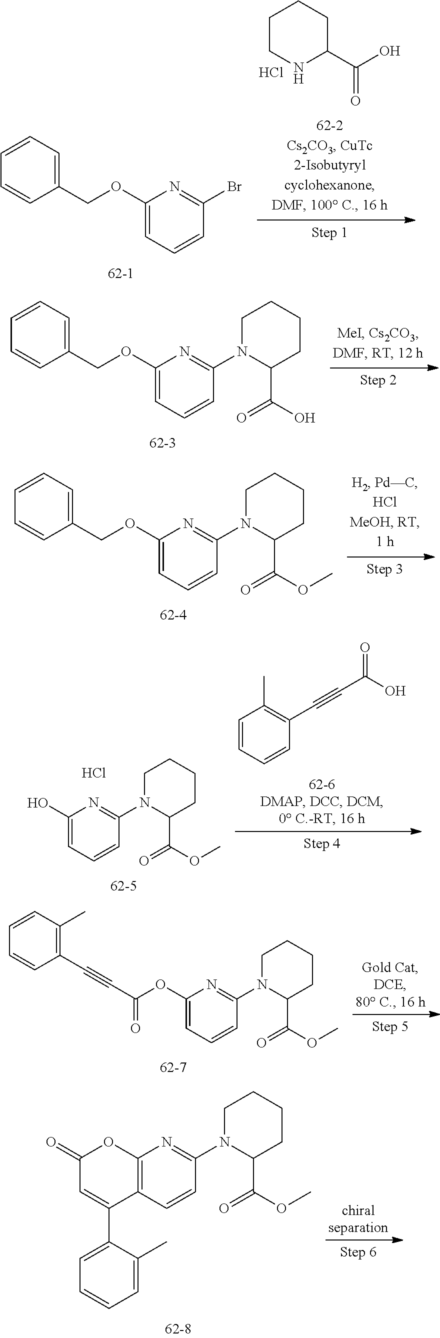

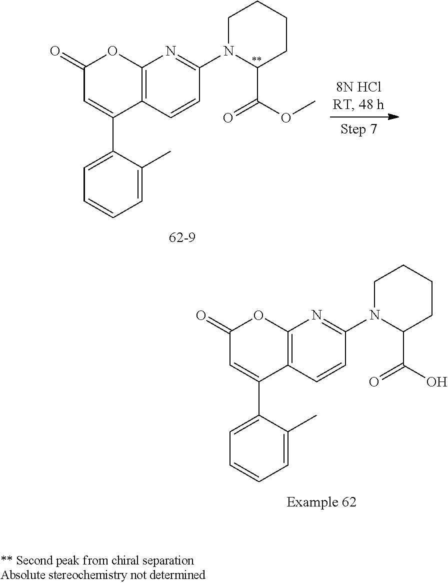 [SMILES: COC(=O)C1CCCCN1c1ccc2c(-c3ccccc3C)cc(=O)oc2n1 ; Cc1ccccc1-c1cc(=O)oc2nc(N3CCCCC3C(=O)O)ccc12]

Synthesis of 1-(6-(benzyloxy)pyridin-2-yl)piperidine-2-carboxylic acid, 62-3 [Step 1]: In a sealed tube, a stirred solution of piperidine-2-carboxylic acid hydrochloride (62-2, 830 mg, 5.0 mmol), 2-(benzyloxy)-6-bromopyridine (62-1, 1.20 g, 4.5 mmol) and cesium carbonate (2.96 g, 9.1 mmol) in DMF (6 mL) was allowed to degass with Argon gas for 15 min. Copper(I) thiophene 2-carboxylate (175 mg, 0.9 mmol) and 2-isobutyrylcyclohexanone (610 mg, 3.6 mmol) were added into the reaction mixture and heated at 100° C. for 16 h. After completion, the reaction mixture was cooled, diluted with water and ethyl acetate. The aqueous layer was acidified to ˜pH 4-5 using aqueous 2(N) HCl. The mixture was extracted with a mixture of 10% methanol in dichloromethane (twice). The combined organic extracts were dried over anhydrous sodium sulfate, filtered and concentrated under reduced pressure to afford 1-(6-(benzyloxy)pyridin-2-yl)piperidine-2-carboxylic acid (62-3, 1.8 g). LCMS (ESI) Calcd. for C₁₈H₂₀N₂O₃: 312, found [M+H]⁺=313.

Synthesis of methyl 1-(6-(benzyloxy)pyridin-2-yl)piperidine-2-carboxylate, 62-4 [Step 2]: A stirred solution of 1-(6-(benzyloxy)pyridin-2-yl)piperidine-2-carboxylic acid (62-3, 1.8 g, 3.9 mmol) and cesium carbonate (2.6 g, 7.8 mmol) in dry DMF (7 mL) was cooled to 0° C. Methyl iodide (0.7 mL, 11.8 mmol) was added dropwise to the mixture. The reaction mixture was allowed to stir at ambient temperature for 16 h. After completion, the reaction mixture was diluted with water and extracted with ethyl acetate. The organic extract was dried over anhydrous Na₂SO₄, filtered and concentrated under reduced pressure. The product was purified by flash-chromatography to afford methyl 1-(6-(benzyloxy)pyridin-2-yl)piperidine-2-carboxylate (62-4, 800 mg). LCMS (ESI) Calcd. for C₁₉H₂₂N₂O₃: 326, found [M+H]⁺=327.

Synthesis of methyl 1-(6-hydroxypyridin-2-yl)piperidine-2-carboxylate hydrochloride, 62-5 [Step 3]: To a stirred solution of methyl 1-(6-(benzyloxy)pyridin-2-yl)piperidine-2-carboxylate (62-4, 800 mg, 2.5 mmol) and 12 N HCl (0.5 mL) in methanol (30 mL), Argon gas was purged for 5 min. To this solution 10% Pd/C (300 mg, 0.3 mmol, 50% moist) was added and the mixture was hydrogenated using H₂ balloon at ambient temperature for 1 h. The reaction mixture was filtered through celite bed and concentrated under reduced pressure to afford methyl 1-(6-hydroxypyridin-2-yl)piperidine-2-carboxylate hydrochloride (62-5, 550 mg). LCMS (ESI) Calcd. for C₁₂H₁₆N₂O₃: 236, found [M+H]⁺=237.

Synthesis of methyl 1-(6-((3-(o-tolyl)propioloyl)oxy)pyridin-2-yl)piperidine-2-carboxylate, 62-7 [Step 4]: To a stirred solution of methyl 1-(6-hydroxypyridin-2-yl)piperidine-2-carboxylate hydrochloride (62-5, 550 mg, 2.0 mmol) and 3-(o-tolyl)propiolic acid (62-6, 390 mg, 2.4 mmol) in dichloromethane (20 mL) was added DMAP (40 mg, 0.4 mmol) and the mixture was cooled to 0° C. DCC (625 mg, 3.0 mmol) in 2 mL dichloromethane was slowly added into the mixture and stirred at ambient temperature for 16 h. The reaction mixture was diluted with dichloromethane, filtered through celite bed and concentrated under reduced pressure. The product was purified by column chromatography to afford methyl 1-(6-((3-(o-tolyl)propioloyl)oxy)pyridin-2-yl)piperidine-2-carboxylate (62-7, 230 mg). LCMS (ESI) Calcd. for C₂₂H₂₂N₂O₄: 378, found [M+H]⁺=379. ¹H NMR (400 MHz, CDCl₃) δ 7.59-7.54 (m, 2H), 7.35 (t, 1H), 7.19 (t, 1H), 6.56 (d, 1H), 6.42 (d, 1H), 5.31 (br s, 1H), 3.88-3.86 (m, 1H), 3.65 (s, 3H), 3.21 (t, 1H), 2.46 (s, 3H), 2.27-2.24 (m, 1H), 1.78-1.69 (m, 3H), 1.36-0.87 (m, 2H).

Synthesis of methyl 1-(2-oxo-4-(o-tolyl)-2H-pyrano[2,3-b]pyridin-7-yl)piperidine-2-carboxylate, 62-8 [Step 5]: A stirred solution of methyl 1-(6-((3-(o-tolyl)propioloyl)oxy)pyridin-2-yl)piperidine-2-carboxylate (62-7, 230 mg, 0.6 mmol) in dichloroethane (3 mL) was degassed with Argon gas for 5 min. Then (acetonitrile)[(2-biphenyl)di-tert-butylphosphine]gold(I) hexafluoroantimonate (50 mg, 0.06 mmol) was added and the mixture was heated at 80° C. for 18 h. After completion, the mixture was diluted with dichloromethane and passed through celite bed and concentrated under reduced pressure. The product was purified by column chromatography to afford methyl 1-(2-oxo-4-(o-tolyl)-2H-pyrano[2,3-b]pyridin-7-yl)piperidine-2-carboxylate (62-8, 140 mg). LCMS (ESI) Calcd. for C₂₂H₂₂N₂O₄: 378, found [M+H]⁺=379. ¹H NMR (400 MHz, CDCl₃) δ 7.44-7.38 (m, 2H), 7.34 (t, 1H), 7.22-3-7.20 (m, 1H), 7.10 (d, 1H), 6.85 (d, 1H), 6.05 (s, 1H), 5.41 (br s, 1H), 4.17 (br s, 1H), 3.67 (s, 3H), 3.05 (t, 1H), 2.22 (d, 1H), 2.12 (d, 3H), 1.78-1.69 (m, 1H), 1.49-1.46 (m, 1H), 1.33-1.23 (m, 3H).

Synthesis of chiral methyl 1-(2-oxo-4-(o-tolyl)-2H-pyrano[2,3-b]pyridin-7-yl)piperidine-2-carboxylate, 62-9 [Step 6]: Racemic methyl 1-(2-oxo-4-(o-tolyl)-2H-pyrano[2,3-b]pyridin-7-yl)piperidine-2-carboxylate (62-8, 270 mg, 0.7 mmol) was used for HPLC chiral SFC separation to afford the second product as chiral methyl 1-(2-oxo-4-(o-tolyl)-2H-pyrano[2,3-b]pyridin-7-yl)piperidine-2-carboxylate (62-9, 80 mg) as Peak 2. LCMS (ESI) Calcd. for C₂₂H₂₂N₂O₄: 378, found [M+H]⁺=379. ¹H NMR (400 MHz, DMSO-d₆) δ 7.44-7.38 (m, 2H), 7.34 (t, 1H), 7.22-3-7.20 (m, 1H), 7.10 (d, 1H), 6.85 (d, 1H), 6.05 (s, 1H), 5.41 (br s, 1H), 4.17 (br s, 1H), 3.67 (s, 3H), 3.05 (t, 1H), 2.22 (d, 1H), 2.12 (d, 3H), 1.78-1.69 (m, 3H), 1.52-1.46 (m, 1H), 1.33-1.23 (m, 1H).

Chiral HPLC prep-Method (SFC): SFC prep purification was done on Waters than SFC-80 equipped Waters UV Detector 2489 by using Column (R,R) WHELK-01 (21.1 mm×250 mm), 5μ operating at 35° C. temperature, maintaining flow rate of 60 gm/min, using 65% CO₂ in super critical state & 35% of (MeOH) as mobile phase, Run this isocratic mixture up to 20 minutes and also maintained the isobaric condition of 100 bar at 230 nm wavelength. The absolute stereochemistry was not determined.

Synthesis of 1-(2-oxo-4-(o-tolyl)-2H-pyrano[2,3-b]pyridin-7-yl)piperidine-2-carboxylic acid, Example 62 [Step 7]: To a stirred solution of chiral methyl 1-(2-oxo-4-(o-tolyl)-2H-pyrano[2,3-b]pyridin-7-yl)piperidine-2-carboxylate (62-9, 80 mg, 0.2 mmol) in tetrahydrofuran (1 mL) was added 8(N) HCl (6 mL). The reaction mixture was stirred at ambient temperature for 48 h. After completion, the volatiles were concentrated under reduced pressure. The product was purified by reverse phase HPLC to afford 1-(2-oxo-4-(o-tolyl)-2H-pyrano[2,3-b]pyridin-7-yl)piperidine-2-carboxylic acid (Example 62, 35 mg). LCMS (ESI) Calcd. for C₂₁H₂₀N₂O₄: 364, found [M+H]⁺=365. ¹H NMR (400 MHz, DMSO-d₆) δ 7.43-7.37 (m, 2H), 7.33 (t, 1H), 7.21 (d, 1H), 7.08 (d, 1H), 6.72 (d, 1H), 5.95 (s, 1H), 5.18 (br s, 1H), 4.19-4.17 (m, 1H), 3.17 (t, 1H), 2.27 (d, 1H), 2.15 (s, 3H), 1.79-1.69 (m, 3H), 1.56-1.27 (m, 2H). The compound racemized during hydrolysis.

### Example 63-64: Synthesis of chiral isomers of 2-(1-(2-oxo-4-(o-tolyl)-2H-pyrano[2,3-b]pyridin-7-yl)pyrrolidin-2-yl)acetamide

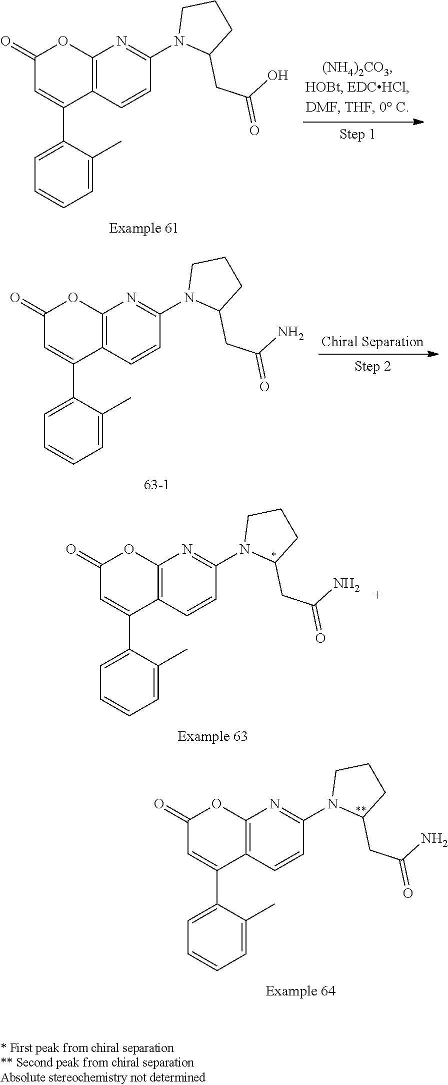 [SMILES: Cc1ccccc1-c1cc(=O)oc2nc(N3CCCC3CC(=O)O)ccc12 ; Cc1ccccc1-c1cc(=O)oc2nc(N3CCCC3CC(N)=O)ccc12 ; Cc1ccccc1-c1cc(=O)oc2nc(N3CCCC3CC(N)=O)ccc12 ; Cc1ccccc1-c1cc(=O)oc2nc(N3CCCC3CC(N)=O)ccc12]

Synthesis of 2-(1-(2-oxo-4-(o-tolyl)-2H-pyrano[2,3-b]pyridin-7-yl)pyrrolidin-2-yl)acetamide, 63-1 [Step 1]: To a stirred solution of 2-(1-(2-oxo-4-(o-tolyl)-2H-pyrano[2,3-b]pyridin-7-yl)pyrrolidin-2-yl)acetic acid (Example 61, 450 mg, 1.2 mmol) and (NH₄)₂CO₃ (475 mg, 5.0 mmol) in DMF (10 mL) and THF (10 mL), HOBt (250 mg, 1.8 mmol), EDC·HCl (710 mg, 3.7 mmol) and DIPEA (1.1 mL, 6.2 mmol) were added subsequently at 0° C. The reaction mixture was stirred at ambient temperature for 16 h. The reaction was diluted with water and extracted with ethyl acetate (twice). The combined organic extract was washed with ice-water and cold brine. The organic extract was dried over anhydrous sodium sulphate, filtered, and concentrated under reduced pressure. The product was purified by flash column-chromatography to afford 2-(1-(2-oxo-4-(o-tolyl)-2H-pyrano[2,3-b]pyridin-7-yl)pyrrolidin-2-yl)acetamide (63-1, 350 mg). LCMS (ESI) Calcd. for C₂₁H₂₁N₃O₃: 363, found [M+H]⁺=364.

Synthesis of chiral isomers of 2-(1-(2-oxo-4-(o-tolyl)-2H-pyrano[2,3-b]pyridin-7-yl)pyrrolidin-2-yl)acetamide, Example 63 and 64 [Step 2]: Racemic 2-(1-(2-oxo-4-(o-tolyl)-2H-pyrano[2,3-b]pyridin-7-yl)pyrrolidin-2-yl)acetamide (63-1, 350 mg. 0.9 mmol) was separated by SFC HPLC chiral purification and lyophilized to afford the first product as chiral 2-(1-(2-oxo-4-(o-tolyl)-2H-pyrano[2,3-b]pyridin-7-yl)pyrrolidin-2-yl)acetamide (Example 63, 90 mg) as Peak 1, and the second product as chiral 2-(1-(2-oxo-4-(o-tolyl)-2H-pyrano[2,3-b]pyridin-7-yl)pyrrolidin-2-yl)acetamide (Example 64, 95 mg) as Peak 2. The absolute stereochemistry of these compounds was not determined.

Example 63: Chiral 2-(1-(2-oxo-4-(o-tolyl)-2H-pyrano[2,3-b]pyridin-7-yl)pyrrolidin-2-yl)acetamide, Peak 1: LCMS (ESI) Calcd. for C₂₁H₂₁N₃O₃: 363, found [M+H]⁺=364. ¹H NMR (400 MHz, DMSO-d₆) δ 7.43-7.31 (m, 3H), 7.20 (d, 1H), 7.05 (d, 1H), 6.45 (d, 1H), 6.90 (br s, 2H), 5.92 (s, 1H), 4.41 (br s, 1H), 3.50 (br s, 1H), 3.45 (br s, 1H), 2.65 (d, 1H), 2.24-2.18 (m, 1H), 2.14 (s, 3H), 2.10-1.93 (m, 4H).

Example 64: Chiral 2-(1-(2-oxo-4-(o-tolyl)-2H-pyrano[2,3-b]pyridin-7-yl)pyrrolidin-2-yl)acetamide, Peak 2: LCMS (ESI) Calcd. for C₂₁H₂₁N₃O₃: 363, found [M+H]⁺=364. ¹H NMR (400 MHz, DMSO-d₆) δ 7.43-7.31 (m, 3H), 7.20 (d, 1H), 7.05 (d, 1H), 6.45 (d, 1H), 6.90 (br s, 2H), 5.92 (s, 1H), 4.41 (br s, 1H), 3.50 (br s, 1H), 3.45 (br s, 1H), 2.65 (d, 1H), 2.24-2.18 (m, 1H), 2.14 (s, 3H), 2.10-1.93 (m, 4H).

SFC chiral HPLC method: Chiral separation was performed on Thar SFC-80 series instrument. The column used was Chiralpak IG (21 mm×250 mm, 5μ) which was operating at a temperature of 35° C. with a flow rate of 60 ml/min. The mobile phase used is 50% CO₂ at super critical state, 50% (0.3% IPAmine in MeOH), held isocratic and isobaric (100 bar) up to 20 min. with detection at a wavelength of 370 nm.

### Example 65-66: Synthesis of chiral isomers of 2-(1-(2-oxo-4-(o-tolyl)-2H-pyrano[2,3-b]pyridin-7-yl)pyrrolidin-2-yl)acetic acid

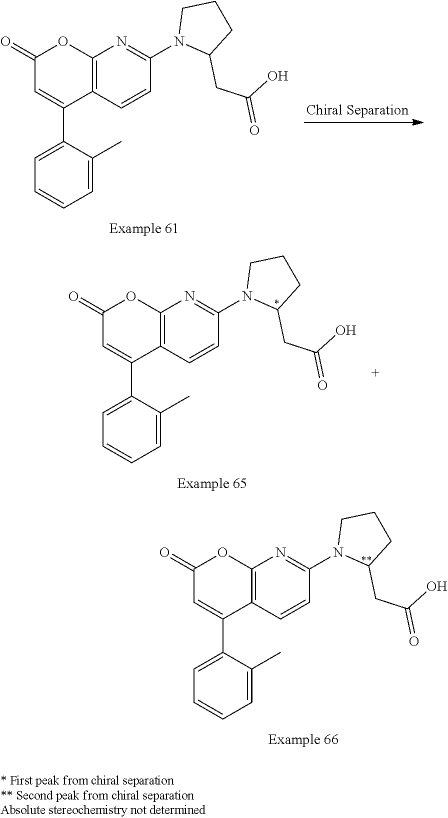 [SMILES: Cc1ccccc1-c1cc(=O)oc2nc(N3CCCC3CC(=O)O)ccc12 ; Cc1ccccc1-c1cc(=O)oc2nc(N3CCCC3CC(=O)O)ccc12 ; Cc1ccccc1-c1cc(=O)oc2nc(N3CCCC3CC(=O)O)ccc12]

Racemic 2-(1-(2-oxo-4-(o-tolyl)-2H-pyrano[2,3-b]pyridin-7-yl)pyrrolidin-2-yl)acetic acid (Example 61, 350 mg. 0.9 mmol) was separated by SFC HPLC chiral purification and lyophilized to afford the first product as chiral 2-(1-(2-oxo-4-(o-tolyl)-2H-pyrano[2,3-b]pyridin-7-yl)pyrrolidin-2-yl)acetic acid (Example 65, 30 mg) as Peak 1, and the second product as chiral 2-(1-(2-oxo-4-(o-tolyl)-2H-pyrano[2,3-b]pyridin-7-yl)pyrrolidin-2-yl)acetic acid (Example 66, 30 mg) as Peak 2. The absolute stereochemistry of these compounds were not determined and was arbitrarily assigned.

Example 65: Chiral 2-(1-(2-oxo-4-(o-tolyl)-2H-pyrano[2,3-b]pyridin-7-yl)pyrrolidin-2-yl)acetic acid, Peak 1: LCMS (ESI) Calcd. for C₂₁H₂₀N₂O₄: 364, found [M+H]⁺=365. ¹H NMR (400 MHz, DMSO-d₆) δ 11.40 (br s, 1H), 7.43-7.31 (m, 3H), 7.20 (d, 1H), 7.05 (d, 1H), 6.42 (d, 1H), 5.92 (s, 1H), 4.45 (br s, 1H), 3.45-3.47 (m, 1H), 3.45-3.40 (m, 1H), 2.85-2.81 (m, 1H), 2.50-2.34 (m, 1H), 2.15 (s, 3H), 2.12-2.06 (m, 2H), 2.04-1.91 (m, 2H).

Example 66: Chiral 2-(1-(2-oxo-4-(o-tolyl)-2H-pyrano[2,3-b]pyridin-7-yl)pyrrolidin-2-yl)acetic acid, Peak 2: LCMS (ESI) Calcd. for C₂₁H₂₀N₂O₄: 364, found [M+H]⁺=365. ¹H NMR (400 MHz, DMSO-d₆) δ 7.42-7.31 (m, 3H), 7.20 (d, 1H), 7.05 (d, 1H), 6.42 (d, 1H), 5.92 (s, 1H), 4.45 (br s, 1H), 3.45-3.47 (m, 1H), 3.45-3.40 (m, 1H), 2.85-2.81 (m, 1H), 2.50-2.34 (m, 1H), 2.15 (s, 3H), 2.12-2.06 (m, 2H), 2.04-1.91 (m, 2H).

NP chiral HPLC method: The column used was Chiralpak IG (21 mm×250 mm, 5μ) which was operating at a temperature of 35° C. with a flow rate of 21 mL/min. The mobile phase used is mixture of 50% hexane, 25% dichloromethane and 25% isopropanol, held isocratic and isobaric up to 20 min. with detection at a wavelength of 365 nm.

### Example 67-68: Synthesis of chiral isomers of methyl 3-(2-oxo-4-(o-tolyl)-2H-pyrano[2,3-b]pyridin-7-yl)-3-azabicyclo[3.1.0]hexane-2-carboxylate

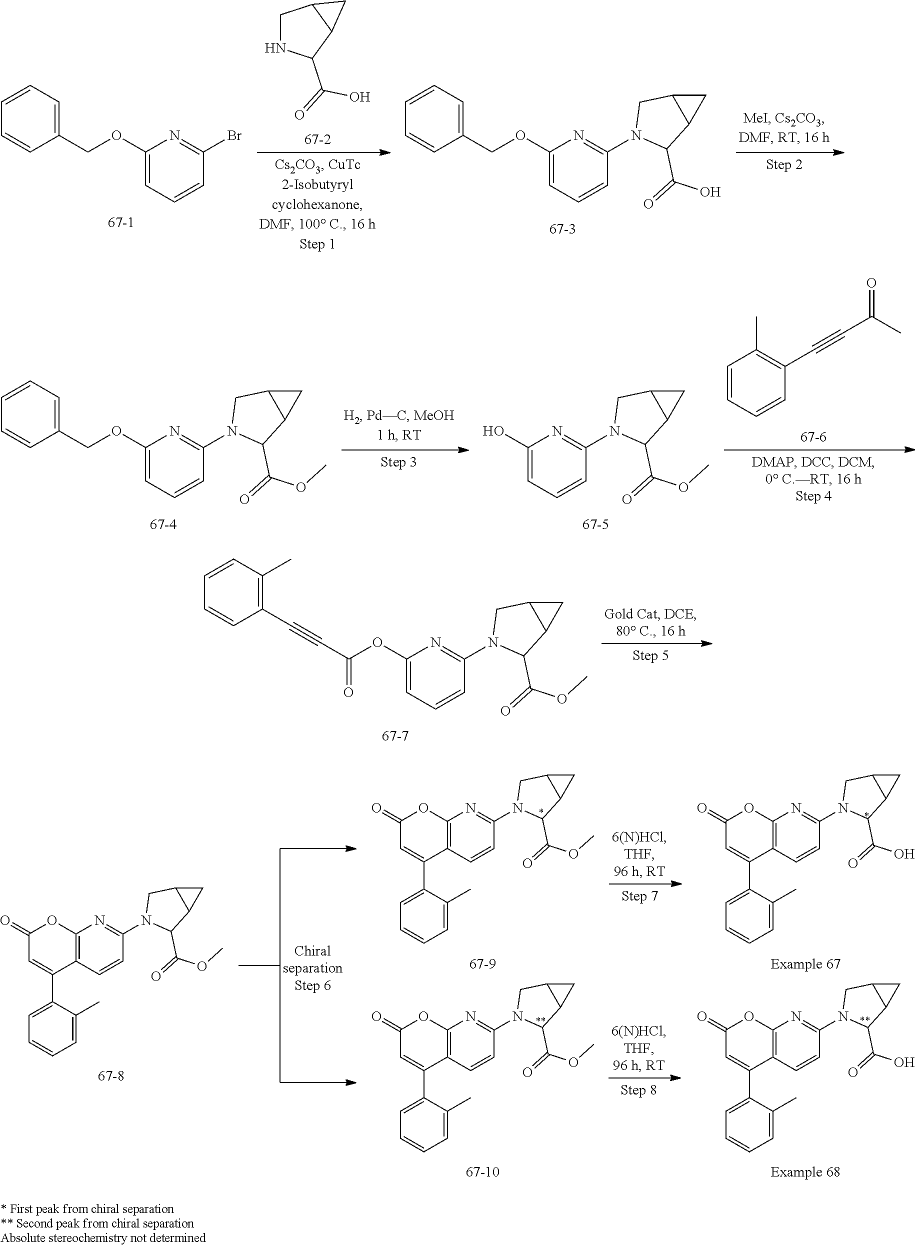

Synthesis of 3-(6-(benzyloxy)pyridin-2-yl)-3-azabicyclo[3.1.0]hexane-2-carboxylic acid, 67-3 [Step 1]: In sealed tube, 2-(benzyloxy)-6-bromopyridine (67-1, 1.0 g, 3.8 mmol) and DMF (6 mL) were taken. Into the reaction mixture Cs₂CO₃ (1.7 g, 5.3 mmol), 3-azabicyclo[3.1.0]hexane-2-carboxylic acid (67-2, 405 mg, 3.2 mmol) were added and the reaction mixture was degassed with Argon gas for 10 min. Copper thiophene 2-carboxylate (200 mg, 1.1 mmol) and 2-isobutyrylcyclohexanone (0.4 mL, 2.3 mmol) were added and the reaction mixture was heated at 100° C. for 16 h. After completion, the reaction mixture was quenched with water and extracted with ethyl acetate. Organic extract was kept aside and the aqueous extract was acidified using 2 (N) HCl up to pH-3 and extracted with ethyl acetate (twice). The combined organic extract was washed with brine, dried over anhydrous Na₂SO₄, filtered and concentrated under reduced pressure to afford 3-(6-(benzyloxy)pyridin-2-yl)-3-azabicyclo[3.1.0]hexane-2-carboxylic acid (67-3, 1.0 g). LCMS (ESI) Calcd. for C₁₈H₁₈N₂O₃: 310, found [M+H]⁺=311.

Synthesis of methyl 3-(6-(benzyloxy)pyridin-2-yl)-3-azabicyclo[3.1.0]hexane-2-carboxylate, 67-4 [Step 2]: A stirred solution of 3-(6-(benzyloxy)pyridin-2-yl)-3-azabicyclo[3.1.0]hexane-2-carboxylic acid (67-3, 1.0 g, 3.8 mmol) and Cs₂CO₃ (2.1 g, 6.4 mmol) in DMF (10 mL) was cooled to 0° C. Into the cold reaction mixture, methyl iodide (0.7 mL, 11.3 mmol) was added dropwise and the reaction mixture was allowed to stir at ambient temperature for 16 h. After completion, the reaction mixture was quenched with water and extracted with ethyl acetate (twice). The combined organic extract was washed with water, brine, dried over anhydrous Na₂SO₄ and concentrated under reduced pressure. The product was purified by flash chromatography to afford methyl 3-(6-(benzyloxy)pyridin-2-yl)-3-azabicyclo[3.1.0]hexane-2-carboxylate (67-4, 300 mg). LCMS (ESI) Calcd. for C₁₉H₂₀N₂O₃: 324, found [M+H]⁺=325.

Synthesis of methyl 3-(6-hydroxypyridin-2-yl)-3-azabicyclo[3.1.0]hexane-2-carboxylate, 67-5 [Step 3]: To a stirred solution of methyl 3-(6-(benzyloxy)pyridin-2-yl)-3-azabicyclo[3.1.0]hexane-2-carboxylate (67-4, 300 mg, 0.9 mmol) in methanol (15 mL) was added 12 (N) HCl (0.2 mL) and the reaction mixture was purged with Argon gas for 5 min. Into the reaction mixture, 10% Pd/C (90 mg, 50% moist) was added and the reaction mixture was hydrogenated using a hydrogen balloon at ambient temperature for 1 h. After completion, the reaction mixture was filtered through celite bed and the filtrate was concentrated under reduced pressure to afford methyl 3-(6-hydroxypyridin-2-yl)-3-azabicyclo[3.1.0]hexane-2-carboxylate (67-5, 200 mg). LCMS (ESI) Calcd. for C₁₂H₁₄N₂O₃: 234, found [M+H]⁺=235.

Synthesis of methyl 3-(6-((3-(o-tolyl)propioloyl)oxy)pyridin-2-yl)-3-azabicyclo[3.1.0]hexane-2-carboxylate, 67-7 [Step 4]: To a stirred solution of methyl 3-(6-hydroxypyridin-2-yl)-3-azabicyclo[3.1.0]hexane-2-carboxylate (67-5, 250 mg, 1.1 mmol) and 4-(o-tolyl)but-3-yn-2-one (67-6, 250 mg, 1.6 mmol) in dichloromethane (10 mL), DMAP (20 mg, 0.2 mmol) was added and the mixture was cooled to 0° C. Into this cold reaction mixture, a dichloromethane (5 mL) solution of DCC (330 mg, 1.6 mmol) was added and continued to stir at ambient temperature for 16 h. After completion, the reaction mixture was diluted with dichloromethane and filtered through celite bed. The filtrate was concentrated under reduced pressure and the product was purified by flash chromatography to afford methyl 3-(6-((3-(o-tolyl)propioloyl)oxy)pyridin-2-yl)-3-azabicyclo[3.1.0]hexane-2-carboxylate (67-7, 150 mg). LCMS (ESI) Calcd. for C₂₂H₂₀N₂O₄: 376, found [M+H]⁺=377.

Synthesis of methyl 3-(2-oxo-4-(o-tolyl)-2H-pyrano[2,3-b]pyridin-7-yl)-3-azabicyclo[3.1.0]hexane-2-carboxylate, 67-8 [Step 5]: A solution of methyl 3-(6-((3-(o-tolyl)propioloyl)oxy)pyridin-2-yl)-3-azabicyclo[3.1.0]hexane-2-carboxylate (67-7, 150 mg, 0.4 mmol) in dichloroethane (2 mL) was degassed with Argon gas for 5 min. Then (Acetonitrile)[(2-biphenyl)di-tert-butylphosphine]gold(I) hexafluoroantimonate (30 mg, 0.04 mmol) was added into the reaction mixture and heated at 80° C. for 16 h. After completion, the mixture was diluted with dichloromethane and filtered through celite bed. The filtrate was concentrated under reduced pressure and purified by flash chromatography to afford methyl 3-(2-oxo-4-(o-tolyl)-2H-pyrano[2,3-b]pyridin-7-yl)-3-azabicyclo[3.1.0]hexane-2-carboxylate (67-8, 110 mg). LCMS (ESI) Calcd. for C₂₂H₂₀N₂O₄: 376, found [M+H]⁺=377.

Synthesis of chiral isomers of methyl 3-(2-oxo-4-(o-tolyl)-2H-pyrano[2,3-b]pyridin-7-yl)-3-azabicyclo[3.1.0]hexane-2-carboxylate, 67-9 and 67-10 [Step 6]: Racemic mixture of methyl 3-(2-oxo-4-(o-tolyl)-2H-pyrano[2,3-b]pyridin-7-yl)-3-azabicyclo[3.1.0]hexane-2-carboxylate (67-8, 110 mg) was separated by HPLC-Chiral normal phase to afford the first product as chiral methyl 3-(2-oxo-4-(o-tolyl)-2H-pyrano[2,3-b]pyridin-7-yl)-3-azabicyclo[3.1.0]hexane-2-carboxylate (67-9, 48 mg) as Peak 1, and the second product as chiral methyl 3-(2-oxo-4-(o-tolyl)-2H-pyrano[2,3-b]pyridin-7-yl)-3-azabicyclo[3.1.0]hexane-2-carboxylate (67-10, 42 mg) as Peak 2. The absolute stereochemistry of these compounds was not determined.

67-9: Chiral methyl 3-(2-oxo-4-(o-tolyl)-2H-pyrano[2,3-b]pyridin-7-yl)-3-azabicyclo[3.1.0]hexane-2-carboxylate, Peak 1: LCMS (ESI) Calcd. for C₂₂H₂₀N₂O₄: 376, found [M+H]⁺=377.

67-10: Chiral methyl 3-(2-oxo-4-(o-tolyl)-2H-pyrano[2,3-b]pyridin-7-yl)-3-azabicyclo[3.1.0]hexane-2-carboxylat, Peak 2: LCMS (ESI) Calcd. for C₂₂H₂₀N₂O₄: 376, found [M+H]⁺=377.

Chiral HPLC Method: Chiral separation was performed on an Agilent 1200 series instrument. Column name: CHIRALPAK IG (250×21 mm) 5μ. Operating at ambient temperature and flow rate is 21.0 mL/min. Mobile phase was mixture of 60% hexane, 20% dichloromethane, 20% ethyl alcohol, and held isocratic for up to 10 min. with wavelength of 358 nm.

Synthesis of chiral 3-(2-oxo-4-(o-tolyl)-2H-pyrano[2,3-b]pyridin-7-yl)-3-azabicyclo[3.1.0]hexane-2-carboxylic acid, Example 67 [Step 7]: A solution of chiral methyl 3-(2-oxo-4-(o-tolyl)-2H-pyrano[2,3-b]pyridin-7-yl)-3-azabicyclo[3.1.0]hexane-2-carboxylate (67-9, 48 mg, 0.1 mmol) in tetrahydrofuran (2 mL) was cooled to 0° C. and into this cold reaction mixture 6(N) HCl (6 mL) was added drop wise and stirring was continued at ambient temperature for 96 h. After completion, it was diluted with water, extracted with ethyl acetate (twice). The combined organic extract was washed with brine, dried over anhydrous Na₂SO₄ and concentrated under reduced pressure. The product was purified by reverse phase prep HPLC purification method and lyophilized to afford chiral 3-(2-oxo-4-(o-tolyl)-2H-pyrano[2,3-b]pyridin-7-yl)-3-azabicyclo[3.1.0]hexane-2-carboxylic acid (Example 67, 18 mg). LCMS (ESI) Calcd. for C₂₁H₁₈N₂O₄: 362, found [M+H]⁺=363. ¹H NMR (400 MHz, DMSO-d₆) δ 12.60 (br s, 1H), 7.43-7.31 (m, 3H), 7.21-7.19 (m, 1H), 7.08-7.06 (m, 1H), 6.45 (br s, 1H), 6.02 (s, 1H), 4.49 (br s, 1H), 3.68-3.59 (m, 2H), 2.10 (s, 3H), 1.86 (br s, 1H), 1.23 (br s, 1H), 0.76-0.59 (m, 2H). The absolute stereochemistry was not determined.

Synthesis of chiral 3-(2-oxo-4-(o-tolyl)-2H-pyrano[2,3-b]pyridin-7-yl)-3-azabicyclo[3.1.0]hexane-2-carboxylic acid, Example 68 [Step 8]: A solution of chiral methyl 3-(2-oxo-4-(o-tolyl)-2H-pyrano[2,3-b]pyridin-7-yl)-3-azabicyclo[3.1.0]hexane-2-carboxylate (67-10, 40 mg, 0.1 mmol) in tetrahydrofuran (2 mL) was cooled to 0° C. and into this cold reaction mixture 6(N) HCl (6 mL) was added drop wise and stirring was continued at ambient temperature for 96 h. After completion, it was diluted with water and extracted with ethyl acetate (twice). The combined organic extract was washed with brine, dried over anhydrous Na₂SO₄ and concentrated under reduced pressure. The product was purified by reverse phase prep HPLC purification method and lyophilized to afford chiral 3-(2-oxo-4-(o-tolyl)-2H-pyrano[2,3-b]pyridin-7-yl)-3-azabicyclo[3.1.0]hexane-2-carboxylic acid (Example 68, 15 mg). LCMS (ESI) Calcd. for C₂₁H₁₈N₂O₄: 362, found [M+H]⁺=363. ¹H NMR (400 MHz, DMSO-d₆) δ 7.43-7.31 (m, 3H), 7.20-7.18 (m, 1H), 7.03 (m, 1H), 6.43-6.41 (m, 1H), 5.99 (br s, 1H), 4.48 (m, 1H), 3.66-3.64 (m, 2H), 2.10-2.05 (m, 3H), 1.90-1.82 (m, 1H), 0.88-0.61 (m, 3H). The absolute stereochemistry was not determined.

### Example 69-70: Synthesis of chiral isomers of 1-(2-oxo-4-(o-tolyl)-2H-pyrano[2,3-b]pyridin-7-yl)piperidine-2-carboxylic acid

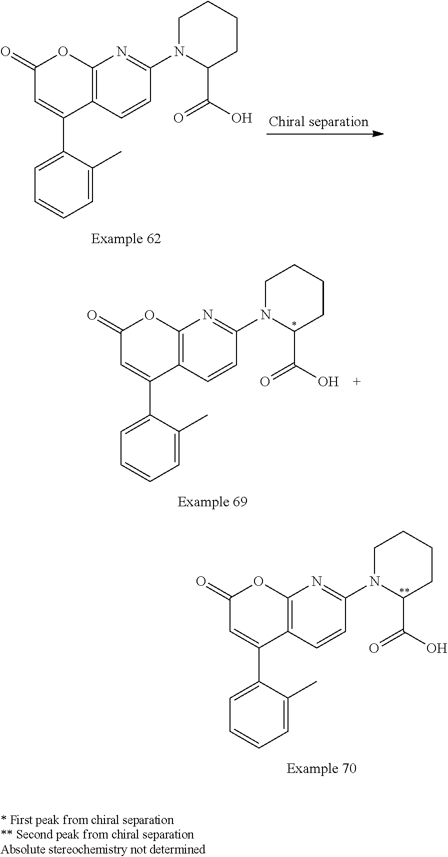 [SMILES: Cc1ccccc1-c1cc(=O)oc2nc(N3CCCCC3C(=O)O)ccc12 ; Cc1ccccc1-c1cc(=O)oc2nc(N3CCCCC3C(=O)O)ccc12 ; Cc1ccccc1-c1cc(=O)oc2nc(N3CCCCC3C(=O)O)ccc12]

Racemic 1-(2-oxo-4-(o-tolyl)-2H-pyrano[2,3-b]pyridin-7-yl)piperidine-2-carboxylic acid (Example 62, 50 mg, 0.1 mmol) was used for SFC chiral HPLC separation to afford the first product as chiral 1-(2-oxo-4-(o-tolyl)-2H-pyrano[2,3-b]pyridin-7-yl)piperidine-2-carboxylic acid as (Example 69, 10 mg) as Peak 1, and the second product as chiral 1-(2-oxo-4-(o-tolyl)-2H-pyrano[2,3-b]pyridin-7-yl)piperidine-2-carboxylic acid (Example 70, 17 mg) as Peak 2. The absolute stereochemistry of these compounds was not determined.

Example 69: Chiral 1-(2-oxo-4-(o-tolyl)-2H-pyrano[2,3-b]pyridin-7-yl)piperidine-2-carboxylic acid, Peak 1: LCMS (ESI) Calcd. for C₂₁H₂₀N₂O₄: 364, found [M+H]⁺=365. ¹H NMR (400 MHz, DMSO-d₆) δ 12.88 (s, 1H), 7.44-7.39 (m, 2H), 7.34 (t, 1H), 7.21 (t, 1H), 7.08 (d, 1H), 6.83 (d, 1H), 6.04 (s, 1H), 5.29 (br s, 1H), 4.17 (br s, 1H), 3.16 (t, 1H), 2.27 (d, 1H), 2.15 (s, 3H), 1.79-1.69 (m, 3H), 1.56-1.27 (m, 2H).

Example 70: Chiral 1-(2-oxo-4-(o-tolyl)-2H-pyrano[2,3-b]pyridin-7-yl)piperidine-2-carboxylic acid, Peak 2: LCMS (ESI) Calcd. for C₂₁H₂₀N₂O₄: 364, found [M+H]⁺=365. ¹H NMR (400 MHz, DMSO-d₆) δ 12.86 (s, 1H), 7.44-7.39 (m, 2H), 7.34 (t, 1H), 7.21 (t, 1H), 7.08 (d, 1H), 6.83 (d, 1H), 6.04 (s, 1H), 5.29 (br s, 1H), 4.17 (br s, 1H), 3.16 (t, 1H), 2.27 (d, 1H), 2.15 (s, 3H), 1.79-1.69 (m, 3H), 1.56-1.27 (m, 2H).

Chiral HPLC prep method (SFC): SFC prep purification was performed on a THAR SFC-80 instrument using CHIRALPAK-IG column (30.0 mm×250 mm), 5p operating at 35° C. temperature, maintaining flow rate of 70 gm/min, using 60% CO₂ in super critical state & 40% of (ACN/Methanol (1:1)) as mobile phase, without using any modifier. Run this isocratic mixture up to 20 min. and also maintained the isobaric condition of 100 bar at 371 nm wavelength.

### Example 71: Synthesis of (R)-2-(2-(methyl(2-oxo-4-(o-tolyl)-2H-pyrano[2,3-b]pyridin-7-yl)amino)acetamido)propanamide

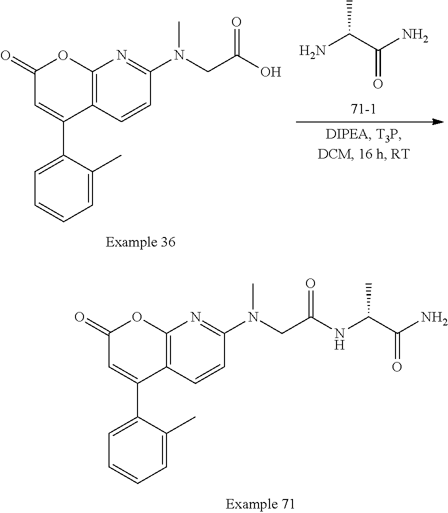

A stirred solution of N-methyl-N-(2-oxo-4-(o-tolyl)-2H-pyrano[2,3-b]pyridin-7-yl)glycine (Example 36, 120 mg, 0.4 mmol) and (R)-2-aminopropanamide (71-1, 40 mg, 0.4 mmol) in dichloromethane (4 mL) was cooled to 0° C. and DIPEA (0.2 mL, 1.1 mmol) was added. Into this cold reaction mixture, T3P (0.3 mL, 0.6 mmol, 50% in ethyl acetate) was added drop wise and the reaction mixture was allowed to warm up to ambient temperature and stirred for 16 h. After completion, the reaction mixture was quenched with ice cold water and extracted with dichloromethane (twice). The combined organic extract was washed with brine, dried over anhydrous Na₂SO₄ and concentrated under reduced pressure. The product was purified by flash chromatography to afford (R)-2-(2-(methyl(2-oxo-4-(o-tolyl)-2H-pyrano[2,3-b]pyridin-7-yl)amino)acetamido)propanamide (Example 71, 50 mg). LCMS (ESI) Calcd. for C₂₁H₂₂N₄O₄: 394, found [M+H]⁺=395. ¹H NMR (400 MHz, DMSO-d₆) δ 8.20-8.19 (m, 1H), 7.43-7.31 (m, 4H), 7.22-7.20 (m, 1H), 7.07-7.01 (m, 2H), 6.62-6.60 (m, 1H), 6.00 (m, 1H), 4.28-4.18 (m, 3H), 3.09 (s, 3H), 2.12 (d, 3H), 1.22-1.20 (d, 3H).

### Example 72: Synthesis of (S)-4,4-difluoro-1-(2-oxo-4-(o-tolyl)-2H-pyrano[2,3-b]pyridin-7-yl)pyrrolidine-2-carboxylic acid

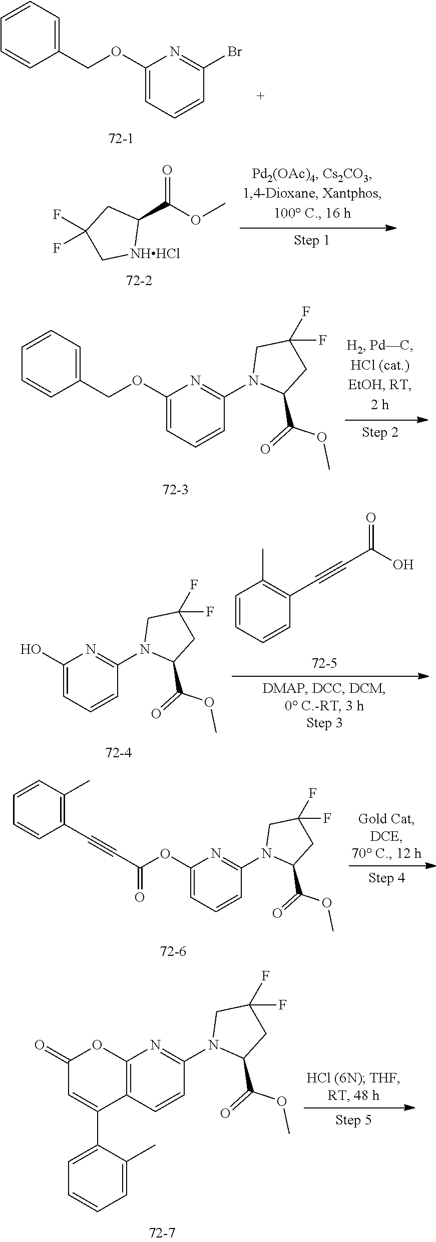

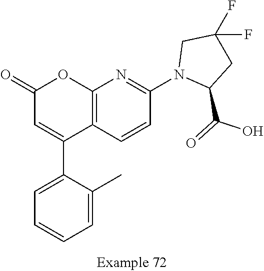 [SMILES: Cc1ccccc1-c1cc(=O)oc2nc(N3CC(F)(F)C[C@H]3C(=O)O)ccc12]

Synthesis of methyl (S)-1-(6-(benzyloxy)pyridin-2-yl)-4,4-difluoropyrrolidine-2-carboxylate, 72-3 [Step 1]: In a sealed tube, a suspension of 2-benzyloxy-6-bromo-pyridine (72-1, 2.6 g, 9.9 mmol), cesium carbonate (9.7 g, 29.8 mmol) and methyl (S)-4,4-difluoropyrrolidine-2-carboxylate hydrochloride (72-2, 2.0 g, 9.9 mmol) in 1,4-dioxane (25 mL) was purged with argon gas for 15 min. Pd(OAc)₂ (0.3 g, 1.5 mmol) and Xantphos (1.7 g, 3.0 mmol) were added. The resulting mixture was heated at 100° C. for 16 h. The reaction mixture was filtered through celite pad and washed with ethyl acetate. The combined filtrate was washed with brine, dried over anhydrous sodium sulphate and concentrated under reduced pressure. The product was purified by combiflash chromatography to afford methyl (S)-1-(6-(benzyloxy)pyridin-2-yl)-4,4-difluoropyrrolidine-2-carboxylate (72-3, 1.4 g). LCMS (ESI) Calcd. for C₁₈H₁₈F₂N₂O₃: 348, found [M+H]⁺=349. ¹H NMR (400 MHz, DMSO-d₆) δ 7.45-7.28 (m, 6H), 6.20 (d, 1H), 5.90 (d, 1H), 5.30-5.27 (m, 1H), 5.20-5.17 (m, 1H), 4.77-4.74 (m, 1H), 3.95-3.83 (m, 2H), 3.67 (s, 3H), 2.81-2.76 (m, 1H), 2.56-2.52 (m, 1H).

Synthesis of methyl (S)-4,4-difluoro-1-(6-hydroxypyridin-2-yl)pyrrolidine-2-carboxylate, 72-4 [Step 2]: To a stirred solution of methyl (S)-1-(6-(benzyloxy)pyridin-2-yl)-4,4-difluoropyrrolidine-2-carboxylate (72-3, 600 mg, 1.7 mmol) in ethanol (15 mL), Pd—C(180 mg, 10% w/w) was added. The reaction mixture was hydrogenated using hydrogen balloon at ambient temperature for 2 h, filtered through a pad of celite and washed with ethanol. The combined filtrate was concentrated under reduced pressure to afford methyl (S)-4,4-difluoro-1-(6-hydroxypyridin-2-yl) pyrrolidine-2-carboxylate (72-4, 350 mg). LCMS (ESI) Calcd. for C₁₁H₁₂F₂N₂O₃: 258, found [M+H]⁺=259.

Synthesis of methyl (S)-4,4-difluoro-1-(6-((3-(o-tolyl)propioloyl)oxy)pyridin-2-yl)pyrrolidine-2-carboxylate, 72-6 [Step 3]: To a solution of methyl (S)-4,4-difluoro-1-(6-hydroxypyridin-2-yl)pyrrolidine-2-carboxylate (72-4, 350 mg, 1.36 mmol), 3-(o-tolyl)prop-2-ynoic acid (72-5, 215 mg, 1.4 mmol) and N,N-dimethyl amino pyridine (35 mg, 0.27 mmol) in dichloromethane (15 mL) at 0° C., a solution of N,N′-dicyclohexylcarbodiimide (560 mg, 2.7 mmol) in dichloromethane (2 mL) was added dropwise. The resulting reaction mixture was allowed to stir at ambient temperature for 3 h, diluted with dichloromethane and washed with water followed by brine. The combined organic extracts was dried over anhydrous sodium sulphate and concentrated under reduced pressure. The product was purified by combiflash chromatography to afford methyl (S)-4,4-difluoro-1-(6-((3-(o-tolyl)propioloyl)oxy)pyridin-2-yl)pyrrolidine-2-carboxylate (72-6, 140 mg). LCMS (ESI) Calcd. for C₂₁H₁₈F₂N₂O₄: 400, found [M+H]⁺=401. ¹H NMR (400 MHz, DMSO-d₆) δ 7.70 (t, 1H), 7.64 (d, 1H), 7.49-7.47 (m, 1H), 7.39 (d, 1H), 7.31 (t, 1H), 6.64-6.58 (m, 2H), 4.79-4.76 (m, 1H), 4.00-3.96 (m, 2H), 3.62 (s, 3H), 4.05-3.95 (m, 1H), 2.60-2.50 (m, 1H), 2.38 (s, 3H).

Synthesis of methyl (S)-4,4-difluoro-1-(2-oxo-4-(o-tolyl)-2H-pyrano[2,3-b]pyridin-7-yl)pyrrolidine-2-carboxylate, 72-7 [Step 4]: A solution methyl (S)-4,4-difluoro-1-(6-((3-(o-tolyl)propioloyl)oxy)pyridin-2-yl)pyrrolidine-2-carboxylate (72-6, 140 mg, 0.35 mmol) in dry dichloroethane (5 mL) was purged with Argon gas for 10 min and (acetonitrile)[(2-biphenyl)di-tert-butylphosphine]gold(I) hexafluoroantimonate (40 mg, 0.05 mmol) was added. The reaction mixture was heated at 70° C. for 4 h. After cooling, the reaction mixture was filtered through a pad of celite, washed with ethyl acetate and the combined filtrates were concentrated under reduced pressure. The product was purified by flash chromatography to afford methyl (S)-4,4-difluoro-1-(2-oxo-4-(o-tolyl)-2H-pyrano[2,3-b]pyridin-7-yl)pyrrolidine-2-carboxylate (72-7, 88 mg). LCMS (ESI) Calcd. for C₂₁H₁₈F₂N₂O₄: 400, found [M+H]⁺=401. ¹H NMR (400 MHz, CDCl₃) δ 7.39-7.36 (m, 1H), 7.32-7.29 (m, 2H), 7.21 (d, 1H), 7.13-7.11 (m, 1H), 6.21 (d, 1H), 6.08 (s, 1H), 4.95 (br s, 1H), 4.02-3.96 (m, 2H), 3.80 (s, 3H), 2.87-2.81 (m, 1H), 2.66-2.62 (m, 1H), 2.14 (s, 3H).

Synthesis of (S)-4,4-difluoro-1-(2-oxo-4-(o-tolyl)-2H-pyrano[2,3-b]pyridin-7-yl)pyrrolidine-2-carboxylic acid, Example 72 [Step 5]: A solution of methyl (S)-4,4-difluoro-1-(2-oxo-4-(o-tolyl)-2H-pyrano[2,3-b]pyridin-7-yl)pyrrolidine-2-carboxylate (72-7, 85 mg, 0.2 mmol) in aqueous hydrochloric acid (4 mL, 6 N) and tetrahydrofuran (2 mL) was stirred at ambient temperature for 48 h. After completion, the volatiles were evaporated under reduced pressure. The product purified by reverse phase prep HPLC to afford (S)-4,4-difluoro-1-(2-oxo-4-(o-tolyl)-2H-pyrano[2,3-b]pyridin-7-yl)pyrrolidine-2-carboxylic acid (Example 72, 20 mg). LCMS (ESI) Calcd. for C₂₀H₁₆F₂N₂O₄: 386, found [M+H]⁺=387. ¹H NMR (400 MHz, DMSO-d₆ at 100° C.) δ 7.42-7.32 (m, 3H), 7.22 (d, 1H), 7.13 (d, 1H), 6.52 (d, 1H), 6.01 (s, 1H), 4.73 (br s, 1H), 4.13-4.07 (m, 1H), 4.00-3.90 (m, 1H), 2.94 (br s, 1H), 2.67-2.63 (m, 2H), 2.54 (br s, 1H), 2.12 (s, 3H). The enantiomeric excess was 90% as seen by chiral HPLC.

### Example 73: Synthesis of (R)-2-methyl-1-(2-oxo-4-(o-tolyl)-2H-pyrano[2,3-b]pyridin-7-yl)pyrrolidine-2-carboxylic acid

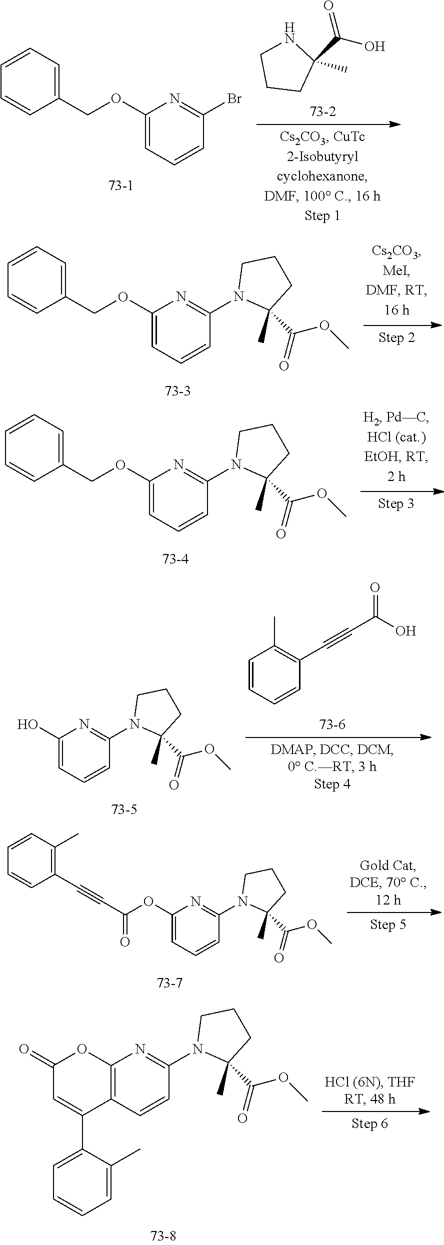

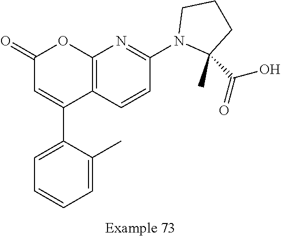 [SMILES: Cc1ccccc1-c1cc(=O)oc2nc(N3CCC[C@]3(C)C(=O)O)ccc12]

Synthesis of (R)-1-(6-(benzyloxy)pyridin-2-yl)-2-methylpyrrolidine-2-carboxylic acid, 73-3 [Step 1]: In a sealed tube, a suspension of 2-(benzyloxy)-6-bromopyridine (73-1, 1.0 g, 3.8 mmol), (R)-2-methylpyrrolidine-2-carboxylic acid (73-2, 830 mg, 6.4 mmol) and cesium carbonate (2.5 g, 7.6 mmol) in dry DMF (30 mL) was purged with Argon gas for 15 min. 2-Isobutyrylcyclohexanone (0.5 mL, 3.0 mmol) and copper thiophene-2-carboxylate (145 mg, 0.8 mmol) were added and the resulting reaction mixture heated at 100° C. for 16 h. After cooling, the reaction mixture was filtered through a pad of celite and washed with ethyl acetate. The combined filtrates were concentrated under reduced pressure and the residue was re-dissolved in water and washed with ethyl acetate. The aqueous phase was acidified to pH=4 with 1 N aqueous hydrochloric acid and extracted with ethyl acetate (twice). The combined extract was washed with brine, dried over anhydrous sodium sulphate and concentrated under reduced pressure to afford (R)-1-(6-(benzyloxy)pyridin-2-yl)-2-methylpyrrolidine-2-carboxylic acid (73-3, 800 mg). LCMS (ESI) Calcd. for C₁₈H₂₀N₂O₃: 312, found [M+H]⁺=313.

Synthesis of methyl (R)-1-(6-(benzyloxy)pyridin-2-yl)-2-methylpyrrolidine-2-carboxylate, 73-4 [Step 2]: To a stirred solution of (R)-1-(6-(benzyloxy)pyridin-2-yl)-2-methylpyrrolidine-2-carboxylic acid (73-3, 600 mg, 1.9 mmol) in DMF (6 mL), cesium carbonate (1.3 g, 3.8 mmol) and iodomethane (0.25 mL, 3.8 mmol) were added. The resulting reaction mixture was allowed to stir at ambient temperature for 16 h, diluted with ethyl acetate and washed with ice-cold water and brine. The organic extract were dried over anhydrous sodium sulphate, filtered and concentrated under reduced pressure. The product was purified by flash chromatography to afford methyl (R)-1-(6-(benzyloxy)pyridin-2-yl)-2-methylpyrrolidine-2-carboxylate (73-4, 350 mg). LCMS (ESI) Calcd. for C₁₉H₂₂N₂O₃: 326, found [M+H]⁺=327.

Synthesis of methyl 1-(6-hydroxypyridin-2-yl)-2-methylpyrrolidine-2-carboxylate, 73-5 [Step 3]: To a stirred solution of methyl (R)-1-(6-(benzyloxy)pyridin-2-yl)-2-methylpyrrolidine-2-carboxylate (73-4, 350 mg, 1.1 mmol) in methanol (5 mL), Pd—C(150 mg, 1.4 mmol, 10% w/w) was added. The reaction mixture was hydrogenated using hydrogen balloon at ambient temperature for 2 h, filtered through a pad of celite and washed with ethanol. The combined filtrate was concentrated under reduced pressure to afford methyl 1-(6-hydroxypyridin-2-yl)-2-methylpyrrolidine-2-carboxylate (73-5, 170 mg). LCMS (ESI) Calcd. for C₁₂H₁₆N₂O₃: 236, found [M+H]⁺=237.

Synthesis of methyl (R)-2-methyl-1-(6-((3-(o-tolyl)propioloyl)oxy)pyridin-2-yl)pyrrolidine-2-carboxylate, 73-7 [Step 4]: To a solution of methyl 1-(6-hydroxypyridin-2-yl)-2-methylpyrrolidine-2-carboxylate (73-5, 170 mg, 0.7 mmol), 3-(o-tolyl)prop-2-ynoic acid (73-6, 115 mg, 0.7 mmol) and N,N-dimethylaminopyridine (20 mg, 0.9 mmol) in dichloromethane (7 mL) at 0° C., a solution of N,N-dicyclohexylcarbodiimide (180 mg, 0.9 mmol) in dichloromethane (1 mL) was added dropwise. The resulting reaction mixture was allowed to stir at ambient temperature for 3 h., diluted with dichloromethane and washed with water followed by brine. The combined organic extracts were dried over anhydrous sodium sulphate and concentrated under reduced pressure. The product was purified by combiflash chromatography to afford methyl (R)-2-methyl-1-(6-((3-(o-tolyl)propioloyl)oxy)pyridin-2-yl)pyrrolidine-2-carboxylate, (73-7, 90 mg). LCMS (ESI) Calcd. for C₂₂H₂₂N₂O₄: 378, found [M+H]⁺=379.

Synthesis of methyl (R)-2-methyl-1-(2-oxo-4-(o-tolyl)-2H-pyrano[2,3-b]pyridin-7-yl)pyrrolidine-2-carboxylate, 73-8 [Step 5]: A solution methyl (R)-2-methyl-1-(6-((3-(o-tolyl)propioloyl)oxy)pyridin-2-yl)pyrrolidine-2-carboxylate (73-7, 90 mg, 0.2 mmol) in dry dichloroethane (3 mL) was purged with Argon gas for 10 min and (acetonitrile)[(2-biphenyl)di-tert-butylphosphine]gold(I) hexafluoroantimonate (25 mg, 0.04 mmol) was added. The reaction mixture was heated at 70° C. for 12 h. After cooling, the reaction mixture was filtered through a pad of celite, washed with ethyl acetate and the combined filtrates were concentrated under reduced pressure to afford methyl (R)-2-methyl-1-(2-oxo-4-(o-tolyl)-2H-pyrano[2,3-b]pyridin-7-yl)pyrrolidine-2-carboxylate (73-8, 80 mg). LCMS (ESI) Calcd. for C₂₂H₂₂N₂O₄: 378, found [M+H]⁺=379.

Synthesis of (R)-2-methyl-1-(2-oxo-4-(o-tolyl)-2H-pyrano[2,3-b]pyridin-7-yl)pyrrolidine-2-carboxylic acid, Example 73 [Step 6]: A solution of methyl (R)-2-methyl-1-(2-oxo-4-(o-tolyl)-2H-pyrano[2,3-b]pyridin-7-yl)pyrrolidine-2-carboxylate (73-8, 80 mg, 0.2 mmol) in tetrahydrofuran (2 mL) and aqueous solution of hydrochloric acid (6 N, 5 mL) was stirred at ambient temperature for 48 h. After completion, the volatiles was evaporated under reduced pressure. The product was purified by reverse phase preparative HPLC to afford (R)-2-methyl-1-(2-oxo-4-(o-tolyl)-2H-pyrano[2,3-b]pyridin-7-yl)pyrrolidine-2-carboxylic acid (Example 73, 20 mg). LCMS (ESI) Calcd. for C₂₁H₂₀N₂O₄: 364, found [M+H]⁺=365. ¹H NMR (400 MHz, DMSO-d₆ and two drops of D₂O at 80° C.) δ 7.42-7.35 (m, 2H), 7.32 (t, 1H), 7.18 (d, 1H), 7.06 (d, 1H), 6.42 (d, 1H), 5.94 (s, 1H), 3.64-3.62 (m, 1H), 3.52 (br s, 1H), 2.25-2.16 (m, 1H), 2.11 (s, 3H), 2.07-2.03 (m, 3H), 1.61 (s, 3H).

### Biological/Biochemical Evaluation

### General Protocol for In Vitro Analysis of Compounds

The inhibitory activity of the compounds of the present invention against POLRMT were determined by assays based on Bergbrede, T., et al., “An adaptable high-throughput technology enabling the identification of specific transcription modulators,” SLAC Discov., 22, 378-386 (2017).

The ability of some compounds of the present invention to inhibit POLRMT were determined in a homogeneous TR-FRET Assay using high-throughput screening in a 384-well plate format. This method is used to monitor the activity of mitochondrial transcription through measurement of its product, a 407 bp long RNA transcript. Detection of the product is facilitated by hybridization of two DNA-oligonucleotide probes to specific and adjacent sequences within the RNA product sequence. Upon annealing of the probes, two fluorophores are coupled directly to an acceptor nucleotide probe (ATTO647, 5′), or introduced via a coupled streptavidin with a biotinylated donor nucleotide probe (Europium cryptate) that is brought into sufficient proximity to serve as a fluorescence-donor-acceptor pair. Thus, a FRET signal at 665 nm is generated upon excitation at 340 nm.

Proteins used as transcription factors (POLRMT: NP_005026.3, TFAM: NP_003192.1, TFB2M: NP_071761.1) are diluted from their stocks to working concentrations of 1 μM, 20 μM and 4 μM respectively, in a dilution buffer containing 20 mM Tris-HCl (pH 8.0), 200 mM NaCl, 10% (v/v) glycerol, 1 mM Dithiothreitol (DTT), 0.5 mM EDTA.

DNA template is a pUC18 plasmid with the mitochondrial light strand promotor sequence (1-477) cloned between HindIII and BamHI sites. The DNA template is restriction linearized proximal to the promotor 3′-end (pUC-LSP).

The reaction mixture (10 uL) containing 7.5 nM POLRMT, 15 nM of TFB2M, 30 nM of TFAM, 0.5 nM of DNA template and 500 μM nucleotide triphosphate mix (NTPs) in a reaction buffer (containing 10 mM Tris-HCl (pH 7.5), 10 mM MgCl₂, 40 mM NaCl, 10 mM DTT, 0.005% (w/v) Tween-20, 160 units/ml Rnase inhibitor and 0.1 mg/mL BSA) are dispensed to compounds in microplates, using a Thermo Multidrop® dispenser, and incubated at 37° C. in a VWR INCU-Line incubator for 60 minutes after mixing. No nucleotide triphosphate mix is added to negative control samples. Microplates with compounds to be tested in the assay are prepared from 10 mM compound stocks in 100% DMSO, equal amounts of DMSO without any compound are added to positive control and negative control samples.

During the incubation, a mix of the detection reagents is prepared in a buffer such that the enzymatic reaction is terminated due to chelating of Mg-ions and increased ionic strength, containing 50 mM Tris-HCl (pH 7.5), 700 mM NaCl, 20 mM EDTA, and 0.01% (w/v) Tween-20. Europium-streptavidin is pre-incubated with a 200-fold molar excess of a random sequence oligonucleotide to block unspecific binding of oligo, for two hours at ambient temperature in the dark. Afterwards, the blocked Europium-streptavidin is kept on ice until use.

At the end of the enzymatic reaction time, 5 μL detection oligo mix in the detection buffer is added, and assay plates are mixed and kept at ambient temperature for one hour, protected from light. The concentration of the Acceptor nucleotide oligo (e.g., ATTO647N-5′-ACAAAGAACCCTAACACCAG-3′) and Donor nucleotide oligo (e.g., bio-5′-AACACATCTCT(-bio)GCCAAACCCCA-bio-3′) in each assay well is 1 nM, and 3 nM, respectively.

After incubation with oligo mix, 5 μL of pre-blocked Europium-streptavidin reagent is dispensed to each assay well, assay plates are again mixed and kept at ambient temperature for one hour, protected from light.

The generated signal is measured with BMG Pherastar microtiter plate reader with a TRF light unit, using excitation at 340 nm, an integration time of 200 μs, and a delay time of 100 μs, before detection at 620 nm and 665 nm. The ratio of donor- and acceptor-fluorescence is used as a measure of the generated transcript product (i.e. enzymatic activity).

TABLE 1
IC₅₀ values for amino chromen-2-one compounds.

| Examples | IC₅₀ (μM) |
|---|---|
| Example 01 | 1 |
| Example 02 | 2 |
| Example 03 | 0.2 |
| Example 04 | 2 |
| Example 05 | 0.4 |
| Example 06 | >100 |
| Example 07 | 0.4 |
| Example 08 | 18 |
| Example 09 | >100 |
| Example 10 | 86 |
| Example 11 | 47 |
| Example 12 | 0.9 |
| Example 13 | 0.3 |
| Example 14 | 0.4 |
| Example 15 | 2 |
| Example 16 | 2 |
| Example 17 | 0.6 |
| Example 18 | 1 |
| Example 19 | 0.3 |
| Example 20 | 2 |
| Example 21 | 3 |
| Example 22 | 3 |
| Example 23 | 0.09 |
| Example 24 | 3 |
| Example 25 | 1 |
| Example 26 | 21 |
| Example 27 | 1 |
| Example 28 | 1 |
| Example 29 | 7 |
| Example 30 | 0.9 |
| Example 31 | 0.4 |
| Example 32 | 1 |
| Example 33 | 7 |
| Example 34 | 20 |
| Example 35 | 0.2 |
| Example 36 | 1 |
| Example 37 | 0.2 |
| Example 38 | 1 |
| Example 39 | 1 |
| Example 40 | 0.5 |
| Example 41 | 7 |
| Example 42 | 3 |
| Example 43 | 7 |
| Example 44 | 5 |
| Example 45 | 1 |
| Example 46 | 2 |
| Example 47 | 57 |
| Example 48 | 0.5 |
| Example 49 | 1 |
| Example 50 | 0.4 |
| Example 51 | 0.1 |
| Example 52 | 10 |
| Example 53 | 1 |
| Example 54 | 0.2 |
| Example 55 | 12 |
| Example 56 | 0.2 |
| Example 60 | 6 |

### General Protocol for In Vivo AML (Acute Myeloid Leukemia) Efficacy Experiment

### Determination of Maximum Tolerated Dose of Test Compound

Immunocompromised mice (6-10-week-old, female NSG mice, strain NOD.Cg-Prkdc^{scid} Il2rg^{tm1Wj1}/Szj, Jackson Laboratories) are treated orally with test compound ranging from 1 to 1000 mg/kg, once or twice per day for the duration of 14 days. Total body weight is measured, and the general condition of mice is monitored routinely. Mice with severe symptoms and moribund are excluded from study. Submental blood collection method (no anesthesia) is used for all samplings. Plasma levels of test compound are determined at intervals ranging from 0.5 to 4 hours post first and last doses in all dosing groups. From these data pharmacokinetic analysis are conducted.

### In Vivo Efficacy Study in AML Mouse Model

MV4-11 AML cell lines (ATCC) are labelled with luciferase tag by viral transduction procedure (MV4-11-luc).

For an AML cell line xenograft efficacy experiment, female NSG mice are given intravenously ˜1×10⁷ MV4-11-luc cells. Mice are flux sorted and randomized into treatment groups 14 days post transplantation. Mice are then treated with vehicle (50 mM Na₂HPO₄), or test compound at a tolerable dose determined from the above study, once or twice per day for 21 days. Tumor progression/regression is monitored by imaging of mice using luciferin as a substrate (from 1 to 1000 mg/kg). Images are taken on a total of 9 time points i.e., one flux sort and once weekly to end date (8 time points). Imaging is performed under anesthesia and using in vivo imaging equipment IVIS. The treatment efficacy is also measured based on proportion of human AML cells, determined by flow cytometry analysis of viable human CD₄₅ positive cell population in peripheral blood of mice one week post last dose. Plasma levels of test compound are determined at intervals ranging from 0.5 to 4 hours post last dose. Animals are monitored individually, and total body weight is measured routinely. The endpoint of the experiment is moribundity. In addition, mice demonstrating tumor-associated symptoms including impairment of hind limb function, ocular proptosis, and weight loss are considered for euthanasia. The remaining mice are euthanized on day 60 of the study.

1. A compound of formula (1), or a pharmaceutically acceptable salt thereof: 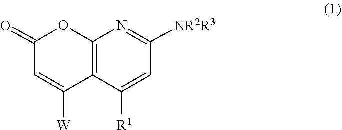
wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² and R³ are independently hydrogen, C₁-C₄ alkoxy, C₃-C₆ cycloalkyl, or C₁-C₄ alkyl optionally substituted with one or more substituents selected from the group consisting of deuterium, fluoro, C₁-C₄ alkoxy, cyano, C(O)OR⁴, and C(O)NR⁴R⁵;
or R² and R³ together with their connecting nitrogen form a 4- to 7-membered heterocyclic ring, a 7- to 12-membered spiro ring, or a 6- to 12-membered fused heterocyclic ring, wherein each ring optionally contains another heteroatom that is N, O, or S, and each ring is optionally substituted with one to four groups each independently selected from the group consisting of fluoro, chloro, C₁-C₄ alkoxy, C₁-C₄ alkyl-C₁-C₄ alkoxy, hydroxy, cyano, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁴ is hydrogen or C₁-C₃ alkyl; and
R⁵ is R⁴ or C₁-C₃ alkyl optionally substituted with C(O)OR⁴, C(O)NR⁴R⁴, or OR⁴.

2. The compound according to claim 1, or a pharmaceutically acceptable salt thereof, wherein:
W is C₆ aryl substituted with one or more groups, each independently selected from the group consisting of fluoro, chloro, C₁-C₄ alkyl, and CD₃;
R¹ is hydrogen;
R² and R³ are independently C₁ alkoxy or C₁-C₄ alkyl optionally substituted with one or more substituents selected from the group consisting of C₁ alkoxy, C(O)OR⁴, and C(O)NR⁴R⁵;
or R² and R³ together with their connecting nitrogen form a 4- to 6-membered heterocyclic ring, a 7-membered spiro ring, or a 6-membered fused heterocyclic ring, wherein each ring optionally contains another heteroatom that is N or O, and each ring is optionally substituted with one to four groups each independently selected from the group consisting of fluoro, C₁ alkoxy, C₁ alkyl-C₁ alkoxy, hydroxy, acyl, oxo, C(O)NR⁴R⁵, amino optionally substituted with one or more CH₃ or acyl, and C₁ alkyl optionally substituted with C(O)OR⁴;
R⁴ is hydrogen or C₁-C₃ alkyl; and
R⁵ is R⁴ or C₁-C₃ alkyl optionally substituted with C(O)OR⁴.

3. The compound according to claim 1, comprising a compound of formula (2), or a pharmaceutically acceptable salt thereof: 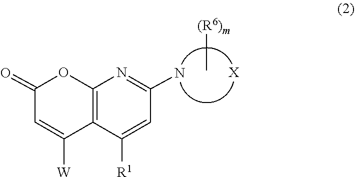
wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
X is C(R⁶)₂, NR⁷, O, or S;
R⁶ is selected from the group consisting of hydrogen, fluoro, C₁-C₄ alkoxy, C₁-C₄ alkyl-C₁-C₄ alkoxy, hydroxy, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁷ is selected from the group consisting of hydrogen, C₁-C₄ alkoxy, C₁-C₄ alkyl-C₁-C₄ alkoxy, hydroxy, acyl, and C₁-C₄ alkyl optionally substituted with C(O)OR⁴ or C(O)NR⁴R⁵;
R⁴ is hydrogen;
R⁵ is hydrogen;
m is 1-3; and
the ring represented by 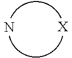
is a 4- to 6-membered heterocyclic ring.

4. The compound of claim 3, or a pharmaceutically acceptable salt thereof, wherein:
W is C₆ aryl substituted with one or more groups, each independently selected from the group consisting of fluoro, chloro, C₁-C₄ alkyl, and CD₃;
R¹ is hydrogen;
X is C(R⁶)₂, NR⁷, or O;
R⁶ is selected from the group consisting of hydrogen, fluoro, C₁ alkoxy, C₁ alkyl-C₁ alkoxy, hydroxy, oxo, C(O)OR⁴, C(O)NR⁴R⁵, amino optionally substituted with one or more CH₃ or acyl, and C₁ alkyl optionally substituted with C(O)OR⁴ or C(O)NR⁴R⁵;
R⁷ is selected from the group consisting of hydrogen, C₁ alkoxy, acyl, and CH₃;
R⁴ is hydrogen;
R⁵ is hydrogen;
m is 1-3; and
the ring represented by 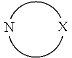
is a 4- to 6-membered heterocyclic ring.

5. The compound of claim 4, wherein
W is C₆ aryl substituted with one or more CH₃;
R¹ is hydrogen;
X is C(R⁶)₂ or O;
R⁶ is selected from the group consisting of hydrogen, C₁ alkoxy, C(O)OR⁴, and CH₃;
R⁴ is hydrogen;
m is 1; and
the ring represented by 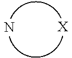
is a 5- to 6-membered heterocyclic ring.

6. The compound according to claim 1 comprising a compound of formula (3), or a pharmaceutically acceptable salt thereof: 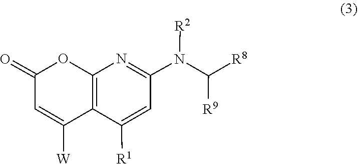
wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² is CH₃;
R⁴ is hydrogen or C₁-C₃ alkyl;
R⁵ is R⁴ or C₁-C₃ alkyl substituted with C(O)OR⁴, C(O)NR⁴R⁴, or OR⁴;
R⁸ is C₁-C₄ alkyl optionally substituted with one or more substituents selected from the group consisting of fluoro, C₁-C₄ alkoxy, C(O)OR⁴, and C(O)NR⁴R⁵; and
R⁹ is hydrogen or CH₃.

7. The compound of claim 6, or a pharmaceutically acceptable salt thereof, wherein:
W is C₆ aryl substituted with one or more groups, each independently selected from the group consisting of fluoro, chloro, CH₃, and CD₃;
R¹ is hydrogen;
R² is CH₃;
R⁴ is hydrogen or C₁-C₃ alkyl;
R⁵ is R⁴ or C₁-C₃ alkyl substituted with C(O)OR⁴, C(O)NR⁴R⁴, or OR⁴;
R⁸ is C₁-C₄ alkyl optionally substituted with one or more substituents selected from the group consisting of fluoro, C₁-C₄ alkoxy, C(O)OR⁴, and C(O)NR⁴R⁵; and
R⁹ is hydrogen or CH₃.

8. The compound of claim 7, or a pharmaceutically acceptable salt thereof, wherein: 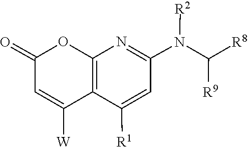
W is C₆ aryl substituted with CH₃;
R¹ is hydrogen;
R² is CH₃;
R⁴ is hydrogen or C₁-C₃ alkyl;
R⁵ is R⁴;
R is C₁ alkyl substituted with one substituent selected from the group consisting of C(O)OR⁴ and C(O)NR⁴R⁵; and
R⁹ is hydrogen or CH₃.

9. A pharmaceutical composition comprising a compound of claim 3, and pharmaceutically acceptable excipients.

10. A pharmaceutical composition comprising a compound of claim 6, and pharmaceutically acceptable excipients.

11. A compound, or a pharmaceutically acceptable salt thereof, selected from the group consisting of:

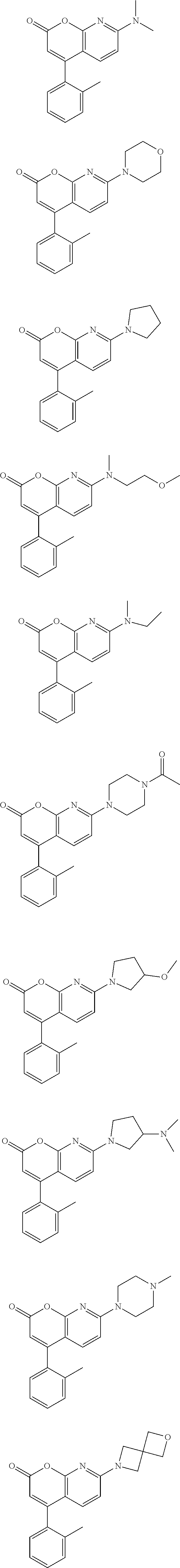

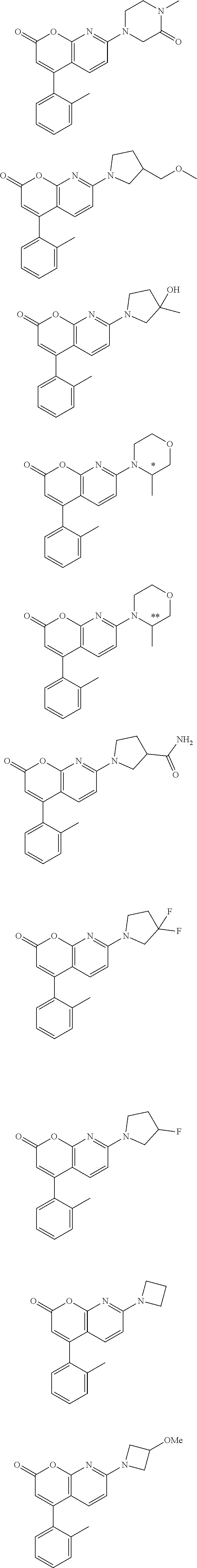

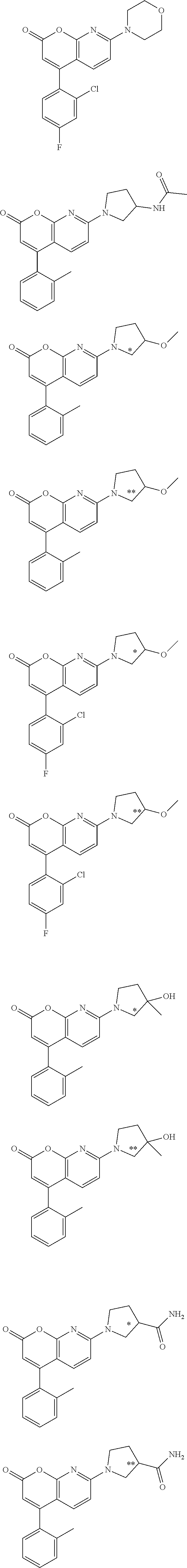

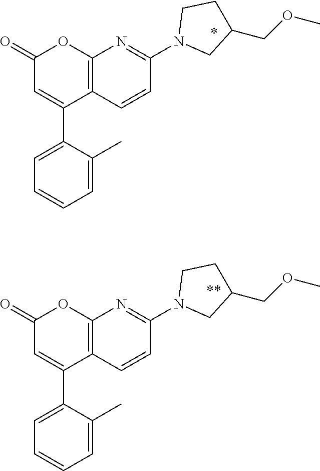 [SMILES: COCC1CCN(c2ccc3c(-c4ccccc4C)cc(=O)oc3n2)C1 ; COCC1CCN(c2ccc3c(-c4ccccc4C)cc(=O)oc3n2)C1]

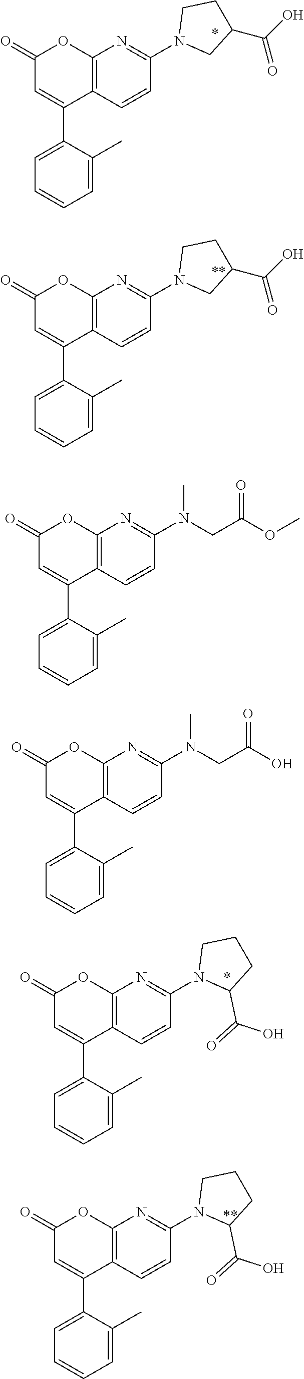 [SMILES: Cc1ccccc1-c1cc(=O)oc2nc(N3CCC(C(=O)O)C3)ccc12 ; Cc1ccccc1-c1cc(=O)oc2nc(N3CCC(C(=O)O)C3)ccc12 ; COC(=O)CN(C)c1ccc2c(-c3ccccc3C)cc(=O)oc2n1 ; Cc1ccccc1-c1cc(=O)oc2nc(N(C)CC(=O)O)ccc12 ; Cc1ccccc1-c1cc(=O)oc2nc(N3CCCC3C(=O)O)ccc12 ; Cc1ccccc1-c1cc(=O)oc2nc(N3CCCC3C(=O)O)ccc12]

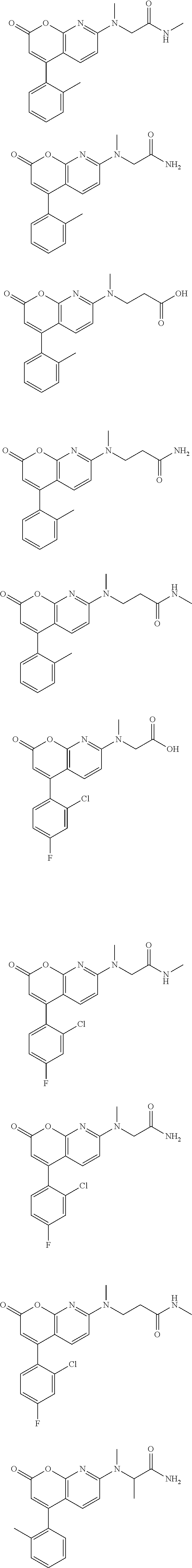

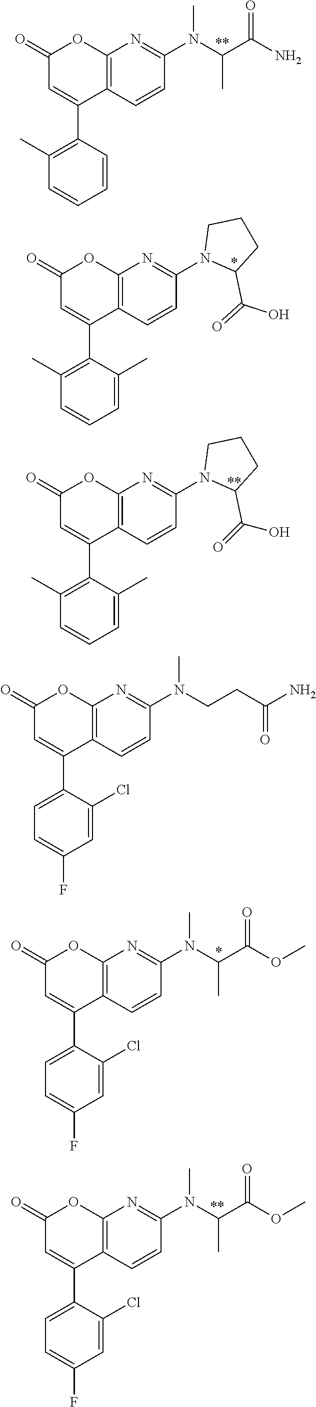 [SMILES: Cc1ccccc1-c1cc(=O)oc2nc(N(C)C(C)C(N)=O)ccc12 ; Cc1cccc(C)c1-c1cc(=O)oc2nc(N3CCCC3C(=O)O)ccc12 ; Cc1cccc(C)c1-c1cc(=O)oc2nc(N3CCCC3C(=O)O)ccc12 ; CN(CCC(N)=O)c1ccc2c(-c3ccc(F)cc3Cl)cc(=O)oc2n1 ; COC(=O)C(C)N(C)c1ccc2c(-c3ccc(F)cc3Cl)cc(=O)oc2n1 ; COC(=O)C(C)N(C)c1ccc2c(-c3ccc(F)cc3Cl)cc(=O)oc2n1]

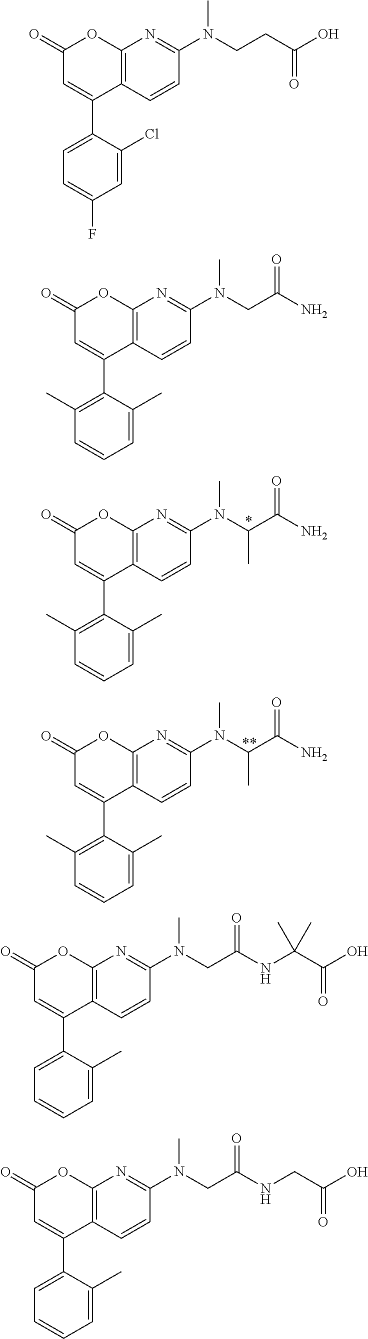 [SMILES: CN(CCC(=O)O)c1ccc2c(-c3ccc(F)cc3Cl)cc(=O)oc2n1 ; Cc1cccc(C)c1-c1cc(=O)oc2nc(N(C)CC(N)=O)ccc12 ; Cc1cccc(C)c1-c1cc(=O)oc2nc(N(C)C(C)C(N)=O)ccc12 ; Cc1cccc(C)c1-c1cc(=O)oc2nc(N(C)C(C)C(N)=O)ccc12 ; Cc1ccccc1-c1cc(=O)oc2nc(N(C)CC(=O)NC(C)(C)C(=O)O)ccc12 ; Cc1ccccc1-c1cc(=O)oc2nc(N(C)CC(=O)NCC(=O)O)ccc12]

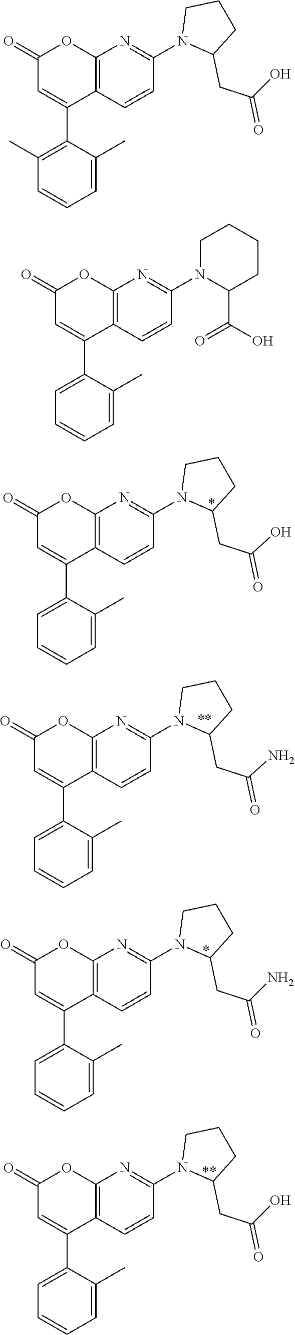 [SMILES: Cc1cccc(C)c1-c1cc(=O)oc2nc(N3CCCC3CC(=O)O)ccc12 ; Cc1ccccc1-c1cc(=O)oc2nc(N3CCCCC3C(=O)O)ccc12 ; Cc1ccccc1-c1cc(=O)oc2nc(N3CCCC3CC(=O)O)ccc12 ; Cc1ccccc1-c1cc(=O)oc2nc(N3CCCC3CC(N)=O)ccc12 ; Cc1ccccc1-c1cc(=O)oc2nc(N3CCCC3CC(N)=O)ccc12 ; Cc1ccccc1-c1cc(=O)oc2nc(N3CCCC3CC(=O)O)ccc12]

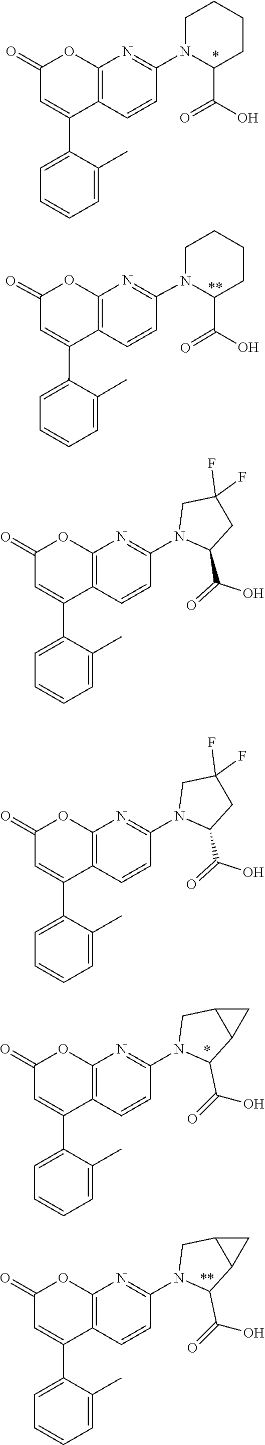 [SMILES: Cc1ccccc1-c1cc(=O)oc2nc(N3CCCCC3C(=O)O)ccc12 ; Cc1ccccc1-c1cc(=O)oc2nc(N3CCCCC3C(=O)O)ccc12 ; Cc1ccccc1-c1cc(=O)oc2nc(N3CC(F)(F)C[C@H]3C(=O)O)ccc12 ; Cc1ccccc1-c1cc(=O)oc2nc(N3CC(F)(F)C[C@@H]3C(=O)O)ccc12 ; Cc1ccccc1-c1cc(=O)oc2nc(N3CC4CC4C3C(=O)O)ccc12 ; Cc1ccccc1-c1cc(=O)oc2nc(N3CC4CC4C3C(=O)O)ccc12]

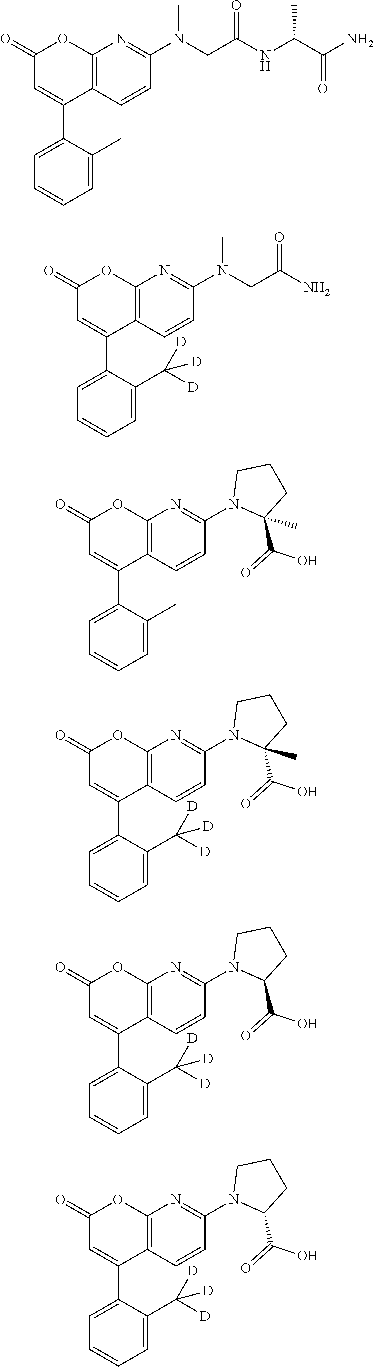 [SMILES: Cc1ccccc1-c1cc(=O)oc2nc(N(C)CC(=O)N[C@H](C)C(N)=O)ccc12 ; [2H]C([2H])([2H])c1ccccc1-c1cc(=O)oc2nc(N(C)CC(N)=O)ccc12 ; Cc1ccccc1-c1cc(=O)oc2nc(N3CCC[C@@]3(C)C(=O)O)ccc12 ; [2H]C([2H])([2H])c1ccccc1-c1cc(=O)oc2nc(N3CCC[C@]3(C)C(=O)O)ccc12 ; [2H]C([2H])([2H])c1ccccc1-c1cc(=O)oc2nc(N3CCC[C@H]3C(=O)O)ccc12 ; [2H]C([2H])([2H])c1ccccc1-c1cc(=O)oc2nc(N3CCC[C@@H]3C(=O)O)ccc12]

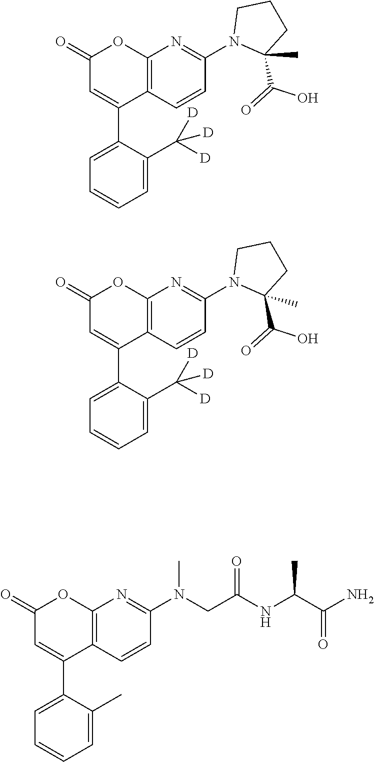 [SMILES: [2H]C([2H])([2H])c1ccccc1-c1cc(=O)oc2nc(N3CCC[C@]3(C)C(=O)O)ccc12 ; [2H]C([2H])([2H])c1ccccc1-c1cc(=O)oc2nc(N3CCC[C@@]3(C)C(=O)O)ccc12 ; Cc1ccccc1-c1cc(=O)oc2nc(N(C)CC(=O)N[C@@H](C)C(N)=O)ccc12]

12. The compound of claim 11, or a pharmaceutically acceptable salt thereof, wherein the compound is selected from the group consisting of:

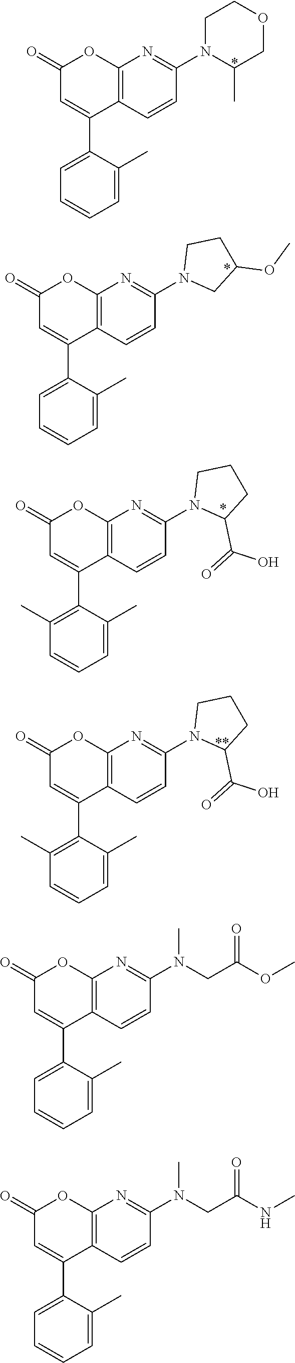 [SMILES: Cc1ccccc1-c1cc(=O)oc2nc(N3CCOCC3C)ccc12 ; COC1CCN(c2ccc3c(-c4ccccc4C)cc(=O)oc3n2)C1 ; Cc1cccc(C)c1-c1cc(=O)oc2nc(N3CCCC3C(=O)O)ccc12 ; Cc1cccc(C)c1-c1cc(=O)oc2nc(N3CCCC3C(=O)O)ccc12 ; COC(=O)CN(C)c1ccc2c(-c3ccccc3C)cc(=O)oc2n1 ; CNC(=O)CN(C)c1ccc2c(-c3ccccc3C)cc(=O)oc2n1]

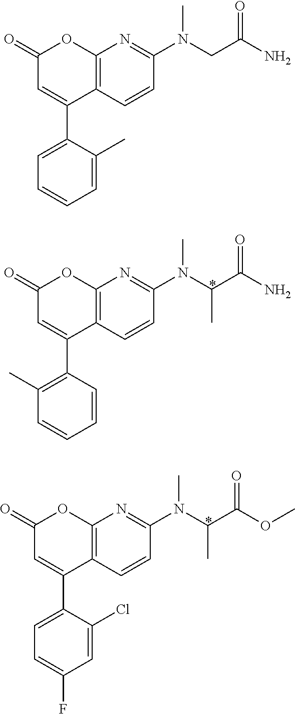 [SMILES: Cc1ccccc1-c1cc(=O)oc2nc(N(C)CC(N)=O)ccc12 ; Cc1ccccc1-c1cc(=O)oc2nc(N(C)C(C)C(N)=O)ccc12 ; COC(=O)C(C)N(C)c1ccc2c(-c3ccc(F)cc3Cl)cc(=O)oc2n1]

13. The compound of claim 12, or a pharmaceutically acceptable salt thereof, wherein the compound is selected from the group consisting of:

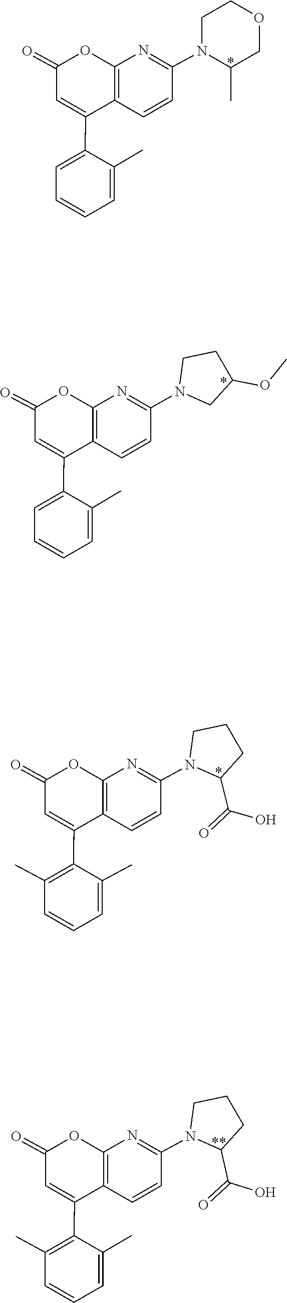

14. The compound of claim 12, or a pharmaceutically acceptable salt thereof, wherein the compound is selected from the group consisting of:

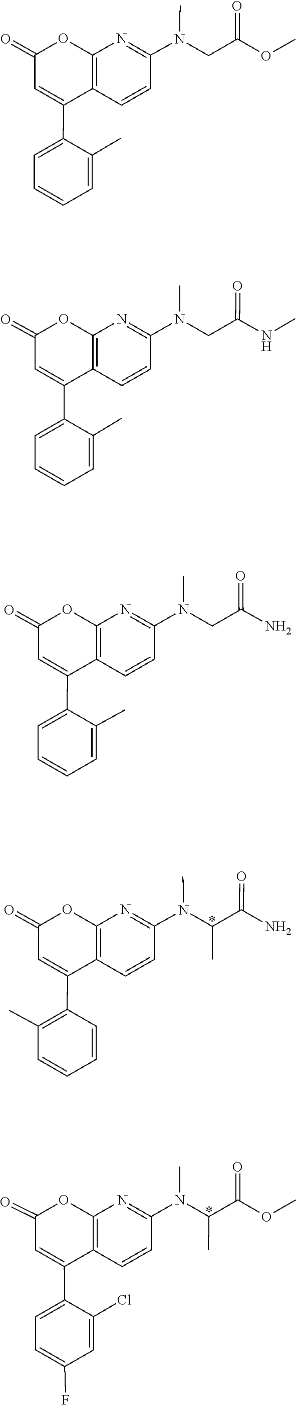

15. A pharmaceutical composition comprising a compound of claim 11, and pharmaceutically acceptable excipients.

16. A method of inhibiting the activity of POLRMT with a compound of formula (1), or a pharmaceutically acceptable salt thereof: 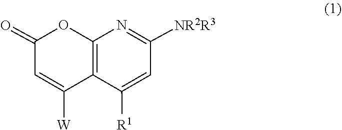
wherein:
W is C₆-C₁₂ aryl or 5-12 membered heteroaryl, either of which is substituted with one or more groups, each independently selected from the group consisting of deuterium, fluoro, chloro, CD₃, trifluoromethyl, difluoromethyl, cyano, hydroxyl, C₁-C₄ alkoxyl, and C₁-C₄ alkyl optionally substituted with OR⁴;
R¹ is hydrogen, deuterium, hydroxyl, cyano, chlorine, C₃-C₄ cycloalkyl, C₁-C₃ alkyl, or C₁-C₃ alkoxyl, wherein the alkyl and alkoxyl groups are optionally substituted with one or more fluorines;
R² and R³ are independently hydrogen, C₁-C₄ alkoxy, C₃-C₆ cycloalkyl, or C₁-C₄ alkyl optionally substituted with one or more substituents selected from the group consisting of deuterium, fluoro, C₁-C₄ alkoxy, cyano, C(O)OR⁴, and C(O)NR⁴R⁵;
or R² and R³ together with their connecting nitrogen form a 4- to 7-membered heterocyclic ring, a 7- to 12-membered spiro ring, or a 6- to 12-membered fused heterocyclic ring, wherein each ring optionally contains another heteroatom that is N, O, or S, and each ring is optionally substituted with one to four groups each independently selected from the group consisting of fluoro, chloro, C₁-C₄ alkoxy, C₁-C₄ alkyl-C₁-C₄ alkoxy, hydroxy, cyano, acyl, oxo, C(O)OR⁴, C(O)NR⁴R⁵, amino optionally substituted with one or more C₁-C₄ alkyl or acyl, and C₁-C₄ alkyl optionally substituted with C(O)OR⁴ or C(O)NR⁴R⁵;
R⁴ is hydrogen or C₁-C₃ alkyl; and
R⁵ is R⁴ or C₁-C₃ alkyl optionally substituted with C(O)OR⁴, C(O)NR⁴R⁴, or OR⁴.

17. The method of claim 16, wherein:
W is C₆ aryl substituted with one or more groups, each independently selected from the group consisting of fluoro, chloro, C₁-C₄ alkyl, and CD₃;
R¹ is hydrogen;
R² and R³ are independently C₁ alkoxy or C₁-C₄ alkyl optionally substituted with one or more substituents selected from the group consisting of C₁ alkoxy, C(O)OR⁴, and C(O)NR⁴R⁵;
or R² and R³ together with their connecting nitrogen form a 4- to 6-membered heterocyclic ring, a 7-membered spiro ring, or a 6-membered fused heterocyclic ring, wherein each ring optionally contains another heteroatom that is N or O, and each ring is optionally substituted with one to four groups each independently selected from the group consisting of fluoro, C₁ alkoxy, C₁ alkyl-C₁ alkoxy, hydroxy, cyano, acyl, oxo, C(O)NR⁴R⁵, amino optionally substituted with one or more CH₃ or acyl, and C₁ alkyl optionally substituted with C(O)OR⁴;
R⁴ is hydrogen or C₁-C₃ alkyl; and
R⁵ is R⁴ or C₁-C₃ alkyl optionally substituted with C(O)OR⁴.

18. The method of claim 16, wherein the compound, or a pharmaceutically acceptable salt thereof, is selected from the group consisting of:

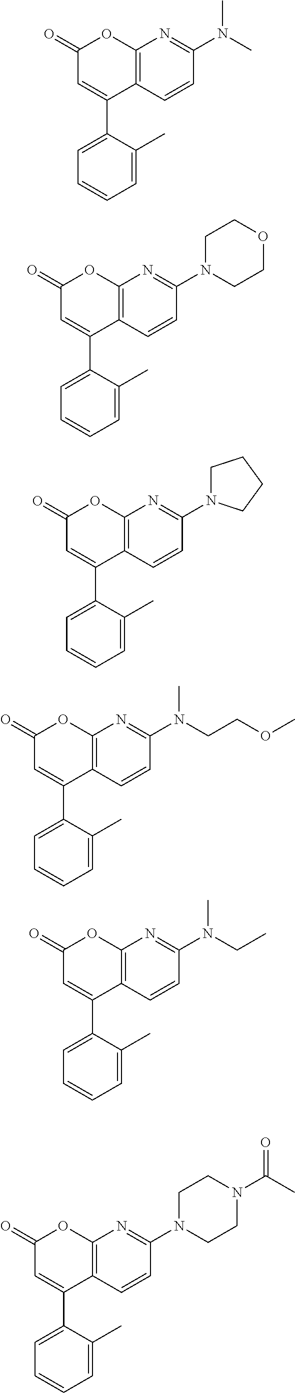 [SMILES: Cc1ccccc1-c1cc(=O)oc2nc(N(C)C)ccc12 ; Cc1ccccc1-c1cc(=O)oc2nc(N3CCOCC3)ccc12 ; Cc1ccccc1-c1cc(=O)oc2nc(N3CCCC3)ccc12 ; COCCN(C)c1ccc2c(-c3ccccc3C)cc(=O)oc2n1 ; CCN(C)c1ccc2c(-c3ccccc3C)cc(=O)oc2n1 ; CC(=O)N1CCN(c2ccc3c(-c4ccccc4C)cc(=O)oc3n2)CC1]

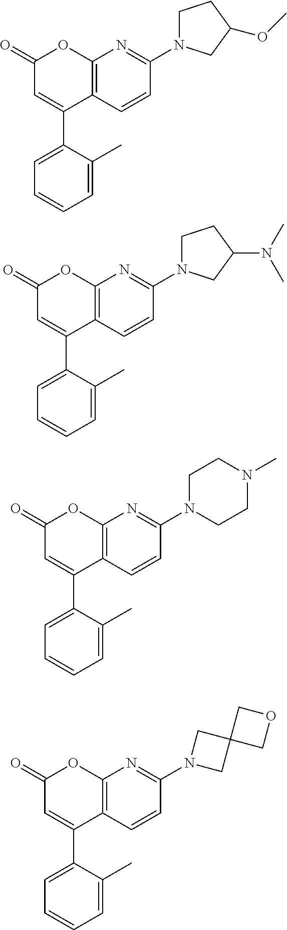 [SMILES: COC1CCN(c2ccc3c(-c4ccccc4C)cc(=O)oc3n2)C1 ; Cc1ccccc1-c1cc(=O)oc2nc(N3CCC(N(C)C)C3)ccc12 ; Cc1ccccc1-c1cc(=O)oc2nc(N3CCN(C)CC3)ccc12 ; Cc1ccccc1-c1cc(=O)oc2nc(N3CC4(COC4)C3)ccc12]

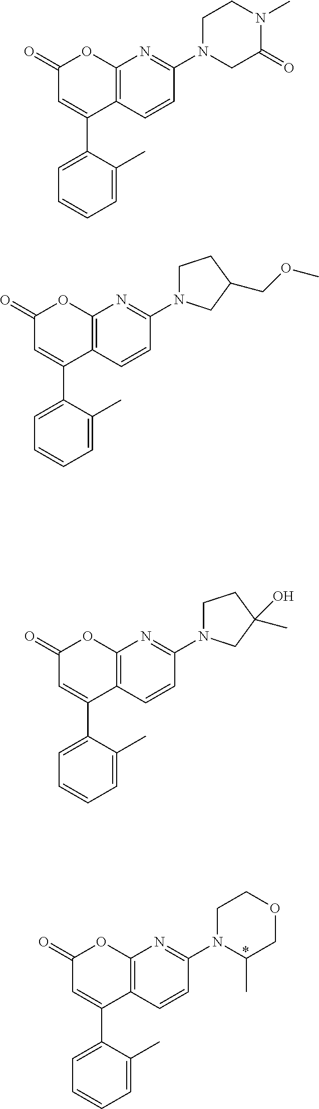 [SMILES: Cc1ccccc1-c1cc(=O)oc2nc(N3CCN(C)C(=O)C3)ccc12 ; COCC1CCN(c2ccc3c(-c4ccccc4C)cc(=O)oc3n2)C1 ; Cc1ccccc1-c1cc(=O)oc2nc(N3CCC(C)(O)C3)ccc12 ; Cc1ccccc1-c1cc(=O)oc2nc(N3CCOCC3C)ccc12]

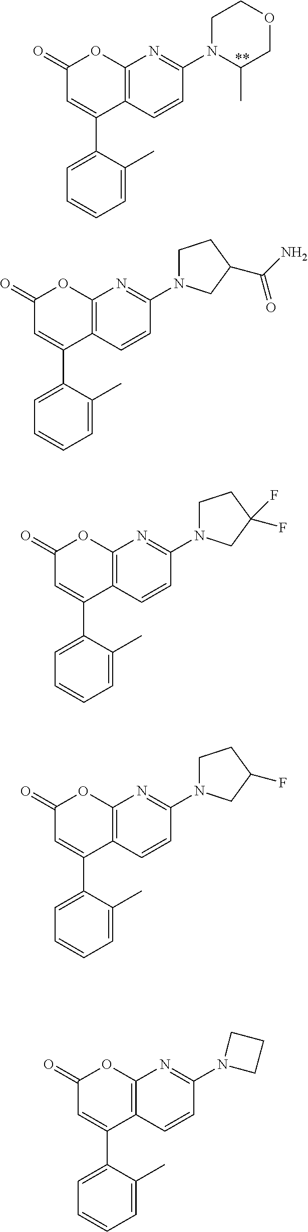 [SMILES: Cc1ccccc1-c1cc(=O)oc2nc(N3CCOCC3C)ccc12 ; Cc1ccccc1-c1cc(=O)oc2nc(N3CCC(C(N)=O)C3)ccc12 ; Cc1ccccc1-c1cc(=O)oc2nc(N3CCC(F)(F)C3)ccc12 ; Cc1ccccc1-c1cc(=O)oc2nc(N3CCC(F)C3)ccc12 ; Cc1ccccc1-c1cc(=O)oc2nc(N3CCC3)ccc12]

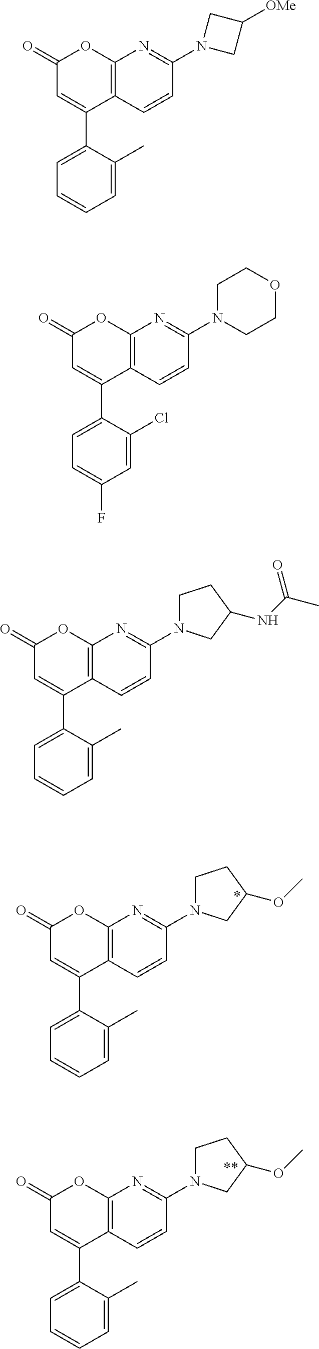 [SMILES: COC1CN(c2ccc3c(-c4ccccc4C)cc(=O)oc3n2)C1 ; O=c1cc(-c2ccc(F)cc2Cl)c2ccc(N3CCOCC3)nc2o1 ; CC(=O)NC1CCN(c2ccc3c(-c4ccccc4C)cc(=O)oc3n2)C1 ; COC1CCN(c2ccc3c(-c4ccccc4C)cc(=O)oc3n2)C1 ; COC1CCN(c2ccc3c(-c4ccccc4C)cc(=O)oc3n2)C1]

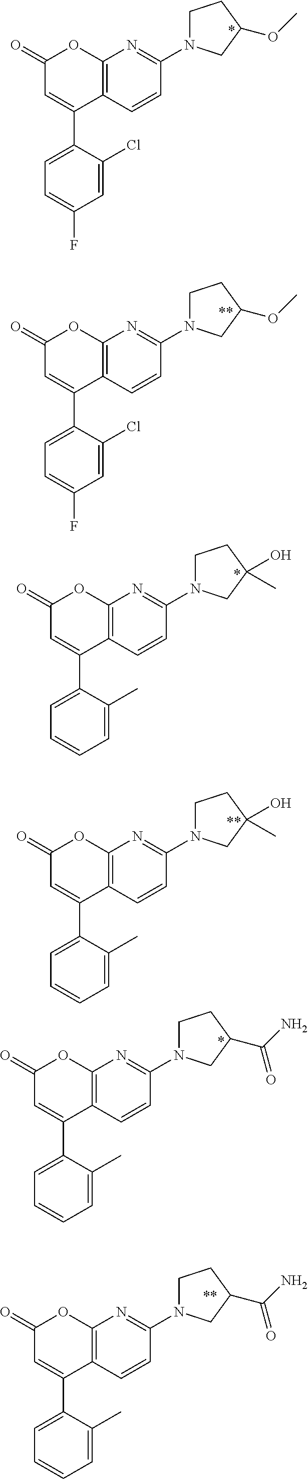 [SMILES: COC1CCN(c2ccc3c(-c4ccc(F)cc4Cl)cc(=O)oc3n2)C1 ; COC1CCN(c2ccc3c(-c4ccc(F)cc4Cl)cc(=O)oc3n2)C1 ; Cc1ccccc1-c1cc(=O)oc2nc(N3CCC(C)(O)C3)ccc12 ; Cc1ccccc1-c1cc(=O)oc2nc(N3CCC(C)(O)C3)ccc12 ; Cc1ccccc1-c1cc(=O)oc2nc(N3CCC(C(N)=O)C3)ccc12 ; Cc1ccccc1-c1cc(=O)oc2nc(N3CCC(C(N)=O)C3)ccc12]

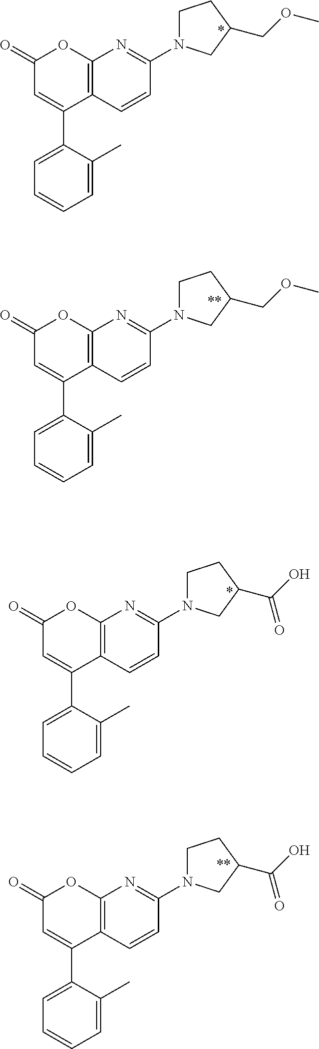 [SMILES: COCC1CCN(c2ccc3c(-c4ccccc4C)cc(=O)oc3n2)C1 ; COCC1CCN(c2ccc3c(-c4ccccc4C)cc(=O)oc3n2)C1 ; Cc1ccccc1-c1cc(=O)oc2nc(N3CCC(C(=O)O)C3)ccc12 ; Cc1ccccc1-c1cc(=O)oc2nc(N3CCC(C(=O)O)C3)ccc12]

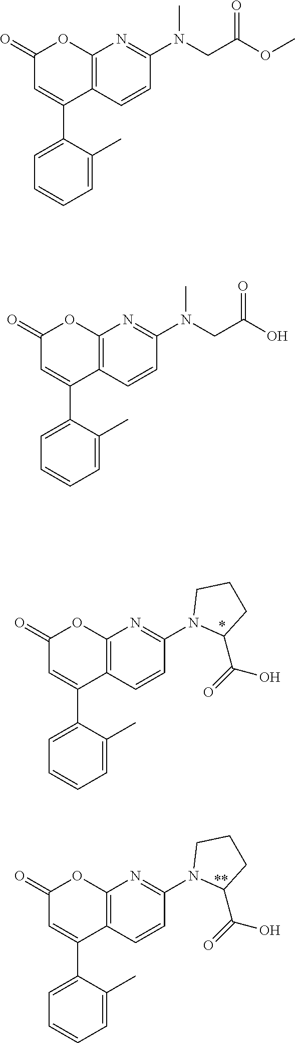 [SMILES: COC(=O)CN(C)c1ccc2c(-c3ccccc3C)cc(=O)oc2n1 ; Cc1ccccc1-c1cc(=O)oc2nc(N(C)CC(=O)O)ccc12 ; Cc1ccccc1-c1cc(=O)oc2nc(N3CCCC3C(=O)O)ccc12 ; Cc1ccccc1-c1cc(=O)oc2nc(N3CCCC3C(=O)O)ccc12]

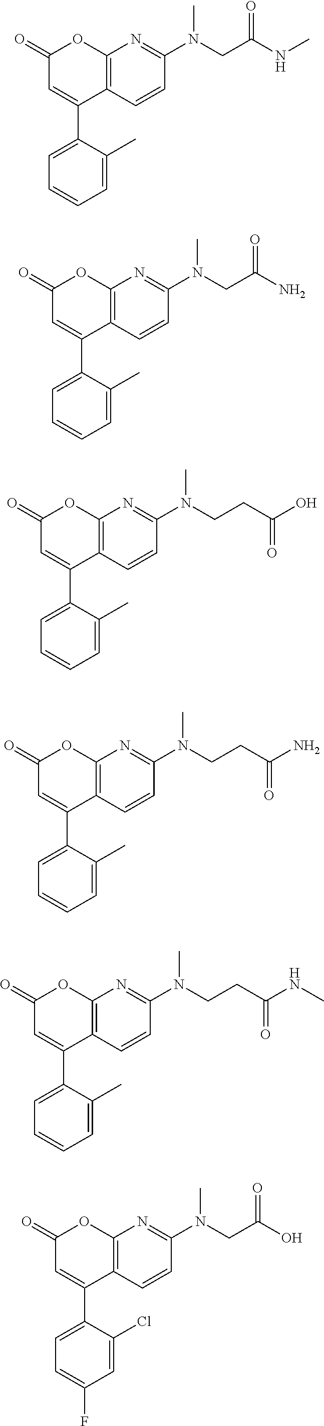 [SMILES: CNC(=O)CN(C)c1ccc2c(-c3ccccc3C)cc(=O)oc2n1 ; Cc1ccccc1-c1cc(=O)oc2nc(N(C)CC(N)=O)ccc12 ; Cc1ccccc1-c1cc(=O)oc2nc(N(C)CCC(=O)O)ccc12 ; Cc1ccccc1-c1cc(=O)oc2nc(N(C)CCC(N)=O)ccc12 ; CNC(=O)CCN(C)c1ccc2c(-c3ccccc3C)cc(=O)oc2n1 ; CN(CC(=O)O)c1ccc2c(-c3ccc(F)cc3Cl)cc(=O)oc2n1]

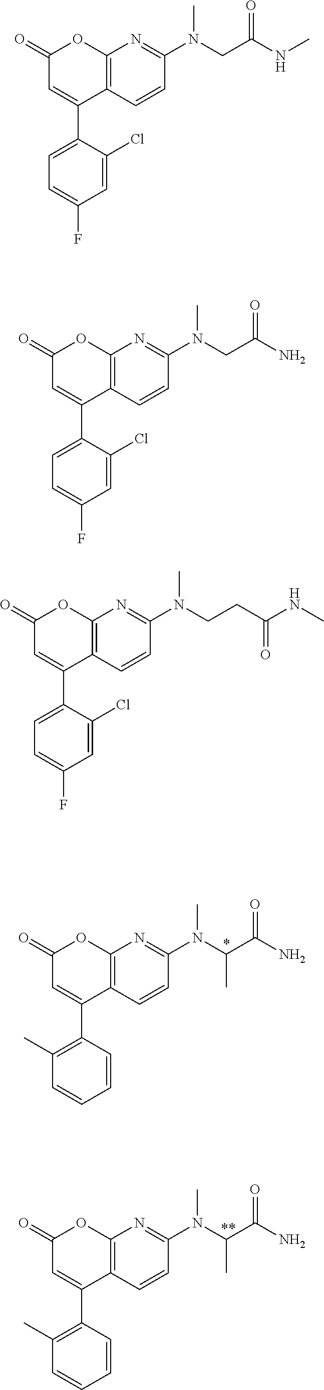 [SMILES: CNC(=O)CN(C)c1ccc2c(-c3ccc(F)cc3Cl)cc(=O)oc2n1 ; CN(CC(N)=O)c1ccc2c(-c3ccc(F)cc3Cl)cc(=O)oc2n1 ; CNC(=O)CCN(C)c1ccc2c(-c3ccc(F)cc3Cl)cc(=O)oc2n1 ; Cc1ccccc1-c1cc(=O)oc2nc(N(C)C(C)C(N)=O)ccc12 ; Cc1ccccc1-c1cc(=O)oc2nc(N(C)C(C)C(N)=O)ccc12]

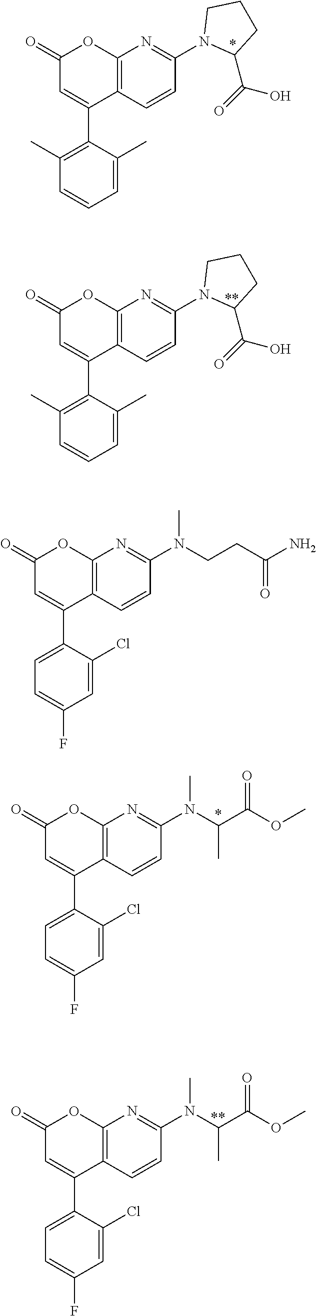 [SMILES: Cc1cccc(C)c1-c1cc(=O)oc2nc(N3CCCC3C(=O)O)ccc12 ; Cc1cccc(C)c1-c1cc(=O)oc2nc(N3CCCC3C(=O)O)ccc12 ; CN(CCC(N)=O)c1ccc2c(-c3ccc(F)cc3Cl)cc(=O)oc2n1 ; COC(=O)C(C)N(C)c1ccc2c(-c3ccc(F)cc3Cl)cc(=O)oc2n1 ; COC(=O)C(C)N(C)c1ccc2c(-c3ccc(F)cc3Cl)cc(=O)oc2n1]

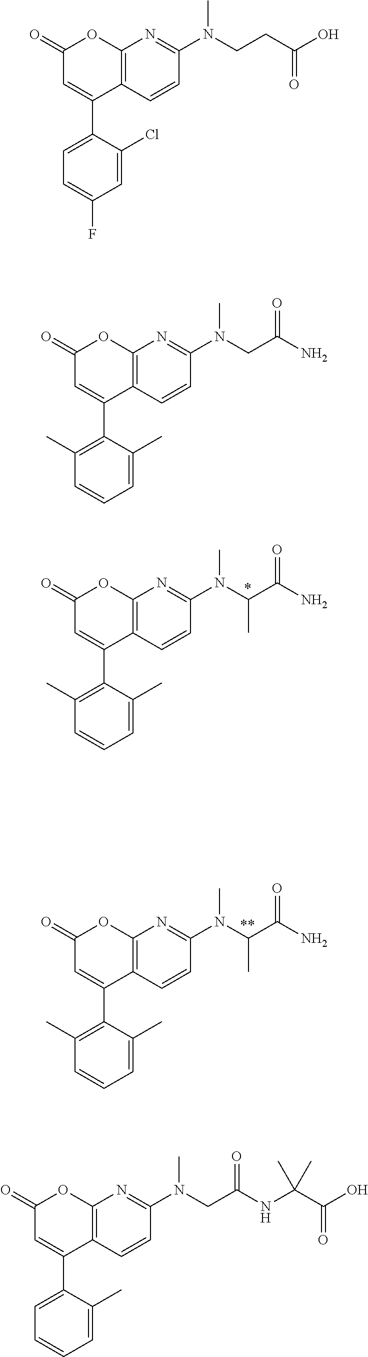 [SMILES: CN(CCC(=O)O)c1ccc2c(-c3ccc(F)cc3Cl)cc(=O)oc2n1 ; Cc1cccc(C)c1-c1cc(=O)oc2nc(N(C)CC(N)=O)ccc12 ; Cc1cccc(C)c1-c1cc(=O)oc2nc(N(C)C(C)C(N)=O)ccc12 ; Cc1cccc(C)c1-c1cc(=O)oc2nc(N(C)C(C)C(N)=O)ccc12 ; Cc1ccccc1-c1cc(=O)oc2nc(N(C)CC(=O)NC(C)(C)C(=O)O)ccc12]

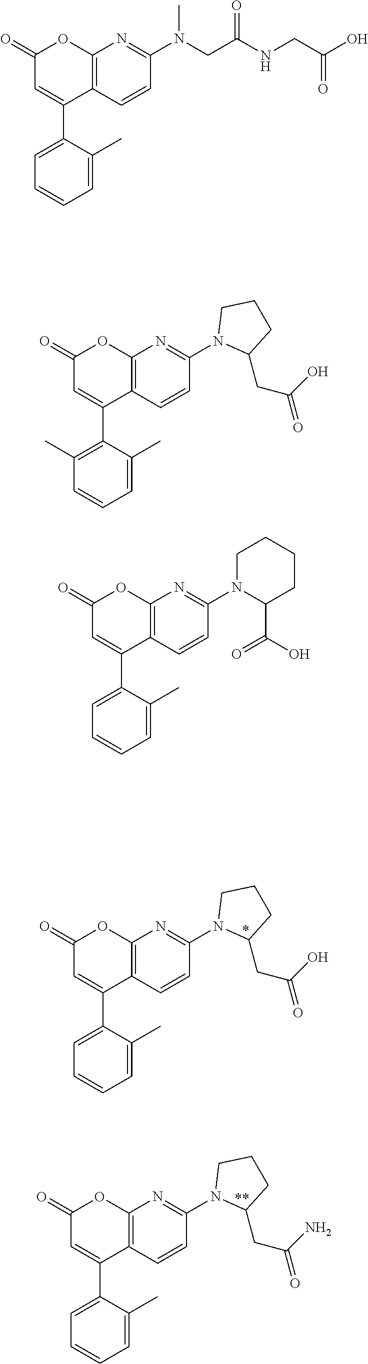 [SMILES: Cc1ccccc1-c1cc(=O)oc2nc(N(C)CC(=O)NCC(=O)O)ccc12 ; Cc1cccc(C)c1-c1cc(=O)oc2nc(N3CCCC3CC(=O)O)ccc12 ; Cc1ccccc1-c1cc(=O)oc2nc(N3CCCCC3C(=O)O)ccc12 ; Cc1ccccc1-c1cc(=O)oc2nc(N3CCCC3CC(=O)O)ccc12 ; Cc1ccccc1-c1cc(=O)oc2nc(N3CCCC3CC(N)=O)ccc12]

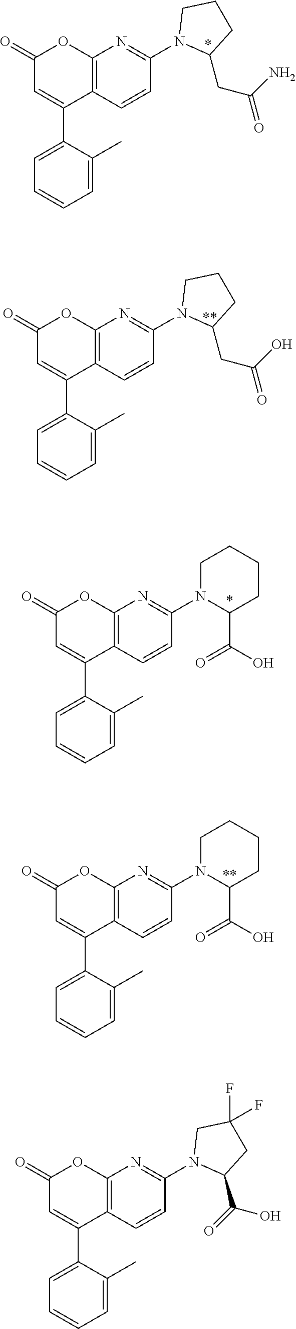 [SMILES: Cc1ccccc1-c1cc(=O)oc2nc(N3CCCC3CC(N)=O)ccc12 ; Cc1ccccc1-c1cc(=O)oc2nc(N3CCCC3CC(=O)O)ccc12 ; Cc1ccccc1-c1cc(=O)oc2nc(N3CCCCC3C(=O)O)ccc12 ; Cc1ccccc1-c1cc(=O)oc2nc(N3CCCCC3C(=O)O)ccc12 ; Cc1ccccc1-c1cc(=O)oc2nc(N3CC(F)(F)C[C@H]3C(=O)O)ccc12]

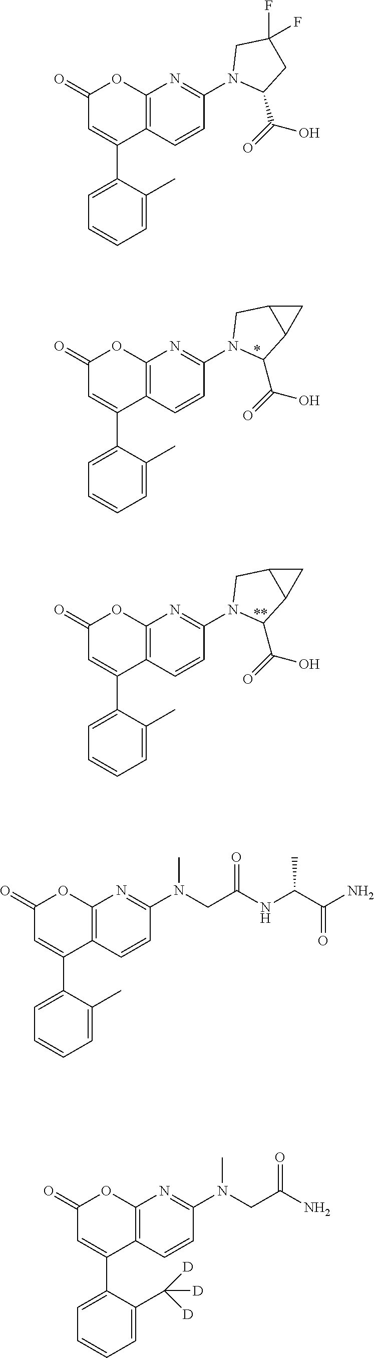 [SMILES: Cc1ccccc1-c1cc(=O)oc2nc(N3CC(F)(F)C[C@@H]3C(=O)O)ccc12 ; Cc1ccccc1-c1cc(=O)oc2nc(N3CC4CC4C3C(=O)O)ccc12 ; Cc1ccccc1-c1cc(=O)oc2nc(N3CC4CC4C3C(=O)O)ccc12 ; Cc1ccccc1-c1cc(=O)oc2nc(N(C)CC(=O)N[C@H](C)C(N)=O)ccc12 ; [2H]C([2H])([2H])c1ccccc1-c1cc(=O)oc2nc(N(C)CC(N)=O)ccc12]

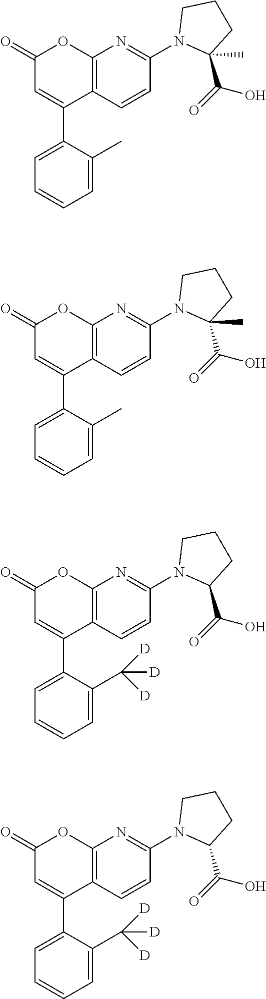 [SMILES: Cc1ccccc1-c1cc(=O)oc2nc(N3CCC[C@@]3(C)C(=O)O)ccc12 ; Cc1ccccc1-c1cc(=O)oc2nc(N3CCC[C@]3(C)C(=O)O)ccc12 ; [2H]C([2H])([2H])c1ccccc1-c1cc(=O)oc2nc(N3CCC[C@H]3C(=O)O)ccc12 ; [2H]C([2H])([2H])c1ccccc1-c1cc(=O)oc2nc(N3CCC[C@@H]3C(=O)O)ccc12]

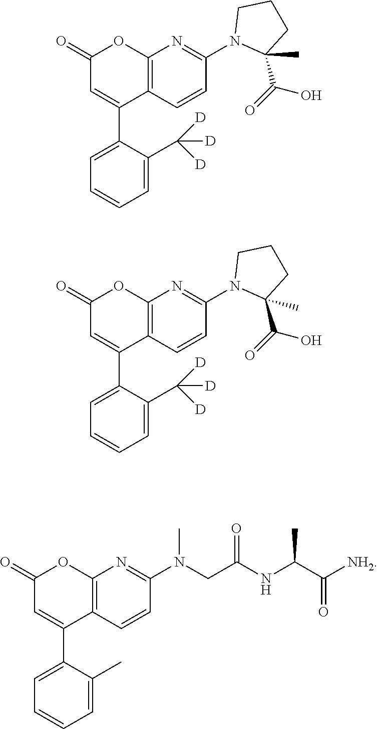 [SMILES: [2H]C([2H])([2H])c1ccccc1-c1cc(=O)oc2nc(N3CCC[C@]3(C)C(=O)O)ccc12 ; [2H]C([2H])([2H])c1ccccc1-c1cc(=O)oc2nc(N3CCC[C@@]3(C)C(=O)O)ccc12 ; Cc1ccccc1-c1cc(=O)oc2nc(N(C)CC(=O)N[C@@H](C)C(N)=O)ccc12]

19. The method of claim 16, wherein the compound, or a pharmaceutically acceptable salt thereof, is selected from the group consisting of:

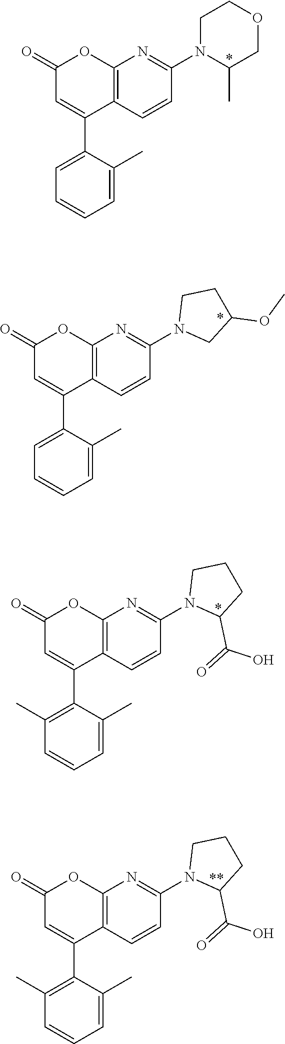 [SMILES: Cc1ccccc1-c1cc(=O)oc2nc(N3CCOCC3C)ccc12 ; COC1CCN(c2ccc3c(-c4ccccc4C)cc(=O)oc3n2)C1 ; Cc1cccc(C)c1-c1cc(=O)oc2nc(N3CCCC3C(=O)O)ccc12 ; Cc1cccc(C)c1-c1cc(=O)oc2nc(N3CCCC3C(=O)O)ccc12]

20. The method of claim 16, wherein the compound, or a pharmaceutically acceptable salt thereof, is selected from the group consisting of: [chemical structure]
